SEQUENCE LISTING (1 sequence)

SEQ ID NO: 1 - protein, 663 aa, Homo sapiens
MAQGTLIRVTPEQPTHAVCVLGTLTQLDICSSAPEDCTSFSINASPGVVVDIAHGPPAKKKSTGSSTWPLDPGVEVTLTMKVASGSTGDQKVQISYYGPKTPPVKALLYLTGVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

(19) United States
(12) Patent Application Publication
(10) Pub. No.: US 2025/0353848 A1
(43) Pub. Date: Nov. 20, 2025

(54) HETEROCYCLIC PAD4 INHIBITORS

(71) Applicant: Celgene Corporation, Summit, NJ (US)

(72) Inventors: Alyssa H. Antropow, Hillsborough, NJ (US); Boris M. Seletsky, Summit, NJ (US); Audrey Graham Ross, Summit, NJ (US); Xiao Zhu, Summit, NJ (US); Paul E. Gormisky, Summit, NJ (US); Guobin Miao, Summit, NJ (US)

(21) Appl. No.: 18/869,248

(22) PCT Filed: May 26, 2023
(86) PCT No.: PCT/US2023/067557

§ 371 Date: Nov. 25, 2024

Related U.S. Application Data
(60) Provisional application No. 63/365,370, filed on May 26, 2022.

(51) Int. Cl. C07D 471/14; A61K 31/4375; A61K 31/444; A61K 31/4545; A61K 31/5377; C07B 59/00; C07D 519/00

(52) U.S. Cl. CPC C07D 471/14; A61K 31/4375; A61K 31/444; A61K 31/4545; A61K 31/5377; C07B 59/002; C07D 519/00

(57) ABSTRACT

The disclosure generally relates to 3,5,6,7-tetrahydro-8H-imidazo[4,5-b][1,6]naphthyridin-8-one compounds of Formula I, which are inhibitors of PAD4, methods for preparing these compounds, pharmaceutical compositions comprising these compounds and uses of these compounds in the treatment of a disease or a disorder associated with PAD4 enzyme activity.

### CROSS-REFERENCE

This application claims the benefit of and priority to U.S. Provisional Patent Application No. 63/365,370, filed on May 26, 2022, the entire contents of which are hereby incorporated by reference herein.

### INCORPORATION BY REFERENCE OF SEQUENCE LISTING

The sequence listing paragraph application contains a Sequence Listing which has been submitted in .XML format via EFS-WEB and is hereby incorporated by reference in its entirety. Said .XML copy, created on May 26, 2023 is named 055920-608001WO_SeqList_ST26.xml and is 3 KB in size.

### FIELD

The invention generally relates to substituted heterocyclic compounds, methods for preparing these compounds, pharmaceutical compositions comprising these compounds, and use of these compounds in the treatment of a disease or a disorder associated with PAD4 enzyme activity.

### BACKGROUND

PAD4 (SEQ ID NO: 1) is a member of the peptidylarginine deiminase (PAD) family of enzymes capable of catalysing the citrullination of arginine into citrulline within peptide sequences. PAD4 is responsible for the deamination or citrullination of a variety of proteins in vitro and in vivo, with consequences of diverse functional responses in a variety of diseases (Jones J. E. et al, Curr. Opin. Drug Discov. Devel., 12(5), (2009), 616-627). Examples of exemplar diseases or disorders include rheumatoid arthritis, diseases with neutrophilic contributions to pathogenesis (for example vasculitis, systemic lupus erythematosus, ulcerative colitis) in addition to oncology indications. PAD4 inhibitors also have wider applicability as tools and therapeutics for human diseases and disorders through epigenetic mechanisms.

Inhibitors of PAD4 have utility against Rheumatoid Arthritis (RA). RA is an autoimmune disease affecting approximately 1% of the population (Wegner N. et al, Immunol. Rev., 233(1), (2010), 34-54). It is characterized by inflammation of articular joints leading to debilitating destruction of bone and cartilage. A weak genetic association between PAD4 polymorphisms and susceptibility to RA has been suggested, albeit inconsistently, in a number of population studies (Kochi Y. et al, Ann. Rheum. Dis., 70, (2011), 512-515). PAD4 (along with family member PAD2) has been detected in synovial tissue where it is responsible for the deamination of a variety of joint proteins. This process is presumed to lead to a break of tolerance to, and initiation of immune responses to, citrullinated substrates such as fibrinogen, vimentin and collagen in RA joints. These anti-citrullinated protein antibodies (ACPA) contribute to disease pathogenesis and may also be used as a diagnostic test for RA (e.g. the commercially available CCP2 or cyclic citrullinated protein 2 test). In addition, increased citrullination may also offer additional direct contributions to disease pathogenesis through its ability to affect directly the function of several joint and inflammatory mediators (e.g. fibrinogen, anti-thrombin, and multiple chemokines). In a smaller subset of RA patients, anti-PAD4 antibodies can be measured and may correlate with a more erosive form of the disease.

PAD4 inhibitors are also useful for the reduction of pathological neutrophil activity in a variety of diseases. Studies suggest that the process of Neutrophil Extracellular Trap (NET) formation, an innate defense mechanism by which neutrophils are able to immobilize and kill pathogens, is associated with histone citrullination and is deficient in a PAD4 knockout mice (Neeli I. et al, J. Immunol., 180, (2008), 1895-1902, and Li P. et al, J. Exp. Med, 207(9), (2010), 1853-1862). PAD4 inhibitors may therefore have applicability for diseases where NET formation in tissues contributes to local injury and disease pathology. Such diseases include, but are not limited to, small vessel vasculitis (Kessenbrock K. et al, Nat. Med., 15(6), (2009), 623-625), systemic lupus erythematosus (Hakkim A. et al, Proc. Natl. Acad. Sci. USA, 107(21), (2010), 9813-9818, and Villanueva E. et al, J. Immunol., 187(1), (2011), 538-52), ulcerative colitis (Savchenko A. et al, Pathol. Int., 61(5), (2011), 290-7), cystic fibrosis, asthma (Dworski R. et al, J. Allergy Clin. Immunol., 127(5), (2011), 1260-6), deep vein thrombosis (Fuchs T. et al, Proc. Natl. Acad. Sci. USA, 107(36), (2010), 15880-5), periodontitis (Vitkov L. et al, Ultrastructural Pathol., 34(1), (2010), 25-30), sepsis (Clark S. R. et al, Nat. Med., 13(4), (2007), 463-9), appendicitis (Brinkmann V. et al, Science, 303, (2004), 1532-5), and stroke. In addition, there is evidence that NETs may contribute to pathology in diseases affecting the skin, e.g., in cutaneous lupus erythematosis (Villanueva E. et al, J. Immunol., 187(1), (2011), 538-52) and psoriasis (Lin A. M. et al., J. Immunol., 187(1), (2011), 490-500), so a PAD4 inhibitor may show benefit to tackle NET skin diseases, when administered by a systemic or cutaneous route. PAD4 inhibitors may affect additional functions within neutrophils and have wider applicability to neutrophilic diseases.

Studies have demonstrated efficacy of tool PAD inhibitors (for example, chloro-amidine) in a number of animal models of disease, including collagen-induced arthritis (Willis V. C. et al, J. Immunol., 186(7), (2011), 4396-4404), dextran sulfate sodium (DSS)-induced experimental colitis (Chumanevich A. A. et al, Am. J. Physiol. Gastrointest. Liver Physiol., 300(6), (2011), G929-G938), spinal cord repair (Lange S. et al, Dev. Biol., 355(2), (2011), 205-14), and experimental autoimmune encephalomyelitis (EAE). The DSS colitis report also demonstrates that chloro-amidine drives apoptosis of inflammatory cells both in vitro and in vivo, suggesting that PAD4 inhibitors may be effective more generally in widespread inflammatory diseases.

PAD4 inhibitors are also useful in the treatment of cancers (Slack J. L. et al, Cell. Mol. Life Sci., 68(4), (2011), 709-720). Over-expression of PAD4 has been demonstrated in numerous cancers (Chang X. et al, BMC Cancer, 9, (2009), 40). An anti-proliferative role has been suggested for PAD4 inhibitors from the observation that PAD4 citrullinates arginine residues in histones at the promoters of p53-target genes such as p21, which are involved in cell cycle arrest and induction of apoptosis (Li P. et al, Mol. Cell Biol., 28(15), (2008), 4745-4758).

The aforementioned role of PAD4 in deaminating arginine residues in histones may be indicative of a role for PAD4 in epigenetic regulation of gene expression. PAD4 is the primary PAD family member observed to be resident in the nucleus as well as the cytoplasm. Early evidence that PAD4 may act as a histone demethyliminase as well as a deiminase is inconsistent and unproven. However, it may reduce histone arginine methylation (and hence epigenetic regulation associated with this mark) indirectly via depletion of available arginine residues by conversion to citrulline. PAD4 inhibitors are useful as epigenetic tools or therapeutics for affecting expression of varied target genes in additional disease settings. Through such mechanisms, PAD4 inhibitors may also be effective in controlling citrullination levels in stem cells and may therefore therapeutically affect the pluripotency status and differentiation potential of diverse stem cells including, but not limited to, embryonic stem cells, neural stem cells, haematopoietic stem cells and cancer stem cells. Accordingly, there remains an unmet need to identify and develop PAD4 inhibitors for the treatment of PAD4-mediated diseases or disorders.

### SUMMARY

Accordingly, there are provided compounds of Formula I:

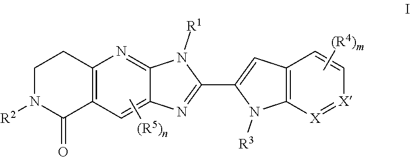

and pharmaceutically acceptable salts, isomers, enantiomers, or tautomers thereof, wherein each of X, X′, R¹, R², R³, R⁴, R⁵, m, and n is as defined below and described herein.

In another aspect are provided compounds of Formula I′:

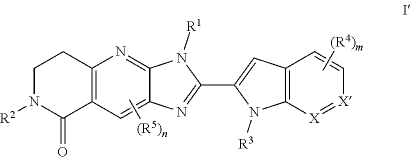

and pharmaceutically acceptable salts, isomers, enantiomers, or tautomers thereof, wherein each of X, X′, R¹, R², R³, R⁴, R⁵, m, and n is as defined below and described herein.

In another aspect, the present disclosure provides a pharmaceutical composition comprising at least one compound of Formula I, or a pharmaceutically acceptable salt thereof, and one or more pharmaceutically acceptable carriers, excipients, or vehicles. In some aspects, a provided pharmaceutical composition is suitable for oral, parenteral, mucosal, transdermal, or topical administration.

In another aspect the present disclosure provides a method of inhibiting a PAD4 enzyme, or mutant thereof, the method comprising contacting a biological sample with a compound of Formula I, or a pharmaceutically acceptable salt thereof.

In another aspect, the present disclosure provides a method of treating a disease or a disorder associated with PAD4 enzyme activity, comprising administering to a subject in need of such treatment, a therapeutically effective amount of at least one compound of Formula I, or a pharmaceutically acceptable salt thereof. Such disorders or conditions include, among others, rheumatoid arthritis, vasculitis, systemic lupus erythematosus, and ulcerative colitis.

### DETAILED DESCRIPTION

### General Description of Compounds of the Disclosure

In some embodiments, the present disclosure provides a compound of formula I:

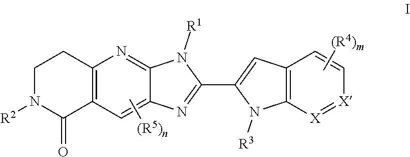

or a pharmaceutically acceptable salt, isomer, enantiomer, or tautomer thereof, wherein:
X is selected from C—R⁶ and N;
X′ is selected from C—R^{6′} and N, wherein X and X′ are not simultaneously N;
R¹ is C₁₋₄ aliphatic;
R² is C₁₋₆ aliphatic substituted by 0-4 instances of R⁷;
R³ is C₁₋₆ aliphatic substituted by 0-3 instances of R¹;
R⁴ is halogen;
R⁵ is halogen;
each R⁶ and R^{6′} is selected from hydrogen, halogen, —OR, —N(R)₂, —OC(O)R, —N(R)C(O)R, —O-L-(R⁹)ₚ, —Cy, and optionally substituted C₁₋₆ aliphatic;
R⁷ is selected from halogen, —OR, —N(R)₂, and —Cy;
R⁸ is selected from halogen, —OR, —N(R)₂, and —Cy;
R⁹ is selected from halogen, —OR, —N(R)₂, and —Cy;
L is a covalent bond or C₁₋₄ aliphatic;
Cy is selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
R¹⁰ is selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
R is hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur;
each m and n is 0 or 1; and
p is 1-4.

### 1. DEFINITIONS

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. Suitable aliphatic groups include, but are not limited to, methyl, ethyl, n-propyl, isopropyl, n-butyl, sec-butyl, isobutyl, tert-butyl, and the like.

In some embodiments, “carbocyclic” (or “cycloaliphatic” or “carbocycle” or “cycloalkyl”) refers to a C3-C8 hydrocarbon, which may be monocyclic or multicyclic, that is completely saturated or that contains one or more units of unsaturation, but which is not aromatic, that has a single point of attachment to the rest of the molecule. The rings of multi-ring carbocyclics may exist as fused, bridged and/or joined through one or more spiro union to 1 or 2 aromatic cycloalkyl or heterocyclic rings. Typical, non-limiting examples of cycloalkyl groups include cyclopropyl, cyclobutyl, cyclopentyl, cyclohexyl, cycloheptyl, cyclooctyl, cyclodecyl, cyclododecyl, cyclopentenyl, cyclohexenyl, cycloheptenyl, cyclooctenyl, cyclohexadienyl, cycloheptadienyl, and the like.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)). In some embodiments, an oxidized form of sulfur includes S═O and S(═O)₂.

The term “unsaturated,” as used herein, means that a moiety has one or more units of unsaturation.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system and exemplary groups include phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, or 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Exemplary heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Examplary groups include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, or one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

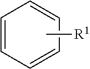

refers to at least

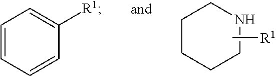

refers to at least

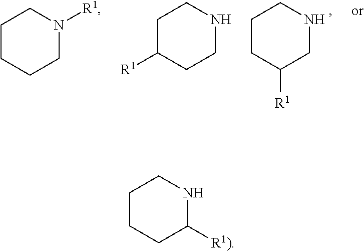

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C_{1- 4} straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR†₂, —C(S)NR†₂, —C(NH)NR†₂, or —N(R^{†})S(O)₂R^{†}; wherein each RT is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)O^{●}*, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “subject” as used herein refers to and includes any human or non-human organism that could potentially benefit from treatment with a PAD4 inhibitor. Exemplary subjects include humans and animals.

The terms “treating” or “treatment” as used herein refer to and include treatment of a disease-state in a subject, for example in a human or animal, and include: (a) inhibiting the disease-state, i.e., arresting it's development; (b) relieving the disease-state, i.e., causing regression of the disease state; and/or (c) preventing the disease-state from occurring in a subject.

The terms “preventing” or “prevention” as used herein refer to and include preventive treatment (i.e. prophylaxis and/or risk reduction) of a subclinical disease-state in a subject, for example in a human or animal, aimed at reducing the probability of the occurrence of a clinical disease-state. Subjects may be selected for preventative therapy based on factors that are known to increase risk of suffering a clinical disease state compared to the general population. “Prophylaxis” therapies can be divided into (a) primary prevention, and (b) secondary prevention. Primary prevention is defined as treatment in a subject that has not yet presented with a clinical disease state, whereas secondary prevention is defined as preventing a second occurrence of the same or similar clinical disease state.

The term “therapeutically effective amount” refers to and includes an amount of a compound or a composition according to the disclosure that is effective when administered alone or in combination to prevent or treat the disease or disorder associated with PAD4 enzyme activity. When applied to a combination, the term refers to combined amounts of the active ingredients that result in the preventive or therapeutic effect, whether administered in combination, serially, or simultaneously.

A “pharmaceutically acceptable carrier” refers to media generally accepted in the art for the delivery of biologically active agents to humans and/or animals. Pharmaceutically acceptable carriers are formulated according to a number of factors well within the purview of those of ordinary skill in the art. These include, without limitation, the type and nature of the active agent being formulated, the subject to which the agent-containing composition is to be administered, the intended route of administration of the compound or composition, and, the therapeutic indication being targeted. Pharmaceutically acceptable carriers include both aqueous and non-aqueous liquid media. Such carriers can include a number of different ingredients and additives in addition to the active agent, such additional ingredients being included in the formulation for a variety of reasons, e.g., stabilization of the active agent, binders, etc., well known to those of ordinary skill in the art. Typical, non-limiting examples of such carriers include diluents, preserving agents, fillers, flow regulating agents, disintegrating agents, wetting agents, emulsifying agents, suspending agents, sweetening agents, flavouring agents, perfuming agents, anti-bacterial agents, anti-fungal agents, lubricating agents, dispensing agents, coating agents, and the like. Descriptions of suitable pharmaceutically acceptable carriers, and factors involved in their selection, are found in a variety of readily available sources such as, for example, Allen, L. V, Jr. et al., Remington: The Science and Practice of Pharmacy (2 Volumes), 22nd Edition, Pharmaceutical Press (2012).

The present disclosure is intended to include all isotopes of atoms occurring in the present compounds. Isotopes include those atoms having the same atomic number but different mass numbers. By way of general example and without limitation, isotopes of hydrogen include deuterium (symbol D or ²H) and tritium (symbol T or ³H). For example, a methyl group may be represented by CH₃ or CD₃. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed.

The compound of Formula I forms salts which are also within the scope of this disclosure. Reference to a compound of the Formula I herein is understood to include reference to salts thereof, unless otherwise indicated. The term “salt(s)”, as employed herein, denotes acidic and/or basic salts formed with inorganic and/or organic acids and bases. In addition, when a compound of Formula I contains both a basic moiety and an acidic moiety, zwitterions (“inner salts”) may be formed and are included within the term “salt(s)” as used herein. Pharmaceutically acceptable salts include those generally acceptable in the art of pharmaceutical sciences for administration in a subject, including humans and animals. In general, the pharmaceutically acceptable salts are non-toxic and physiologically acceptable salts. Salts of the compounds according to the disclosure may be formed, for example, by reacting the compound with an amount of acid or base, such as an equivalent amount, in a medium such as one in which the salt precipitates or in an aqueous medium followed by lyophilization.

The compounds of Formula I which contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of Formula I which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)-ethylenediamine), N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The disclosure encompasses compounds of Formula I, or pharmaceutically acceptable salts thereof, methods for preparing these compounds, pharmaceutical compositions comprising these compounds, and use of these compounds in the treatment of diseases or disorders associated with PAD4 enzyme activity.

### 2. DESCRIPTION OF EXEMPLARY EMBODIMENTS

In some embodiments, the present disclosure provides a compound of Formula I:

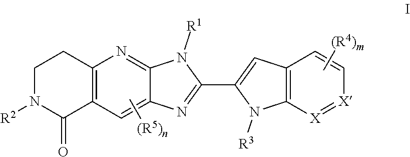

or a pharmaceutically acceptable salt, isomer, enantiomer, or tautomer thereof, wherein:
X is selected from C—R⁶ and N;
X′ is selected from C—R^{6′} and N, wherein X and X′ are not simultaneously N;
R¹ is C₁₋₄ aliphatic;
R² is C₁₋₆ aliphatic substituted by 0-4 instances of R⁷;
R³ is C₁₋₆ aliphatic substituted by 0-3 instances of R¹;
R⁴ is halogen;
R⁵ is halogen;
each R⁶ and R^{6′} is independently selected from hydrogen, halogen, —OR, —N(R)₂, —OC(O)R, —N(R)C(O)R, —O-L-(R⁹)ₚ, —Cy, and optionally substituted C₁₋₆ aliphatic;
each R⁷ is independently selected from halogen, —OR, —N(R)₂, and —Cy;
each R⁸ is independently selected from halogen, —OR, —N(R)₂, and —Cy;
each R⁹ is independently selected from halogen, —OR, —N(R)₂, and —Cy;
L is a covalent bond or C14 aliphatic;
each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
each R is independently hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur;
each m and n is independently 0 or 1; and
each p is independently 1-4.

In some embodiments, the present disclosure provides a compound of formula I:

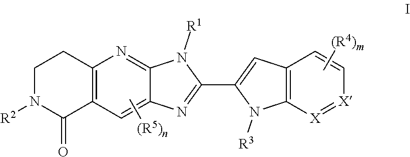

or a pharmaceutically acceptable salt, isomer, enantiomer, or tautomer thereof, wherein:
X is selected from C—R⁶ and N;
X′ is selected from C—R^{6′} and N, wherein X and X′ are not simultaneously N;
R¹ is C₁₋₄ aliphatic;
R² is C₁₋₆ aliphatic substituted by 0-4 instances of R⁷;
R³ is C₁₋₆ aliphatic substituted by 0-3 instances of R⁸;
R⁴ is halogen;
R⁵ is halogen;
each R⁶ and R^{6′} is independently selected from hydrogen, halogen, —OR, —N(R)₂, —OC(O)R, —N(R)C(O)R, —N(R)C(O)-L-(R⁹)ₚ, —O-L-(R⁹)ₚ, —Cy, optionally substituted C₁₋₆ aliphatic, and C₁₋₆ aliphatic-OH;
each R⁷ is independently selected from halogen, —OR, —N(R)₂, and —Cy;
each R⁸ is independently selected from halogen, —OR, —N(R)₂, and —Cy;
each R⁹ is independently selected from halogen, —OR, —N(R)₂, and —Cy;
L is a covalent bond or C₁₋₄ aliphatic;
each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R⁰;
each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
each R is independently hydrogen or an optionally substituted group selected from C1.6 aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulphur, wherein each R is independently substituted with 0-3 instances of halogen, —OCH₃, or —OH;
each m and n is independently 0 or 1; and
   p is independently 1-4.

In some embodiments, the present disclosure provides a compound selected from a compound of any of Formulae I-a, I-b, I-c, I-d, I-e, I-f, I-g, and I-h:

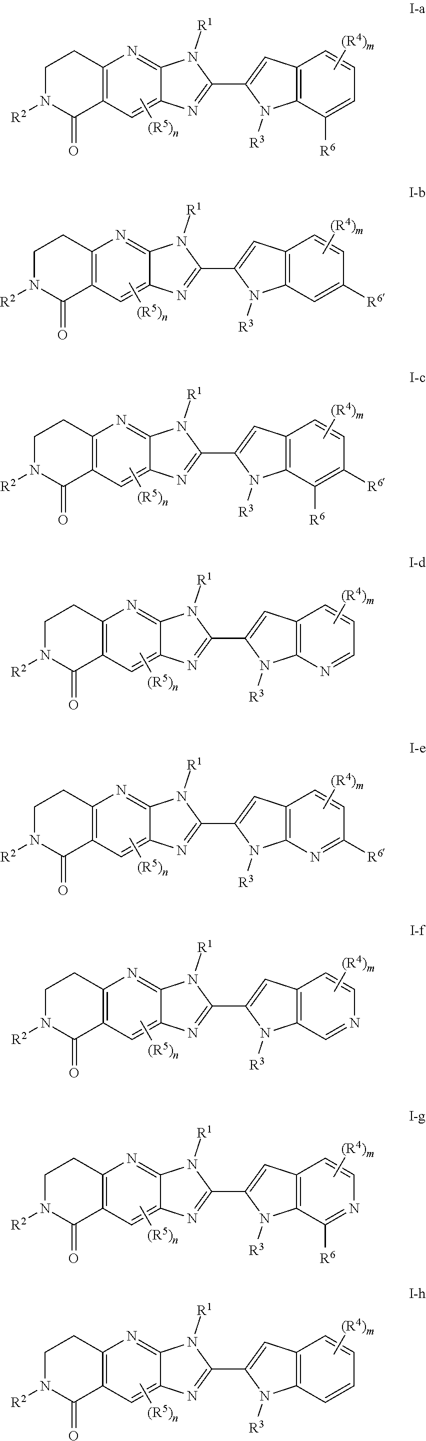

or a pharmaceutically acceptable salt thereof.

In some embodiments, the present disclosure provides a compound selected from a compound of any of Formulae I-a-i, I-b-i, I-c-i, I-d-i, I-e-i, I-f-i, I-g-i, and I-h-i:

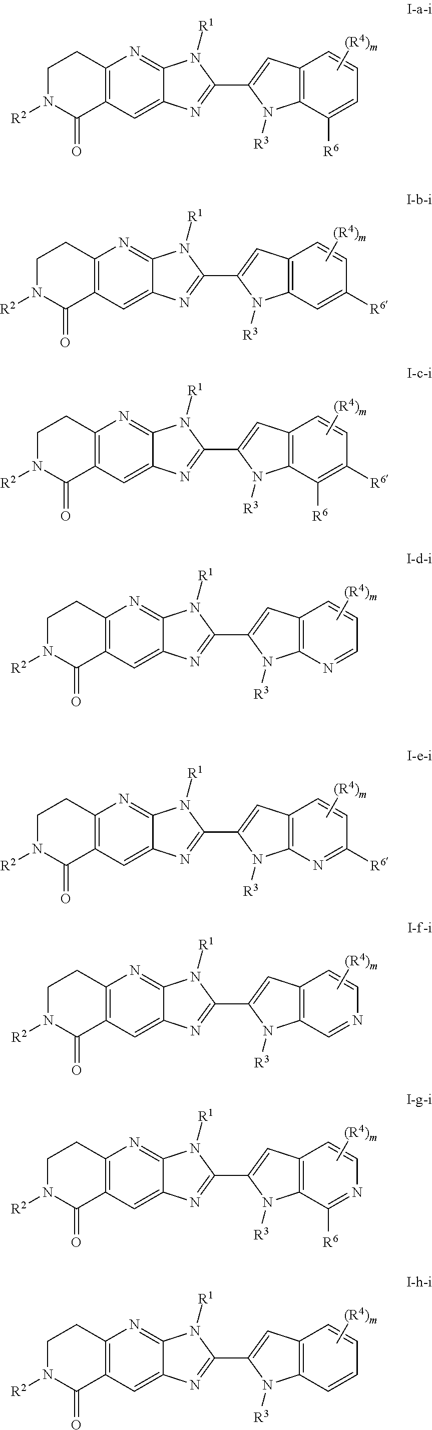

or a pharmaceutically acceptable salt thereof.

As defined generally above, X is selected from C—R⁶ and N. In some embodiments of Formulae I, X is C—R⁶. In some embodiments of any of Formulae I, X is N.

As defined generally above, X′ is selected from C—R^{6′} and N, wherein X and X′ are not simultaneously N. In some embodiments of Formulae I, X′ is C—R^{6′}. In some embodiments of Formulae I, X′ is N.

In some embodiments of Formula I, X is C—R⁶ and X′ is C—R^{6′}. In some embodiments of Formula I, X is N and X′ is C—R^{6′}. In some embodiments of Formula I, X is C—R⁶ and X′ is N′.

As defined generally above, R¹ is C₁₋₄ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹ is methyl.

As defined generally above, R² is C₁₋₆ aliphatic substituted by 0-4 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₄ aliphatic substituted by 0-4 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₂ aliphatic substituted by 0-4 instances of R⁷.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₆ aliphatic substituted by 1-2 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₄ aliphatic substituted by 1-2 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₂ aliphatic substituted by 1-2 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₆ aliphatic substituted by 3-4 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₄ aliphatic substituted by 3-4 instances of R⁷. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is C₁₋₂ aliphatic substituted by 3-4 instances of R⁷.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is selected from

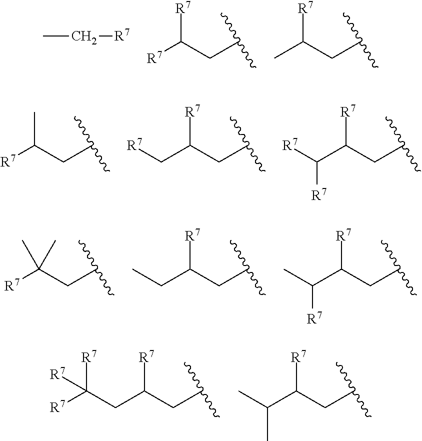

As defined generally above, R³ is C₁₋₆ aliphatic substituted by 0-3 instances of R⁸. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is C₁₋₄ aliphatic substituted by 0-3 instances of R⁸. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is C₁₋₂ aliphatic substituted by 0-3 instances of R⁸.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is C₁₋₆ aliphatic substituted by 1-3 instances of R⁸. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is C₁₋₄ aliphatic substituted by 1-3 instances of R⁸. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is C₁₋₂ aliphatic substituted by 1-3 instances of R⁸.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is selected from:

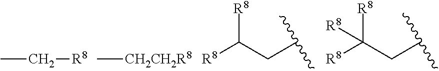

As defined generally above, R⁴ is halogen. In various embodiments, any substitutable position of the fused bicyclic ring moiety of any of Formulae I, I′, I-a, I-a-i, I-b, I-b-i, I-c, I-c-i, I-d, I-d-i, I-e, I-e-i, I-f, I-f-i, I-g, I-g-i, I-h, and I-h-i is substituted with R⁴. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁴ is fluoro or chloro.

As defined generally above, R⁵ is halogen. In some embodiments of any of Formulae I, I-a, I-b, I-c, I-d, I-e, I-f, I-g, and I-h, R⁵ is fluoro.

As defined generally above, each R⁶ and R^{6′} is selected from hydrogen, halogen, —OR, —N(R)₂, —OC(O)R, —N(R)C(O)R, —O-L-(R⁹)ₚ, —Cy, and optionally substituted C₁₋₆ aliphatic. In some embodiments of Formula I, R⁶ is hydrogen.

In some embodiments of any of Formulae I, I-a, I-a-i, I-c, I-c-i, I-g, and I-g-i, R⁶ is halogen. In some such embodiments of any of Formulae I, I-a, I-a-i, I-c, I-c-i, I-g, and I-g-i, R⁶ is fluoro.

In some embodiments of any of Formulae I, I-a, I-a-i, I-c, I-c-i, I-g, and I-g-i, R⁶ is —OR.

In some embodiments of any of Formulae I, I-a, I-a-i, I-c, I-c-i, I-g, and I-g-i, R⁶ is —N(R)₂.

In some embodiments of any of Formulae I, I-a, I-a-i, I-c, I-c-i, I-g, and I-g-i, R⁶ is —OC(O)R.

In some embodiments of any of Formulae I, I-a, I-a-i, I-c, I-c-i, I-g, and I-g-i, R⁶ is —N(R)C(O)R. In some embodiments of any of Formulae I, I-a, I-a-i, I-c, I-c-i, I-g, and I-g-i, R⁶ is —NHC(O)R. In some such embodiments of any of Formulae I, I-a, I-a-i, I-c, I-c-i, I-g, and I-g-i, R is C₁₋₆ aliphatic optionally substituted with R^{∘}. In some embodiments of any of Formulae I, I-a, I-a-i, I-c, I-c-i, I-g, and I-g-i, R⁶ is —NHC(O)R, wherein R is C₁₋₆ aliphatic optionally substituted with R^{∘}, wherein R^{∘} is a 5- to 6-membered heteroaryl ring having 1-3 heteroatoms independently selected from nitrogen, oxygen, and sulfur.

In some embodiments of any of Formulae I, I-a, I-a-i, I-c, I-c-i, I-g, and I-g-i, R⁶ is —Cy.

In some embodiments of any of Formulae I, I-a, I-a-i, I-c, I-c-i, I-g, and I-g-i, R⁶ is —O-L-(R⁹)ₚ.

In some embodiments of any of Formulae I, I-a, I-a-i, I-c, I-c-i, I-g, and I-g-i, R⁶ is optionally substituted C₁₋₆ aliphatic. In some such embodiments of any of Formulae I, I-a, I-a-i, I-c, I-c-i, I-g, and I-g-i, R⁶ is —CH₃.

In some embodiments of any of Formulae I, I-a, I-a-i, I-c, I-c-i, I-g, and I-g-i, R⁶ is selected from —CH₃, —OCH,

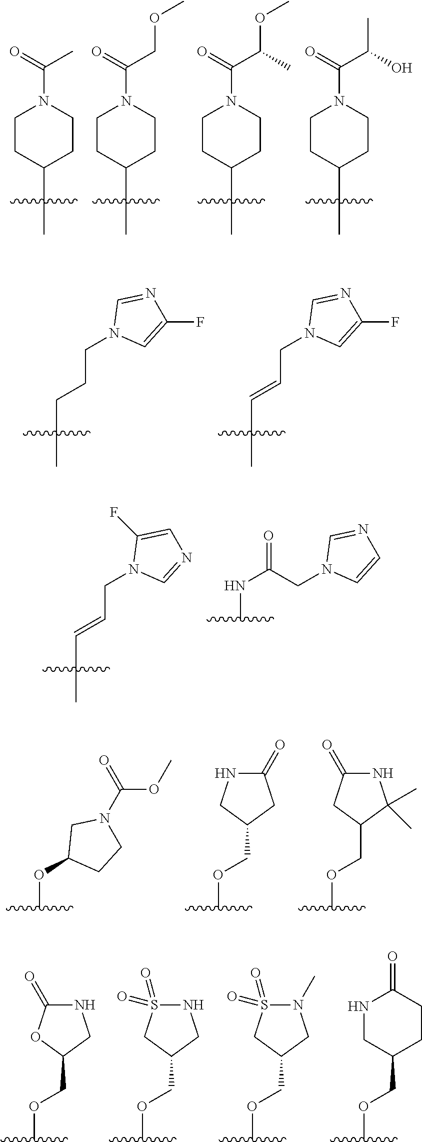

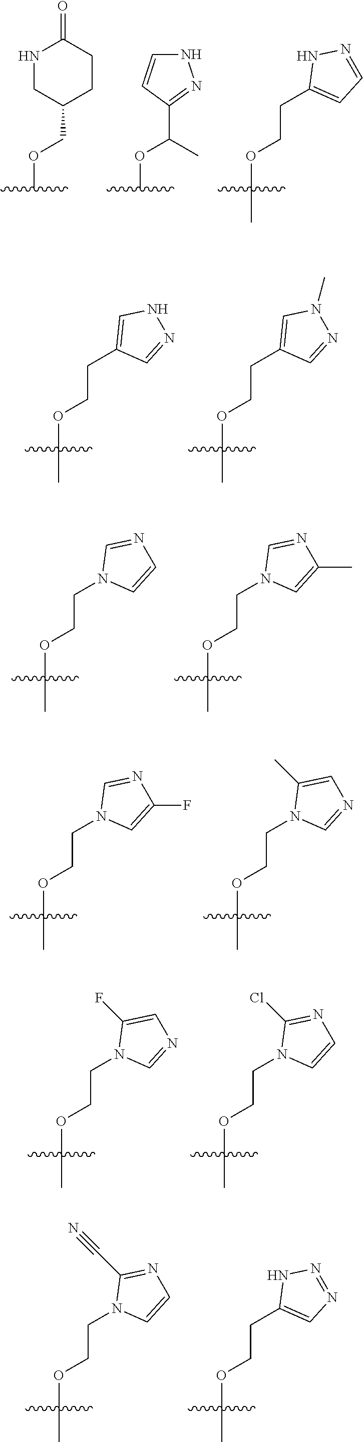

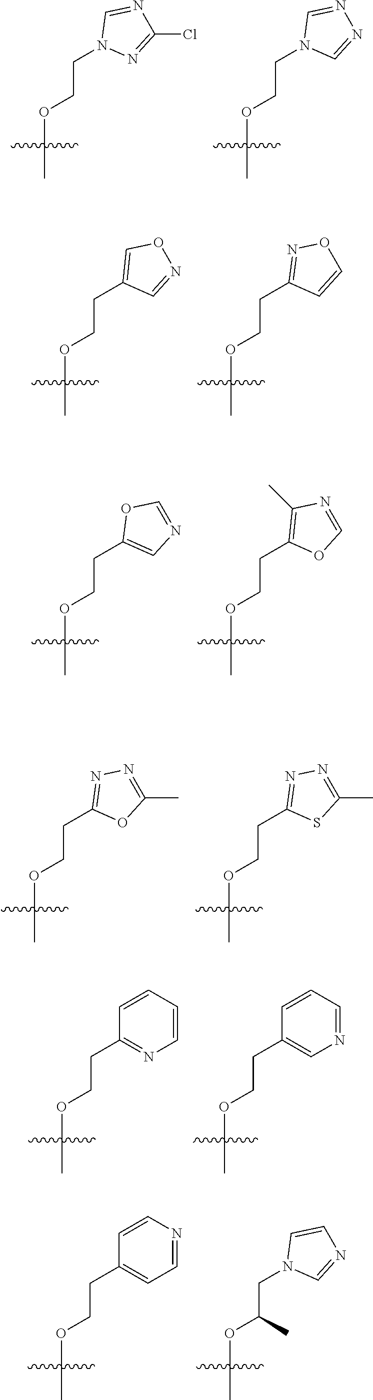

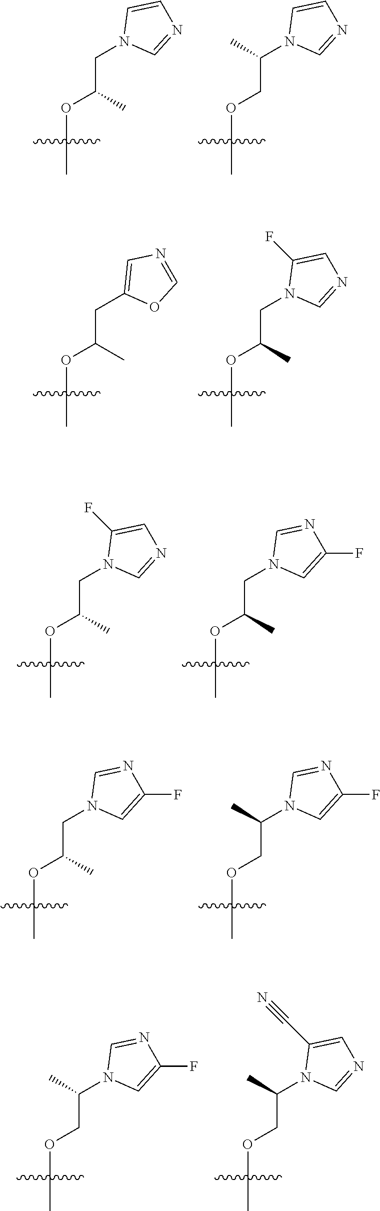

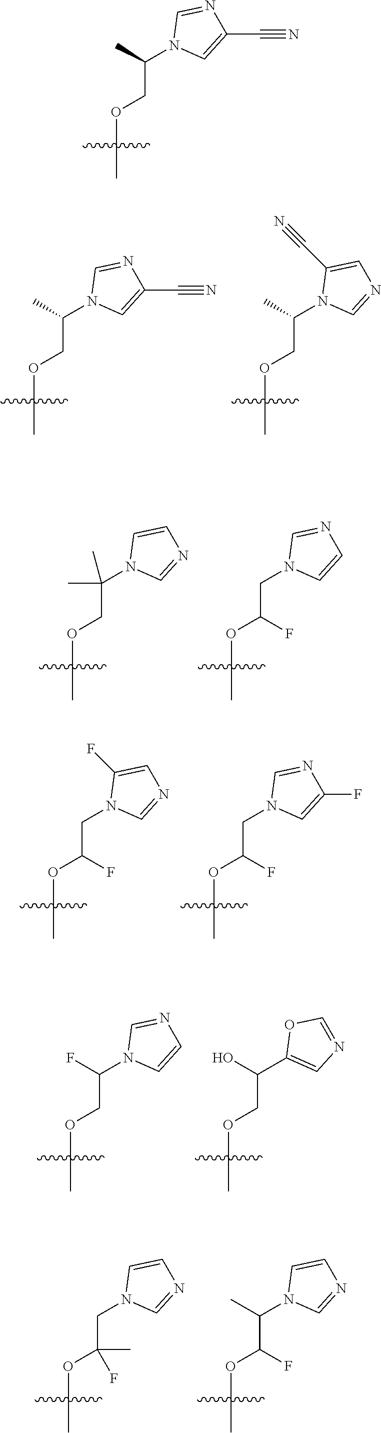

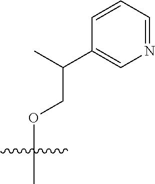 [SMILES: *OCC(C)c1cccnc1]

In some embodiments of any of Formulae I, R^{6′} is hydrogen.

In some embodiments of any of Formulae I, I-b, I-b-i, I-c, I-c-i, I-e, and I-e-i, R^{6′} is halogen. In some such embodiments, R^{6′} is fluoro.

In some embodiments of any of Formulae I, I-b, I-b-i, I-c, I-c-i, I-e, and I-e-i, R^{6′} is optionally substituted C₁₋₆ aliphatic. In some embodiments of any of Formulae I, I-b, I-b-i, I-c, I-c-i, I-e, and I-e-i, R^{6′} is optionally substituted C₁₋₄ aliphatic. In some embodiments of any of Formulae I, I-b, I-b-i, I-c, I-c-i, I-e, and I-e-i, R^{6′} is optionally substituted C₁₋₂ aliphatic. In some embodiments of any of Formulae I, I-b, I-b-i, I-c, I-c-i, I-e, and I-e-i, R^{6′} is optionally substituted C₂₋₃ aliphatic. In some such embodiments of any of Formulae I, I-b, I-b-i, I-c, I-c-i, I-e, and I-e-i, R^{6′} is —C(CH₃)₂OH.

In some embodiments of any of Formulae I, I-b, I-b-i, I-c, I-c-i, I-e, and I-e-i, R^{6′} is —OR.

In some embodiments of any of Formulae I, I-b, I-b-i, I-c, I-c-i, I-e, and I-e-i, R^{6′} is —N(R)₂.

In some embodiments of any of Formulae I, I-b, I-b-i, I-c, I-c-i, I-e, and I-e-i, R^{6′} is —OC(O)R.

In some embodiments of any of Formulae I, I-b, I-b-i, I-c, I-c-i, I-e, and I-e-i, R^{6′} is —N(R)C(O)R.

In some embodiments of any of Formulae I, I-b, I-b-i, I-c, I-c-i, I-e, and I-e-i, R^{6′} is —Cy.

In some embodiments of any of Formulae I, I-b, I-b-i, I-c, I-c-i, I-e, and I-e-i, R^{6′} is —O-L-(R⁹)ₚ.

In some embodiments of any of Formulae I, I-b, I-b-i, I-c, I-c-i, I-e, and I-e-i, R^{6′} is selected from fluoro, —OCH₃, and —C(CH₃)₂OH.

As defined generally above, R⁷ is selected from halogen, —OR, —N(R)₂, and —Cy. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is halogen. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is fluoro. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is —OR. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is —N(R)₂. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is hydrogen. Accordingly, in some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is —NH₂. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is —Cy. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁷ is selected from fluoro, —NH₂, and —Cy.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R² is selected from

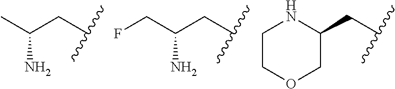

As defined generally above, R⁸ is selected from halogen, —OR, —N(R)₂, and —Cy. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁸ is —Cy. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁸ is —OR. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁸ is halogen. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R⁸ is fluoro.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R³ is selected from

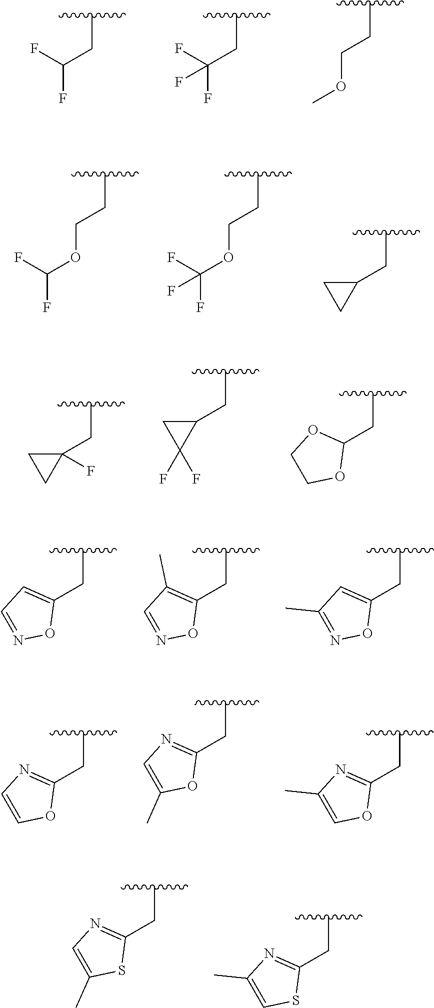

As defined generally above, R⁹ is selected from halogen, —OR, —N(R)₂, and —Cy. In some embodiments of any of Formulae I, I-a, I-a-i, I-b, I-b-i, I-c, I-c-i, I-e, I-e-i, I-g, and I-g-i, R⁹ is halogen. In some such embodiments of any of Formulae I, I-a, I-a-i, I-b, I-b-i, I-c, I-c-i, I-e, I-e-i, I-g, and I-g-i, R⁹ is fluoro. In some embodiments of any of Formulae I, I-a, I-a-i, I-b, I-b-i, I-c, I-c-i, I-e, I-e-i, I-g, and I-g-i, R⁹ is —OR. In some embodiments of any of Formulae I, I-a, I-a-i, I-b, I-b-i, I-c, I-c-i, I-e, I-e-i, I-g, and I-g-i, R⁹ is —N(R)₂. In some such embodiments of any of Formulae I, I-a, I-a-i, I-b, I-b-i, I-c, I-c-i, I-e, I-e-i, I-g, and I-g-i, R is hydrogen. Accordingly, in some embodiments any of Formulae I, I-a, I-a-i, I-b, I-b-i, I-c, I-c-i, I-e, I-e-i, I-g, and I-g-i, R⁹ is —NH₂. In some embodiments of any of Formulae I, I-a, I-a-i, I-b, I-b-i, I-c, I-c-i, I-e, I-e-i, I-g, and I-g-i, R⁹ is —Cy.

As defined generally above, L is a covalent bond or C₁₋₄ aliphatic. In some embodiments of any of Formulae I, I-a, I-a-i, I-b, I-b-i, I-c, I-c-i, I-e, I-e-i, I-g, and I-g-i, L is a covalent bond. In some embodiments of any of Formulae I, I-a, I-a-i, I-b, I-b-i, I-c, I-c-i, I-e, I-e-i, I-g, and I-g-i, L is C₁₋₄ aliphatic. In some embodiments of any of Formulae I, I-a, I-a-i, I-b, I-b-i, I-c, I-c-i, I-e, I-e-i, I-g, and I-g-i, L is C₁₋₃ aliphatic. In some embodiments of any of Formulae I, I-a, I-a-i, I-b, I-b-i, I-c, I-c-i, I-e, I-e-i, I-g, and I-g-i, L is C₁₋₂ aliphatic. In some embodiments of any of Formulae I, I-a, I-a-i, I-b, I-b-i, I-c, I-c-i, I-e, I-e-i, I-g, and I-g-i, L is C₂₋₃ aliphatic.

In some embodiments of any of Formulae I, I-a, I-a-i, I-b, I-b-i, I-c, I-c-i, I-e, I-e-i, I-g, and I-g-i, -L-(R⁹)ₚ is selected from

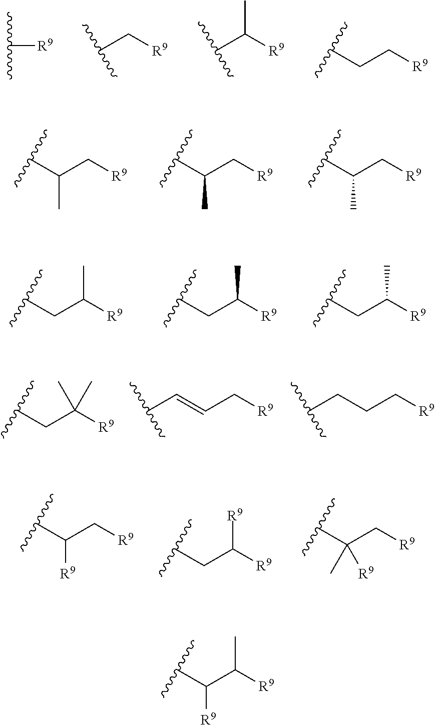

In some embodiments of any of Formulae I, I-a, I-a-i, I-b, I-b-i, I-c, I-c-i, I-e, I-e-i, I-g, and I-g-i, R⁹ is selected from fluoro,

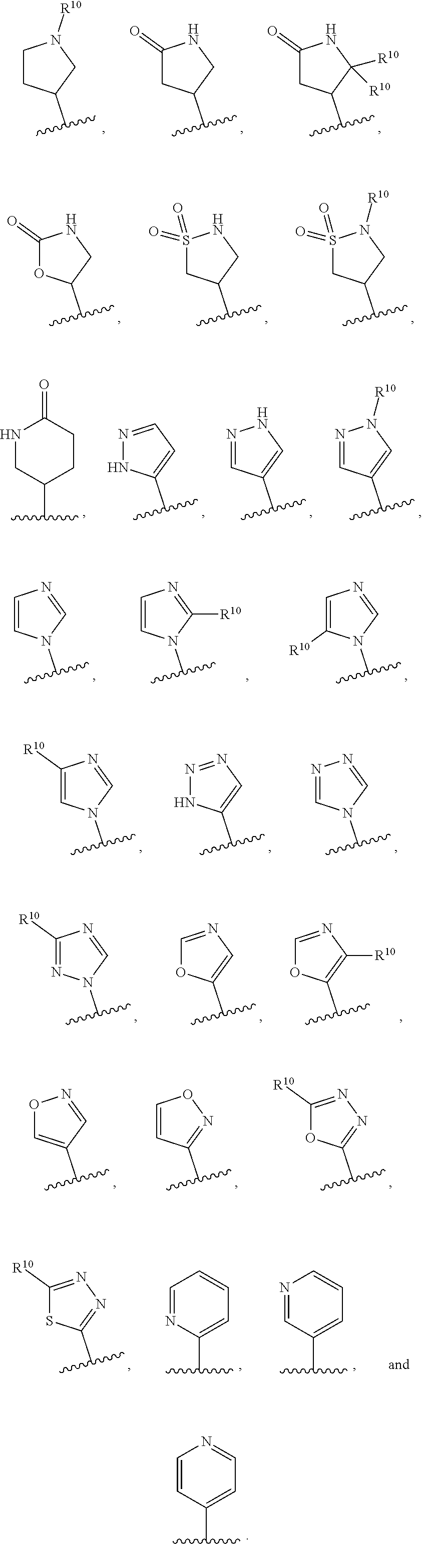

As defined generally above, Cy is selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is substituted by 1-2 instances of R¹⁰. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is substituted by 1-3 instances of R¹⁰.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 3- to 7-membered saturated or partially unsaturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 3-membered saturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 4-membered saturated carbocyclic ring. In some embodiments of any of Formulae I, I-a, I-a-i, I-b, I-b-i, I-c, I-c-i, I-d, I-d-i, I-e, I-e-i, I-f I-f-i, I-g, I-g-i, I-h, and I-h-i, Cy is a 5-membered saturated or partially unsaturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered saturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 6-membered saturated or partially unsaturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 6-membered partially unsaturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 7-membered saturated or partially unsaturated carbocyclic ring. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 7-membered saturated carbocyclic ring.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 3- to 7-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 3-membered saturated heterocyclic ring having 1 heteroatom selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 4-membered saturated heterocyclic ring having 1 heteroatom selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered saturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 6-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 6-membered saturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 7-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 7-membered saturated heterocyclic ring having 1-2 heteroatoms independently selected from nitrogen, oxygen, and sulfur.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is phenyl.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5- to 6-membered heteroaryl ring having 1-4 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered heteroaryl ring having 1-4 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered heteroaryl ring having 2-4 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 5-membered heteroaryl ring having 2-3 heteroatoms independently selected from nitrogen, oxygen, and sulfur.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is a 6-membered heteroaryl ring having 1-2 nitrogen atoms.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, Cy is selected from

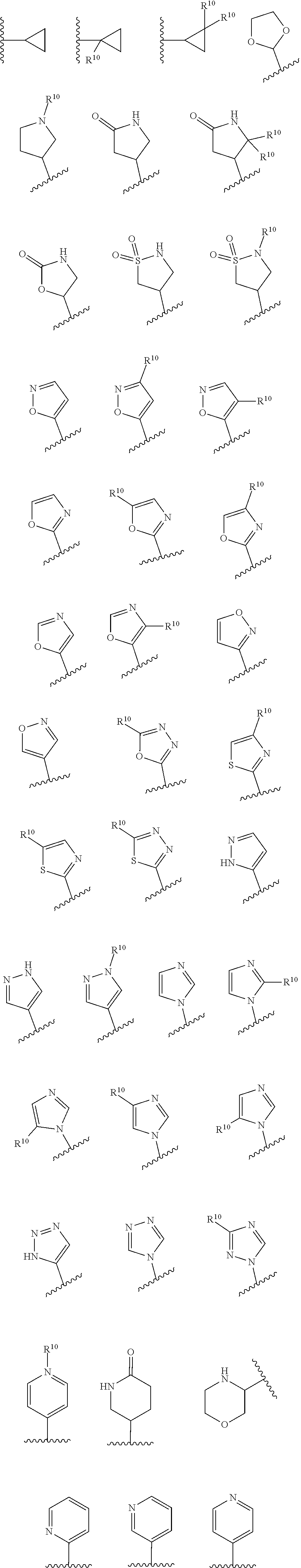

As defined generally above, R¹⁰ is selected from halogen, —OR, —N(R)₂, —CN, —C(O)R, —C(O)OR, —C(O)N(R)₂, oxo, and an optionally substituted group selected from C1.6 aliphatic and a 3- to 7-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is halogen. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is fluoro or chloro.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —OR.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —N(R)₂.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —CN.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₄ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₂ aliphatic.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is optionally substituted C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₆ aliphatic optionally substituted with —OR^{∘}. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R^{∘} is hydrogen or C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₄ aliphatic optionally substituted with —OR^{∘}. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)R, wherein R is C₁₋₂ aliphatic optionally substituted with —OR^{∘}.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)OR.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —C(O)N(R)₂.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is oxo.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted C₁₋₄ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted C₁₋₂ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is —CH₃.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 3- to 7-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 3-membered saturated or partially unsaturated heterocyclic ring having 1 heteroatom selected from nitrogen, oxygen, and sulfur In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 4-membered saturated or partially unsaturated heterocyclic ring having 1 heteroatom selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 5-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 5-membered saturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 6-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 6-membered saturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 7-membered saturated or partially unsaturated heterocyclic ring having 1-2 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is optionally substituted 7-membered saturated heterocyclic ring having 1-2 heteroatoms independently selected from nitrogen, oxygen, and sulfur.

In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R¹⁰ is selected from oxo, fluoro, chloro, —CN, —CH₃,

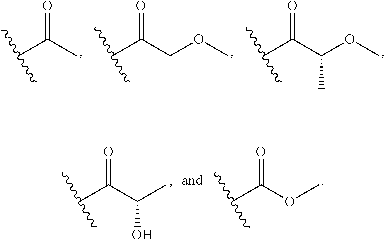

As defined generally above, R is hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is hydrogen. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is an optionally substituted group selected from C1.6 aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is optionally substituted C₁₋₆ aliphatic. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is C₁₋₆ aliphatic optionally substituted with halogen or —OR^{∘}. In some such embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R^{∘} is hydrogen or C₁₋₆ aliphatic. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, R is C₁₋₆ aliphatic optionally substituted with halogen or —OR^{∘}, wherein R^{∘} is hydrogen or C₁₋₆ aliphatic.

As defined generally above, each m and n is 0 or 1. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, m is 0. In some embodiments of any of Formulae I, I-a, I-a-i, I-b, I-b-i, I-c, I-c-i, I-d, I-d-i, I-e, I-e-i, I-f, I-f-i, I-g, I-g-i, I-h, and I-h-i, m is 1. In some embodiments of any of Formulae I, I-a, I-b, I-c, I-d, I-e, I-f, I-g, and I-h, n is 0. In some embodiments of any of Formulae I, I-a, I-b, I-c, I-d, I-e, I-f, I-g, and I-h, n is 1. In some embodiments of Formula I, m is 1 and n is 0. In some embodiments of Formula I, each of m and n is 0.

As defined generally above, p is 1-4. In some embodiments of any of Formulae I, I-a, I-a-i, I-b, I-b-i, I-c, I-c-i, I-e, I-e-i, I-g, and I-g-i, p is 1. In some embodiments of any of Formulae I, I-a, I-a-i, I-b, I-b-i, I-c, I-c-i, I-e, I-e-i, I-g, and I-g-i, p is 1-2. In some embodiments of any of Formulae I, I-a, I-a-i, I-b, I-b-i, I-c, I-c-i, I-e, I-e-i, I-g, and I-g-i, p is 2. In some embodiments of any of Formulae I, I-a, I-a-i, I-b, I-b-i, I-c, I-c-i, I-e, I-e-i, I-g, and I-g-i, p is 3.

In some embodiments, a compound of Formula I is selected from

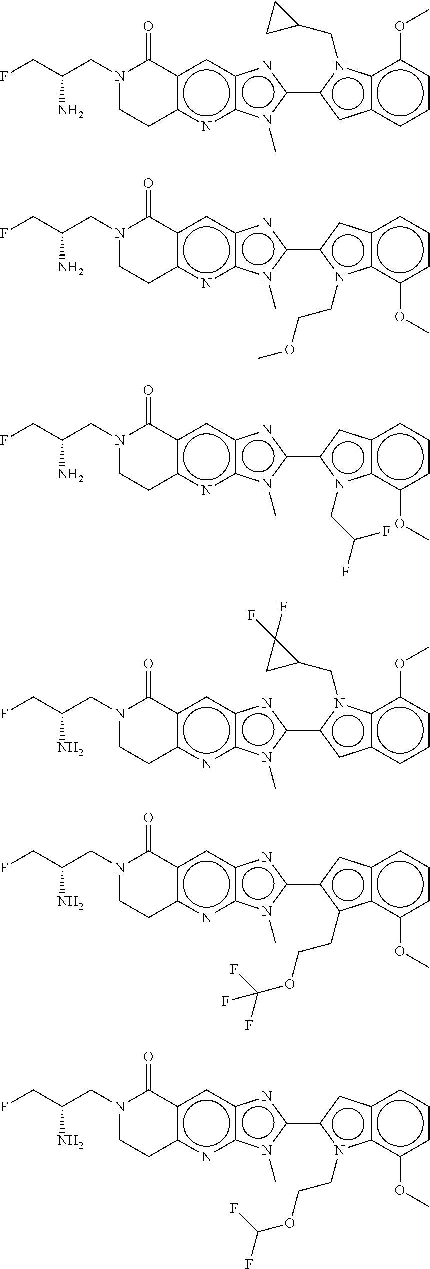

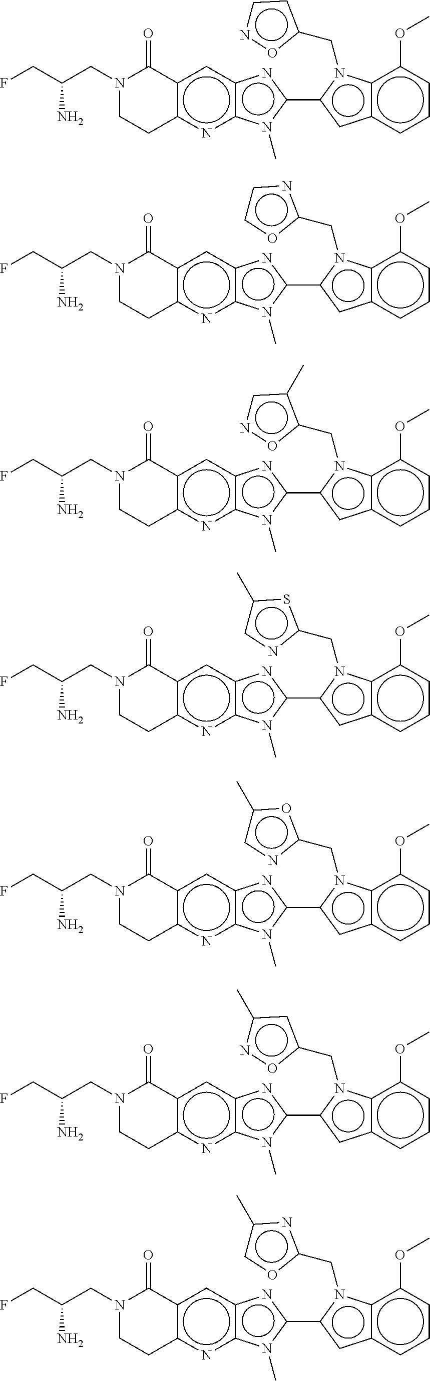

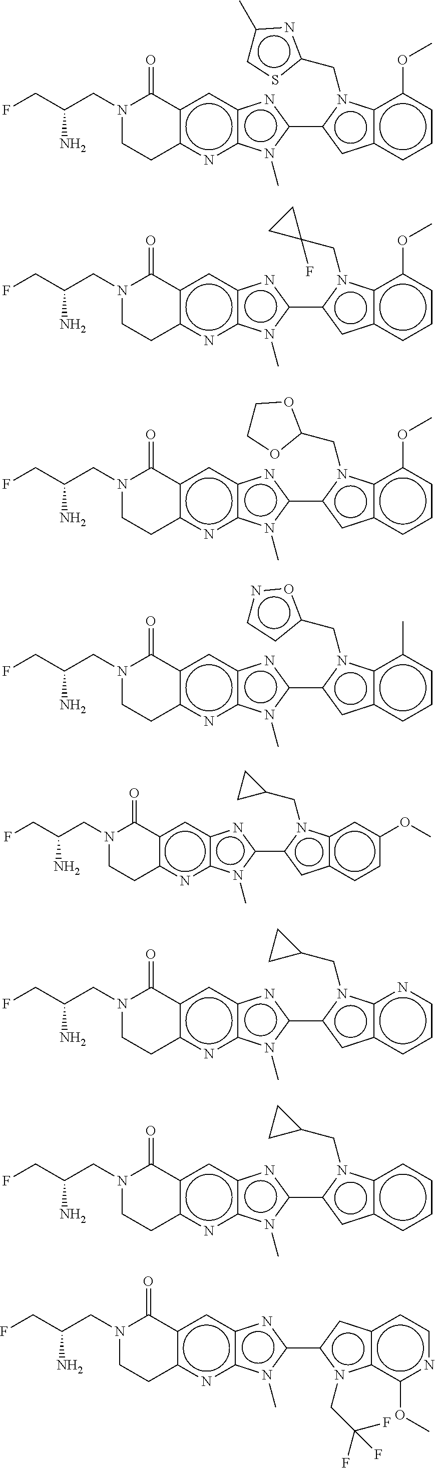

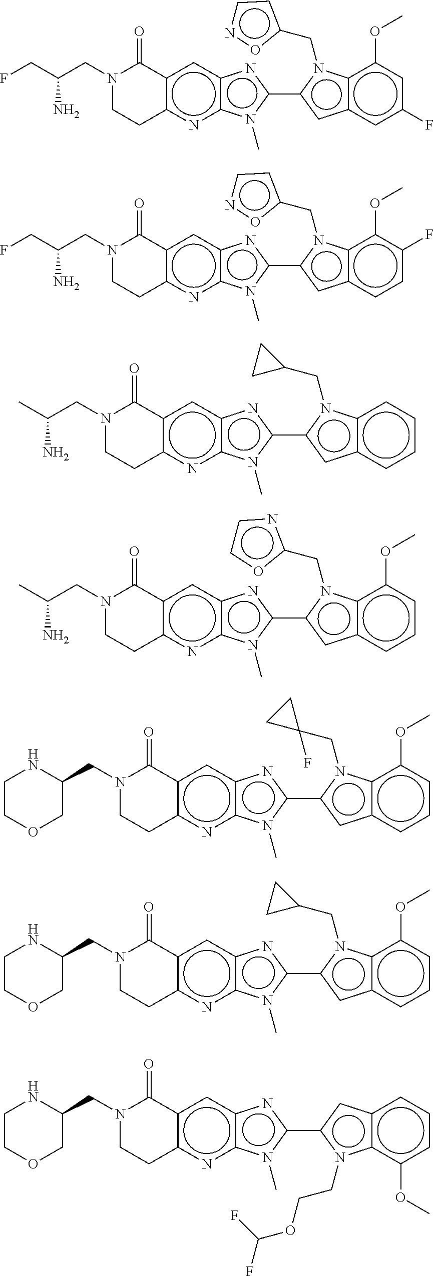

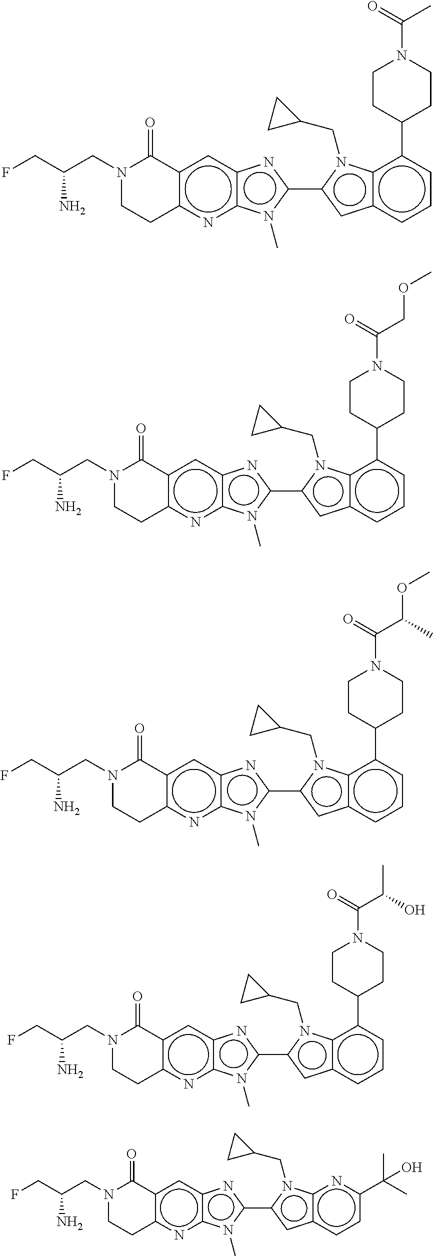

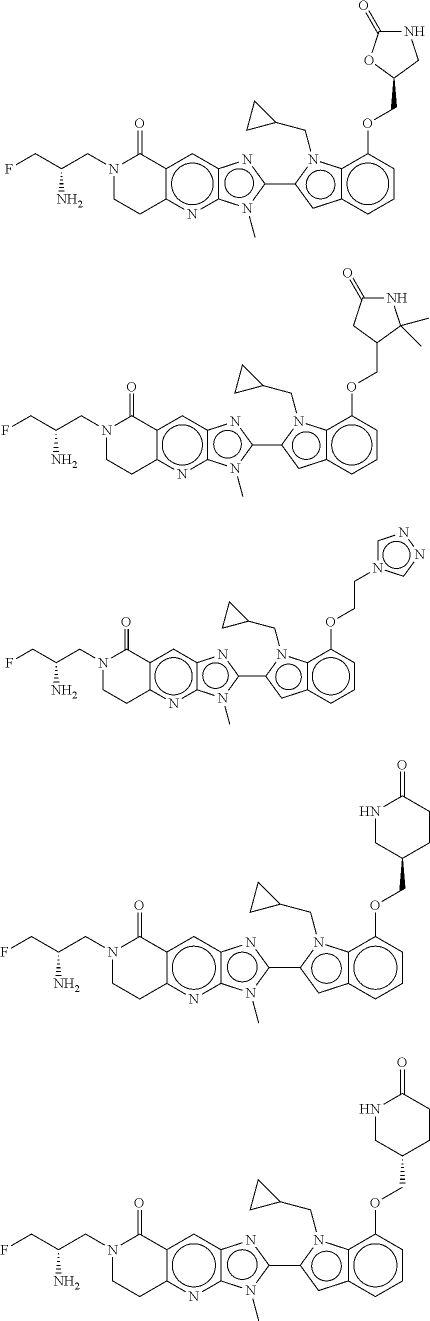

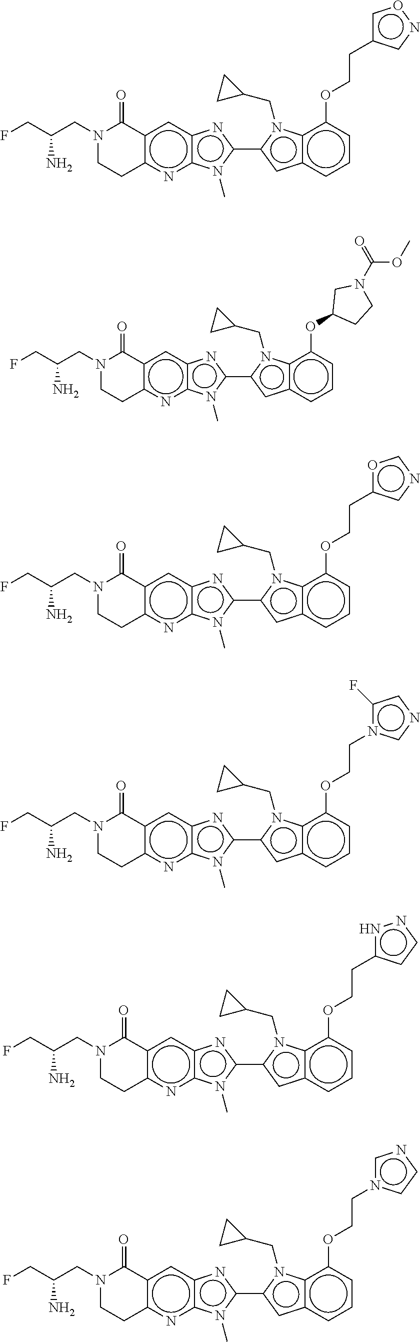

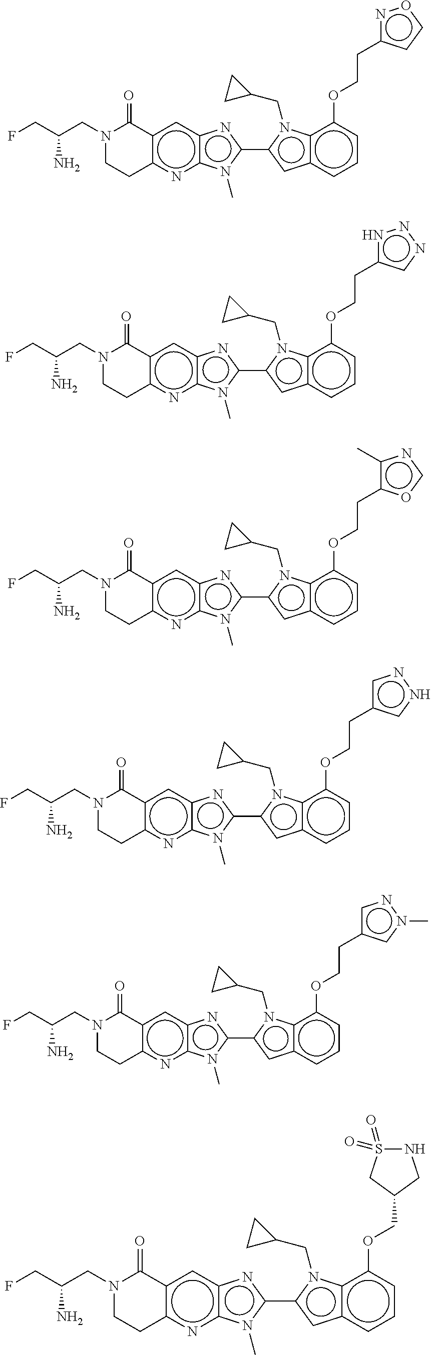

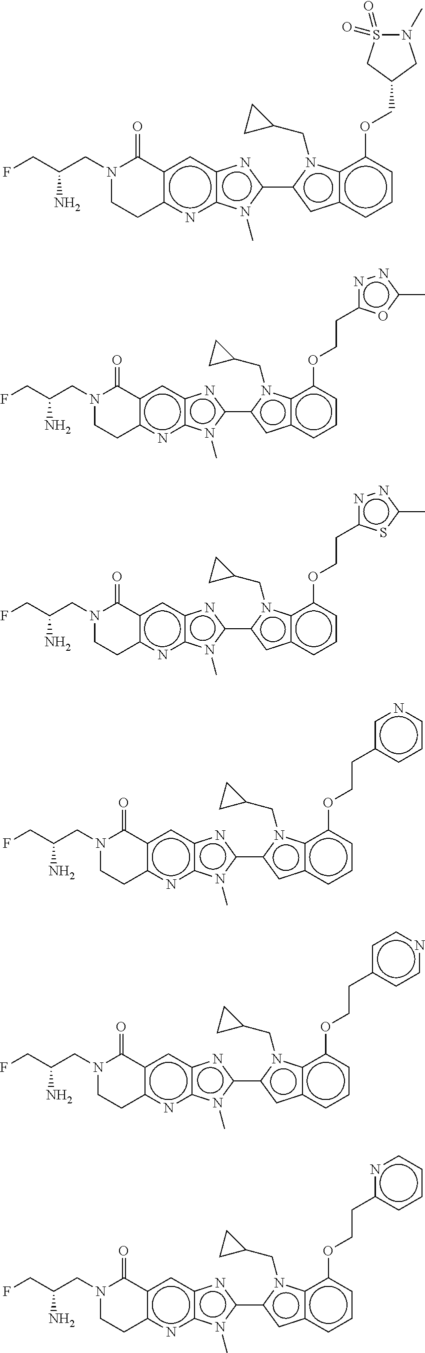

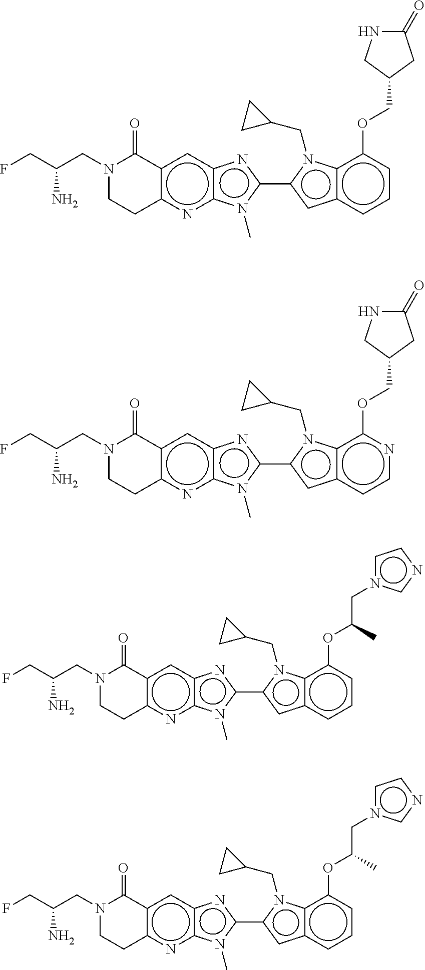

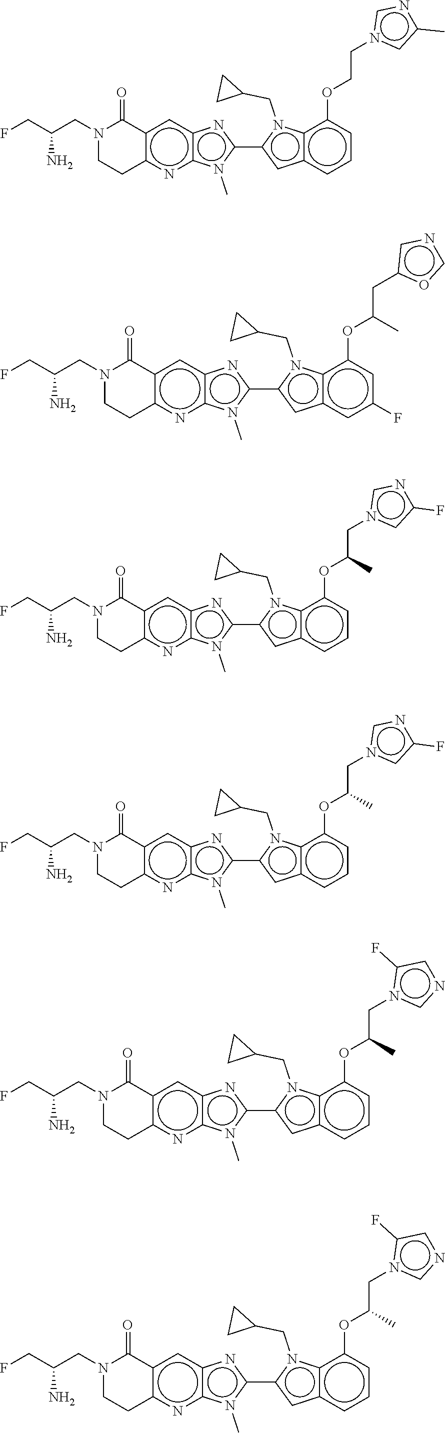

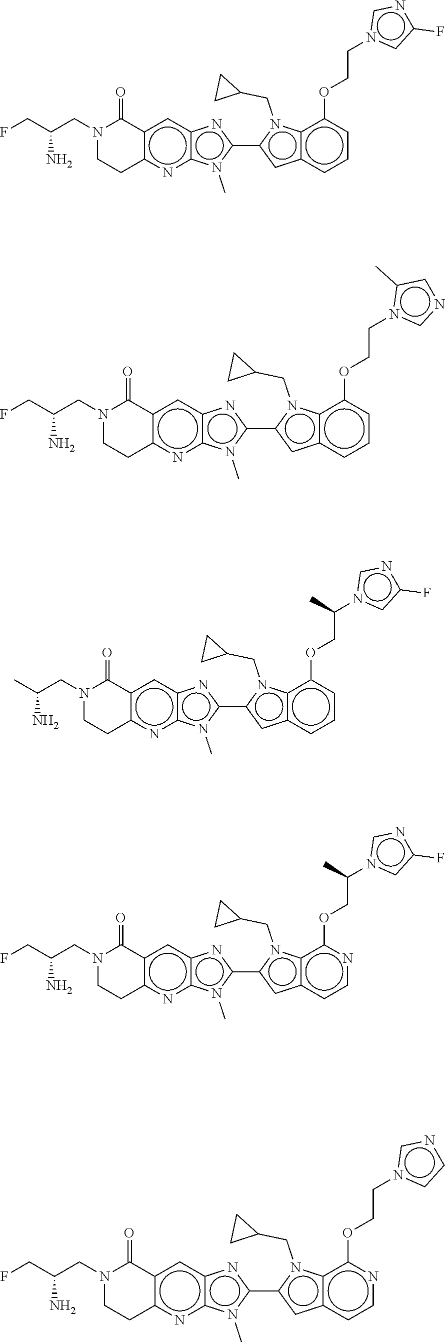

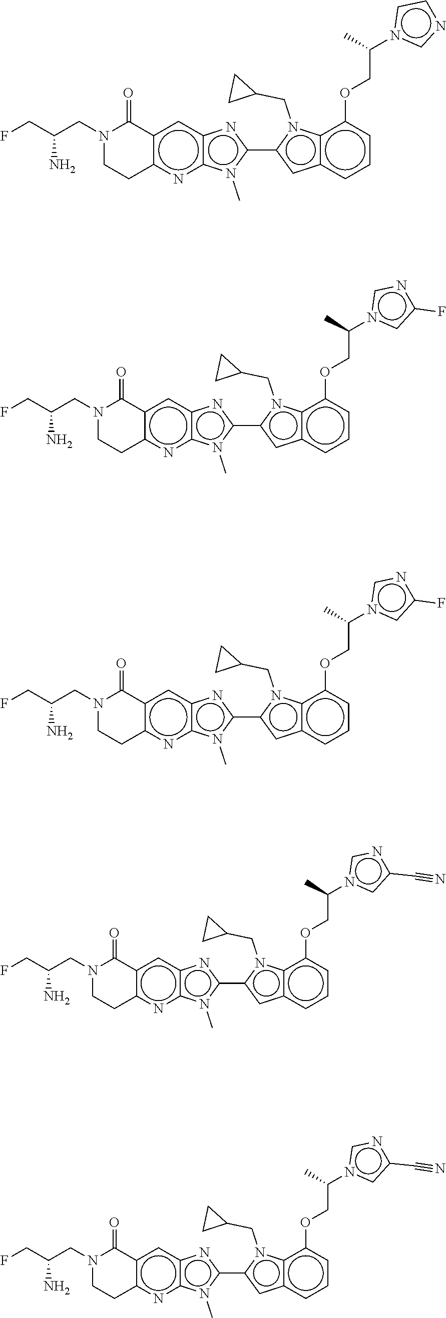

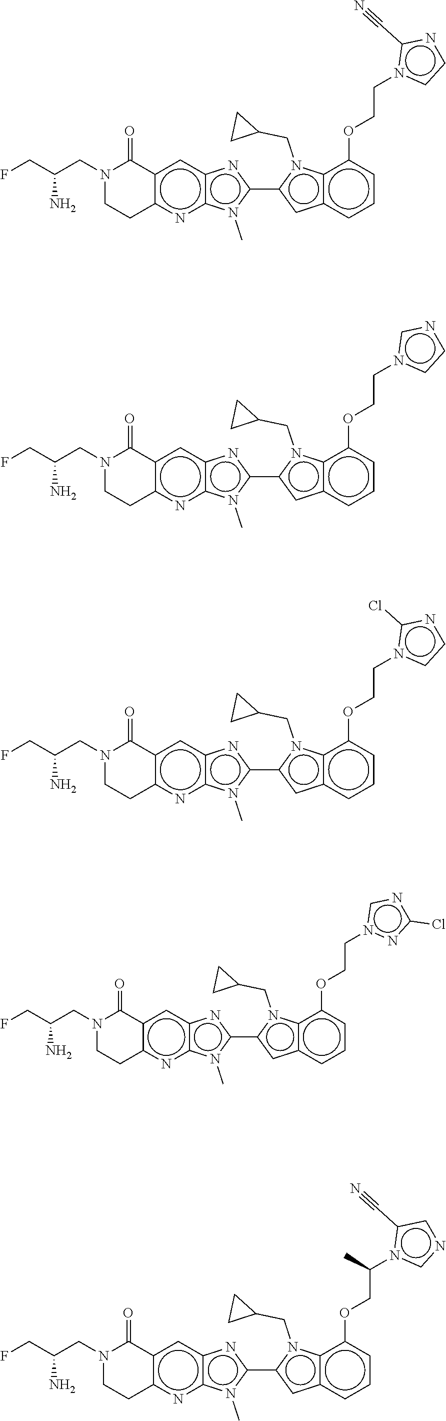

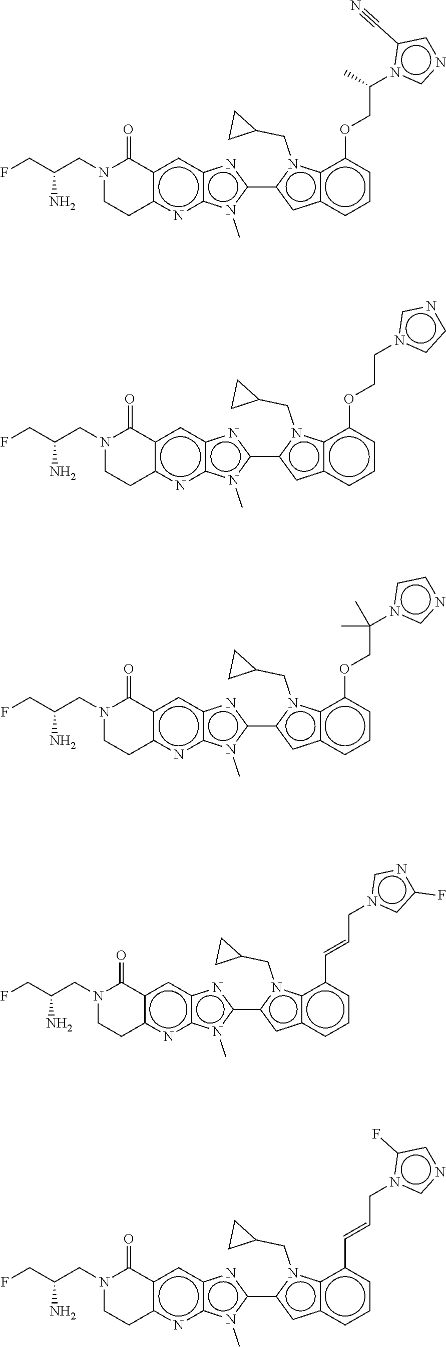

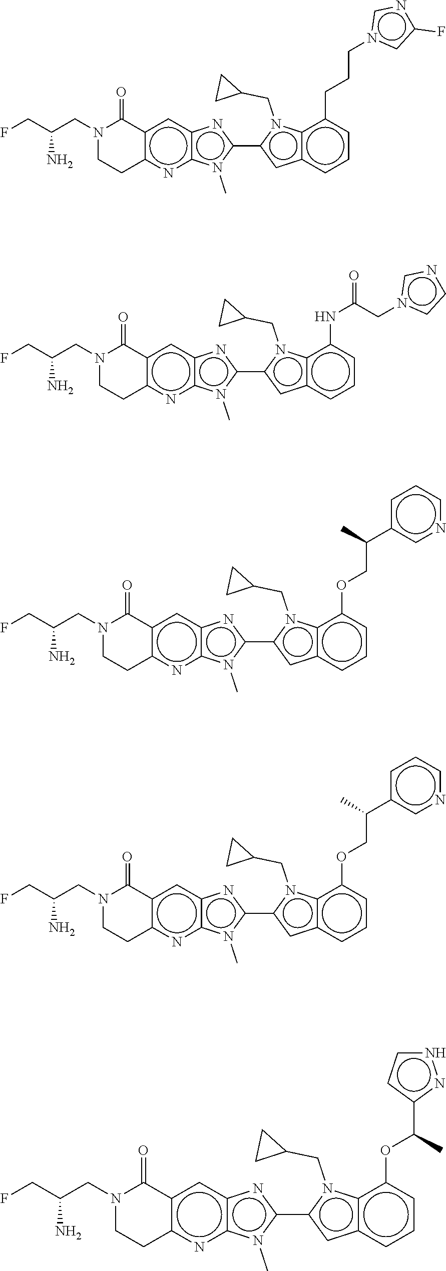

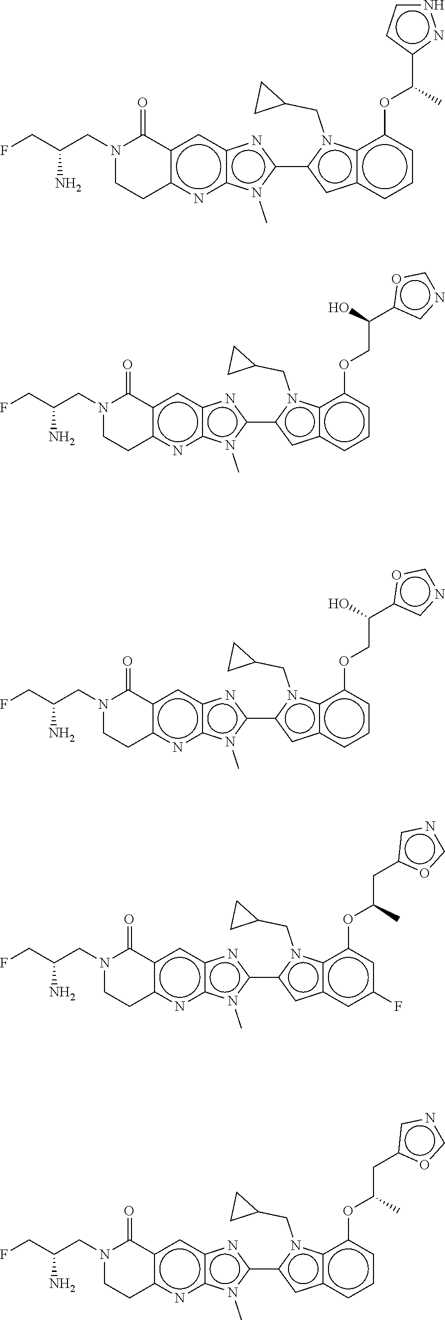

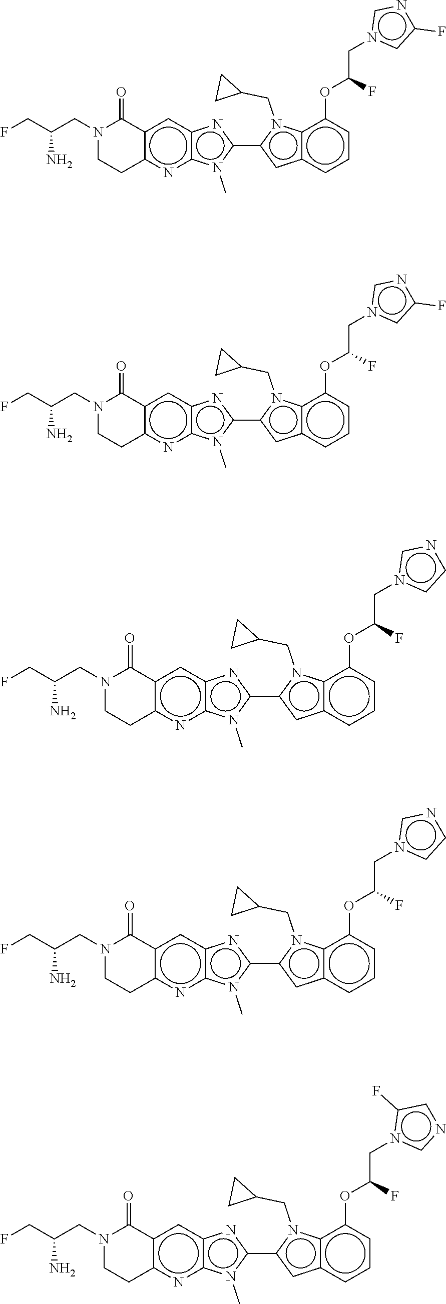

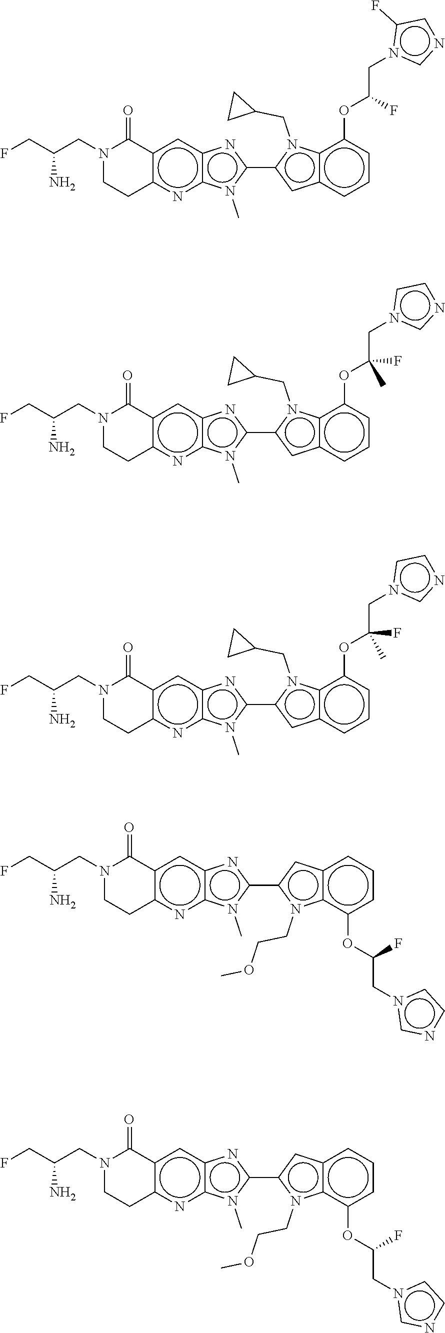

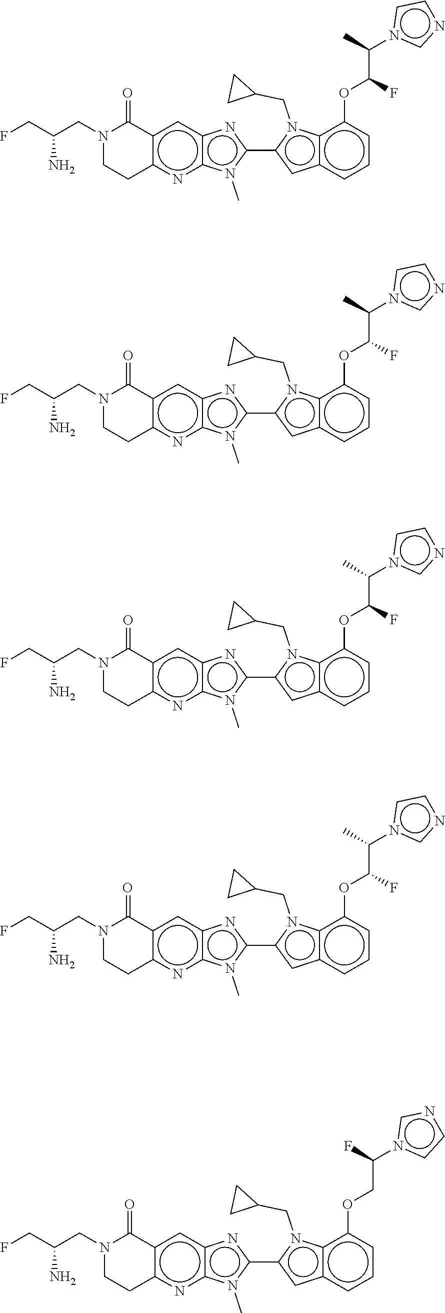

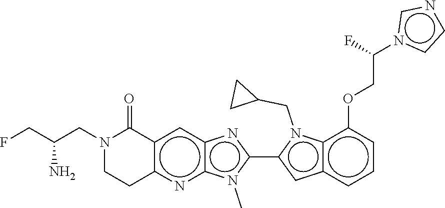

or a pharmaceutically acceptable salt thereof.

### 4. PHARMACEUTICAL COMPOSITIONS

In some embodiments, the present disclosure provides a composition comprising a compound provided by this disclosure, or a pharmaceutically acceptable salt thereof, and a pharmaceutically acceptable carrier, adjuvant, or vehicle. The amount of a compound in the compositions of this disclosure is such that it is effective to measurably inhibit PAD4 in a biological sample or in a patient. In certain embodiments, the amount of compound in compositions of this disclosure is such that it is effective to measurably inhibit PAD4, in a biological sample or in a patient. In certain embodiments, a composition provided by this disclosure is formulated for administration to a patient in need of such composition. In some embodiments, a composition provided by this disclosure is formulated for oral administration to a patient.

The term “subject,” as used herein, is used interchangeably with the term “patient” and means an animal, or a mammal. In some embodiments, a subject or patient is a human. In other embodiments, a subject (or patient) is a veterinary subject (or patient). In some embodiments, a veterinary subject (or patient) is a canine, a feline, or an equine subject.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions provided by this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions provided by this disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. The compositions can be administered orally, intraperitoneally or intravenously. Sterile injectable forms of the compositions provided by this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions provided by this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions provided by this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions provided by this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used. For topical applications, provided pharmaceutically acceptable compositions may be formulated in a suitable ointment containing the active component suspended or dissolved in one or more carriers. Carriers for topical administration of compounds provided by this disclosure include, but are not limited to, mineral oil, liquid petrolatum, white petrolatum, propylene glycol, polyoxyethylene, polyoxypropylene compound, emulsifying wax and water.

Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions provided by this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Furthermore pharmaceutically acceptable compositions provided by this disclosure maybe formulated for oral administration. Such formulations maybe administered with or without food. In some embodiments, pharmaceutically acceptable compositions provided by this disclosure are administered without food. In other embodiments, pharmaceutically acceptable compositions provided by this disclosure are administered with food.

Pharmaceutically acceptable compositions provided by this disclosure can be administered to humans and other animals orally, rectally, parenterally, intracisternally, intravaginally, intraperitoneally, topically (as by powders, ointments, or drops), bucally, as an oral or nasal spray, or the like, as required. In certain embodiments, the compounds provided by this disclosure may be administered orally or parenterally at dosage levels of about 0.01 mg/kg to about 50 mg/kg or from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid may also be used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound provided by this disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration may be suppositories which can be prepared by mixing the compounds provided by this disclosure with suitable non-irritating excipients or carriers such as cocoa butter, polyethylene glycol or a suppository wax which are solid at ambient temperature but liquid at body temperature, and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound provided by this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The amount of compounds of the present disclosure that may be combined with the carrier materials to produce a composition in a single dosage form will vary depending upon the host treated, the particular mode of administration. Provided compositions should be formulated so that a dosage of between 0.01-100 mg/kg body weight/day of the inhibitor can be administered to a patient receiving these compositions.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. Exemplary of such other therapeutic agents include corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal antiinflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, Prograf); cytotoxic drugs such as azathiprine and cyclophosphamide; TNF-α inhibitors such as tenidap, anti-TNF antibodies or soluble TNF receptor, and rapamycin (sirolimus or Rapamune) or derivatives thereof. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refer to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both a provided compound and additional therapeutic agent (in those compositions which comprise an additional therapeutic agent as described above) that may be combined with the carrier materials to produce a single dosage form will vary depending upon the host treated and the particular mode of administration. Compositions of this disclosure should be formulated so that a dosage of between 0.01-100 mg/kg body weight/day of a provided compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or disorder being treated. The amount of a compound of the present disclosure in the composition will also depend upon the particular compound in the composition.

### 5. USES OF COMPOUNDS

Compounds and compositions described herein are generally useful for the inhibition of PAD4.

The activity of a compound utilized in this disclosure as an inhibitor of PAD4, may be assayed in vitro, in vivo or in a cell line. In vitro assays include assays that determine the inhibition of PAD4. Detailed conditions for assaying a compound utilized in this disclosure as an inhibitor of PAD4 are set forth in the Examples below. In some embodiments, a provided compound inhibits PAD4 selectively as compared to PAD2.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of PAD4 and are therefore useful for treating one or more diseases or disorders associated with PAD4 enzyme activity. Thus, in certain embodiments, the present disclosure provides a method for treating a disease or a disorder associated with PAD4 enzyme activity, comprising the step of administering to a patient in need thereof a compound of the present disclosure, or a pharmaceutically acceptable composition thereof.

In one embodiment, a disease or a disorder associated with PAD4 enzyme activity is a disease, condition, or disorder mediated by inappropriate PAD4 activity. In some embodiments, a disease or a disorder associated with PAD4 enzyme activity is selected from the group consisting of rheumatoid arthritis, vasculitis, systemic lupus erythematosus, ulcerative colitis, cancer, cystic fibrosis, asthma, cutaneous lupus erythematosus, and psoriasis. In a further embodiment, the disease or a disorder associated with PAD4 enzyme activity is rheumatoid arthritis. In a further embodiment, the disease or a disorder associated with PAD4 enzyme activity is systemic lupus. In a further embodiment, the disease or a disorder associated with PAD4 enzyme activity is vasculitis. In a further embodiment, the disease or a disorder associated with PAD4 enzyme activity cutaneous lupus erythematosus. In a further embodiment, the disease or a disorder associated with PAD4 enzyme activity is psoriasis.

In one embodiment there is provided a method of treatment of rheumatoid arthritis, vasculitis, systemic lupus erythematosus, ulcerative colitis, cancer, cystic fibrosis, asthma, cutaneous lupus erythematosus, or psoriasis, which method comprises administering to a human subject in need thereof, a therapeutically effective amount of a provided compound or a stereoisomer, an enantiomer, a diastereomer, a tautomer, or a pharmaceutically acceptable salt thereof.

In one embodiment there is provided a method of treatment of rheumatoid arthritis, which method comprises administering to a human subject in need thereof, a therapeutically effective amount of a provided compound, a stereoisomer, an enantiomer, a diastereomer, a tautomer, or a pharmaceutically acceptable salt thereof. In one embodiment there is provided a method of treatment of systemic lupus, which method comprises administering to a human subject in need thereof, a therapeutically effective amount of a provided compound, a stereoisomer, an enantiomer, a diastereomer, a tautomer, or a pharmaceutically acceptable salt thereof. In one embodiment there is provided a method of treatment of vasculitis, which method comprises administering to a human subject in need thereof, a therapeutically effective amount of a provided compound, a stereoisomer, an enantiomer, a diastereomer, a tautomer, or a pharmaceutically acceptable salt thereof. In one embodiment there is provided a method of treatment of cutaneous lupus erythematosus, which method comprises administering to a human subject in need thereof, a therapeutically effective amount of a provided compound, a stereoisomer, an enantiomer, a diastereomer, a tautomer, or a pharmaceutically acceptable salt thereof. In one embodiment there is provided a method of treatment of psoriasis, which method comprises administering to a human subject in need thereof, a therapeutically effective amount of a provided compound, a stereoisomer, an enantiomer, a diastereomer, a tautomer, or a pharmaceutically acceptable salt thereof.

In some embodiments, a disease or a disorder associated with PAD4 enzyme activity is selected from the group consisting of acid-induced lung injury, acne (PAPA), acute lymphocytic leukemia, acute respiratory distress syndrome, Addison's disease, adrenal hyperplasia, adrenocortical insufficiency, ageing, AIDS, alcoholic hepatitis, alcoholic liver disease, allergen induced asthma, allergic bronchopulmonary, aspergillosis, allergic conjunctivitis, alopecia, Alzheimer's disease, amyloidosis, amyotropic lateral sclerosis, weight loss, angina pectoris, angioedema, anhidrotic ecodermal dysplasia-ID, ankylosing spondylitis, anterior segment, inflammation, antiphospholipid syndrome, aphthous stomatitis, appendicitis, arthritis, asthma, atherosclerosis, atopic dermatitis, autoimmune diseases, autoimmune hepatitis, bee sting-induced inflammation, Bechet's disease, Bechet's syndrome, Bells Palsey, berylliosis, Blau syndrome, bone pain, bronchiolitis, burns, bursitis, cancer, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic lymphocytic leukemia, chronic obstructive pulmonary disease, colitis, complex regional pain syndrome, connective tissue disease, corneal ulcer, crohn's disease, cryopyrin-associated periodic syndromes, cyrptococcosis, cystic fibrosis, deficiency of the interleukin-1-receptor antagonist (DIRA), dermatitis, dermatitis endotoxemia, dermatomyositis, diffuse intrinsic pontine glioma, endometriosis, endotoxemia, epicondylitis, erythroblastopenia, familial amyloidotic polyneuropathy, familial cold urticarial, familial Mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Hodgkin's disease, Huntington's disease, hyaline membrane disease, hyperammonemia, hypercalcemia, hypercholesterolemia, hyperimmunoglobulinemia D with recurrent fever (HIDS), hypoplastic and other anemias, hypoplastic anemia, idiopathic thrombocytopenic purpura, incontinentia pigmenti, infectious mononucleosis, inflammatory bowel disease, inflammatory lung disease, inflammatory neuropathy, inflammatory pain, insect bite-induced inflammation, iritis, irritant-induced inflammation, ischemia/reperfusion, juvenile rheumatoid arthritis, keratitis, kidney disease, kidney injury caused by parasitic infections, kidney injury caused by parasitic infections, kidney transplant rejection prophylaxis, leptospiriosis, leukemia, Loeffler's syndrome, lung injury, lupus, lupus nephritis, lymphoma, meningitis, mesothelioma, mixed connective tissue disease, Muckle-Wells syndrome (urticaria deafness amyloidosis), multiple sclerosis, muscle wasting, muscular dystrophy, myasthenia gravis, myocarditis, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osteoarthritis, otitis media, Paget's disease, pain, pancreatitis, Parkinson's disease, pemphigus, pericarditis, periodic fever, periodontitis, peritoneal endometriosis, pertussis, pharyngitis and adenitis (PFAPA syndrome), plant irritant-induced inflammation, pneumonia, pneumonitis, pneumosysts infection, poison ivy/urushiol oil-induced inflammation, polyarteritis nodosa, polychondritis, polycystic kidney disease, polymyositis, psoriasis, psychosocial stress diseases, pulmonary disease, pulmonary hypertension, pulmonary fibrosis, pyoderma gangrenosum, pyogenic sterile arthritis, renal disease, retinal disease, rheumatic carditis, rheumatic disease, rheumatoid arthritis, sarcoidosis, seborrhea, sepsis, severe pain, sickle cell, sickle cell anemia, silica-induced disease, Sjogren's syndrome, skin diseases, sleep apnea, solid tumors, spinal cord injury, Stevens-Johnson syndrome, stroke, subarachnoid hemorrhage, sunburn, temporal arteritis, tenosynovitis, thrombocytopenia, thyroiditis, tissue transplant, TNF receptor associated periodic syndrome (TRAPS), toxoplasmosis, transplant, traumatic brain injury, tuberculosis, type 1 diabetes, type 2 diabetes, ulcerative colitis, urticarial, uveitis, Wegener's granulomatosis, interstitial lung disease, psoriatic arthritis, juvenile idiopathic arthritis, Sjögren's syndrome, antineutrophil cytoplasmic antibody (ANCA)-associated vasculitis, antiphospholipid antibody syndrome, sepsis, deep vein thrombosis, fibrosis, Alzheimer's, scleroderma and CREST syndrome.

In one embodiment, the disclosure provides a compound, or a pharmaceutically acceptable salt thereof, for use in therapy. In another embodiment, the disclosure provides a compound, or a pharmaceutically acceptable salt thereof, for use in the treatment of a disease or a disorder mediated by inappropriate PAD4 activity. In another embodiment, the disclosure provides a compound, or a stereoisomer, an enantiomer, a diastereomer, a tautomer, or a pharmaceutically acceptable salt thereof, for use in the treatment of rheumatoid arthritis, vasculitis, systemic lupus erythematosus, ulcerative colitis, cancer, cystic fibrosis, asthma, cutaneous lupus erythematosus, or psoriasis. In another embodiment, the disclosure provides a compound, or a stereoisomer, an enantiomer, a diastereomer, a tautomer, or a pharmaceutically acceptable salt thereof, for use in the treatment of rheumatoid arthritis. In another embodiment, the disclosure provides a compound, or a stereoisomer, an enantiomer, a diastereomer, a tautomer, or a pharmaceutically acceptable salt thereof, for use in the treatment of systemic lupus. In another embodiment, the disclosure provides a compound, or a stereoisomer, an enantiomer, a diastereomer, a tautomer, or a pharmaceutically acceptable salt thereof, for use in the treatment of vasculitis. In another embodiment, the disclosure provides a compound, or a stereoisomer, an enantiomer, a diastereomer, a tautomer, or a pharmaceutically acceptable salt thereof, for use in the treatment of cutaneous lupus erythematosus. In another embodiment, the disclosure provides a compound, or a stereoisomer, an enantiomer, a diastereomer, a tautomer, or a pharmaceutically acceptable salt thereof, for use in the treatment of psoriasis. In another embodiment, the disclosure provides the use of a compound, or a stereoisomer, an enantiomer, a diastereomer, a tautomer, or a pharmaceutically acceptable salt thereof, in the manufacture of a medicament for use in the treatment of a disorder mediated by inappropriate PAD4 activity. In another embodiment, the disclosure provides the use of a compound, or a stereoisomer, an enantiomer, a diastereomer, a tautomer, or a pharmaceutically acceptable salt thereof, in the manufacture of a medicament for use in the treatment of rheumatoid arthritis, vasculitis, systemic lupus erythematosus, ulcerative colitis, cancer, cystic fibrosis, asthma, cutaneous lupus erythematosus, or psoriasis. In another embodiment, the disclosure provides the use of a compound, or a stereoisomer, an enantiomer, a diastereomer, a tautomer, or a pharmaceutically acceptable salt thereof, in the manufacture of a medicament for use in the treatment of rheumatoid arthritis. In another embodiment, the disclosure provides the use of a compound, or a stereoisomer, an enantiomer, a diastereomer, a tautomer, or a pharmaceutically acceptable salt thereof, in the manufacture of a medicament for use in the treatment of systemic lupus. In another embodiment, the disclosure provides the use of a compound, or a stereoisomer, an enantiomer, a diastereomer, a tautomer, or a pharmaceutically acceptable salt thereof, in the manufacture of a medicament for use in the treatment of vasculitis. In another embodiment, the in disclosure vention provides the use of a compound, or a stereoisomer, an enantiomer, a diastereomer, a tautomer, or a pharmaceutically acceptable salt thereof, in the manufacture of a medicament for use in the treatment of cutaneous lupus erythematosus. In another embodiment, the disclosure provides the use of a compound, or a stereoisomer, an enantiomer, a diastereomer, a tautomer, or a pharmaceutically acceptable salt thereof, in the manufacture of a medicament for use in the treatment of psoriasis. In a further embodiment, the disclosure provides a pharmaceutical composition for the treatment or prophylaxis of a disease or a disorder mediated by inappropriate PAD4 activity comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of rheumatoid arthritis, vasculitis, systemic lupus erythematosus, ulcerative colitis, cancer, cystic fibrosis, asthma, cutaneous lupus erythematosus, or psoriasis, comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of rheumatoid arthritis comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of systemic lupus comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of vasculitis comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of cutaneous lupus erythematosus comprising a provided compound, or a stereoisomer, an enantiomer, a diastereomer, a tautomer, or a pharmaceutically acceptable salt thereof. In a further embodiment, the disclosure provides a pharmaceutical composition for the treatment or prophylaxis of psoriasis comprising a provided compound, or a stereoisomer, an enantiomer, a diastereomer, a tautomer, or a pharmaceutically acceptable salt thereof.

All features of each of the aspects of the invention apply to all other aspects mutatis mutandis. In order that the invention described herein may be more fully understood, the following examples are set forth. It should be understood that these examples are for illustrative purposes only and are not to be construed as limiting this invention in any manner.

### 6. EXAMPLES

The following Examples have been prepared, isolated and characterized using the methods disclosed herein. The following examples demonstrate a partial scope of the disclosure and are not meant to be limiting of the scope of the disclosure.

### Examples

Abbreviations

| | |
|---|---|
| AcOH or HOAc | acetic acid |
| ACN, MeCN or CH₃CN | acetonitrile |
| Bn | benzyl |
| Boc | tert-butyloxycarbonyl |
| Cbz | carbobenzyloxy |
| CDCl₃ | deutero-chloroform |
| CD₃OD or Methanol-d₄ | deutero-methanol |
| CCl₄ | carbontetrachloride |
| CH₂Cl₂ | dichloromethane |
| CH₃CN | acetonitrile |
| CHCl₃ | chloroform |
| Cs₂CO₃ | cesium carbonate |
| DCM | dichloromethane |
| DEA | diethanolamine |
| DIEA, DIPEA or Hunig's | diisopropylethylamine |
| base | |
| DMF | dimethyl formamide |
| DMSO | dimethyl sulfoxide |
| DMSO-d₆ | hexadeutero-dimethylsulfoxide |
| DTBAD | di-tert-butyl azodicarboxylate |
| ESI | electrospray ionization |
| Et₃N or TEA | triethylamine |
| Et₂O | diethyl ether |
| EtOAc | ethyl acetate |
| EtOH | ethanol |
| HATU | 1-[Bis(dimethylamino)methylene]- |
| | 1H-1,2,3-triazolo[4,5-b]pyridinium |
| | 3-oxid hexafluorophosphate |
| HCl | hydrochloric acid |
| HPLC | high-performance liquid chromatography |
| IPA | isopropyl alcohol |
| Ir(dF(CF₃)ppy)2(dtbbpy)PF₆ | (4,4′-Di-t-butyl-2,2′-bipyridine)bis[3,5- |
| | difluoro-2-(5-trifluoromethyl-2-pyridinyl- |
| | kN)phenyl-kC]iridum(III) |
| | hexafluorophosphate |
| H₂SO₄ | sulfuric acid |
| K₂CO₃ | potassium carbonate |
| KNO₃ | potassium nitrate |
| LCMS | liquid chromatography mass spectrometry |
| LiAlH₄ | lithium aluminiumhydride |
| LiBH₄ | lithium borohydride |
| MeOH or CH₃OH | methanol |
| MgSO4 | magnesium sulfate |
| MHz | megahertz |
| mL | mililiter |
| mm | milimeter |
| mM | milimolar |
| μm or uM | micrometer |
| MnO₂ | manganese dioxide |
| min or min. | minutes |
| MS | mass spectrometry |
| MsOH or MSA | methylsulfonic acid |
| MsCl | methanesulfonyl chloride |
| NaCl | sodium chloride |
| Na₂CO₃ | sodium carbonate |
| NaH | sodium hydride |
| NaHCO₃ | sodium bicarbonate |
| NaIO₄ | sodium periodate |
| NaOH | sodium hydroxide |
| Na₂SO₄ | sodium sulfate |
| Na₂S₂O₄ | sodium thionate |
| NH₃ | ammonia |
| NH₄Cl | ammonium chloride |
| NH₄OAc | ammonium acetate |
| nm | nanometer |
| Pd/C | palladium on carbon |
| Pd(OAc)₂ | palladium(II) acetate |
| Pd(dppf)Cl₂ | [1,1′-Bis(diphenylphosphino)ferro- |
| | cene]palladium(II) dichloride |
| Pd(PPh₃)₄ | tetrakis(triphenylphosphine)palladium(0) |
| PPh₃ | triphenylphosphine |
| i-PrOH or IPA | isopropanol |
| Rt or RT | retention time |
| RuCl₃ | ruthenium trichloride |
| SFC | supercritical fluid chromatography |
| TBAI | tetrabutylammonium iodide |
| TBAF | tetrabutylammonium fluoride |
| TBME or MTBE | tert-butyl methyl ether |
| TBSCl | tert-butyldiemthylsilyl chloride |
| TsCl | 4-toluenensulfonyl chloride |
| THF | tetrahydrofuran |
| UV | ultraviolet |

### Description of Prep HPLC and Analytical LCMS Methods:

Method A: Prep HPLC. Column: XBridge C18 OBD Prep, 100 Å, 10 μm, 19 mm×250 mm; mobile phase A: water+0.05% TFA, mobile phase B: ACN; flow rate: 25 mL/min.; gradient: 35% B to 60% B in 4.8 min; detection: UV (210/254 nm).

Method B: Analytical LCMS. Column: Shim-pack XR-ODS, 3×50 mm, 2.2 μm; mobile phase A: water+0.05% TFA, mobile phase B: ACN+0.05% TFA; flow rate: 1.2 mL/min; gradient: 5% B to 95% B in 2 min, hold at 95% for 0.7 min, 95% B to 5% B in 0.05 min; detection: MS and UV (254 nm).

Method C: Analytical LCMS. Column: L-column3 C18, 3.0 mm×30 mm, 2.0 m particles; mobile phase A: water+5 mM ammonium bicarbonate, mobile phase B: ACN; flow rate: 1.5 mL/min; gradient: 10% B to 95% B over 1.2 min, then a 0.60 min hold at 95% B; detection: UV (254/220 nm).

Method D: Analytical LCMS. Column: HALO C18, 3×30 mm, 2.7 um; mobile phase A: water+0.05% TFA, mobile phase B: acetonitrile+0.05% TFA; flow rate: 1.5 mL/min; gradient: 5% B to 95% B in 2.5 min, hold at 95% for 1 min, 95% B to 5% B in 0.05 min.; detection MS and UV (254 nm).

Method E: Prep HPLC. Column: XSelect CSH Prep C18 OBD, 19×250 mm, 5 um; mobile phase A: water(0.05% TFA), mobile phase B: ACN; flow rate: 25 mL/min; gradient: 22% B to 50% B in 5.5 min; detection: UV (254 nm).

Method F: Prep HPLC. Column: SunFire C18 OBD Prep 19×250 mm, 5 μm; mobile phase A: water (0.05% TFA), mobile phase B: ACN; flow rate: 20 mL/min; gradient: 30% B to 55% B in 5.5 min; detection: UV (254 nm).

Method G: Prep HPLC. Column: Atlantis Prep T3 OBD Column, 19×250 mm 10 um; mobile phase A: water+0.05% TFA, mobile phase B: ACN; flow rate: 20 mL/min.; gradient: 20% B to 50% B in 6 min., 50% B; detection: UV (210/254 nm).

Method H: Analytical LCMS. Column: YMCMeteoricCore C18 BIO Column 3×50 mm, 2.6 μm; mobile phase A: Water+5 mM NH₄HCO₃, mobile phase B: ACN; flow rate: 1.2 mL/min.; gradient: 10% B to 95% B in 1.20 min., hold at 95% for 0.68 min., 95% B to 10% B in 0.05 min; detection: UV (254 nm).

Method I: Analytical LCMS. Column: HALO C18, 3×30 mm, 2.7 mm; mobile phase A: water+0.05% TFA, mobile phase B: acetonitrile+0.05% TFA; flow rate: 1.5000 mL/min; gradient: 5% B to 100% B in 1.3 min, hold at 100% for 0.5 min, 100% B to 5% B in 0.03 min; Detection: UV (254 nm).

Method J: Column: Waters BEH C18, 2.1×50 mm, 1.7 μm; mobile phase A: 95%/5% water/ACN+10 mMol Ammonium Acetate, mobile phase B: 5%/95% acetonitrile: water+10 mM Ammonium Acetate; flow rate: 0.8 mL/min.; gradient: 5% B to 95% B in 1.5 min., hold at 95% for 0.50 min., 95% B to 5% B in 0.1 min.; detection: PDA (210 to 400 nm)/MS (Total Ion Count positive/negative modes).

Method K: Column: Waters BEH C18 Column 2.1×50 mm, 1.7 m; mobile phase A: water+0.1% formic acid (v/v), mobile phase B: acetonitrile+0.1% formic acid (v/v); flow rate: 0.8 mL/min.; gradient: 5% B to 95% B in 1.5 min., hold at 95% for 0.50 min., 95% B to 5% B in 0.1 min.; detection: PDA (210 to 400 nm)/MS (Total Ion Count positive/negative modes).

### Synthetic Examples

General Procedure 1: Lactam Alkylation with Cyclic Sulfamidates

To a stirred solution of lactam (1.0 equiv) in DMF (0.05 M) were added NaH (60%) (2.0 equiv) and cyclicsulfamidate (1.4 equiv) at 0° C. The reaction was allowed to warm to room temperature for 2 h. TLC showed the reaction was complete, and the mixture was quenched with water, extracted with ethyl acetate (×3). The organic layer was washed with brine (×2), concentrated and purified by column chromatography to obtain the desired product.

General Procedure 2: N-Boc Deprotection with TFA

Stirred solution of N-Boc amine (1.0 equiv)) in DCM: TFA (3:1, 0.02 M) at room temperature for 30 min. TLC showed the reaction was complete, and the mixture was concentrated. The product was purified by Prep HPLC or column chromatography.

General Procedure 3: Ester or Acid Reduction with Lithium Aluminium Hydride

To a stirred solution of acid or ester (1.0 equiv) in THE (0.3 M) were added LiAlH₄ (2.0 equiv) at 0° C. The resulting solution was stirred at room temperature for 1 h. The reaction was monitored by LCMS. The reaction was then quenched by water (0.5 mL)/NaOH (15%) (1:4 ratio) and stirred at room temperature for 20 min. Solids were filtered out and the resulting solution concentrated under vacuum. The crude product was purified by column chromatography.

General Procedure 4: Alcohol Oxidation with Manganese Dioxide

To a stirred solution of alcohol (1.0 equiv) in DCM (0.1 M) was added MnO₂ (10 equiv) at room temperature under nitrogen atmosphere. The resulting mixture was stirred at room temperature for 12 h. LCMS showed the reaction was complete, and the solids were filtered out. The resulting mixture was concentrated to afford crude product, which was used without further purification or purified by column chromatography.

General Procedure 5: Alkylation of Heterocycle N—H or Phenol O—H with Cesium Carbonate Base

To a stirred solution of substituted indole (1.0 equiv) 250 mg, 1.47 mmol) in DMF (0.2 M) was added Cs₂CO₃ (3.0 equiv), TBAI (0.1 equiv) and alkyl electrophile (1.5 equiv) at room temperature and was warmed to 50° C. for 1.5 h. TLC showed the reaction was complete, and the mixture was quenched with water (20 mL), extracted with ethyl acetate (×3). The organic layer was washed with brine (×2), dried over sodium sulfate, concentrated, and purified by column chromatography.

General Procedure 6: Cyclization to Form Tricyclic Benzimidazoles from Nitro-Aniline

To a stirred solution of aldehyde (1.0 equiv) in ethanol:water (2:1, 0.08 M) were added aniline (1.0 equiv) and Na₂S₂O₄ (3.0 equiv) at room temperature and was warmed to 90° C. for 2 h. TLC showed the reaction was complete. The reaction mixture was concentrated under the vacuum to clean up ethanol and was diluted with water (20 mL), then extracted with DCM/MeOH(10:1) (×3). The organic layer was dried over sodium sulfate, concentrated, and purified by Prep-TLC or column chromatography.

General Procedure 7: Alkylation of Indole N—H with Sodium Hydride Base

To a stirred solution of substituted indole (1.0 equiv) in DMF (0.15 M) was added NaH (1.5 equiv) in portions at 0° C. and was warmed to room temperature under nitrogen atmosphere. The resulting mixture was stirred at room temperature for 0.5 h. Then was added alkyl electrophile (1.5 equiv) in portions at 0° C. and was warmed to room temperature under nitrogen atmosphere. The resulting mixture was stirred at room temperature for 2 h. The reaction was monitored by TLC or LCMS. The reaction was then quenched by adding water and extracted with ethyl acetate (×2). The combined organic extracts were washed with brine, dried over anhydrous sodium sulfate, and concentrated under vacuum. The crude product was purified by column chromatography.

General Procedure 8. Deprotection of N-Sulfonic Acid with HCl

To a stirred solution of the N-sulfonic acid (60 mg, 0.09 mmol) in THF (2 mL) was added 2M HCl (1 mL). The mixture was stirred at 50° C. for 30 min. LCMS showed the reaction was complete. The final compound was purified as described in the examples.

### General Procedure 9: Benzyloxy Ether Deprotection Via Hydrogenation

To a stirred solution of benzyloxy ether (1.0 equiv) in solvent mixture (0.02-0.1 M) was added 10% Pd/C (1.0 equiv) in portions at room temperature under nitrogen atmosphere. The resulting solution was stirred at room temperature overnight under a H₂ atmosphere. The reaction was monitored by TLC and LCMS. The solids were filtered out and the resulting mixture was concentrated. The crude product was used without further purification or purified by column chromatography.

### General Procedure 10: Mitsunobu Reaction

To a solution of the phenol (2.1 g, 6.33 mmol, 1.0 equiv)) and the alcohol (3.0 equiv) in toluene (0.15M), was added PPh₃ (2.0 equiv)) and DTBAD (2.0 equiv) at room temperature under nitrogen atmosphere. The resulting mixture was stirred at 80° C. for 6 h. The reaction was monitored by TLC and LCMS. The reaction was quenched by adding water and extracted with ethyl acetate (×2). The combined organic extracts were washed with water and brine, dried over anhydrous sodium sulfate, and concentrated under vacuum. The crude product was purified as described in the individual procedures.

General Procedure 11: Cyclization to Form Tricyclic Benzimidazoles from Dianiline

To a solution of the aldehyde (1 equiv) and the dianiline (1 equiv) in DMF:water (30:1, 0.25 M) was added oxone (4.6 g, 7.5 mmol). The resulting solution was stirred at room temperature for 16 h. The reaction was then quenched by adding water and extracted with ethyl acetate (×2). The combined organic extracts were water (×2) and washed with brine, dried over anhydrous sodium sulfate and concentrated under vacuum. The crude product was purified as described in the individual procedures.

### General Procedure 12: Phenol Alkylation Via Epoxide Opening

To a solution of the phenol (1 equiv) in ethanol (0.05-0.3 M) was added the epoxide (3-10 equiv) and TEA (5 equiv). The mixture was irradiated with microwave radiation at 100° C. for 2 h. The reaction was monitored by LCMS. The mixture was concentrated under vacuum. The crude product was purified as described in the individual procedures.

General Procedure 13: Alcohol Activation with Sulfonyl Chloride

To a solution of alcohol (x equiv) in DCM (0.01-0.2 M) was added TEA (2-4 equiv) and sulfonyl chloride (1.2-2.0 equiv) at 0° C. The resulting solution was stirred at room temperature for 1-2 h. The reaction was monitored by LCMS. The reaction was then quenched by adding saturated aqueous NH₄C1 at 0° C. and extracted with DCM (×3). The combined organic extracts were washed with brine, dried over anhydrous sodium sulfate, and concentrated under vacuum at 0° C. to afford the title compound.

General Procedure 14: Alkylation of Imidazole NH with Cesium Carbonate Base

To a solution of substituted indole (1 equiv) in DMF (0.1 M) was added imidazole reagent (1-9 equiv) and cesium carbonate (3 equiv). The reaction was stirred at 50° C. for 1-16 h. The reacted was monitored by LCMS. The mixture was quenched with water, extracted with ethyl acetate (×2). The organic laywer was washed with water (×2) and brine (×2), dried over anhydrous sodium sulfate and concentrated under vacuum. The crude product was purified as described in the individual procedures.

### Intermediate 1. tert-butyl (S)-4-(fluoromethyl)-1,2,3-oxathiazolidine-3-carboxylate 2,2-dioxide

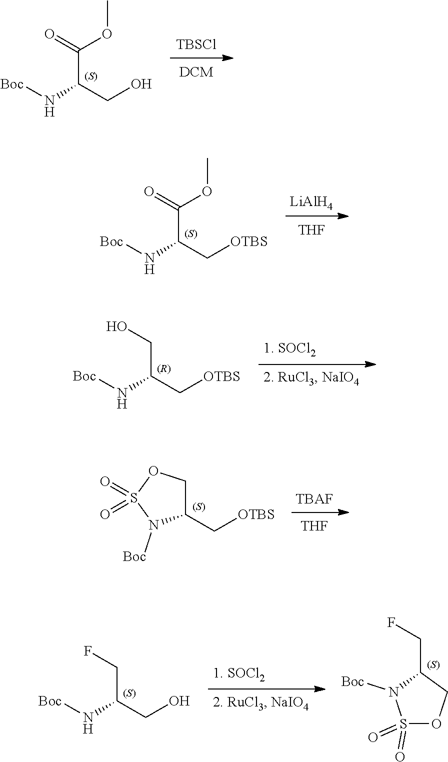

### Step 1: Synthesis of (S)-methyl 2-((tert-butoxycarbonyl)amino)-3-((tert-butyldimethylsilyl)oxy)propanoate

To a solution of methyl (2S)-2-(tert-butoxycarbonylamino)-3-hydroxy-propanoate (90 g, 410 mmol) and imidazole (30.7 g, 452 mmol) in DCM (900 mL) was added TBSCl (80.4 g, 534 mmol) at 0° C. After addition, the reaction mixture was stirred at 20° C. for 2 hrs. TLC showed starting material was consumed completely. The reaction was quenched with water (1 L) at 0° C. and extracted with DCM (800 mL×3). Combined organic layers were washed with brine (1 L×2), dried over anhydrous Na₂SO₄, filtered and concentrated to give the title compound (140 g, crude) as alight yellow oil. ¹H NMR (400 MHz, CDCl₃) δ: 5.34 (d, J=8.4 Hz, 1H), 4.36 (d, J=8.8 Hz, 1H), 4.06 (d, J=2.4 Hz, 1H), 4.03 (d, J=2.4 Hz, 1H), 3.75 (s, 3H), 1.46 (s, 9H), 0.87 (s, 9H), 0.03 (d, J=5.2 Hz, 6H).

### Step 2: Synthesis of tert-butyl N-[(1R)-1-[[tert-butyl(dimethyl)silyl]oxymethyl]-2-hydroxy-ethyl]carbamate

To a suspension of LiAlH₄ (27.9 g, 734 mmol) in THE (1.2 L) was added a solution of (S)-methyl 2-((tert-butoxycarbonyl)amino)-3-((tert-butyldimethylsilyl)oxy)propanoate (144 g, 432 mmol) in THF (300 mL) at 0° C. After addition, then reaction mixture was stirred at 0° C. for 1 hr. TLC showed starting material was consumed completely. The reaction was quenched with saturated aqueous NH₄C1 (800 mL) and filtered. Filtrate was added ethyl acetate (800 mL×2) and washed with brine (800 mL), dried over anhydrous Na₂SO₄. The mixture was filtered and concentrated to give the title compound (110 g, crude) as light yellow oil. ¹H NMR (400 MHz, CDCl₃) δ: 5.06 (s, 1H), 3.75-3.58 (m, 4H), 2.70-2.61 (m, 1H), 1.38 (s, 9H), 0.82 (s, 9H), 0.00 (s, 6H).

### Step 3: Synthesis of tert-butyl (3R)-4-[[tert-butyl(dimethyl)silyl]oxymethyl]-2-oxo-oxathiazolidine-3-carboxylate

To a solution of imidazole (147 g, 2.16 mol) in DCM (700 mL) was added a solution of SOCl₂ (77.1 g, 648 mmol) in DCM (400 mL) at 0° C. After addition, the reaction mixture was stirred at 18° C. for 1 hr. Then a solution of tert-butyl N-[(1R)-1-[[tert-butyl(dimethyl)silyl]oxymethyl]-2-hydroxy-ethyl]carbamate (110 g, 360 mmol) in DCM (600 mL) was added to the reaction at −10° C. After addition, the reaction mixture was stirred at 18° C. for 1 hr. TLC showed starting material was consumed completely. The reaction was quenched with aqueous citric acid (10%, 800 mL) and extracted with DCM (1 L×2). Combined organic layers were washed with water (1 L×2), dried over anhydrous Na₂SO₄, filtered and concentrated to give the title compound (104 g, crude) as light yellow oil. ¹H NMR (400 MHz, CDCl₃) δ: 5.00-4.98 (m, 1H), 4.83-4.72 (m, 2H), 4.07-4.03 (m, 1H), 3.79-3.72 (m, 1H), 1.53 (s, 9H), 0.89 (s, 9H), 0.07 (s, 6H).

### Step 4. Synthesis of tert-butyl (3R)-4-[[tert-butyl(dimethyl)silyl]oxymethyl]-2,2-dioxo-oxathiazolidine-3-carboxylate

To a solution of tert-butyl (3R)-4-[[tert-butyl(dimethyl)silyl]oxymethyl]-2-oxo-oxathiazolidine-3-carboxylate (52 g, 148 mmol) in MeCN (1000 mL) was added RuCl₃ (30.7 mg, 148 umol) and a solution of NaIO₄ (31.6 g, 148 mmol) in H₂O (500 mL) at 18° C. After addition, the rection mixture was stirred at 18° C. for 1 hr. TLC showed starting material was consumed completely. Two batches reaction was combined and diluted with water (1 L). The aqueous phase was extracted with DCM (1 L×2). The combined organic layer was washed with water (500 mL×2), dried over anhydrous Na₂SO₄ filtered and concentrated to the tile compound (80 g, crude) as a light yellow solid. ¹H NMR (400 MHz, CDCl₃) δ: 4.64-4.58 (m, 2H), 4.28-4.27 (m, 1H), 3.89-3.76 (m, 2H), 1.56 (s, 9H), 0.90 (s, 9H), 0.09 (s, 6H).

### Step 5: Synthesis of tert-butyl N-[(1S)-1-(fluoromethyl)-2-hydroxy-ethyl]carbamate

To a solution of tert-butyl (4S)-4-[[tert-butyl(dimethyl)silyl]oxymethyl]-2,2-dioxo-oxathiazolidine-3-carboxylate (85 g, 231 mmol) in THE (850 mL) was added TBAF (1 M, 277 mL) and stirred at 20° C. for 1 hr. TLC showed starting material was consumed completely. The reaction was quenched with saturated aqueous NH₄Cl (100 ml) and extracted with ethyl acetate (800 mL×2). Combined organic layers were washed with water (500 mL×2), dried over anhydrous Na₂SO₄, filtered and concentrated. The residue was purified by MPLC (petroleum ether/ethyl acetate=2/1) to give the title compound (20 g, 45% yield) as light yellow oil. ¹H NMR (400 MHz, CDCl₃) δ: 5.05 (d, J=7.6 Hz, 2H), 4.57-4.44 (m, 2H), 3.81-3.73 (m, 3H), 1.45 (s, 9H).

### Step 6: Synthesis of tert-butyl (4S)-4-(fluoromethyl)-2-oxo-oxathiazolidine-3-carboxylate

To a solution of imidazole (42.3 g, 621 mmol) in DCM (200 mL) was added a solution of SOCl₂ (22.2 g, 186 mmol) in DCM (200 mL) drop wise at 0° C., then the reaction mixture was stirred at 20° C. for 1 hr, then added tert-butyl N-[(1S)-1-(fluoromethyl)-2-hydroxy-ethyl]carbamate (20 g, 103 mmol) dissolved in DCM (200 mL) drop wise at −10° C., finally the reaction mixture was stirred at 20° C. for 1 hr. TLC showed starting material was consumed completely. The reaction was quenched with aqueous citric acid (10%) to pH=5 and extracted with DCM (300 mL×3). Combined organic layers were washed with brine (200 mL×2), dried over anhydrous Na₂SO₄, filtered and concentrated to give the title compound (22 g, crude) as yellow oil. ¹H NMR (400 MHz, CDCl₃) δ: 5.29-4.97 (m, 2H), 4.86-4.57 (m, 2H), 4.42-4.10 (m, 3H), 1.52 (s, 9H).

### Step 7: Synthesis of tert-butyl (S)-4-(fluoromethyl)-1,2,3-oxathiazolidine-3-carboxylate 2,2-dioxide

To a solution of tert-butyl (4S)-4-(fluoromethyl)-2-oxo-oxathiazolidine-3-carboxylate (22 g, 91.9 mmol) in MeCN (440 mL) was added RuCl₃ (191 mg, 919 umol), then added the solution of NaIO₄ (19.7 g, 91.9 mmol) in water (220 mL) drop wise under N₂. Finally the reaction mixture was stirred at 20° C. for 1 hrs. TLC showed the starting material was consumed completely and a new spot appeared. The reaction mixture was filtered, filter cake was washed with DCM (300 mL), the mixture was added water (300 mL) and DCM (800 mL), then separated, the combined organic layer was washed with water (300 mL×2), brine (200 mL×2), dried over anhydrous Na₂SO₄, filtered and concentrated to give the title compound (17.5 g, crude) as a yellow solid. ¹H NMR (400 MHz, CDCl₃) δ: 4.73-4.53 (m, 5H), 1.57 (s, 9H).

### Intermediate 2. tert-butyl (S)-(1-fluoro-3-(2-(methylamino)-3-nitro-5-oxo-7,8-dihydro-1,6-naphthyridin-6(5H)-yl)propan-2-yl)carbamate

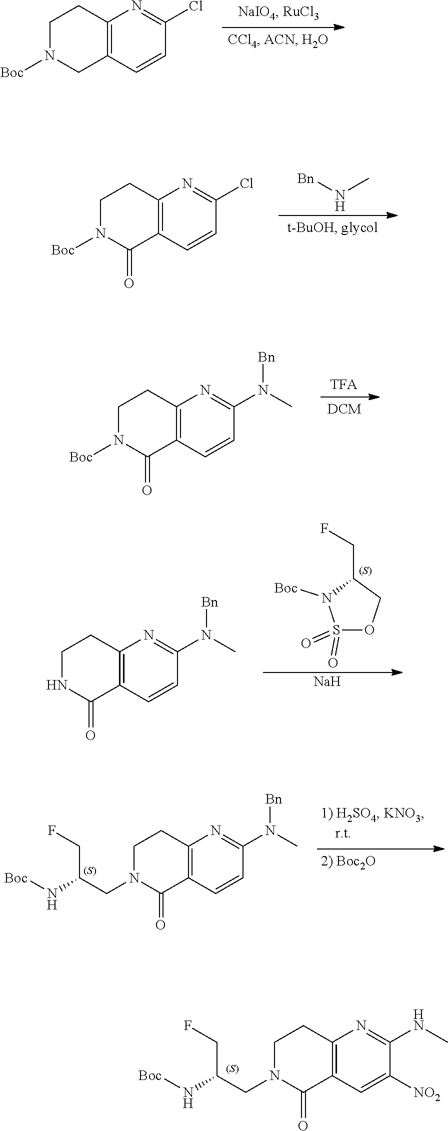

### Step 1: Synthesis of tert-butyl 2-chloro-5-oxo-7,8-dihydro-1,6-naphthyridine-6-carboxylate

Two batches run in parallel. To a solution of tert-butyl 2-chloro-7,8-dihydro-5H-1,6-naphthyridine-6-carboxylate (25 g, 93.0 mmol) in CCl₄ (250 mL) and MeCN (25 mL) was added the solution of NaIO₄ (59.7 g, 279 mmol) in water (175 mL), then added RuCl₃ (193 mg, 930 umol) under N₂. Finally the reaction mixture was stirred at 15° C. for 16 hrs. The two batches were combined; the mixture was filtered and concentrated to remove the solvent. The residue was added water (800 mL) and extracted with ethyl acetate (400 mL×3), the combined organic layers were washed with Na₂SO aq. (500 mL), brine (300 mL×2), dried over anhydrous Na₂SO₄, filtered and concentrated to give the title compound (40 g, crude) as a gray solid. ¹H NMR (400 MHz, DMSO-d₆) δ: 8.28 (d, J=8.0 Hz, 1H), 7.60 (d, J=8.4 Hz, 1H), 3.98 (t, J=6.0 Hz, 2H), 3.13 (t, J=6.4 Hz, 2H), 1.46 (s, 9H).

### Step 2: Synthesis of tert-butyl 2-[benzyl (methyl)amino]-5-oxo-7,8-dihydro-1,6-naphthyridine-6-carboxylate

Two batches run in parallel. To a solution of tert-butyl 2-chloro-5-oxo-7,8-dihydro-1,6-naphthyridine-6-carboxylate (20 g, 70.7 mmol) in tert-butanol (400 mL) and glycol (400 mL) was added N-methyl-1-phenyl-methanamine (42.9 g, 354 mmol), then the mixture was stirred at 50° C. for 16 hrs. TLC showed the starting material was consumed completely and a new spot appeared. The reaction mixture was concentrated to remove the t-BuOH. The residue was purified by silica gel column (petroleum ether: ethyl acetate=30:1 to 5:1) to give the title compound (40 g, 77% yield) as a yellow solid. ¹H NMR (400 MHz, DMSO-d₆) δ: 7.40 (d, J=9.2 Hz, 1H), 7.35-7.21 (m, 5H), 6.67 (d, J=9.2 Hz, 1H), 4.87 (s, 2H), 3.87 (t, J=6.0 Hz, 2H), 3.11 (s, 3H), 2.91 (t, J=6.0 Hz, 2H), 1.48 (s, 9H).

### Step 3: Synthesis of 2-[benzyl(methyl) amino]-7,8-dihydro-6H-1,6-naphthyridin-5-one

To a solution of tert-butyl 2-[benzyl(methyl)amino]-5-oxo-7,8-dihydro-1,6-naphthyridine-6-carboxylate (28 g, 76.2 mmol) in DCM (300 mL) was added TFA (158 g, 1.39 mol) drop wise at 0° C., then the mixture was stirred at 20° C. for 1 hrs. TLC showed the starting material was consumed completely and a new spot appeared. The reaction mixture was concentrated, the residue was diluted with DCM (200 mL), and was adjusted pH with Na₂CO₃ aq. to pH=8. The solution was extracted with DCM (200 mL×2) and washed with brine (400 mL). The combined organic layers were dried over anhydrous Na₂SO₄, filtered and concentrated to give the tile compound (17.5 g, 86% yield) as a yellow solid. ¹H NMR (400 MHz, DMSO-d₆) δ: 7.83 (d, J=8.8 Hz, 1H), 7.54 (s, 1H), 7.33-7.20 (m, 5H), 6.58 (d, J=8.8 Hz, 1H), 4.84 (s, 2H), 3.38-3.35 (m, 2H), 3.07 (s, 3H), 2.81 (t, J=6.8 Hz, 2H).

### Step 4: Synthesis of tert-butyl N-[(1S)-1-[[2-[benzyl(methyl)amino]-5-oxo-7,8-dihydro-1,6-naphthyridin-6-yl]methyl]-2-fluoro-ethyl]carbamate

Intermediate 1 (11 g, 43.1 mmol) was reacted with 2-[benzyl(methyl)amino]-7,8-dihydro-6H-1,6-naphthyridin-5-one (7.68 g, 28.7 mmol) according to General Procedure 1. The residue was purified by silica gel column (DCM:MeOH=50:1 to 20:1) to give the title compound (9.7 g, 76% yield) as a yellow oil. ¹H NMR (400 MHz, DMSO-d₆) δ: 7.87 (d, J=8.8 Hz, 1H), 7.33-7.29 (m, 2H), 7.24-7.19 (m, 3H), 6.94 (d, J=8.8 Hz, 1H), 6.25 (d, J=8.8 Hz, 1H), 4.86 (s, 2H), 4.50-4.31 (m, 2H), 3.59-3.54 (m, 4H), 3.08 (s, 3H), 1.32 (s, 9H).

### Step 5: Synthesis of tert-butyl (S)-(1-fluoro-3-(2-(methylamino)-3-nitro-5-oxo-7,8-dihydro-1,6-naphthyridin-6(5H)-yl)propan-2-yl)carbamate

Two batches were run in parallel. To a solution of tert-butyl N-[(1S)-1-[[2-[benzyl(methyl)amino]-5-oxo-7,8-dihydro-1,6-naphthyridin-6-yl]methyl]-2-fluoro-ethyl]carbamate (6 g, 13.6 mmol) in H₂SO₄ (110 g, 60 mL) was added KNO₃ (5.50 g, 54.4 mmol) drop wise at 0° C. then the mixture was stirred at 20° C. for 3 hrs. LCMS showed the starting material was consumed completely. The two batches were was powered into ice-water (1 L) and extracted with ethyl acetate (300 mL×2), aqueous phase was slowly and basified to pH=9˜10 using Na₂CO₃ followed by addition of THF (1.2 L), Boc₂O (3.55 g, 16.27 mmol) was added into this mixture and stirred at 25° C. for 16 hrs. LCMS showed the reaction was finished. The mixture was extracted with ethyl acetate (2 L×2), dried over anhydrous sodium sulfate, filtered and concentrated under reduced pressure to give a residue. The residue was purified by silica gel column (petroleum ether: ethyl acetate=10:1 to 1:1) to give the title compound (4.13 g, 38% yield) as a yellow solid. ¹H NMR (400 MHz, DMSO-d₆) δ: 8.86 (d, J=4.8 Hz, 1H), 8.65 (s, 1H), 6.98 (d, J=8.8 Hz, 1H), 4.45 (d, J=5.6 Hz, 1H), 4.35 (d, J=5.2 Hz, 1H), 3.68-3.32 (m, 4H), 3.08 (s, 3H), 3.07-3.02 (m, 2H), 1.31 (s, 9H).

### Intermediate 3. 2-(methylamino)-3-nitro-7,8-dihydro-1,6-naphthyridin-5(6H)-one

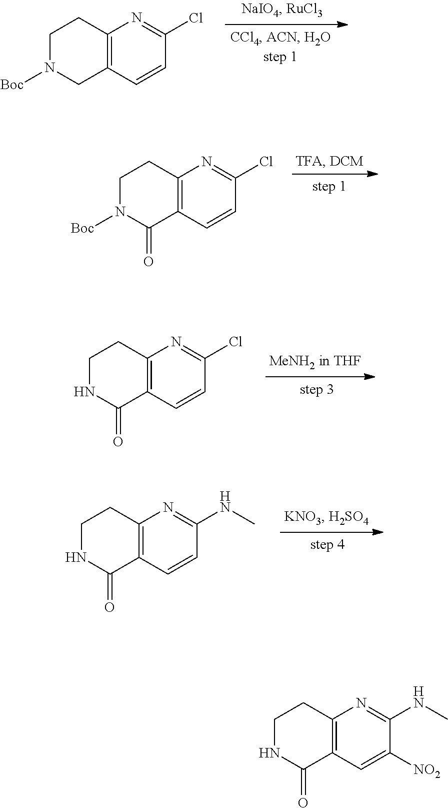 [SMILES: CC(C)(C)OC(=O)N1CCc2nc(Cl)ccc2C1 ; CC(C)(C)OC(=O)N1CCc2nc(Cl)ccc2C1=O ; O=C1NCCc2nc(Cl)ccc21 ; CNc1ccc2c(n1)CCNC2=O ; CNc1nc2c(cc1[N+](=O)[O-])C(=O)NCC2]

### Step 1: Synthesis of tert-butyl 2-chloro-5-oxo-7,8-dihydro-1,6-naphthyridine-6(5H)-carboxylate

To a stirred solution of tert-butyl 2-chloro-7,8-dihydro-5H-1,6-naphthyridine-6-carboxylate (500 mg, 1.86 mmol) in CCl₄ (7.5 mL) and MeCN (0.75 mL) were added NaIO₄ (1.2 g, 5.58 mmol) and Water (2.5 mL). RuCl₃·H₂O (126 mg, 0.56 mmol) was added followed, and the reaction mixture was stirred vigorously at room temperature for 2 hours. LCMS showed the reaction was complete, and the mixture was filtrated and extracted with DCM (3×20 mL). The organic was dried over anhydrous sodium sulfate and concentrated under vacuum. The residue was passed though silica gel (petroleum ether:ethyl acetate=2:1) to obtain the title compound (447 mg, 85%) as a white solid. LCMS (EST, m/z): 283 [M+H]⁺.

### Step 2: Synthesis of 2-chloro-7,8-dihydro-1,6-naphthyridin-5(6H)-one

Tert-butyl 2-chloro-5-oxo-7,8-dihydro-1,6-naphthyridine-6(5H)-carboxylate (100 mg, 0.35 mmol) was reacted according to General Procedure 2. The product was used in the next step directly without further purification. LCMS (ESI, m/z): 183 [M+H]*.

### Step 3: Synthesis of 2-(methylamino)-7,8-dihydro-1,6-naphthyridin-5(6H)-one

To a stirred solution of 2-chloro-7,8-dihydro-6H-1,6-naphthyridin-5-one (200 mg, 1.1 mmol) in Ethylene glycol (7 mL) and tert-butanol (7 mL) were added methylamine (2 M in THF) (2.8 mL, 5.48 mmol). The resulting mixture was stirred at 220° C. for 2 h. LCMS showed the reaction was complete, and the mixture was quenched with water (20 mL), extracted with ethyl acetate (3×20 mL). The organic layers was dried over anhydrous sodium sulfate and concentrated under vacuum. The residue was purified by silica gel column chromatography (petroleum ether:ethyl acetate=1:2) to obtain the title compound (170 mg, 88%) as a white solid. LCMS (ESI, m/z): 178 [M+H]⁺.

### Step 4: Synthesis of 2-(methylamino)-3-nitro-7,8-dihydro-1,6-naphthyridin-5(6H)-one

To a stirred solution of 2-(methylamino)-7,8-dihydro-6H-1,6-naphthyridin-5-one (170 mg, 0.96 mmol) in H₂SO₄ (2 mL) was added KNO₃ (194 mg, 1.92 mmol) at 0° C. The resulting solution was stirred at room temperature for 2 h. LCMS showed the reaction was complete, and the mixture was quenched with water (20 mL) at 0° C. The resulting solution was adjusted to pH 8˜9 with sodium bicarbonate, then extracted with ethyl acetate (3×30 mL). The organic layers was dried over anhydrous sodium sulfate and concentrated under vacuum. The residue was purified by silica gel column chromatography (petroleum ether: ethyl acetate=1:2) to obtain the title compound (200 mg, 94%) as a yellow solid. LCMS (ESI, m/z): 223 [M+H]⁺.

### Intermediate 4. (R)-tetrahydro-3H-[1,2,3]oxathiazolo[4,3-c][1,4]oxazine 1,1-dioxide

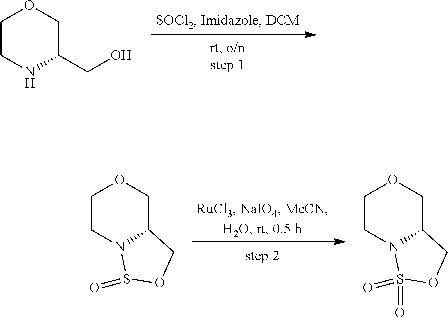

### Step 1: (3aR)-tetrahydro-3H-[1,2,3]oxathiazolo[4,3-c][1,4]oxazine 1-oxide

To a solution of imidazole (17.4 g, 256 mmol) in DCM (40 mL) was added the solution of SOCl₂ (9.14 g, 76.82 mmol) in DCM (80 mL) dropwise at room temperature under nitrogen atmosphere. The resulting mixture was stirred at room temperature for 1 h. To the above mixture was added the solution of (S)-morpholin-3-ylmethanol (5 g, 42.68 mmol) in DCM (80 mL) dropwise under nitrogen atmosphere. The resulting mixture was stirred at room temperature for overnight. The reaction was monitored by LCMS. The resulting mixture was diluted with water (200 mL) and acidated to pH 6 with citric acid. The reaction was then extracted with DCM (3×200 mL). The combined organic extracts were washed with brine (200 mL), dried over anhydrous sodium sulfate and concentrated under vacuum to afford the title compound (6 g, 86%) as a yellow oil. LCMS (ESI, m/z): 164 [M+H]+.

### Step 2: (R)-tetrahydro-3H-[1,2,3]oxathiazolo[4,3-c][1,4]oxazine 1,1-dioxide

To a solution of (3aR)-tetrahydro-3H-[1,2,3]oxathiazolo[4,3-c][1,4]oxazine 1-oxide (6 g, 36.77 mmol) and RuCl₃ (938 mg, 3.68 mmol) in MeCN (200 mL), was added the solution of NaIO₄ (8.65 g, 40.44 mmol) in water (200 mL) at room temperature under nitrogen atmosphere. The mixture was stirred at room temperature for 0.5 h under nitrogen atmosphere. The reaction was monitored by LCMS. The mixture solution was concentrated under vacuum. The crude product was extracted with ethyl acetate (2×300 mL). The combined organic extracts were washed with brine (300 mL), dried over anhydrous sodium sulfate, concentrated under vacuum. The crude product was purified by column chromatography (petroleum ether/ethyl acetate=1:1) to afford the title compound (3.2 g, 48%) as a white solid. LCMS (ESI, m/z): 180 [M+H]+.

### Intermediate 5. tert-butyl (S)-(1-(2-(1-(cyclopropylmethyl)-7-hydroxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

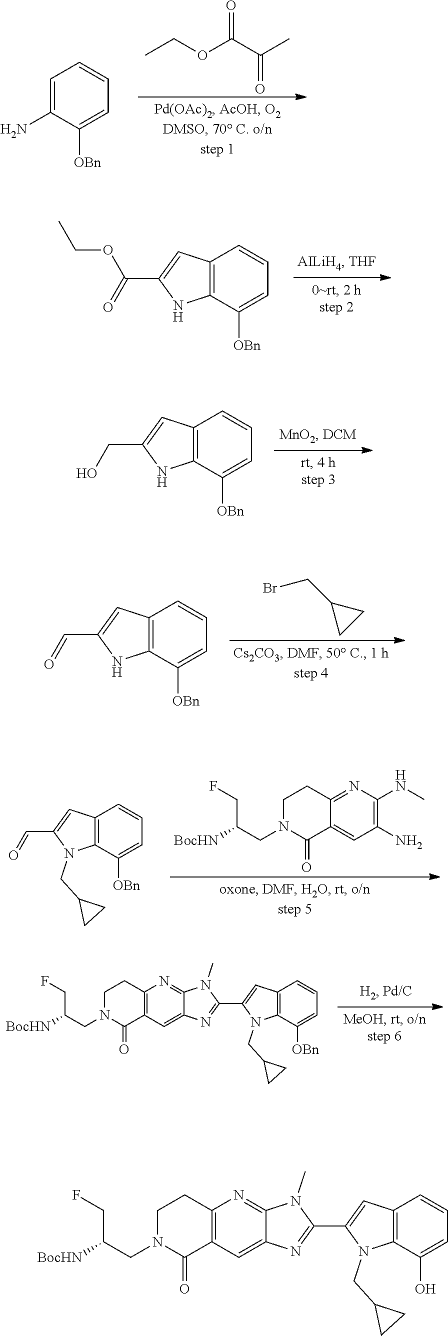

### Step 1: ethyl 7-benzyloxy-1H-indole-2-carboxylate

To a solution of 2-benzyloxyaniline (15 g, 75.28 mmol) and ethyl 2-oxopropanoate (19.23 g, 165.6 mmol) in DMSO (600 mL), was added AcOH (4.52 g, 75.3 mmol) and Pd(OAc)₂ (1.7 g, 7.5 mmol) under nitrogen atmosphere. The resulting solution was stirred at 70° C. for 16 h under oxygen atmosphere. The reaction was then quenched by adding water (1 L) and extracted with ethyl acetate (2×1 L), washed with water (2×1 L) and brine (2×1 L), dried over anhydrous sodium sulfate, and concentrated under vacuum. The crude product was purified by column chromatography (petroleum ether/ethyl acetate=10:1) to afford the title compound (8 g, 35.9%) as a light yellow solid. LCMS (ESI, m/z): 296 [M+H]+.

### Step 2: (7-(benzyloxy)-1H-indol-2-yl)methanol

Ethyl 7-benzyloxy-1H-indole-2-carboxylate (8 g, 27.1 mmol) was reacted according to General Procedure 3. The crude product was purified by column chromatography (petroleum ether/ethyl acetate=3:1) to afford the title compound (4.5 g, 62%) as a light yellow solid. LCMS (ESI, m/z): 254 [M+H]+.

### Step 3: 7-(benzyloxy)-1H-indole-2-carbaldehyde

(7-benzyloxy-1H-indol-2-yl)methanol (4.5 g, 17.7 mmol) was reacted according to General Procedure 4. The crude product was purified by column chromatography (petroleum ether/ethyl acetate=5:1) to afford the title compound (4 g, 90%) as a light yellow solid. LCMS (ESI, m/z): 252 [M+H]+.

### Step 4: 7-(benzyloxy)-1-(cyclopropylmethyl)-1H-indole-2-carbaldehyde

7-benzyloxy-1H-indole-2-carbaldehyde (4 g, 15.9 mmol) was reacted with Cs₂CO₃ (15.6 g, 47.7 mmol) and bromomethylcyclopropane (3.2 g, 23.9 mmol) according to General Procedure 5. The crude product was purified by column chromatography (petroleum ether/ethyl acetate=5:1) to afford the title compound (4.1 g, 84%) as a yellow solid. LCMS (ESI, m/z): 306 [M+H]+.

### Step 5: tert-butyl (S)-(1-(2-(7-(benzyloxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of 7-benzyloxy-1-(cyclopropylmethyl)indole-2-carbaldehyde (2.3 g, 7.5 mmol) and Intermediate 7 (3 g, 7.5 mmol) were reacted according to General Procedure 11. The crude product was purified by column chromatography (petroleum ether/ethyl acetate=1:1) to afford the title compound (2.4 g, 49%) as a light yellow solid. LCMS (ESI, m/z): 653 [M+H]+.

### Step 6: tert-butyl (S)-(1-(2-(1-(cyclopropylmethyl)-7-hydroxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of tert-butyl (S)-(1-(2-(7-(benzyloxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (2.4 g, 3.7 mmol) was reacted according to General Procedure 9. The crude product was purified by column chromatography (Petroleum ether/ethyl acetate=1:3) to afford the title compound (1.5 g, 73% yield) as a light brown solid. LCMS (ESI, m/z): 563 [M+H]+.

### Intermediate 6. 3-amino-2-(methylamino)-7,8-dihydro-1,6-naphthyridin-5(6H)-one

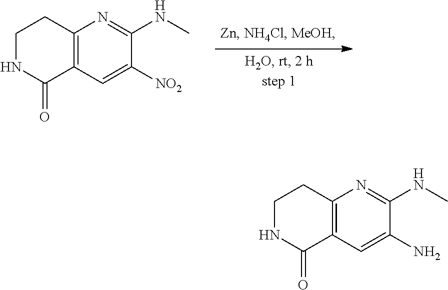 [SMILES: CNc1nc2c(cc1[N+](=O)[O-])C(=O)NCC2 ; CNc1nc2c(cc1N)C(=O)NCC2]

To a solution of Intermediate 3 (13 g, 58.5 mmol) in methanol (300 mL), was added a solution of NH₄Cl (31.3 g, 585 mmol) in water (100 mL). The mixture was added Zn (38 g, 585 mmol). The mixture was stirred at room temperature for 2 h. Solids were filtered out and the solvent was evaporated under vacuum. The crude product was purified by flash column chromatography on C18 silica to afford the title compound (3 g, 27%) as a yellow solid. LCMS (ESI, m/z): 193 [M+H]+.

### Intermediate 7. tert-butyl (S)-(1-(3-amino-2-(methylamino)-5-oxo-7,8-dihydro-1,6-naphthyridin-6(5H)-yl)-3-fluoropropan-2-yl)carbamate

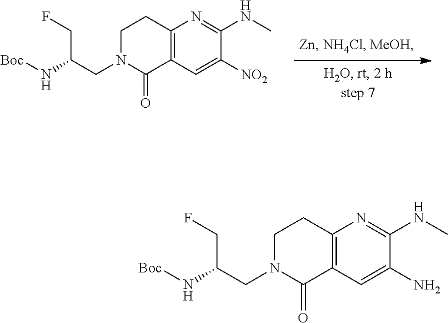 [SMILES: CNc1nc2c(cc1[N+](=O)[O-])C(=O)N(C[C@@H](CF)NC(=O)OC(C)(C)C)CC2 ; CNc1nc2c(cc1N)C(=O)N(C[C@@H](CF)NC(=O)OC(C)(C)C)CC2]

To a solution of tert-butyl (S)-(1-fluoro-3-(2-(methylamino)-3-nitro-5-oxo-7,8-dihydro-1,6-naphthyridin-6(5H)-yl)propan-2-yl)carbamate (10 g, 22.6 mmol) in methanol (200 mL), was added a solution of NH₄Cl (12.1 g, 226 mmol) in water (50 mL). The mixture was added Zn (14.5 g, 226 mmol) portion wise slowly. The mixture was stirred at room temperature for 2 h. Solids were filtered out and the solvent was evaporated under vacuum. The crude product was purified by flash column chromatography on C18 silica to afford the title compound (7.6 g, 92%) as a light yellow solid. LCMS (ESI, m/z): 368 [M+H]+.

### Example 1. (S)-7-(2-amino-3-fluoropropyl)-2-(1-(cyclopropylmethyl)-7-methoxy-1H-indol-2-yl)-3-methyl-3,5,6,7-tetrahydro-8H-imidazo[4,5-b][1,6]naphthyridin-8-one

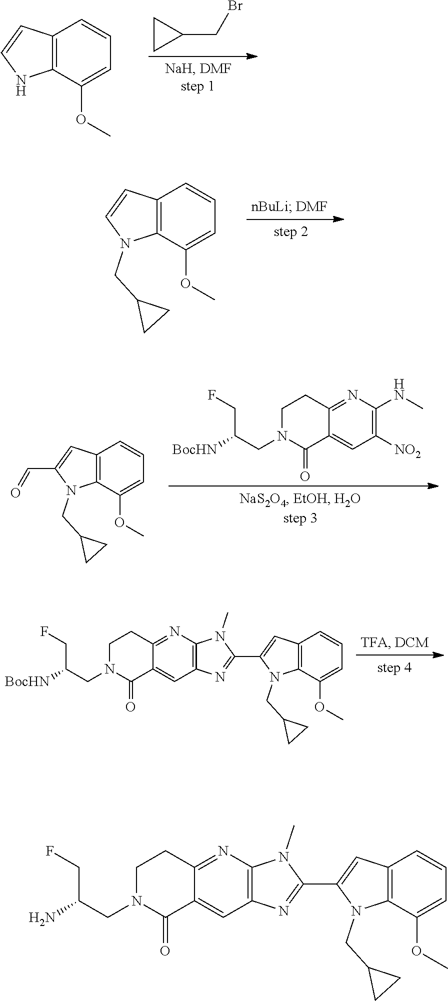

### Step 1: Synthesis of 1-(cyclopropylmethyl)-7-methoxy-1H-indole

7-methoxy-1H-indole (1 g, 6.79 mmol) was reacted with bromomethylcyclopropane (1.3 g, 10.19 mmol) according to General Procedure 7. The crude product was purified by column chromatography (petroleum ether/ethyl acetate=100:1) to afford 1-(cyclopropylmethyl)-7-methoxy-indole (1.35 g, 99%) as a yellow oil. LCMS (ESI, m/z): 202 [M+H]+.

### Step 2: Synthesis of 1-(cyclopropylmethyl)-7-methoxy-1H-indole-2-carbaldehyde

To a solution of 1-(cyclopropylmethyl)-7-methoxy-indole (500 mg, 2.48 mmol) in THE (20 mL) was added n-butyllithium (2.5 M in n-hexane) (1.97 mL, 4.97 mmol) in portions at −78° C. under nitrogen atmosphere. The mixture was stirred at room temperature for 1 h. To the above mixture was added DMF (0.38 mL, 4.97 mmol) drop wise at −78° C. under nitrogen atmosphere. The mixture was stirred at room temperature for 1 h. The reaction was monitored by LCMS. The reaction was then quenched by adding saturated aqueous NH₄Cl (20 mL) at −58° C., extracted with ethyl acetate (2×20 mL). The combined organic extracts were washed with brine (40 mL), dried over anhydrous sodium sulfate and concentrated under vacuum. The crude product was purified by column chromatography (petroleum ether/ethyl acetate=15:1) to afford the title compound (133 mg, 23%) as a yellow oil. LCMS (ESI, m/z): 230 [M+H]+.

### Step 3: Synthesis of tert-butyl (S)-(1-(2-(1-(cyclopropylmethyl)-7-methoxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 2 (50 mg, 0.13 mmol) was reacted with 1-(cyclopropylmethyl)-7-methoxy-indole-2-carbaldehyde (29 mg, 0.13 mmol) according to General Procedure 6. The crude product was purified by column chromatography (petroleum ether/ethyl acetate=1:3) to afford the title compound (32 mg, 44%) as a light yellow solid. LCMS (ESI, m/z): 577 [M+H]+.

### Step 4: Synthesis of (S)-7-(2-amino-3-fluoropropyl)-2-(1-(cyclopropylmethyl)-7-methoxy-1H-indol-2-yl)-3-methyl-3,5,6,7-tetrahydro-8H-imidazo[4,5-b][1,6]naphthyridin-8-one

tert-butyl (S)-(1-(2-(1-(cyclopropylmethyl)-7-methoxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (32 mg, 0.06 mmol) was reacted according to General Procedure 2. The crude product was purified by Prep-HPLC (Method A) to afford the title compound (14.3 mg, 53%). as a white solid. LCMS (ESI, m/z): 477 [M+H]+. LCMS RT: 1.942 min. (Method B).

Compounds of Examples 2 to 16 in Table 1 were obtained following a procedure similar to the preparation of a compound of Example 1 using the appropriate alkylating agent in step 1.

TABLE 1

| | | LCMS m/z [M + H]+; LCMS RT |
|---|---|---|
| Example | | (LCMS method); |
| Number | Structure | ¹H NMR (MHz, solvent) |
| 2 | 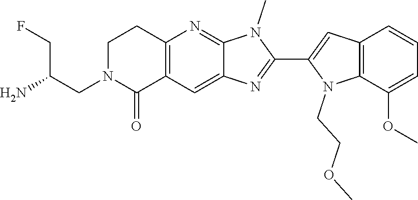 [SMILES: COCCn1c(-c2nc3cc4c(nc3n2C)CCN(C[C@H](N)CF)C4=O)cc2cccc(OC)c21] | (S)-7-(2-amino-3-fluoropropyl)-2- (7-methoxy-1-(2-methoxyethyl)-1H- indol-2-yl)-3-methyl-3,5,6,7- tetrahydro-8H-imidazo[4,5- b][1,6]naphthyridin-8-one LCMS (ESI, m/z): 481 [M + H]+; LCMS RT: 1.451 min. (Method B) ¹H NMR (400 MHz, DMSO-d₆): δ 8.48 (s, 1H), 7.28 (d, J = 8.0 Hz, 1H), 7.08 (t, J = 12.3 Hz, 2H), 6.86 (d, J = 8.2 Hz, 1H), 4.94 (t, J = 11.8 Hz, 2H), 4.47-4.38 (m, 1H), 4.34-4.26 (m, 1H), 3.94 (d, J = 12.0 Hz, 6H), 3.83- |
| | | 3.70 (m, 2H), 3.64-3.57 (m, 1H), |
| | | 3.52-3.44 (m, 3H), 3.31-3.24 (m, |
| | | 3H), 2.89 (s, 3H), 2.22-1.93 (m, |
| | | 2H). |
| 3 | 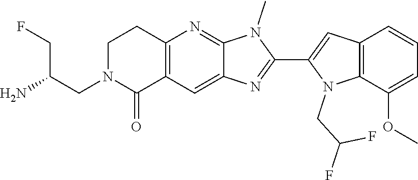 [SMILES: COc1cccc2cc(-c3nc4cc5c(nc4n3C)CCN(C[C@H](N)CF)C5=O)n(CC(F)F)c12] | (S)-7-(2-amino-3-fluoropropyl)-2- (1-(2,2-difluoroethyl)-7-methoxy- 1H-indol-2-yl)-3-methyl-3,5,6,7- tetrahydro-8H-imidazo[4,5- b][1,6]naphthyridin-8-one LCMS (ESI, m/z): 487 [M + H]+; LCMS RT: 1.443 min. (Method D) ¹H NMR (400 MHz, DMSO-d₆) δ 8.52 (s, 1H), 8.29 (s, 3H), 7.33- 7.31 (m, 2H), 7.15-7.11 (m, 1H), 6.93 (d, J = 7.6 Hz, 1H), 6.43-6.13 (m, 1H), 5.45-5.36 (m, 2H), 4.81- |
| | | 4.59 (m, 2H), 3.98 (d, J = 2.8 Hz, |
| | | 6H), 3.87-3.80 (m, 2H), 3.79- |
| | | 3.70 (m, 3H), 3.33 (t, J = 6.4 Hz, |
| | | 1H) |
| 4 | 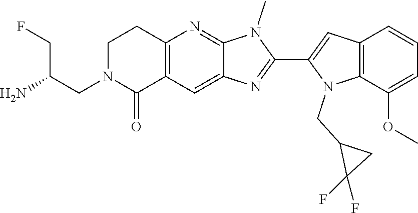 [SMILES: COc1cccc2cc(-c3nc4cc5c(nc4n3C)CCN(C[C@H](N)CF)C5=O)n(CC3CC3(F)F)c12] | 7-((S)-2-amino-3-fluoropropyl)-2- (1-((2,2- difluorocyclopropyl)methyl)-7- methoxy-1H-indol-2-yl)-3-methyl- 3,5,6,7-tetrahydro-8H-imidazo[4,5- b][1,6]naphthyridin-8-one LCMS (ESI, m/z): 513 [M + H]+; LCMS RT: 1.389 min. (Method D) ¹H NMR (400 MHz, DMSO-d₆) δ 8.51 (s, 1H), 8.31 (s, 3H), 7.32 (d, J = 7.6 Hz, 1H), 7.24 (s, 1H), 7.11 (t, J = 7.6 Hz, 1H), 6.90 (d, J = 7.6 Hz, 1H), 5.04-4.93 (m, 2H), 4.78-4.62 (m, 2H), 3.98 (s, 3H), 3.97 (s, 3H), |
| | | 3.88-3.83 (m, 2H), 3.77-3.71 (m, |
| | | 3H), 3.33 (t, J = 6.4 Hz, 2H), 2.16- |
| | | 2.11 (m, 1H), 1.45-1.42 (m, 1H), |
| | | 1.23-1.18 (m, 1H). |
| 5 | 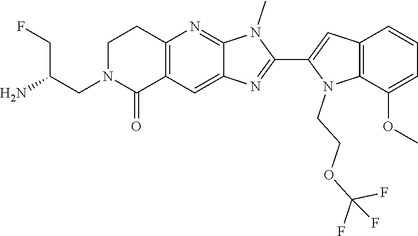 [SMILES: COc1cccc2cc(-c3nc4cc5c(nc4n3C)CCN(C[C@H](N)CF)C5=O)n(CCOC(F)(F)F)c12] | (S)-7-(2-amino-3-fluoropropyl)-2- (7-methoxy-1-(2- (trifluoromethoxy)ethyl)-1H-indol- 2-yl)-3-methyl-3,5,6,7-tetrahydro- 8H-imidazo[4,5- b][1,6]naphthyridin-8-one LCMS (ESI, m/z): 535 [M + H]+; LCMS RT: 0.946 min. (Method D) ¹H NMR (300 MHz, DMSO-d₆): δ 8.50 (s, 1H), 8.31 (s, 3H), 7.31 (d, J = 7.6 Hz, 1H), 7.24 (s, 1H), 7.11 (t, J = 7.6 Hz, 1H), 6.90 (d, J = 7.6 Hz, 1H), 5.16 (t, J = 5.6 Hz, 2H), 4.81- 4.63 (m, 2H), 4.37 (t, J = 5.6 Hz, 2H), 4.02-3.82 (m, 8H), 3.77-3.70 (m, 3H), 3.34 (t, J = 6.4 Hz, 2H). |
| 6 | 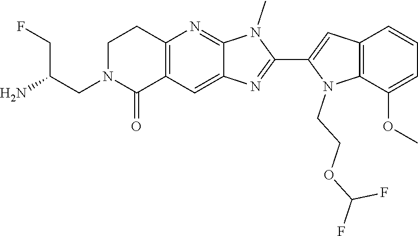 [SMILES: COc1cccc2cc(-c3nc4cc5c(nc4n3C)CCN(C[C@H](N)CF)C5=O)n(CCOC(F)F)c12] | (S)-7-(2-amino-3-fluoropropyl)-2- (1-(2-(difluoromethoxy)ethyl)-7- methoxy-1H-indol-2-yl)-3-methyl- 3,5,6,7-tetrahydro-8H-imidazo[4,5- b][1,6]naphthyridin-8-one LCMS (ESI, m/z): 517 [M + H]+; LCMS RT: 0.897 min. (Method D) ¹H NMR (300 MHz, DMSO-d₆): δ 8.51 (s, 1H), 8.32 (s, 3H), 7.30 (d, J = 7.8 Hz, 1H), 7.18 (s, 1H), 7.10 (t, J = 7.8 Hz, 1H), 6.89 (d, J = 7.8 Hz, 1H), 6.43 (t, J = 75.9 Hz, 1H), 5.07 (t, J = 5.1 Hz, 2H), 4.83-4.73 (m, 1H), 4.68-4.59 (m, 1H), 4.07 (t, J = 5.1 Hz, 2H), 3.96 (t, J = 4.5 Hz, 6H), 3.88-3.72 (m, 5H), 3.33 (t, J = 6.3 Hz, 2H). |
| 7 | 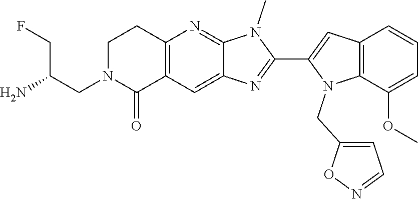 [SMILES: COc1cccc2cc(-c3nc4cc5c(nc4n3C)CCN(C[C@H](N)CF)C5=O)n(Cc3ccno3)c12] | (S)-7-(2-amino-3-fluoropropyl)-2- (1-(isoxazol-5-ylmethyl)-7- methoxy-1H-indol-2-yl)-3-methyl- 3,5,6,7-tetrahydro-8H-imidazo[4,5- b][1,6]naphthyridin-8-one LCMS (ESI, m/z): 504 [M + H]+; LCMS RT: 2.670 min. (Method B) ¹H NMR (400 MHz, DMSO-d₆): δ 8.53 (s, 1H), 8.35-8.27 (m, 3H), 7.37-7.30 (m, 2H), 7.13 (t, J = 7.8 Hz, 1H), 6.91 (d, J = 7.7 Hz, 1H), 6.36 (s, 2H), 5.96 (d, J = 1.8 Hz, 1H), 4.84-4.56 (m, 2H), 3.96 (d, J = 13.4 Hz, 6H), 3.90-3.69 (m, 5H), 3.34 (t, J = 6.6 Hz, 2H). |
| 8 | 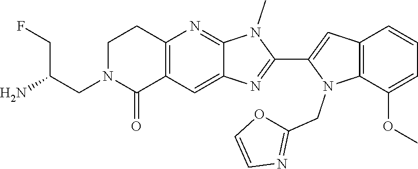 [SMILES: COc1cccc2cc(-c3nc4cc5c(nc4n3C)CCN(C[C@H](N)CF)C5=O)n(Cc3ncco3)c12] | (S)-7-(2-amino-3-fluoropropyl)-2- (7-methoxy-1-(oxazol-2-ylmethyl)- 1H-indol-2-yl)-3-methyl-3,5,6,7- tetrahydro-8H-imidazo[4,5- b][1,6]naphthyridin-8-one LCMS (ESI, m/z): 504 [M + H]+; LCMS RT: 1.570 min. (Method C). |
| 9 | 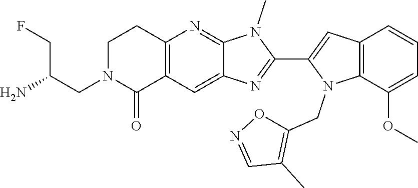 [SMILES: COc1cccc2cc(-c3nc4cc5c(nc4n3C)CCN(C[C@H](N)CF)C5=O)n(Cc3oncc3C)c12] | (S)-7-(2-amino-3-fluoropropyl)-2- (7-methoxy-1-((4-methylisoxazol-5- yl)methyl)-1H-indol-2-yl)-3-methyl- 3,5,6,7-tetrahydro-8H-imidazo[4,5- b][1,6]naphthyridin-8-one LCMS (ESI, m/z): 518 [M + H]+; LCMS RT: 1.636 min. (Method B) ¹H NMR (400 MHz, DMSO-d₆): δ 8.51 (s, 1H), 8.30 (br, 3H), 8.19 (s, 1H), 7.30 (d, 2H), 7.11 (t, J = 7.9 Hz, 1H), 6.91 (d, J = 7.7 Hz, 1H), 6.29 (s, 2H), 4.82-4.58 (m, 3H), 3.94 (s, |
| | | 2H), 3.92 (s, 2H), 3.88-3.79 (m, |
| | | 3H), 3.75 (t, J = 6.2 Hz, 3H), 3.33 (t, |
| | | J = 6.6 Hz, 2H), 1.63 (s, 3H). |
| 10 | 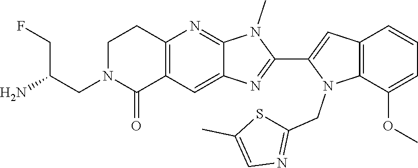 [SMILES: COc1cccc2cc(-c3nc4cc5c(nc4n3C)CCN(C[C@H](N)CF)C5=O)n(Cc3ncc(C)s3)c12] | (S)-7-(2-amino-3-fluoropropyl)-2- (7-methoxy-1-((5-methylthiazol-2- yl)methyl)-1H-indol-2-yl)-3-methyl- 3,5,6,7-tetrahydro-8H-imidazo[4,5- b][1,6]naphthyridin-8-one LCMS (ESI, m/z): 534 [M + H]+; LCMS RT: 1.770 min. (Method D) ¹H NMR (400 MHz, DMSO-d₆) δ 8.51 (s, 1H), 8.27 (s, 3H), 7.30 (d, J = 7.2 Hz, 2H), 7.18 (d, J = 8.0 Hz, 1H), 7.11 (t, J = 8.0 Hz, 1H), 6.90 |
| | | (d, J = 7.6 Hz, 1H), 6.34 (s, 2H), |
| | | 4.80-4.59 (m, 2H), 3.92 (d, J = 4.4 |
| | | Hz, 6H), 3.87-3.27 (m, 5H), 3.18- |
| | | 3.11 (m, 2H), 2.23 (s, 3H). |
| 11 | 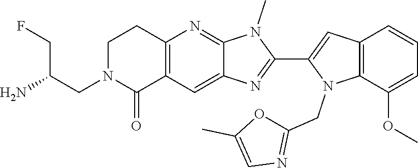 [SMILES: COc1cccc2cc(-c3nc4cc5c(nc4n3C)CCN(C[C@H](N)CF)C5=O)n(Cc3ncc(C)o3)c12] | (S)-7-(2-amino-3-fluoropropyl)-2- (7-methoxy-1-((5-methyloxazol-2- yl)methyl)-1H-indol-2-yl)-3-methyl- 3,5,6,7-tetrahydro-8H-imidazo[4,5- b][1,6]naphthyridin-8-one LCMS (ESI, m/z): 518 [M + H]+; LCMS RT 1.502 min. (Method C) ¹H NMR (400 MHz, DMSO-d6): δ 8.54 (br, 3H), 7.31 (m, 2H), 7.10 (t, J = 7.9 Hz, 1H), 6.88 (d, J = 7.8 Hz, 1H), 6.52 (d, J = 1.5 Hz, 1H), 6.19 (s, |
| | | 2H), 4.90-4.53 (m, 2H), 3.94 (s, |
| | | 3H), 3.89 (s, 3H), 3.79-3.73 (m, |
| | | 5H), 3.33 (t, J = 6.6 Hz, 2H), 2.07 (s, |
| | | 3H). |
| 12 | 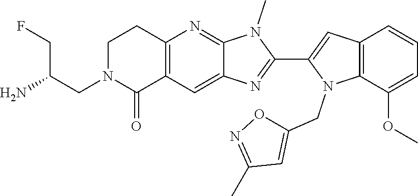 [SMILES: COc1cccc2cc(-c3nc4cc5c(nc4n3C)CCN(C[C@H](N)CF)C5=O)n(Cc3cc(C)no3)c12] | (S)-7-(2-amino-3-fluoropropyl)-2- (7-methoxy-1-((3-methylisoxazol-5- yl)methyl)-1H-indol-2-yl)-3-methyl- 3,5,6,7-tetrahydro-8H-imidazo[4,5- b][1,6]naphthyridin-8-one LCMS (ESI, m/z): 518 [M + H]+; LCMS RT: 1.634 min. (Method C) ¹H NMR (400 MHz, DMSO-d₆): δ 8.51 (s, 1H), 8.30 (s, 3H), 7.32 (s, 1H), 7.30 (d, J = 0.9 Hz, 1H), 7.11 (t, J = 7.9 Hz, 1H), 6.90 (d, J = 7.7 Hz, 1H), 6.27 (s, 2H), 5.81 (s, 1H), 4.85- 4.52 (m, 2H), 3.97 (s, 3H), 3.94 (s, |
| | | 3H), 3.88-3.79 (m, 2H), 3.79-3.65 |
| | | (m, 3H), 3.33 (t, J = 6.6 Hz, 2H), |
| | | 2.03 (s, 3H). |
| 13 | 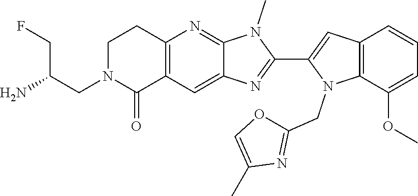 [SMILES: COc1cccc2cc(-c3nc4cc5c(nc4n3C)CCN(C[C@H](N)CF)C5=O)n(Cc3nc(C)co3)c12] | (S)-7-(2-amino-3-fluoropropyl)-2- (7-methoxy-1-((4-methyloxazol-2- yl)methyl)-1H-indol-2-yl)-3-methyl- 3,5,6,7-tetrahydro-8H-imidazo[4,5- b][1,6]naphthyridin-8-one LCMS (ESI, m/z): 518 [M + H]+; LCMS RT: 1.881 min. (Method B) ¹H NMR (400 MHz, DMSO-d₆) δ 8.47 (s, 1H), 8.33 (s, 3H), 7.61 (s, 1H), 7.53-7.28 (m, 2H), 7.12-7.07 (m, 1H), 6.88 (d, J = 7.6 Hz, 1H), 6.21 (s, 2H), 4.78-4.59 (m, 2H), 3.95 (s, 3H), 3.90-3.81 (m, 5H), 3.76-3.68 (m, 3H), 3.34-3.31 (m, 2H), 1.85 (m, 3H). |
| 14 | 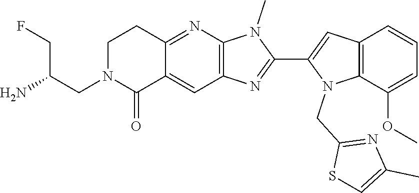 [SMILES: COc1cccc2cc(-c3nc4cc5c(nc4n3C)CCN(C[C@H](N)CF)C5=O)n(Cc3nc(C)cs3)c12] | (S)-7-(2-amino-3-fluoropropyl)-2- (7-methoxy-1-((4-methylthiazol-2- yl)methyl)-1H-indol-2-yl)-3-methyl- 3,5,6,7-tetrahydro-8H-imidazo[4,5- b][1,6]naphthyridin-8-one LCMS (ESI, m/z): 534 [M + H]+; LCMS RT: 1.833 min. (Method C) ¹H NMR (400 MHz, DMSO-d6): δ 8.50 (s, 1H), 8.31 (d, J = 16.1 Hz, 3H), 7.32-7.29 (m, 2H), 7.11 (t, J = 7.9 Hz, 1H), 6.96 (d, J = 1.2 Hz, 1H), 6.89 (d, J = 7.8 Hz, 1H), 6.34 (s, 2H), 4.83-4.78 (m, 1H), 4.70- |
| | | 4.54 (m, 1H), 3.93 (s, 6H), 3.89- |
| | | 3.78 (m, 2H), 3.78-3.68 (m, 3H), |
| | | 3.32 (t, J = 6.6 Hz, 2H), 2.12 (s, |
| | | 3H). |
| 15 | 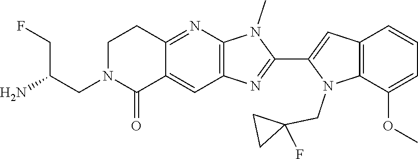 [SMILES: COc1cccc2cc(-c3nc4cc5c(nc4n3C)CCN(C[C@H](N)CF)C5=O)n(CC3(F)CC3)c12] | (S)-7-(2-amino-3-fluoropropyl)-2- (1-((1-fluorocyclopropyl)methyl)-7- methoxy-1H-indol-2-yl)-3-methyl- 3,5,6,7-tetrahydro-8H-imidazo[4,5- b][1,6]naphthyridin-8-one LCMS (ESI, m/z): 495 [M + H]+; LCMS RT: 1.559 min. (Method B) ¹H NMR (400 MHz, DMSO-d₆): δ 8.52 (s, 1H), 8.31 (s, 3H), 7.35-7.24 (m, 2H), 7.11 (t, J = 7.9 Hz, 1H), 6.89 (d, J = 7.7 Hz, 1H), 5.46-5.32 (m, |
| | | 2H), 4.82-4.57 (m, 2H), 4.02-3.88 |
| | | (m, 6H), 3.88-3.79 (m, 2H), 3.80- |
| | | 3.68 (m, 3H), 3.38-3.29 (m, 2H), |
| | | 0.80-0.67 (m, 2H), 0.56-0.45 (m, |
| | | 2H). |
| 16 | 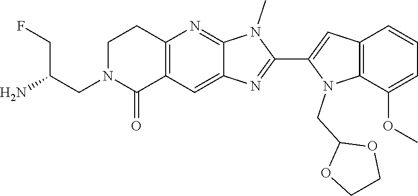 [SMILES: COc1cccc2cc(-c3nc4cc5c(nc4n3C)CCN(C[C@H](N)CF)C5=O)n(CC3OCCO3)c12] | (S)-2-(1-((1,3-dioxolan-2- yl)methyl)-7-methoxy-1H-indol-2- yl)-7-(2-amino-3-fluoropropyl)-3- methyl-3,5,6,7-tetrahydro-8H- imidazo[4,5-b][1,6]naphthyridin-8- one LCMS (ESI, m/z): 509 [M + H]+; LCMS RT: 0.822 min. (Method D) ¹H NMR (400 MHz, DMSO-d₆) δ 8.52 (s, 1H), 8.28 (s, 3H), 7.28 (d, J = 7.2 Hz, 1H), 7.18 (s, 1H), 7.08- 7.06 (m, 1H), 6.87 (d, J = 7.2 Hz, 1H), 5.05 (d, J = 4.0 Hz, 2H), 4.95 (t, |
| | | J = 4.4 Hz, 1H), 4.79-4.63 (m, 2H), |
| | | 3.96 (s, 3H), 3.95-3.70 (m, 7H), |
| | | 3.52-3.49 (m, 3H), 3.39-3.32 (m, |
| | | 2H), 3.28-3.25 (m, 1H). |

### Example 17. (S)-7-(2-amino-3-fluoropropyl)-2-(1-(isoxazol-5-ylmethyl)-7-methyl-1H-indol-2-yl)-3-methyl-3,5,6,7-tetrahydro-8H-imidazo[4,5-b][1,6]naphthyridin-8-one

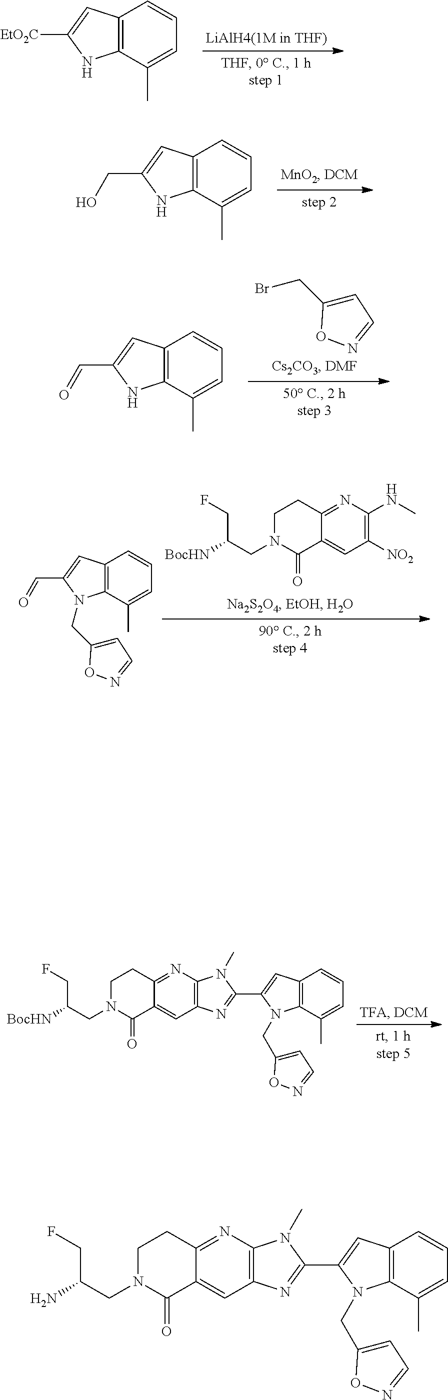

### Step 1: Synthesis of (7-methyl-1H-indol-2-yl)methanol

7-methyl-1H-indole-2-carboxylate (1 g, 4.9 mmol) was reacted according to General Procedure 3. The crude product was purified by column chromatography (petroleum ether/ethyl acetate=4:1) to afford the title compound (700 mg, 88%) as a yellow solid. LCMS (ESI, m/z): 162 [M+H]+.

### Step 2: Synthesis of 7-methyl-1H-indole-2-carbaldehyde

(7-methyl-1H-indol-2-yl)methanol (700 mg, 4.3 mmol) was reacted according to General Procedure 4. The crude product was purified by column chromatography (petroleum ether/ethyl acetate=2:1) to afford the title compound (500 mg, 73%) as a yellow solid. LCMS (ESI, m/z): 160 [M+H]+.

### Step 3: Synthesis of 1-(isoxazol-5-ylmethyl)-7-methyl-1H-indole-2-carbaldehyde

7-methyl-1H-indole-2-carbaldehyde (100 mg, 0.63 mmol) was reacted with 5-(bromomethyl)isoxazole (204 mg, 1.26 mmol) according to General Procedure 5. The resulting solution was stirred at 50° C. for 2 h. The reaction was monitored by LCMS. The reaction was then quenched by adding water (30 mL) and extracted with ethyl acetate (30 mL), washed with water (2×30 mL) and brine (2×30 mL), dried over anhydrous sodium sulfate and concentrated under vacuum. The crude product was purified by column chromatography (petroleum ether/ethyl acetate=10:1) to afford the title compound (95 mg, 63%) as a yellow oil. LCMS (ESI, m/z): 241 [M+H]+.

### Step 4: Synthesis of tert-butyl (S)-(1-fluoro-3-(2-(1-(isoxazol-5-ylmethyl)-7-methyl-1H-indol-2-yl)-3-methyl-8-oxo-3,5,6,8-tetrahydro-7H-imidazo[4,5-b][1,6]naphthyridin-7-yl)propan-2-yl)carbamate

1-(isoxazol-5-ylmethyl)-7-methyl-1H-indole-2-carbaldehyde (95 mg, 0.39 mmol) was reacted with Intermediate 2 (186 mg, 0.47 mmol) according to General Procedure 6. The crude product was purified by column chromatography (petroleum ether/ethyl acetate=1:2) to afford the title compound (80 mg, 340) as a yellow solid. LCMS (ESI, m/z): 588 [M+H]+.

### Step 5: Synthesis of (S)-7-(2-amino-3-fluoropropyl)-2-(1-(isoxazol-5-ylmethyl)-7-methyl-1H-indol-2-yl)-3-methyl-3,5,6,7-tetrahydro-8H-imidazo[4,5-b][1,6]naphthyridin-8-one

tert-butyl (S)-(1-fluoro-3-(2-(1-(isoxazol-5-ylmethyl)-7-methyl-1H-indol-2-yl)-3-methyl-8-oxo-3,5,6,8-tetrahydro-7H-imidazo[4,5-b][1,6]naphthyridin-7-yl)propan-2-yl)carbamate (80 mg, 0.14 mmol) was reacted according to General Procedure 2. The crude product was purified by Prep-HPLC (Method E) to afford the title compound (16.1 mg, 240%) as a white solid. LCMS (ESI, m/z): 488 [M+H]+. LCMS RI: 1.467 min. (Method B).

Compounds of Examples 18 to 21 in Table 2 were obtained following a procedure similar to the preparation of a compound of Example 17 using the appropriate indole-2-carbaldehyde and alkylation agent in step 3.

TABLE 2

| | | LCMS m/z [M + H]+; LCMS RT |
|---|---|---|
| Example | | (LCMS method); |
| Number | Structure | ¹H NMR (MHz, solvent) |
| 18 | 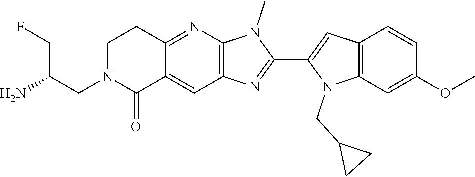 [SMILES: COc1ccc2cc(-c3nc4cc5c(nc4n3C)CCN(C[C@H](N)CF)C5=O)n(CC3CC3)c2c1] | (S)-7-(2-amino-3-fluoropropyl)-2- (1-(cyclopropylmethyl)-6-methoxy- 1H-indol-2-yl)-3-methyl-3,5,6,7- tetrahydro-8H-imidazo[4,5- b][1,6]naphthyridin-8-one LCMS (ESI, m/z): 477 [M + H]+; LCMS RT: 1.550 min. (Method B) ¹H NMR (400 MHz, DMSO-d₆) δ 8.44 (s, 1H), 7.58 (d, J = 8.8 Hz, 1H), 7.18 (d, J = 12 Hz, 2H), 6.82 (d, J = 12 Hz, 1H), 4.58 (d, J = 6.8 Hz, 2H), 4.41-4.39 (m, 1H), 4.29- 4.27 (m, 1H), 3.99-3.87 (m, 3H), 3.79-3.76 (m, 3H), 3.74-3.72 (m, 2H), 3.70-3.60 (m, 1H), 3.59- 3.42 (m, 1H), 3.31-3.26 (m, 3H), 1.79-1.68 (m, 2H), 1.21-1.19 (m, 1H), 0.35-0.29 (m, 2H), 0.12- −0.05 (m, 2H). |
| 19 | 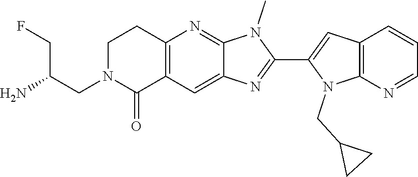 [SMILES: Cn1c(-c2cc3cccnc3n2CC2CC2)nc2cc3c(nc21)CCN(C[C@H](N)CF)C3=O] | (S)-7-(2-amino-3-fluoropropyl)-2- (1-(cyclopropylmethyl)-1H- pyrrolo[2,3-b]pyridin-2-yl)-3- methyl-3,5,6,7-tetrahydro-8H- imidazo[4,5-b][1,6]naphthyridin-8- one LCMS (ESI, m/z): 448 [M + H]+; RT: 1.462 min. (Method B) ¹H NMR (300 MHz, Methanol-d4) δ 8.66 (s, 1H), 8.42 (dd, J = 4.2, 0.9 Hz, 1H), 8.21 (dd, J = 4.5, 3.3 Hz, 1H), 7.27 (dd, J = 6.3, 1.5 Hz, 1H), 7.20 (s, 1H), 4.89-4.62 (m, 4H), 4.08-3.77 (m, 8H), 3.43-3.39 (m, 2H), 1.05 (s, br 1H), 0.32-0.28 (m, 2H), 0.06-0.00 (s, 2H). |
| 20 | 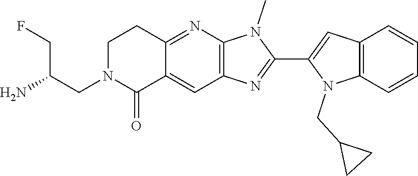 [SMILES: Cn1c(-c2cc3ccccc3n2CC2CC2)nc2cc3c(nc21)CCN(C[C@H](N)CF)C3=O] | (S)-7-(2-amino-3-fluoropropyl)-2- (1-(cyclopropylmethyl)-1H-indol-2- yl)-3-methyl-3,5,6,7-tetrahydro-8H- imidazo[4,5-b][1,6]naphthyridin-8- one LCMS (ESI, m/z): 447 [M + H]+; LCMS RT: 1.872 min. (Method B). |
| 21 | 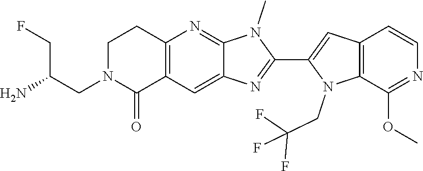 [SMILES: COc1nccc2cc(-c3nc4cc5c(nc4n3C)CCN(C[C@H](N)CF)C5=O)n(CC(F)(F)F)c12] | (S)-7-(2-amino-3-fluoropropyl)-2- (7-methoxy-1-(2,2,2-trifluoroethyl)- 1H-pyrrolo[2,3-c]pyridin-2-yl)-3- methyl-3,5,6,7-tetrahydro-8H- imidazo[4,5-b][1,6]naphthyridin-8- one LCMS (ESI, m/z): 506 [M + H]+; RT: 0.816 min. (Method B) ¹H NMR (400 MHz, DMSO-d₆) δ 8.58 (s, 1H), 8.30 (s, 3H), 7.87 (d, J = 5.6 Hz, 1H), 7.49 (s, 1H), 7.38 (d, J = 6.0 Hz, 1H), 5.94 (d, J = 8.4 Hz, 2H), 4.80-4.60 (m, 2H), 4.10 (s, 3H), 4.01 (s, 3H), 3.97-3.72 (m, 5H), 3.35 (t, J = 6.8 Hz, 2H). |

### Example 22. (S)-7-(2-amino-3-fluoropropyl)-2-(5-fluoro-1-(isoxazol-5-ylmethyl)-7-methoxy-1H-indol-2-yl)-3-methyl-3,5,6,7-tetrahydro-8H-imidazo[4,5-b][1,6]naphthyridin-8-one

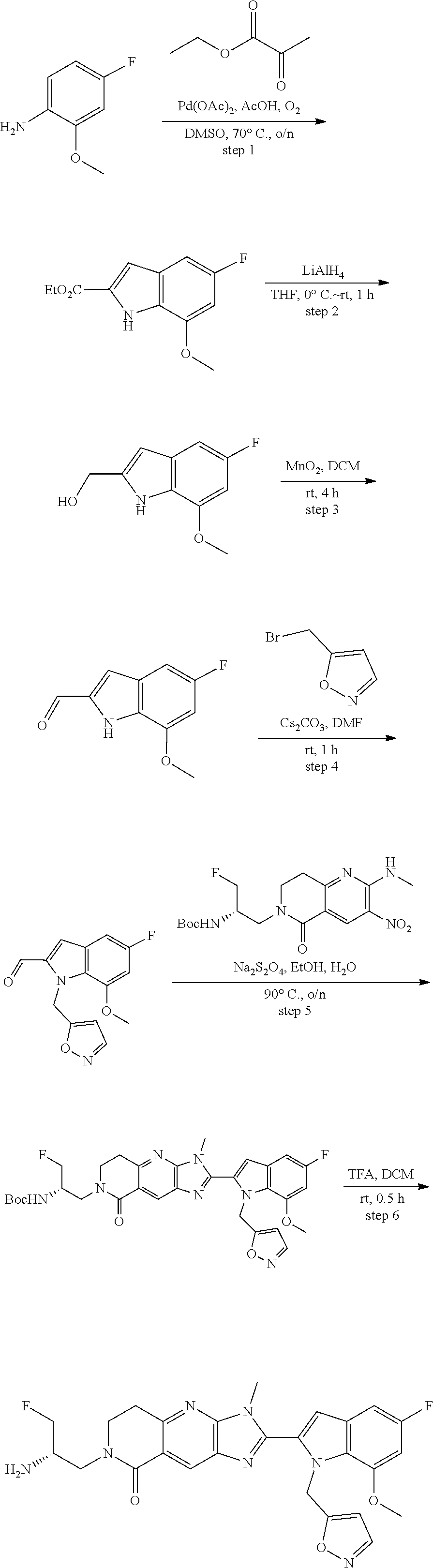

### Step 1: Synthesis of ethyl 5-fluoro-7-methoxy-1H-indole-2-carboxylate

To a solution of 4-fluoro-2-methoxy-aniline (2.8 g, 19.8 mmol) in DMSO (200 mL), was added ethyl 2-oxopropanoate (4.6 g, 39.6 mmol), Pd(OAc)₂ (889 mg, 3.97 mmol) and AcOH (1.2 mL, 19.8 mmol) under nitrogen atmosphere. The resulting mixture was stirred at 70° C. for overnight under oxygen atmosphere. The reaction was monitored by LCMS. The reaction was then quenched by adding water (600 mL) and extracted with ethyl acetate (600 mL), washed with water (2×600 ml) and brine (2×600 mL), dried over anhydrous sodium sulfate and concentrated under vacuum. The crude product was purified by column chromatography (petroleum ether/ethyl acetate=5:1) to afford the title compound (2.5 g, 53%) as a yellow solid. LCMS (ESI, m/z): 238 [M+H]+.

### Step 2: Synthesis of (5-fluoro-7-methoxy-1H-indol-2-yl)methanol

To a solution of ethyl 5-fluoro-7-methoxy-1H-indole-2-carboxylate (200 mg, 0.84 mmol) was reacted according to General Procedure 3. The crude product was purified by column chromatography (petroleum ether/ethyl acetate=3:1) to afford the title compound (120 mg, 73%) as yellow oil. LCMS (ESI, m/z): 196 [M+H]+.

### Step 3: Synthesis of 5-fluoro-7-methoxy-1H-indole-2-carbaldehyde

(5-fluoro-7-methoxy-1H-indol-2-yl)methanol (164 mg, 0.84 mmol) was reacted according to General Procedure 4. The crude product was purified by column chromatography (petroleum ether/ethyl acetate=3:1) to afford the title compound (100 mg, 62%) as a yellow solid. LCMS (ESI, m/z): 194 [M+H]+

### Step 4: Synthesis of 5-fluoro-1-(isoxazol-5-ylmethyl)-7-methoxy-1H-indole-2-carbaldehyde

5-fluoro-7-methoxy-1H-indole-2-carbaldehyde (100 mg, 0.52 mmol) was reacted with 5-(bromomethyl)isoxazole (126 mg, 0.78 mmol) according to General Procedure 5. The crude product was purified by column chromatography (petroleum ether/ethyl acetate=3:1) to afford the title compound (120 mg, 84%) as a yellow solid. LCMS (ESI, m/z): 275 [M+H]+.

### Step 5: Synthesis of tert-butyl (S)-(1-fluoro-3-(2-(5-fluoro-1-(isoxazol-5-ylmethyl)-7-methoxy-1H-indol-2-yl)-3-methyl-8-oxo-3,5,6,8-tetrahydro-7H-imidazo[4,5-b][1,6]naphthyridin-7-yl)propan-2-yl)carbamate

5-fluoro-1-(isoxazol-5-ylmethyl)-7-methoxy-1H-indole-2-carbaldehyde (50 mg, 0.18 mmol) was reracted with Intermediate 2 (144 mg, 0.36 mmol) according to General Procedure 6. The crude product was purified by column chromatography (petroleum ether/ethyl acetate=1:2) to afford the title compound (60 mg, 53%) as a yellow solid. LCMS (ESI, m/z): 622 [M+H]+.

### Step 6: Synthesis of (S)-7-(2-amino-3-fluoropropyl)-2-(5-fluoro-1-(isoxazol-5-ylmethyl)-7-methoxy-1H-indol-2-yl)-3-methyl-3,5,6,7-tetrahydro-8H-imidazo[4,5-b][1,6]naphthyridin-8-one

tert-butyl (S)-(1-fluoro-3-(2-(5-fluoro-1-(isoxazol-5-ylmethyl)-7-methoxy-1H-indol-2-yl)-3-methyl-8-oxo-3,5,6,8-tetrahydro-7H-imidazo[4,5-b][1,6]naphthyridin-7-yl)propan-2-yl)carbamate (50 mg, 0.08 mmol) was reacted according to General Procedure 2. The crude product was purified by Prep HPLC (Method F) to afford the title compound (21.7 mg, 50%) as a white solid. ¹H NMR (400 MHz, DMSO-d6): δ 8.52 (s, 1H), 8.33-8.30 (m, 4H), 7.32 (s, 1H), 7.09 (d, J=7.0 Hz, 1H), 6.86 (d, J=7.0 Hz, 1H), 6.30 (s, 2H), 5.99 (d, J=1.8 Hz, 1H), 4.82-4.57 (m, 2H), 3.95 (d, J=1.9 Hz, 6H), 3.89-3.80 (m, 2H), 3.78-3.69 (m, 3H), 3.33 (t, J=6.4 Hz, 2H). LCMS (ESI, m/z): 522 [M+H]+. LCMS RT: 1.489 min. (Method B).

### Example 23. (S)-7-(2-amino-3-fluoropropyl)-2-(6-fluoro-1-(isoxazol-5-ylmethyl)-7-methoxy-1H-indol-2-yl)-3-methyl-3,5,6,7-tetrahydro-8H-imidazo[4,5-b][1,6]naphthyridin-8-one

Example 23 was synthesized via a similar synthetic route to Example 22 starting from 4-fluoro-2-methoxy-aniline in step 1. The crude product was purified by Prep-HPLC (Method F) to afford the title compound (15.8 mg, 36%) as a white solid. LCMS (ESI, m/z): 522 [M+H]+. LCMS RT: 1.491 min. (Method D).

### Example 24. (R)-7-(2-aminopropyl)-2-(1-(cyclopropylmethyl)-1H-indol-2-yl)-3-methyl-3,5,6,7-tetrahydro-8H-imidazo[4,5-b][1,6]naphthyridin-8-one

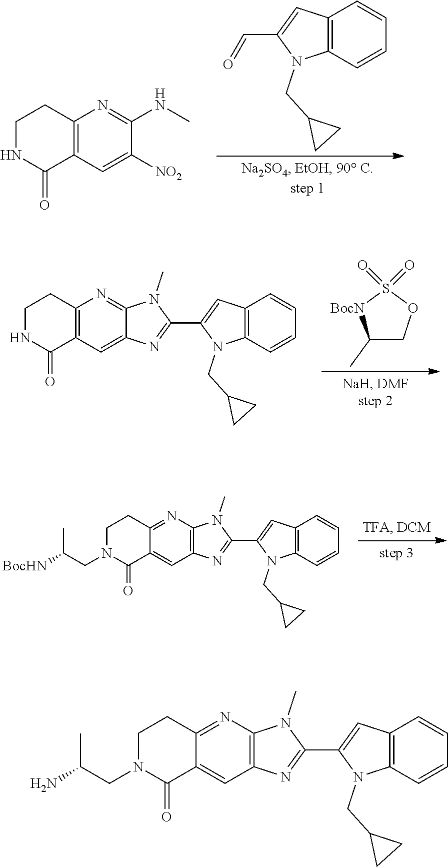 [SMILES: CNc1nc2c(cc1[N+](=O)[O-])C(=O)NCC2 ; O=Cc1cc2ccccc2n1CC1CC1 ; Cn1c(-c2cc3ccccc3n2CC2CC2)nc2cc3c(nc21)CCNC3=O ; C[C@@H]1COS(=O)(=O)N1C(=O)OC(C)(C)C ; C[C@H](CN1CCc2nc3c(cc2C1=O)nc(-c1cc2ccccc2n1CC1CC1)n3C)NC(=O)OC(C)(C)C ; C[C@@H](N)CN1CCc2nc3c(cc2C1=O)nc(-c1cc2ccccc2n1CC1CC1)n3C]

### Step 1: Synthesis of 2-(1-(cyclopropylmethyl)-1H-indol-2-yl)-3-methyl-3,5,6,7-tetrahydro-8H-imidazo[4,5-b][1,6]naphthyridin-8-one

Intermediate 3 (170 mg, 0.77 mmol) was reacted with 1-(cyclopropylmethyl)indole-2-carbaldehyde (168 mg, 0.84 mmol) according to General Procedure 6. The residue was passed though Prep-TLC (DCM:MeOH=25:1) to obtain 2-[1-(cyclopropylmethyl)indol-2-yl]-3-methyl-6,7-dihydro-5H-imidazo[4,5-b][1,6]naphthyridin-8-one (170 mg, 60%) as yellow oil. LCMS (ESI, m/z): 372 [M+H]+.

### Step 2: Synthesis of tert-butyl (R)-(1-(2-(1-(cyclopropylmethyl)-1H-indol-2-yl)-3-methyl-8-oxo-3,5,6,8-tetrahydro-7H-imidazo[4,5-b][1,6]naphthyridin-7-yl)propan-2-yl)carbamate

2-(1-(cyclopropylmethyl)-1H-indol-2-yl)-3-methyl-3,5,6,7-tetrahydro-8H-imidazo[4,5-b][1,6]naphthyridin-8-one (60 mg, 0.16 mmol) was reacted with tert-butyl (R)-4-methyl-1,2,3-oxathiazolidine-3-carboxylate 2,2-dioxide (58 mg, 0.24 mmol) according to General Procedure 1. The residue was purified by Prep-TLC (DCM:MeOH=25:1) to obtain the tile compound (50 mg, 57%) as a yellow solid. LCMS (ESI, m/z): 529 [M+H]+.

### Step 3: Synthesis of (R)-7-(2-aminopropyl)-2-(1-(cyclopropylmethyl)-1H-indol-2-yl)-3-methyl-3,5,6,7-tetrahydro-8H-imidazo[4,5-b][1,6]naphthyridin-8-one

tert-butyl (R)-(1-(2-(1-(cyclopropylmethyl)-1H-indol-2-yl)-3-methyl-8-oxo-3,5,6,8-tetrahydro-7H-imidazo[4,5-b][1,6]naphthyridin-7-yl)propan-2-yl)carbamate (50 mg, 0.09 mmol) was reacted according to General Procedure 2. The residue was purified by Prep HPLC (Method E) to obtain the title compound (27.1 mg, 67%) as a white solid. ¹H NMR (400 MHz, DMSO-d₆) δ 8.51 (s, 1H), 7.88 (s, 3H), 7.72 (dd, J=8.1 Hz, J=6.6 Hz, 2H), 7.36-7.30 (m, 1H), 7.26 (s, 1H), 7.19-7.14 (m, 1H), 4.59 (d, J=7.0 Hz, 2H), 4.01 (s, 3H), 3.85-3.67 (m, 3H), 3.65-3.50 (m, 2H), 3.32 (t, J=6.6 Hz, 2H), 1.26 (d, J=6.2 Hz, 3H), 1.17-1.07 (m, 1H), 0.33-0.26 (m, 2H), 0.10-0.04 (m, 2H). LCMS (ESI, m/z): 429 [M+H]⁻. LCMS RT: 1.118 min. (Method D).

### Example 25. (R)-7-(2-aminopropyl)-2-(7-methoxy-1-(oxazol-2-ylmethyl)-1H-indol-2-yI)-3-methyl-3,5,6,7-tetrahydro-8H-imidazo[4,5-b][1,6]naphthyridin-8-one

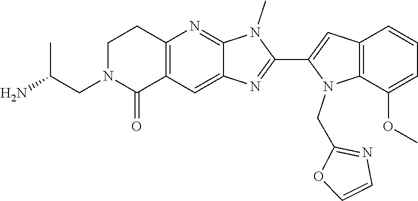 [SMILES: COc1cccc2cc(-c3nc4cc5c(nc4n3C)CCN(C[C@@H](C)N)C5=O)n(Cc3ncco3)c12]

Example 25 was synthesized via a similar synthetic route to Example 24 using the appropriately substituted indole (7-methoxy-1-(oxazol-2-ylmethyl)-1H-indole-2-carbaldehyde (in step 1). The crude product was purified by Prep HPLC (Method F) to afford the title compound (10.3 mg, 62%0) as a yellow solid. ¹H NMR (400 MHz, Methanol-d4) δ 8.62 (s, 1H), 7.68 (s, 1H), 7.33 (d, J=8 Hz, 1H), 7.21 (s, 1H), 7.18 (t, 1=21.6 Hz, 1H), 6.92 (s, 1H), 6.87 (d, J=7.6 Hz, 1H), 6.29 (s, 2H), 4.01 (s, 3H), 4.00-3.90 (m, 1H), 3.89 (s, 3H), 3.81-3.72 (m, 2H), 3.71-3.63 (m, 1H), 3.62-3.50 (m, 1H), 3.40 (t, 1=6.8 Hz, 2H), 1.40 (s, 3H). LCMS (ESI, m/z): 486 [M+H]+. LCMS RT: 0.815 min. (Method D).

### Example 26. (S)-2-(1-((1-fluorocyclopropyl)methyl)-7-methoxy-1H-indol-2-yl)-3-methyl-7-(morpholin-3-ylmethyl)-3,5,6,7-tetrahydro-8H-imidazo[4,5-b][1,6]naphthyridin-8-one

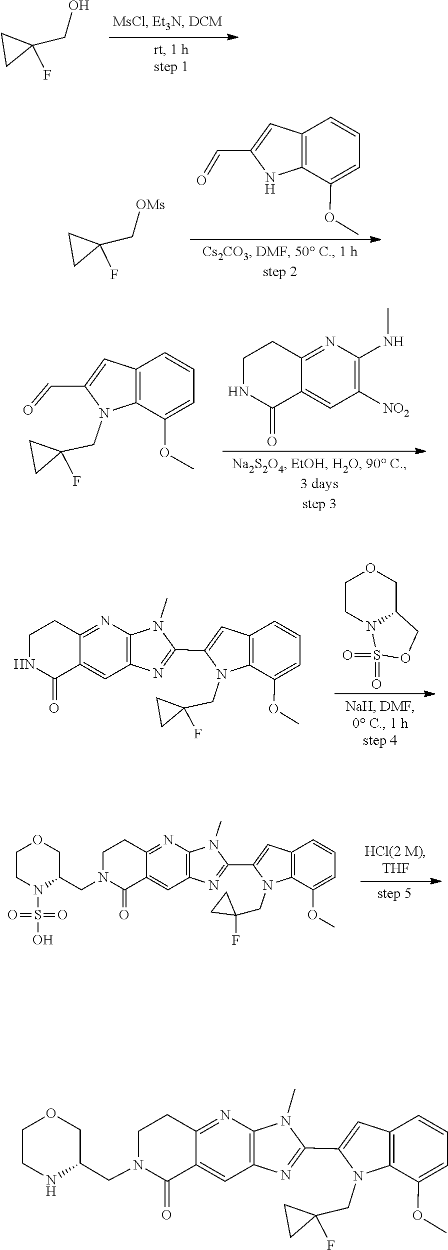

### Step 1: Synthesis of (1-fluorocyclopropyl)methyl methanesulfonate

To a stirred solution of (1-fluorocyclopropyl)methanol (200 mg, 2.22 mmol) in DCM (10 mL) were added Et₃N (0.62 mL, 4.44 mmol) and MsCl (0.21 mL, 2.66 mmol) at 0° C. under nitrogen atmosphere. The resulting mixture was stirred at room temperature for 1 h. The reaction was monitored by TLC and LCMS. The reaction was quenched by adding water (20 mL) and extracted with ethyl acetate (3×20 mL). The combined organic extracts were washed with brine (2×40 mL), dried over anhydrous sodium sulfate and concentrated to afford the title compound (200 mg, 53%) as yellow oil. LCMS (ESI, m/z): 169 [M+H]+.

### Step 2: Synthesis of 1-((1-fluorocyclopropyl)methyl)-7-methoxy-1H-indole-2-carbaldehyde

7-methoxy-1H-indole-2-carbaldehyde (140 mg, 0.8 mmol) was reacted with 1-fluorocyclopropyl)methyl methanesulfonate (201 mg, 1.2 mmol) according to General Procedure 5. The crude product was purified by silica column chromatography (Petroleum ether/ethyl acetate=4:1) to afford the title compound (110 mg, 55%) as a yellow solid. LCMS (ESI, m/z): 248 [M+H]+.

### Step 3: Synthesis of 2-(1-((1-fluorocyclopropyl)methyl)-7-methoxy-1H-indol-2-yl)-3-methyl-3,5,6,7-tetrahydro-8H-imidazo[4,5-b][1,6]naphthyridin-8-one

Intermediate 3 (110 mg, 0.5 mmol) was reacted with 1-((1-fluorocyclopropyl)methyl)-7-methoxy-1H-indole-2-carbaldehyde (122 mg, 0.5 mmol) according to General Procedure 6. The crude product was purified by Prep-TLC (DCM/methanol=30:1) to afford the title compound (30 mg, 14%) as a yellow solid. LCMS (ESI, m/z): 420 [M+H]+.

### Step 4: Synthesis of (S)-3-((2-(1-((1-fluorocyclopropyl)methyl)-7-methoxy-1H-indol-2-yl)-3-methyl-8-oxo-3,5,6,8-tetrahydro-7H-imidazo[4,5-b][1,6]naphthyridin-7-yl)methyl)morpholine-4-sulfonic acid

2-(1-((1-fluorocyclopropyl)methyl)-7-methoxy-1H-indol-2-yl)-3-methyl-3,5,6,7-tetrahydro-8H-imidazo[4,5-b][1,6]naphthyridin-8-one (30 mg, 0.07 mmol) was reacted with Intermediate 4 (15 mg, 0.09 mmol) according to General Procedure 1. The crude product was purified by flash chromatography to afford the title compound (40 mg, 93%) as a yellow solid. LCMS (ESI, m/z): 599 [M+H]+.

### Step 5: Synthesis of (S)-2-(1-((1-fluorocyclopropyl)methyl)-7-methoxy-1H-indol-2-yl)-3-methyl-7-(morpholin-3-ylmethyl)-3,5,6,7-tetrahydro-8H-imidazo[4,5-b][1,6]naphthyridin-8-one

(S)-3-((2-(1-((1-fluorocyclopropyl)methyl)-7-methoxy-1H-indol-2-yl)-3-methyl-8-oxo-3,5,6,8-tetrahydro-7H-imidazo[4,5-b][1,6]naphthyridin-7-yl)methyl)morpholine-4-sulfonic acid (40 mg, 0.07 mmol) was reacted according to General Procedure 8. The crude was purified by Prep HPLC (Method F) to afford the title compound (21.8 mg, 62%) as a white solid. ¹H NMR (400 MHz, DMSO-d₆+D₂O): δ 8.53 (s, 1H), 7.34-7.29 (m, 1H), 7.24 (s, 1H), 7.11 (t, J=7.9 Hz, 1H), 6.91-6.84 (m, 1H), 5.36 (d, J=21.6 Hz, 2H), 4.04-3.99 (m, 1H), 3.93 (d, J=1.7 Hz, 7H), 3.82-3.65 (m, 4H), 3.63-3.47 (m, 3H), 3.34-3.24 (m, 3H), 3.12-3.01 (m, 1H), 0.79-0.67 (m, 2H), 0.53-0.45 (m, 2H). LCMS (ESI, m/z): 519 [M+H]+. LCMS RT: 1.550 min. (Method B).

Compounds of Examples 27 and 28 in Table 3 were obtained following a procedure similar to the preparation of a compound of Example 26 using the appropriate alkylating agent in step 2.

TABLE 3

| Example | | LCMS m/z [M + H]+; LCMS RT (LCMS method); |
|---|---|---|
| Number | Structure | ¹H NMR (MHz, solvent) |
| 27 | 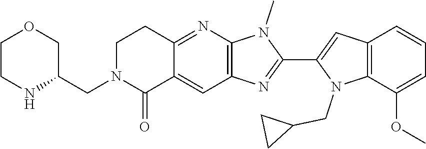 [SMILES: COc1cccc2cc(-c3nc4cc5c(nc4n3C)CCN(C[C@H]3COCCN3)C5=O)n(CC3CC3)c12] | (S)-2-(1-(cyclopropylmethyl)-7-methoxy-1H-indol-2-yl)- 3-methyl-7-(morpholin-3-ylmethyl)-3,5,6,7-tetrahydro-8H- imidazo[4,5-b][1,6]naphthyridin-8-one LCMS (ESI, m/z): 501 [M + H]⁺; LCMS RT: 1.118 min. (Method D). |
| 28 | 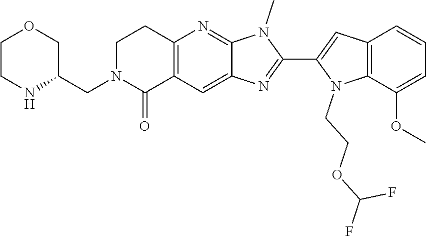 [SMILES: COc1cccc2cc(-c3nc4cc5c(nc4n3C)CCN(C[C@H]3COCCN3)C5=O)n(CCOC(F)F)c12] | (S)-2-(1-(2-(difluoromethoxy)ethyl)-7-methoxy-1H-indol- 2-yl)-3-methyl-7-(morpholin-3-ylmethyl)-3,5,6,7- tetrahydro-8H-imidazo[4,5-b][1,6]naphthyridin-8-one LCMS (ESI, m/z): 541 [M + H]+; RT: 0.921 min. (Method D) ¹H NMR (400 MHz, Methanol-d4) δ 8.68 (s, 1H), 7.32 (d, J = 8.0 Hz, 1H), 7.13 (t, J = 7.6 Hz, 1H), 7.07 (s, 1H), 6.90 (d, J = 7.6 Hz, 1H), 6.06 (t, J = 75.6 Hz, 1H), 5.08 (t, J = 5.2 Hz, 2H), 4.22-4.10 (m, 2H), 4.06-4.02 (m, 9H), 3.95- 3.74 (m, 3H), 3.74-3.65 (m, 2H), 3.64-3.57 (m, 1H), 3.47- 3.39 (m, 3H), 3.30-3.19 (m, 1H). |

### Example 29. (S)-2-(7-(1-acetylpiperidin-4-yl)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

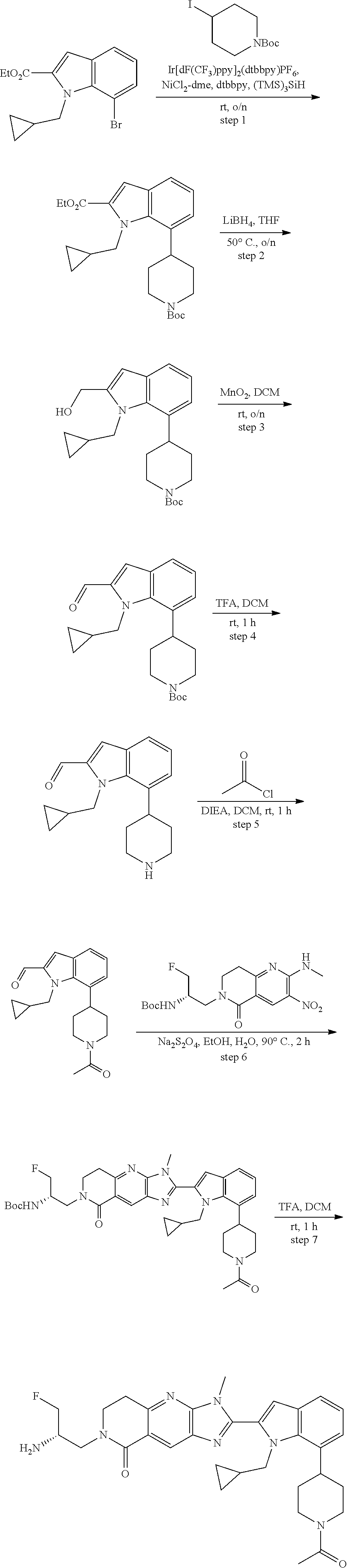

### Step 1: Synthesis of ethyl 7-(1-(tert-butoxycarbonyl)piperidin-4-yl)-1-(cyclopropylmethyl)-1H-indole-2-carboxylate

To a solution of ethyl 7-bromo-1-(cyclopropylmethyl)indole-2-carboxylate (150 mg, 0.47 mmol) and tert-butyl 4-iodopiperidine-1-carboxylate (145 mg, 0.47 mmol) in DMF (6 mL), was added Ir[dF(CF₃)ppy]₂(dtbbpy)PF₆ (52.7 mg, 0.05 mmol), NiCl₂-dme (5.17 mg, 0.02 mmol), dtbbpy (6.3 mg, 0.02 mmol), (TMS)₃SiH (116 mg, 0.47 mmol) and Na₂CO₃ (98.7 mg, 0.93 mmol). The reaction was stirred and irradiated with blue LED lamp (7 cm away, with cooling fan to keep the reaction temperature at 25° C.) for overnight under nitrogen atmosphere. The reaction was monitored by LCMS. The reaction was then quenched by adding water (20 mL) and extracted with ethyl acetate (20 mL), washed with water (2×20 mL) and brine (2×20 mL), dried over anhydrous sodium sulfate and concentrated under vacuum. The crude product was purified by column chromatography (petroleum ether/ethyl acetate=5:1) to afford the title compound (30 mg, 15%) as a light yellow solid. LCMS (ESI, m/z): 427 [M+H]+.

### Step 2: Synthesis of tert-butyl 4-(1-(cyclopropylmethyl)-2-(hydroxymethyl)-1H-indol-7-yl)piperidine-1-carboxylate

To a solution of ethyl 7-(1-(tert-butoxycarbonyl)piperidin-4-yl)-1-(cyclopropylmethyl)-1H-indole-2-carboxylate (110 mg, 0.26 mmol) in THF (3 mL), was added LiBH₄ (0.02 mL, 1.03 mmol) at 0° C. The resulting solution was stirred at 50° C. for overnight. The reaction was monitored by LCMS. The reaction was then quenched by adding water (10 mL) and extracted with ethyl acetate (2×10 mL). The combined organic extracts were dried over anhydrous sodium sulfate and concentrated under vacuum. The crude product was purified by column chromatography (petroleum ether/ethyl acetate=5:1) to afford the title compound (40 mg, 40%) as a light yellow solid. LCMS (ESI, m/z): 385 [M+H]+.

### Step 3: Synthesis of tert-butyl 4-(1-(cyclopropylmethyl)-2-formyl-1H-indol-7-yl)piperidine-1-carboxylate

tert-butyl 4-(1-(cyclopropylmethyl)-2-(hydroxymethyl)-1H-indol-7-yl)piperidine-1-carboxylate (55 mg, 0.14 mmol) was reacted according to General Procedure 4. The crude product was purified by column chromatography (petroleum ether/ethyl acetate=6:1) to afford the title compound (50 mg, 91%) as a light yellow solid. LCMS (ESI, m/z): 383 [M+H]+.

### Step 4: Synthesis of 1-(cyclopropylmethyl)-7-(piperidin-4-yl)-1H-indole-2-carbaldehyde

tert-butyl 4-(1-(cyclopropylmethyl)-2-formyl-1H-indol-7-yl)piperidine-1-carboxylate (50 mg, 0.13 mmol) was reacted according to General Procedure 2. The resulting solution was concentrated under vacuum to afford the title compound (35 mg, 95%) as a light yellow solid. LCMS (ESI, m/z): 283 [M+H]+.

### Step 5: Synthesis of 7-(1-acetylpiperidin-4-yl)-1-(cyclopropylmethyl)-1H-indole-2-carbaldehyde

To a solution of 1-(cyclopropylmethyl)-7-(piperidin-4-yl)-1H-indole-2-carbaldehyde (50 mg, 0.18 mmol) in DCM (5 mL), was added acetyl chloride (27.8 mg, 0.35 mmol) and DIEA (0.05 mL, 0.71 mmol). The resulting solution was stirred at room temperature for 1 h. The reaction was monitored by LCMS. The reaction was then quenched by adding water (10 mL) and extracted with DCM (3×10 mL). The combined organic extracts were dried over anhydrous sodium sulfate and concentrated under vacuum. The crude product was purified by column chromatography to afford the title compound (30 mg, 52%) as a light yellow solid. LCMS (ESI, m/z): 325 [M+H]+.

### Step 6: Synthesis of tert-butyl (S)-(1-(2-(7-(1-acetylpiperidin-4-yl)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

7-(1-acetylpiperidin-4-yl)-1-(cyclopropylmethyl)-1H-indole-2-carbaldehyde (55 mg, 0.17 mmol) was reacted with Intermediate 2 (61.3 mg, 0.15 mmol) according to General Procedure 6. The crude product was purified by column chromatography (DCM/MeOH=20:1) to afford the title compound (25 mg, 24%) as a light yellow solid. LCMS (ESI, m/z): 672 [M+H]+.

### Step 7: Synthesis of (S)-2-(7-(1-acetylpiperidin-4-yl)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

tert-butyl (S)-(1-(2-(7-(1-acetylpiperidin-4-yl)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (20 mg, 0.03 mmol) was reacted according to General Procedure 2. The crude product was purified by Prep HPLC (Method F) to afford the title compound (12.1 mg, 68%) as a white solid. LCMS (ESI, m/z): 572 [M+H]+. LCMS RT: 2.489 min. (Method B).

Compounds of Examples 30 to 32 in Table 4 were obtained following a procedure similar to the preparation of a compound of Example 29 using the appropriate acylating agent in step 5.

TABLE 4

| Example | | LCMS m/z [M + H]+; LCMS RT (LCMS method); |
|---|---|---|
| Number | Structure | ¹H NMR (MHz, solvent) |
| 30 | 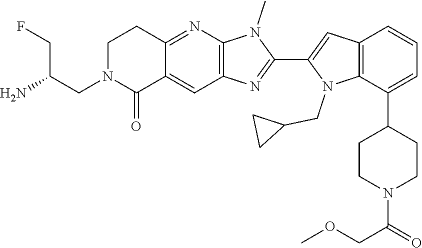 [SMILES: COCC(=O)N1CCC(c2cccc3cc(-c4nc5cc6c(nc5n4C)CCN(C[C@H](N)CF)C6=O)n(CC4CC4)c23)CC1] | (S)-7-(2-amino-3-fluoropropyl)-2-(1- (cyclopropylmethyl)-7-(1-(2-methoxyacetyl)piperidin- 4-yl)-1H-indol-2-yl)-3-methyl-3,5,6,7-tetrahydro-8H- imidazo[4,5-b][1,6]naphthyridin-8-one LCMS (ESI, m/z): 602 [M + H]+; LCMS RT: 1.426 min. (Method B). |
| 31 | 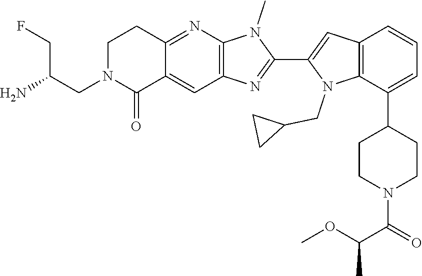 [SMILES: CO[C@H](C)C(=O)N1CCC(c2cccc3cc(-c4nc5cc6c(nc5n4C)CCN(C[C@H](N)CF)C6=O)n(CC4CC4)c23)CC1] | 7-((S)-2-amino-3-fluoropropyl)-2-(1- (cyclopropylmethyl)-7-(1-((R)-2- methoxypropanoyl)piperidin-4-yl)-1H-indol-2-yl)-3- methyl-3,5,6,7-tetrahydro-8H-imidazo[4,5- b][1,6]naphthyridin-8-one LCMS (ESI, m/z): 616 [M + H]+; LCMS RT: 0.847 min. (Method D) ¹H NMR (400 MHz, DMSO-d₆) δ 8.51 (s, 1H), 8.28 (s, br 3H), 7.60 (d, J = 8 Hz, 1H), 7.25 (s, 2H), 7.18-7.14 (m, 1H), 4.81-4.69 (m, 2H), 4.65-4.63 (m, 3H), 4.30- 4.26 (m, 2H), 3.97 (s, 3H), 3.87-3.66 (m, 7H), 3.34 (t, J = 6.4 Hz, 2H), 3.25 (s, 3H), 2.83-2.77 (m, 1H), 2.04 (s, br 2H), 1.77-1.64 (m, 2H), 1.30-1.25 (m, 3H), 0.95-0.93 (m, 1H), 0.24 (d, J = 4.4 Hz, 2H), −0.30 (d, J = 4.4 Hz, 2H). |
| 32 | 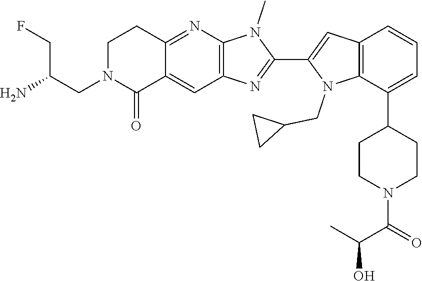 [SMILES: C[C@H](O)C(=O)N1CCC(c2cccc3cc(-c4nc5cc6c(nc5n4C)CCN(C[C@H](N)CF)C6=O)n(CC4CC4)c23)CC1] | 7-((S)-2-amino-3-fluoropropyl)-2-(1- (cyclopropylmethyl)-7-(1-((S)-2- hydroxypropanoyl)piperidin-4-yl)-1H-indol-2-yl)-3- methyl-3,5,6,7-tetrahydro-8H-imidazo[4,5- b][1,6]naphthyridin-8-one LCMS (ESI, m/z): 602 [M + H]+; LCMS RT: 3.721 min. (Method D) ¹H NMR (400 MHz, DMSO-d₆) δ 8.49 (s, 1H), 8.30 (s, 3H), 7.57 (d, J = 7.6 Hz, 1H), 7.22 (s, 2H), 7.22 (s, 2H), 7.15-7.11 (m, 1H), 4.78-4.48 (m, 5H), 4.19- 4.02 (m, 1H), 3.95 (s, 3H), 3.84-3.30 (m, 7H), 2.93- 2.80 (m, 1H), 2.05-2.00 (m, 2H), 1.77-1.64 (m, 2H), 1.33-1.22 (m, 4H), 0.92-0.83 (m, 1H), 0.23- 0.21 (m, 2H), −0.31-−0.32 (m, 2H). |

### Example 33. (S)-7-(2-amino-3-fluoropropyl)-2-(1-(cyclopropylmethyl)-6-(2-hydroxypropan-2-yl)-1H-pyrrolo[2,3-b]pyridin-2-yl)-3-methyl-3,5,6,7-tetrahydro-8H-imidazo[4,5-b][1,6]naphthyridin-8-one

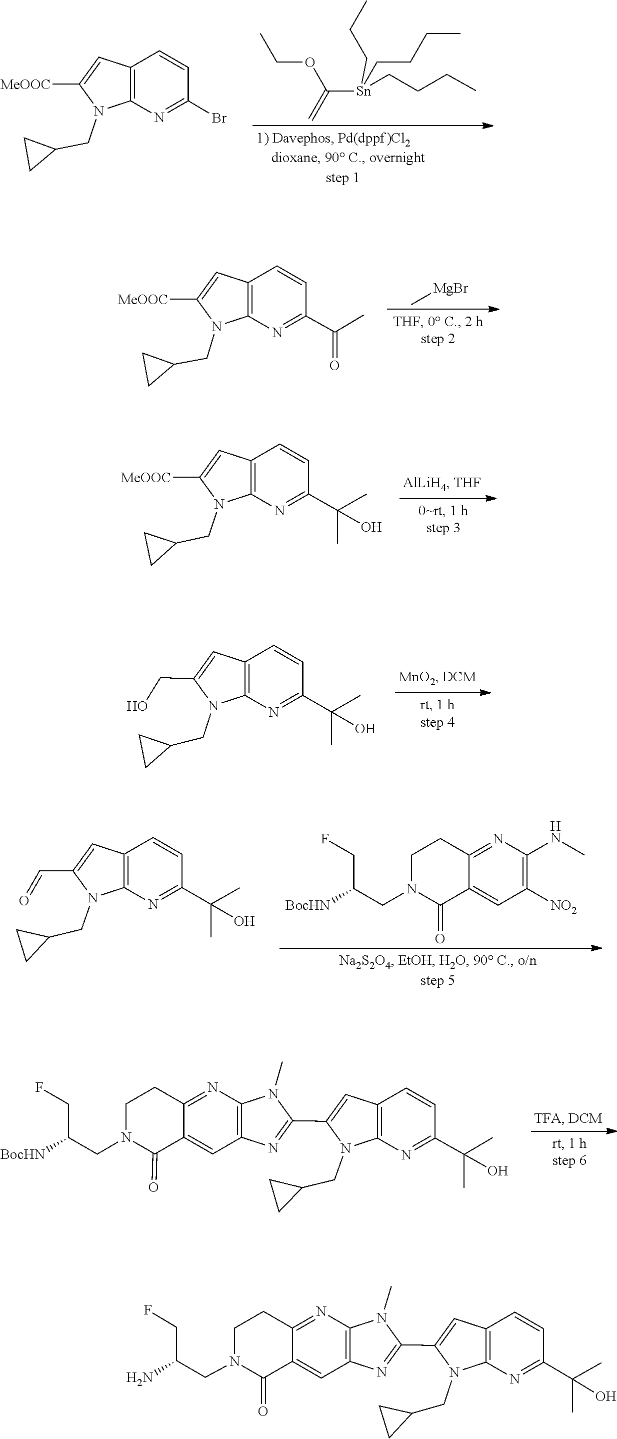

### Step 1 Synthesis of methyl 6-acetyl-1-(cyclopropylmethyl)pyrrolo[2,3-b]pyridine-2-carboxylate

To a solution of methyl 6-chloro-1-(cyclopropylmethyl)pyrrolo[2,3-b]pyridine-2-carboxylate (264 mg, 1 mmol) and tributyl(1-ethoxyvinyl)stannane (720 mg, 1.9 mmol) in 1,4-dioxane (10 mL), was added Davephos (78 mg, 0.2 mmol) and Pd(dppf)Cl₂ (73 mg, 0.1 mmol) under nitrogen atmosphere. The mixture was stirred at room temperature for 0.5 h. To the above mixture was added HCl (1 M aq.) at 0° C. The mixture was stirred at room temperature for 2 h. The reaction was concentrated under vacuum. The crude product was purified by column chromatography (petroleum ether/ethyl acetate=3:1) to afford the title compound (130 mg, 47.8%) as a yellow oil. LCMS (ESI, m/z): 273 [M+H]+.

### Step 2 Synthesis of methyl 1-(cyclopropylmethyl)-6-(1-hydroxy-1-methyl-ethyl)pyrrolo[2,3-b]pyridine-2-carboxylate

To a solution of methyl 6-acetyl-1-(cyclopropylmethyl)pyrrolo[2,3-b]pyridine-2-carboxylate (130 mg, 0.5 mmol) in THE (5 mL), was added methylmagnesium bromide (3 M in diethyl ether, 0.3 mL, 0.9 mmol) at 0° C. The mixture was stirred at room temperature at 0° C. for 4 h. The reaction was then quenched by adding saturated aqueous NH₄Cl (10 mL) and extracted with ethyl acetate (20 mL), washed with water (2×10 mL) and brine (10 mL), dried over anhydrous sodium sulfate and concentrated under vacuum. The crude product was purified by column chromatography (petroleum ether/ethyl acetate=1:1) to afford the title compound (110 mg, 79.9%) as a light yellow solid. LCMS (ESI, m/z): 289 [M+H]+.

### Step 3: Synthesis of 2-[1-(cyclopropylmethyl)-2-(hydroxymethyl)pyrrolo[2,3-b]pyridin-6-yl]propan-2-ol

Methyl 1-(cyclopropylmethyl)-6-(1-hydroxy-1-methyl-ethyl)pyrrolo[2,3-b]pyridine-2-carboxylate (80 mg, 0.3 mmol) was reacted according to General Procedure 3. The crude product was purified by column chromatography (petroleum ether/ethyl acetate=1:1) to afford the title compound (70 mg, 96.9%) as a light yellow solid. LCMS (ESI, m/z): 261 [M+H]+

### Step 4: Synthesis of 1-(cyclopropylmethyl)-6-(1-hydroxy-1-methyl-ethyl)pyrrolo[2,3-b]pyridine-2-carbaldehyde

2-[1-(cyclopropylmethyl)-2-(hydroxymethyl)pyrrolo[2,3-b]pyridin-6-yl]propan-2-ol (70 mg, 0.3 mmol) was reacted according to General Procedure 4. The crude product was purified by column chromatography (petroleum ether/ethyl acetate=1:1) to afford the title compound (50 mg, 71.9%) as a light yellow solid. LCMS (ESI, m/z): 259 [M+H]+.

### Step 5: Synthesis of tert-butyl (S)-(1-(2-(1-(cyclopropylmethyl)-6-(2-hydroxypropan-2-yl)-1H-pyrrolo[2,3-b]pyridin-2-yl)-3-methyl-8-oxo-3,5,6,8-tetrahydro-7H-imidazo[4,5-b][1,6]naphthyridin-7-yl)-3-fluoropropan-2-yl)carbamate

1-(cyclopropylmethyl)-6-(1-hydroxy-1-methyl-ethyl)pyrrolo[2,3-b]pyridine-2-carbaldehyde (40 mg, 0.15 mmol) was reacted with Intermediate 2 (74 mg, 0.2 mmol) according to General Procedure 6. The crude product was purified by column chromatography (ethyl acetate) to afford the title compound (30 mg, 31.9%) as a light yellow solid. LCMS (ESI, m/z): 606 [M+H]+.

### Step 6 Synthesis of (S)-7-(2-amino-3-fluoropropyl)-2-(1-(cyclopropylmethyl)-6-(2-hydroxypropan-2-yl)-1H-pyrrolo[2,3-b]pyridin-2-yl)-3-methyl-3,5,6,7-tetrahydro-8H-imidazo[4,5-b][1,6]naphthyridin-8-one

Tert-butyl (S)-(1-(2-(1-(cyclopropylmethyl)-6-(2-hydroxypropan-2-yl)-1H-pyrrolo[2,3-b]pyridin-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.05 mmol) was reacted according to General Procedure 2. The crude product was purified by Prep-HPLC (Method F) to afford the title compound (15.6 mg, 61.6%) as a white solid. ¹H NMR (400 MHz, DMSO-d₆) δ 8.54 (s, 1H), 8.29 (s, 3H), 8.11 (d, J=8.0 Hz, 1H), 7.57 (d, J=8.0 Hz, 1H), 7.25 (s, 1H), 5.28 (s, 1H), 4.81-4.70 (m, 1H), 4.72-4.60 (m, 3H), 4.04 (s, 3H), 3.88-3.70 (m, 5H), 3.4-3.3 (m, 2H), 1.55 (s, 6H), 1.29-1.25 (m, 1H), 0.37-0.23 (m, 4H). LCMS (ESI, m/z): 506 [M+H]+. LCMS RT: 0.819 min. (Method B).

### Example 34. (S)-5-(((2-(7-((S)-2-amino-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)methyl)oxazolidin-2-one

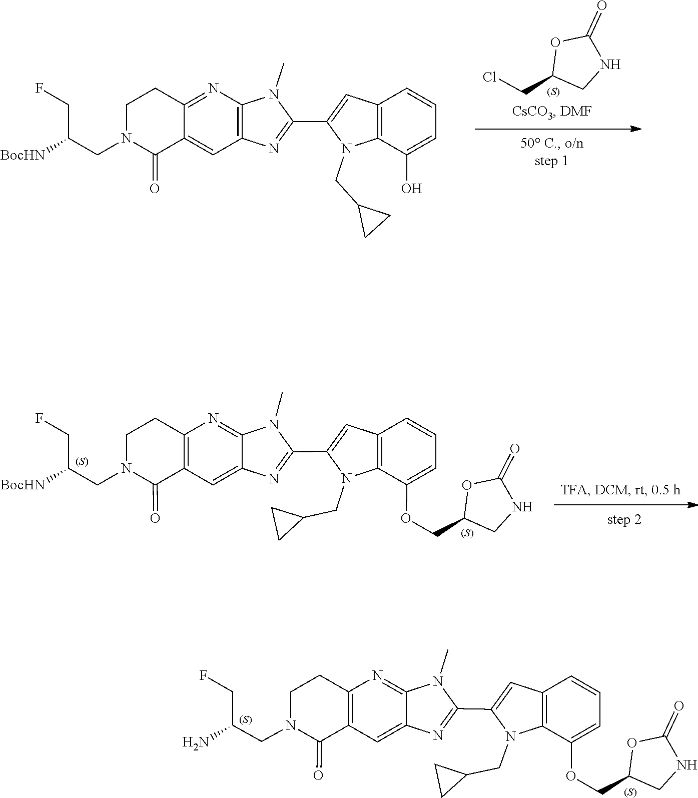

### Step 1: Synthesis of tert-butyl ((S)-1-(2-(1-(cyclopropylmethyl)-7-(((S)-2-oxooxazolidin-5-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 5 (50 mg, 0.09 mmol) was reacted with (S)-5-(chloromethyl)oxazolidin-2-one (23 mg, 0.17 mmol) according to General Procedure 5. The crude product was purified by column chromatography (DCM/MeOH=10:1) to afford the title compound (40 mg, 68%) as a yellow oil. LCMS (ESI, m/z): 662 [M+H]+.

### Step 2: Synthesis of (S)-5-(((2-(7-((S)-2-amino-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)methyl)oxazolidin-2-one

Tert-butyl ((S)-1-(2-(1-(cyclopropylmethyl)-7-(((S)-2-oxooxazolidin-5-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (40 mg, 0.06 mmol) was reacted according to General Procedure 4. The crude product was purified by Prep-HPLC (Method E) to afford the title compound (12.5 mg, 36%) as a light yellow solid. ¹H NMR (400 MHz, DMSO-d₆): δ 8.53 (s, 1H), 8.29 (s, 3H), 7.66 (s, 1H), 7.33 (d, J=7.9 Hz, 1H), 7.21 (s, 1H), 7.08 (t, J=7.8 Hz, 1H), 6.90 (d, J=7.8 Hz, 1H), 5.10-5.00 (m, 1H), 4.84-4.58 (m, 4H), 4.44-4.38 (m, 1H), 4.34-4.28 (m, 1H), 3.97 (s, 3H), 3.93-3.81 (m, 2H), 3.81-3.67 (m, 4H), 3.55-3.42 (m, 1H), 3.34 (t, J=6.6 Hz, 2H), 1.02-0.93 (m, 1H), 0.26-0.13 (m, 2H), −0.07-−0.16 (m, 2H). LCMS (ESI, m/z): 562 [M+H]+. LCMS RT: 1.385 min (Method B).

### Example 35. 7-((S)-2-amino-3-fluoropropyl)-2-(1-(cyclopropylmethyl)-7-((2,2-dimethyl-5-oxopyrrolidin-3-yl)methoxy)-1H-indol-2-yl)-3-methyl-3,5,6,7-tetrahydro-8H-imidazo[4,5-b][1,6]naphthyridin-8-one

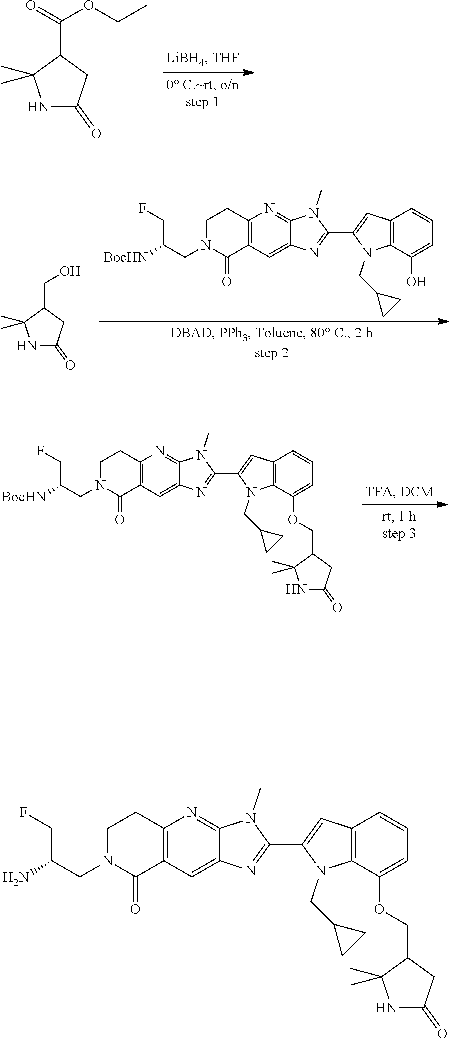

### Step 1: Synthesis of 4-(hydroxymethyl)-5,5-dimethyl-pyrrolidin-2-one

To a stirred solution of ethyl 2,2-dimethyl-5-oxo-pyrrolidine-3-carboxylate (100 mg, 0.54 mmol) in THF (5 mL) at 0° C. under a nitrogen atmosphere was added LiBH₄ (2 M in THF, 0.1 mL, 2.16 mmol) dropwise at 0′° C. The resulting mixture was stirred overnight at room temperature under a nitrogen atmosphere. The reaction was monitored by TLC and LCMS. The reaction was quenched by the addition of aqueous saturated NH₄Cl. The resulting mixture was concentrated under reduced pressure and purified by flash column chromatography to afford the title compound (80 mg, 103.5%) as a white solid. LCMS (ESI, m/z): 144 [M+H]*.

### Step 2: Synthesis of tert-butyl ((2S)-1-(2-(1-(cyclopropylmethyl)-7-((2,2-dimethyl-5-oxopyrrolidin-3-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 5 (50 mg, 0.09 mmol) was reacted with 4-(hydroxymethyl)-5,5-dimethyl-pyrrolidin-2-one (64 mg, 0.44 mmol) according to General Procedure 10. The crude product was purified by Prep-TLC to afford the title compound (40 mg, 65.4%) as yellow oil. LCMS (ESI, m/z): 688 [M+H]⁺.

### Step 3: Synthesis of 7-((S)-2-amino-3-fluoropropyl)-2-(1-(cyclopropylmethyl)-7-((2,2-dimethyl-5-oxopyrrolidin-3-yl)methoxy)-1H-indol-2-yl)-3-methyl-3,5,6,7-tetrahydro-8H-imidazo[4,5-b][1,6]naphthyridin-8-one

Tert-butyl ((2S)-1-(2-(1-(cyclopropylmethyl)-7-((2,2-dimethyl-5-oxopyrrolidin-3-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (40 mg, 0.306 mmol) was reacted according to General Procedure 2. The crude material was purified by Prep HPLC (Method E) to afford the title compound (28.5 mg, 82.7%) as an off-white solid. ¹H NMR (400 MHz, DMSO-d₆) δ 8.51 (s, 1H), 8.39-8.22 (m, 3H), 7.78 (s, 1H), 7.31 (d, J=7.9 Hz, 1H), 7.19 (s, 1H), 7.08 (t, J=7.8 Hz, 1H), 6.94 (d, J=7.8 Hz, 1H), 4.84-4.71 (m, 2H), 4.70-4.59 (m, 1H), 4.40-4.34 (m, 1H), 4.14-4.07 (m, 2H), 3.97 (s, 3H), 3.91-3.82 (m, 2H), 3.79-3.70 (m, 3H), 3.33 (t, J=6.6 Hz, 2H), 2.69-2.59 (m, 1H), 2.45-2.39 (m, 1H), 2.38-2.29 (m, 1H), 1.35 (s, 3H), 1.21 (s, 3H), 1.06-0.92 (m, 1H), 0.25-0.15 (m, 2H), −0.11-−0.27 (m, 2H). LCMS (ESI, m/z): 588 [M+H]⁺. LCMS RT: 1.447 min. (Method D).

Compounds of Examples 36-58 in Table 5 were obtained following a procedure similar to the preparation of a compound of Example 35 using the appropriate ester or alcohol starting material.

TABLE 5

| | | LCMS m/z [M + H]+; LCMS RT (LCMS |
|---|---|---|
| Example | | method); |
| Number | Structure | ¹H NMR (MHz, solvent) |
| 36 | 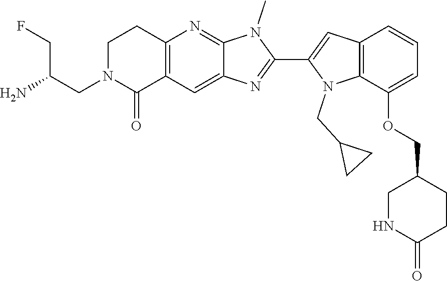 [SMILES: Cn1c(-c2cc3cccc(OC[C@H]4CCC(=O)NC4)c3n2CC2CC2)nc2cc3c(nc21)CCN(C[C@H](N)CF)C3=O] | 7-((S)-2-amino-3-fluoropropyl)-2-(1- (cyclopropylmethyl)-7-(((S)-6- oxopiperidin-3-yl)methoxy)-1H-indol-2- yl)-3-methyl-3,5,6,7-tetrahydro-8H- imidazo[4,5-b][1,6]naphthyridin-8-one LCMS (ESI, m/z): 574 [M + H]+; LCMS RT: 1.382 min. (Method B). |
| 37 | 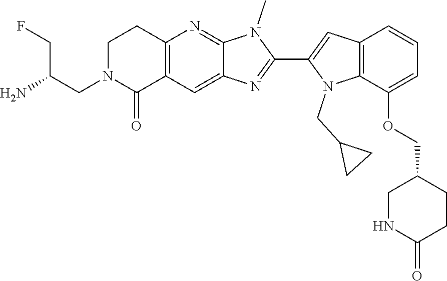 [SMILES: Cn1c(-c2cc3cccc(OC[C@@H]4CCC(=O)NC4)c3n2CC2CC2)nc2cc3c(nc21)CCN(C[C@H](N)CF)C3=O] | 7-((S)-2-amino-3-fluoropropyl)-2-(1- (cyclopropylmethyl)-7-(((R)-6- oxopiperidin-3-yl)methoxy)-1H-indol-2- yl)-3-methyl-3,5,6,7-tetrahydro-8H- imidazo[4,5-b][1,6]naphthyridin-8-one LCMS (ESI, m/z): 574 [M + H]+; LCMS RT: 1.376 min (Method B) ¹H NMR (400 MHz, DMSO-d₆) δ 8.51 (s, 1H), 7.30 (d, J = 7.9 Hz, 1H), 7.16 (s, 1H), 7.07 (t, J = 7.8 Hz, 1H), 6.88 (d, J = 7.8 Hz, 1H), 4.84-4.59 (m, 4H), 4.20-4.14 (m, 2H), 3.96 (s, 3H), 3.87-3.73 (m, 5H), 3.48- 3.42 (m, 1H), 3.34 (t, J = 6.6 Hz, 2H), 3.15 (t, J = 10.8 Hz, 1H), 2.38-2.25 (m, 3H), 2.05-1.94 (m, 1H), 1.77-1.64 (m, 1H), 1.01-0.09 (m, 1H), 0.27-0.18 (m, 2H), −0.12-−0.20 (m, 2H). |
| 38 | 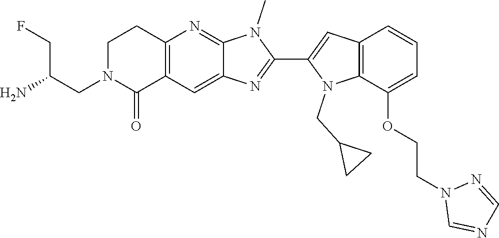 [SMILES: Cn1c(-c2cc3cccc(OCCn4cncn4)c3n2CC2CC2)nc2cc3c(nc21)CCN(C[C@H](N)CF)C3=O] | (S)-2-(7-(2-(4H-1,2,4-triazol-4-yl)ethoxy)- 1-(cyclopropylmethyl)-1H-indol-2-yl)-7- (2-amino-3-fluoropropyl)-3-methyl- 3,5,6,7-tetrahydro-8H-imidazo[4,5- b][1,6]naphthyridin-8-one LCMS (ESI, m/z): 558 [M + H]+; LCMS RT: 1.286 min (Method B) ¹H NMR (400 MHz, DMSO-d₆) δ 8.66 (s, 2H), 8.48 (s, 1H), 7.31 (d, J = 7.9 Hz, 1H), 7.15 (s, 1H), 7.07 (t, J = 7.8 Hz, 1H), 6.88 (d, J = 7.7 Hz, 1H), 4.64-4.51 (m, 6H), 4.46-4.37 (m, 1H), 4.29 (m, 1H), 3.94 (s, 3H), 3.81-3.71 (m, 2H), 3.64-3.56 (m, 1H), 3.51-3.43 (m, 1H), 3.32-3.28 (m, 3H), 0.80-0.77 (m, 1H), 0.17-0.05 (m, 2H), −0.24-−0.27 (m, 2H). |
| 39 | 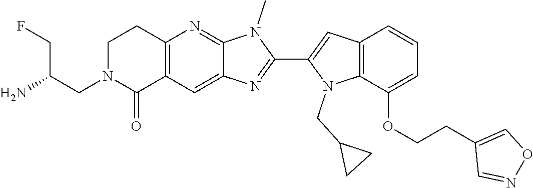 [SMILES: Cn1c(-c2cc3cccc(OCCc4cnoc4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(isoxazol-4- yl)ethoxy)-1H-indol-2-yl)-3-methyl- 3,5,6,7-tetrahydro-8H-imidazo[4,5- b][1,6]naphthyridin-8-one LCMS (ESI, m/z): 558 [M + H]⁺; LCMS RT: 1.118 min (Method D) ¹H NMR (400 MHz, DMSO-d₆ + D₂O): δ 8.88 (s, 1H), 8.67 (s, 1H), 8.50 (s, 1H), 8.42- 8.26 (m, 3H), 7.29 (d, J = 7.9 Hz, 1H), 7.16 (s, 1H), 7.06 (t, J = 7.8 Hz, 1H), 6.89 (d, J = 7.8 Hz, 1H), 4.82-4.66 (m, 2H), 4.63 (d, J = 7.1 Hz, 2H), 4.41 (t, J = 6.3 Hz, 2H), 3.94 (s, 3H), 3.91-3.80 (m, 2H), 3.79- 3.70 (m, 3H), 3.32 (t, J = 6.6 Hz, 2H), 3.05 (t, J = 6.3 Hz, 2H), 0.95-0.80 (m, 1H), 0.18-0.10 (m, 2H), −0.19-−0.31 (m, 2H). |
| 40 | 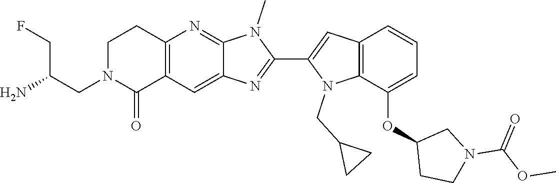 [SMILES: COC(=O)N1CC[C@@H](Oc2cccc3cc(-c4nc5cc6c(nc5n4C)CCN(C[C@H](N)CF)C6=O)n(CC4CC4)c23)C1] | methyl (R)-3-((2-(7-((S)-2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)pyrrolidine-1-carboxylate LCMS (ESI, m/z): 590 [M + H]+; LCMS RT: 1.452 min (Method B). |
| 41 | 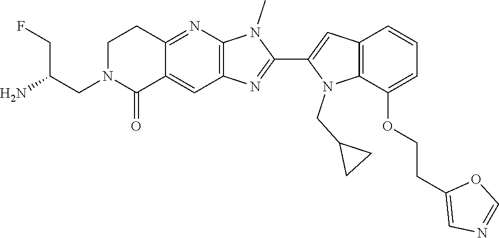 [SMILES: Cn1c(-c2cc3cccc(OCCc4cnco4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(oxazol-5- yl)ethoxy)-1H-indol-2-yl)-3-methyl- 3,5,6,7-tetrahydro-8H-imidazo[4,5- b][1,6]naphthyridin-8-one LCMS (ESI, m/z): 558 [M + H]+; LCMS RT: 1.457 min (Method B). |
| 42 | 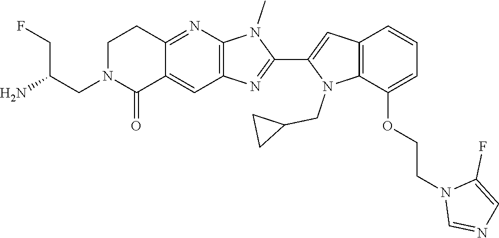 [SMILES: Cn1c(-c2cc3cccc(OCCn4cncc4F)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(5-fluoro-1H- imidazol-1-yl)ethoxy)-1H-indol-2-yl)-3- methyl-3,5,6,7-tetrahydro-8H- imidazo[4,5-b][1,6]naphthyridin-8-one LCMS (ESI, m/z): 575 [M + H]+; LCMS RT: 1.246 min (Method B) ¹H NMR (400 MHz, DMSO-d₆): δ 8.51 (s, 1H), 8.32-8.27 (m, 4H), 7.36-7.29 (m, 1H), 7.19 (s, 1H), 7.14-7.04 (m, 2H), 6.99- 6.95 (m, 1H), 4.84-4.61 (m, 2H), 4.59 (s, 6H), 3.98-3.81 (m, 8H), 3.33 (t, J = 6.6 Hz, 2H), 0.89-0.76 (m, 1H), 0.18-0.09 (m, 2H), −0.27-−0.34 (m, 2H). |
| 43 | 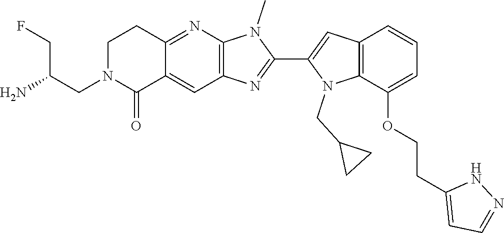 [SMILES: Cn1c(-c2cc3cccc(OCCc4ccn[nH]4)c3n2CC2CC2)nc2cc3c(nc21)CCN(C[C@H](N)CF)C3=O] | (S)-2-(7-(2-(1H-pyrazol-5-yl)ethoxy)-1- (cyclopropylmethyl)-1H-indol-2-yl)-7-(2- amino-3-fluoropropyl)-3-methyl-3,5,6,7- tetrahydro-8H-imidazo[4,5- b][1,6]naphthyridin-8-one LCMS (ESI, m/z): 557 [M + H]+; LCMS RT: 0.828 min (Method B) ¹H NMR (400 MHz, DMSO-d₆) δ 8.51 (s, 1H), 8.35-8.30 (m, 3H), 7.58 (d, J = 2.1 Hz, 1H), 7.28 (d, J = 7.9 Hz, 1H), 7.15 (s, 1H), 7.06 (t, J = 7.8 Hz, 1H), 6.88 (d, J = 7.8 Hz, 1H), 6.24 (d, J = 2.1 Hz, 1H), 4.84- 4.69 (m, 1H), 4.72-4.58 (m, 4H), 4.45 (m, 2H), 3.95 (s, 3H), 3.76 (m, 5H), 3.33 (m, 2H), 3.18 (m, 2H), 0.89 (m, 1H), 0.19- 0.08 (m, 2H), −0.18 (m, 2H). |
| 44 | 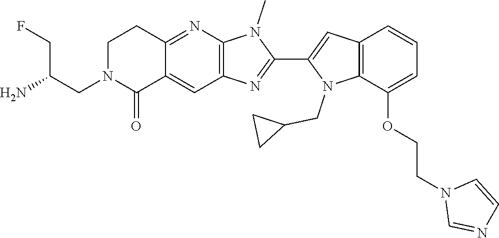 [SMILES: Cn1c(-c2cc3cccc(OCCn4ccnc4)c3n2CC2CC2)nc2cc3c(nc21)CCN(C[C@H](N)CF)C3=O] | (S)-2-(7-(2-(1H-imidazol-1-yl)ethoxy)-1- (cyclopropylmethyl)-1H-indol-2-yl)-7-(2- amino-3-fluoropropyl)-3-methyl-3,5,6,7- tetrahydro-8H-imidazo[4,5- b][1,6]naphthyridin-8-one LCMS (ESI, m/z): 557 [M + H]+; LCMS RT: 0.651 min (Method D) ¹H NMR (400 MHz, DMSO-d6) δ 9.21 (s, 1H), 8.49 (s, 1H), 7.92 (s, 1H), 7.72 (s, 1H), 7.33 (d, J = 7.9 Hz, 1H), 7.18 (s, 1H), 7.10 (t, J = 7.8 Hz, 1H), 6.92 (d, J = 7.8 Hz, 1H), 4.80-4.72 (m, 3H), 4.71-4.65 (m, 3H), 4.52 (d, J = 7.0 Hz, 2H), 3.95 (s, 3H), 3.85- 3.71 (m, 5H), 3.29-3.17 (m, 2H), 0.69 (m, 1H), 0.14-0.08 (m, 2H), −0.31-−0.40 (m, 2H). |
| 45a Chiral sep 1 | 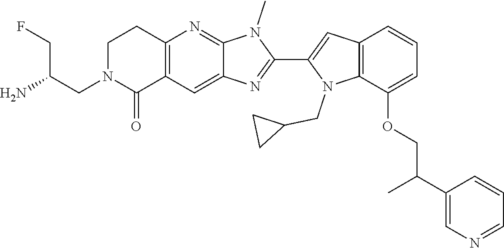 [SMILES: CC(COc1cccc2cc(-c3nc4cc5c(nc4n3C)CCN(C[C@H](N)CF)C5=O)n(CC3CC3)c12)c1cccnc1] | 7-((S)-2-amino-3-fluoropropyl)-2-(1- (cyclopropylmethyl)-7-(2-(pyridin-3- yl)propoxy)-1H-indol-2-yl)-3-methyl- 3,5,6,7-tetrahydro-8H-imidazo[4,5- b][1,6]naphthyridin-8-one LCMS (ESI, m/z): 582 [M + H]+; LCMS RT: 0.782 min (Method B) ¹H NMR (400 MHz, DMSO-d₆) δ 8.90 (s, 1H), 8.71 (d, J = 4.8 Hz, 1H), 8.49 (s, 1H), 8.39-8.33 (m, 4H), 7.81 (t, J = 5.6 Hz, 1H), 7.28 (d, J = 8.0 Hz, 1H), 7.15 (s, 1H), 7.07 (t, J = 7.6 Hz, 1H), 6.91 (d, J = 8.0 Hz, 1H), 4.78-4.62 (m, 2H), 4.53-4.40 (m, 4H), 3.93-3.73 (m, 8H), 3.62-3.59 (m, 1H), 3.33 (t, J = 6.4 Hz, 2H), 1.46 (d, J = 7.2 Hz, 3H), 0.76-0.72 (m, 1H), 0.10-0.01 (m, 2H), −0.36-−0.37 (m, 2H). Prep Chiral HPLC: Column: CHIRALPAK IF, 2 × 25 cm, 5 μm; Mobile Phase A: MTBE (0.5% 2M NH₃ in MeOH), Mobile Phase B: MeOH:DCM (1:1)(0.1% 2M NH₃ in MeOH); Flow rate: 20 mL/min; Gradient: isocratic 50% B in 18 min; Detection: UV (220/254 nm); RT: 11.61 min. |
| 45b Chiral sep 2 | 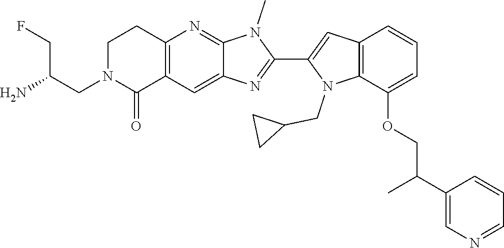 [SMILES: CC(COc1cccc2cc(-c3nc4cc5c(nc4n3C)CCN(C[C@H](N)CF)C5=O)n(CC3CC3)c12)c1cccnc1] | 7-((S)-2-amino-3-fluoropropyl)-2-(1- (cyclopropylmethyl)-7-(2-(pyridin-3- yl)propoxy)-1H-indol-2-yl)-3-methyl- 3,5,6,7-tetrahydro-8H-imidazo[4,5- b][1,6]naphthyridin-8-one LCMS (ESI, m/z): 582 [M + H]+; LCMS RT: 1.566 min (Method B) ¹H NMR (400 MHz, DMSO-d₆) δ 8.65 (d, J = 2.0 Hz, 1H), 8.50-8.47 (m, 2H), 8.31 (s, 3H), 7.90-7.88 (m, 1H), 7.42-7.40 (m, 1H), 7.27 (d, J = 8.0 Hz, 1H), 7.14 (s, 1H), 7.06 (t, J = 8.0 Hz, 1H), 6.90 (d, J = 7.6 Hz, 1H), 4.82-4.44 (m, 4H), 4.42- 4.37 (m, 2H), 3.93 (s, 3H), 3.87-3.83 (m, 2H), 3.78-3.72 (m, 3H), 3.40- 3.39 (m, 1H), 3.32-3.31 (m, 2H), 1.43 (d, J = 7.2 Hz, 3H), 0.82-0.72 (m, 1H), 0.12-0.01 (m, 2H), −0.32-−0.33 (m, 2H). Prep Chiral HPLC RT: 14.2 min. (Method see example 45a). |
| 46 | 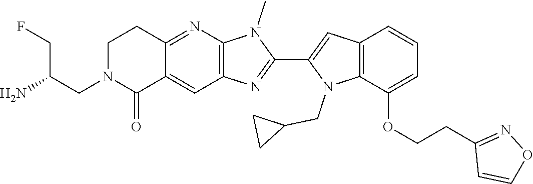 [SMILES: Cn1c(-c2cc3cccc(OCCc4ccon4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(isoxazol-3- yl)ethoxy)-1H-indol-2-yl)-3-methyl- 3,5,6,7-tetrahydro-8H-imidazo[4,5- b][1,6]naphthyridin-8-one LCMS (ESI, m/z): 558 [M + H]+; LCMS RT: 1.520 min (Method B) ¹H NMR (400 MHz, DMSO-d₆ + D₂O): δ 8.79 (d, J = 1.7 Hz, 1H), 8.50 (s, 1H), 7.31- 7.26 (m, 1H), 7.13 (s, 1H), 7.06 (t, J = 7.8 Hz, 1H), 6.90-6.85 (m, 1H), 6.64 (d, J = 1.7 Hz, 1H), 4.78-4.49 (m, 6H), 3.91 (s, 3H), 3.88-3.82 (m, 2H), 3.79-3.73 (m, 3H), 3.31 (t, J = 6.6 Hz, 2H), 3.24 (t, J = 5.9 Hz, 2H), 0.79-0.71 (m, 1H), 0.12- 0.06 (m, 2H), −0.24-−0.32 (m, 2H). |
| 47 | 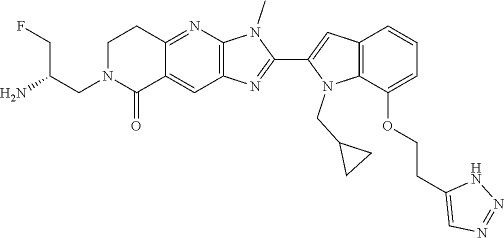 [SMILES: Cn1c(-c2cc3cccc(OCCc4cnn[nH]4)c3n2CC2CC2)nc2cc3c(nc21)CCN(C[C@H](N)CF)C3=O] | (S)-2-(7-(2-(1H-1,2,3-triazol-5-yl)ethoxy)- 1-(cyclopropylmethyl)-1H-indol-2-yl)-7- (2-amino-3-fluoropropyl)-3-methyl- 3,5,6,7-tetrahydro-8H-imidazo[4,5- b][1,6]naphthyridin-8-one LCMS (ESI, m/z): 558 [M + H]+; LCMS RT: 1.398 min (Method D) ¹H NMR (400 MHz, DMSO-d₆) δ 8.51 (s, 1H), 8.30 (s, 3H), 7.74 (s, 1H), 7.28 (d, J = 7.9 Hz, 1H), 7.16 (s, 1H), 7.06 (t, J = 7.8 Hz, 1H), 6.89 (d, J = 7.8 Hz, 1H), 4.84- 4.66 (m, 2H), 4.62 (dd, J= 8.4, 5.7 Hz, 2H), 4.48 (t, J = 6.3 Hz, 2H), 3.95 (s, 3H), 3.91-3.80 (m, 2H), 3.78-3.71 (m, 3H), 3.35-3.32 (m, 2H), 3.28-3.24 (m, 2H), 0.90-0.78 (m, 1H), 0.17-0.08 (m, 2H), −0.18-−0.25 (m, 2H). |
| 48 | 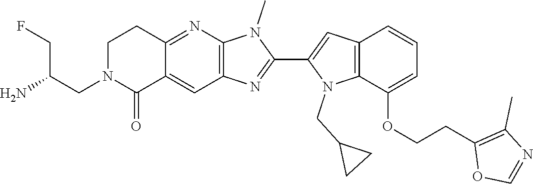 [SMILES: Cc1ncoc1CCOc1cccc2cc(-c3nc4cc5c(nc4n3C)CCN(C[C@H](N)CF)C5=O)n(CC3CC3)c12] | (S)-7-(2-amino-3-fluoropropyl)-2-(1- (cyclopropylmethyl)-7-(2-(4- methyloxazol-5-yl)ethoxy)-1H-indol-2- yl)-3-methyl-3,5,6,7-tetrahydro-8H- imidazo[4,5-b][1,6]naphthyridin-8-one LCMS (ESI, m/z): 572 [M + H]+; LCMS RT: 1.520 min (Method B) ¹H NMR (400 MHz, DMSO-d₆): δ 8.52 (s, 1H), 8.30 (s, 3H), 8.17 (s, 1H), 7.33-7.27 (m, 1H), 7.16 (s, 1H), 7.06 (t, J = 7.8 Hz, 1H), 6.93-6.87 (m, 1H), 4.80-4.59 (m, 4H), 4.42 (t, J = 5.9 Hz, 2H), 3.95 (s, 3H), 3.90-3.82 (m, 2H), 3.78-3.72 (m, 3H), 3.33 (t, J = 6.6 Hz, 2H), 3.24 (t, J = 5.9 Hz, 2H), 2.11 (s, 3H), 0.88-0.81 (m, 1H), 0.17- 0.11 (m, 2H), −0.22-−0.30 (m, 2H). |
| 49a earlier eluting peak | 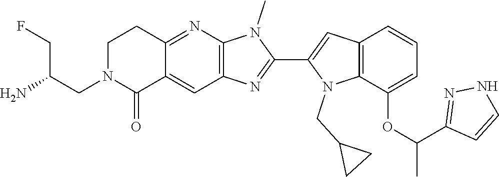 [SMILES: CC(Oc1cccc2cc(-c3nc4cc5c(nc4n3C)CCN(C[C@H](N)CF)C5=O)n(CC3CC3)c12)c1cc[nH]n1] | 2-(7-(1-(1H-pyrazol-3-yl)ethoxy)-1- (cyclopropylmethyl)-1H-indol-2-yl)-7- ((S)-2-amino-3-fluoropropyl)-3-methyl- 3,5,6,7-tetrahydro-8H-imidazo[4,5- b][1,6]naphthyridin-8-one LCMS (ESI, m/z): 557 [M + H]+; LCMS RT: 1.333 min (Method B) ¹H NMR (400 MHz, DMSO-d₆) δ 12.75 (s, 1H), 8.49 (s, 1H), 7.67 (s, 1H), 7.23 (d, J = 8.0 Hz, 1H), 7.13 (s, 1H), 6.99 (d, J = 8.0 Hz, 1H), 6.95 (d, J = 8.0 Hz, 1H), 6.34 (s, 1H), 5.76-5.74 (m, 1H), 4.81-4.78 (m, 2H), 4.56-4.44 (m, 2H), 3.94 (s, 3H), 3.78- 3.47 (m, 7H), 1.72 (d, J = 8.0 Hz, 3H), 1.09-1.02 (m, 1H), 0.21-0.19 (m, 2H), −0.06-−0.11 (m, 2H). Prep Chiral HPLC: Column: CHIRALPAK IF, 5 × 25 cm, 5 μm; Mobile Phase A: MTBE (0.5% 2M NH₃ in MeOH); Mobile Phase B: MeOH; Flow rate: 1.2 mL/min; Gradient: isocratic 30% B in 30 min; Detection: UV (220/254 nm); RT: 13.482 min. |
| 49b later eluting peak | 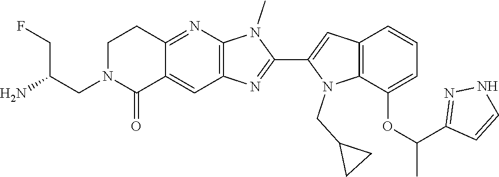 [SMILES: CC(Oc1cccc2cc(-c3nc4cc5c(nc4n3C)CCN(C[C@H](N)CF)C5=O)n(CC3CC3)c12)c1cc[nH]n1] | 2-(7-(1-(1H-pyrazol-3-yl)ethoxy)-1- (cyclopropylmethyl)-1H-indol-2-yl)-7- ((S)-2-amino-3-fluoropropyl)-3-methyl- 3,5,6,7-tetrahydro-8H-imidazo[4,5- b][1,6]naphthyridin-8-one LCMS (ESI, m/z): 557 [M + H]+; LCMS RT: 1.347 min (Method HALO) ¹H NMR (400 MHz, DMSO-d₆) δ 12.76 (s, 1H), 8.50 (s, 1H), 7.66 (s, 1H), 7.23 (d, J = 7.6 Hz, 1H), 7.13 (s, 1H), 7.01-6.93 (m, 2H), 6.34 (d, J = 2.0 Hz, 1H), 5.76-5.74 (m, 1H), 4.80 (d, J = 6.8 Hz, 2H), 4.61- 4.46 (m, 2H), 3.95 (s, 3H), 3.78-3.47 (m, 7H), 1.72 (d, J = 6.4 Hz, 3H), 1.07-1.04 (m, 1H), 0.21-0.19 (m, 2H), −0.08-−0.10 (m, 2H). Prep Chiral HPLC RT: 18.374 min. (Method see example 49a). |
| 50 | 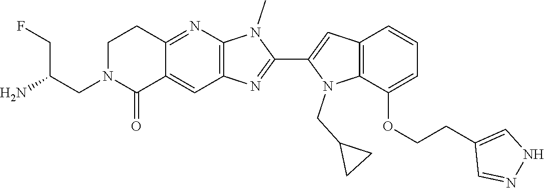 [SMILES: Cn1c(-c2cc3cccc(OCCc4cn[nH]c4)c3n2CC2CC2)nc2cc3c(nc21)CCN(C[C@H](N)CF)C3=O] | (S)-2-(7-(2-(1H-pyrazol-4-yl)ethoxy)-1- (cyclopropylmethyl)-1H-indol-2-yl)-7-(2- amino-3-fluoropropyl)-3-methyl-3,5,6,7- tetrahydro-8H-imidazo[4,5- b][1,6]naphthyridin-8-one LCMS (ESI, m/z): 557 [M + H]⁺; LCMS RT: 0.845 min. (Method C) ¹H NMR (400 MHz, DMSO-d₆ + D₂O) δ 8.55 (s, 1H), 7.66 (s, 2H), 7.31 (d, J = 7.7 Hz, 1H), 7.15 (s, 1H), 7.09 (t, J = 7.9 Hz, 1H), 6.89 (d, J = 7.8 Hz, 1H), 4.85-4.58 (m, 4H), 4.36 (t, J = 6.3 Hz, 2H), 3.99 (s, 3H), 3.91-3.82 (m, 2H), 3.77 (t, J = 8.2 Hz, 3H), 3.34 (t, J = 6.6 Hz, 2H), 3.06 (t, J = 6.3 Hz, 2H), 0.91-0.78 (m, 1H), 0.22-0.12 (m, 2H), −0.21-−0.31 (m, 2H). |
| 51 | 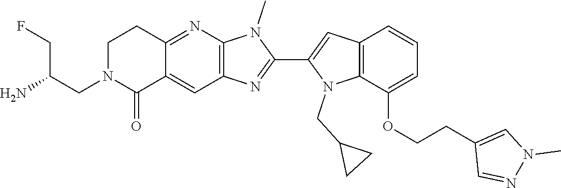 [SMILES: Cn1cc(CCOc2cccc3cc(-c4nc5cc6c(nc5n4C)CCN(C[C@H](N)CF)C6=O)n(CC4CC4)c23)cn1] | (S)-7-(2-amino-3-fluoropropyl)-2-(1- (cyclopropylmethyl)-7-(2-(1-methyl-1H- pyrazol-4-yl)ethoxy)-1H-indol-2-yl)-3- methyl-3,5,6,7-tetrahydro-8H- imidazo[4,5-b][1,6]naphthyridin-8-one LCMS (ESI, m/z): 571 [M + H]⁺; LCMS RT: 0.885 min. (Method C) ¹H NMR (400 MHz, DMSO-d₆) δ 8.51 (s, 1H), 8.36-8.23 (m, 3H), 7.62 (s, 1H), 7.39 (s, 1H), 7.27 (d, J = 7.9 Hz, 1H), 7.16 (s, 1H), 7.05 (t, J = 7.8 Hz, 1H), 6.87 (d, J = 7.7 Hz, 1H), 4.82-4.59 (m, 4H), 4.32 (t, J = 6.6 Hz, 2H), 3.95 (s, 3H), 3.91-3.82 (m, 2H), 3.79 (s, 3H), 3.77-3.65 (m, 3H), 3.33 (t, J = 6.6 Hz, 2H), 2.98 (t, J = 6.5 Hz, 2H), 1.00-0.89 (m, 1H), 0.20-0.12 (m, 2H), −0.14-−0.24 (m, 2H). |
| 52 | 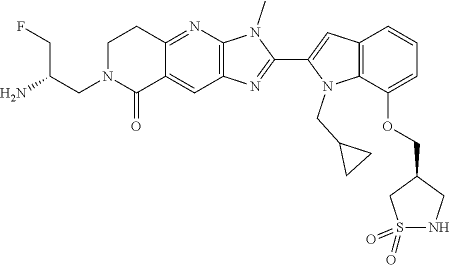 [SMILES: Cn1c(-c2cc3cccc(OC[C@H]4CNS(=O)(=O)C4)c3n2CC2CC2)nc2cc3c(nc21)CCN(C[C@H](N)CF)C3=O] | 7-((S)-2-amino-3-fluoropropyl)-2-(1- (cyclopropylmethyl)-7-(((R)-1,1- dioxidoisothiazolidin-4-yl)methoxy)-1H- indol-2-yl)-3-methyl-3,5,6,7-tetrahydro- 8H-imidazo[4,5-b][1,6]naphthyridin-8-one LCMS (ESI, m/z): 596 [M + H]+; LCMS RT: 0.834 min (Method D) ¹H NMR (400 MHz, DMSO-d₆) δ 8.51 (s, 1H), 8.26 (s, 3H), 7.31 (d, J = 2.4 Hz, 1H), 7.18 (s, 1H), 7.08-7.02 (m, 2H), 6.85- 6.87 (m, 1H), 4.78-4.62 (m, 4H), 4.45- 3.72 (m, 10H), 3.35-3.18 (m, 6H), 3.09- 3.04 (m, 1H), 1.01 (s, 1H), 0.22-0.20 (m, 2H), −0.14-−0.17 (m, 2H). |
| 53 | 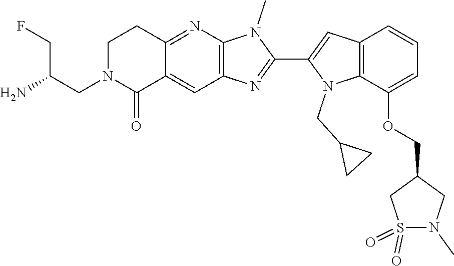 [SMILES: CN1C[C@H](COc2cccc3cc(-c4nc5cc6c(nc5n4C)CCN(C[C@H](N)CF)C6=O)n(CC4CC4)c23)CS1(=O)=O] | 7-((S)-2-amino-3-fluoropropyl)-2-(1- (cyclopropylmethyl)-7-(((R)-2-methyl- 1,1-dioxidoisothiazolidin-4-yl)methoxy)- 1H-indol-2-yl)-3-methyl-3,5,6,7- tetrahydro-8H-imidazo[4,5- b][1,6]naphthyridin-8-one LCMS (ESI, m/z): 610 [M + H]+; LCMS RT: 1.434 min (Method D) ¹H NMR (400 MHz, DMSO-d₆) δ 8.52 (s, 1H), 8.34-8.31 (m, 3H), 7.32 (d, J = 7.6 Hz, 1H), 7.19 (s, 1H), 7.08 (t, J = 7.6 Hz, 1H), 6.86 (d, J = 8.0 Hz, 1H), 4.80-4.63 (m, 4H), 4.27 (d, J = 5.2 Hz, 2H), 3.97 (s, 3H), 3.88-3.72 (m, 5H), 3.59-3.15 (m, 7H), 2.59 (s, 3H), 1.01-0.91 (m, 1H), 0.22- 0.20 (m, 2H), −0.17-−0.21 (m, 2H). |
| 54 | 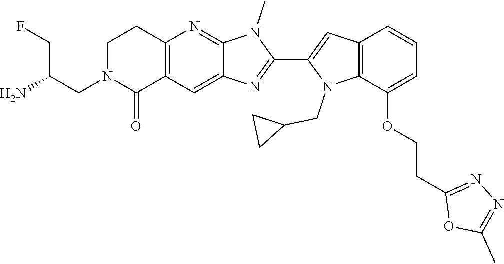 [SMILES: Cc1nnc(CCOc2cccc3cc(-c4nc5cc6c(nc5n4C)CCN(C[C@H](N)CF)C6=O)n(CC4CC4)c23)o1] | (S)-7-(2-amino-3-fluoropropyl)-2-(1- (cyclopropylmethyl)-7-(2-(5-methyl-1,3,4- oxadiazol-2-yl)ethoxy)-1H-indol-2-yl)-3- methyl-3,5,6,7-tetrahydro-8H- imidazo[4,5-b][1,6]naphthyridin-8-one LCMS (ESI, m/z): 573 [M + H]+; LCMS RT: 1.30 min (Method J) ¹H NMR (400 MHz, DMSO-d₆) δ 8.49- 8.53 (m, 1H), 8.19-8.33 (m, 3H), 7.28- 7.32 (m, 1H), 7.16-7.18 (m, 1H), 7.04- 7.10 (m, 1H), 6.88-6.93 (m, 1H), 4.63- 4.82 (m, 2H), 4.53-4.62 (m, 4H), 3.93- 3.97 (m, 3H), 3.81-3.90 (m, 2H), 3.69- 3.79 (m, 3H), 3.44-3.46 (m, 2H), 3.32- 3.34 (m, 2H), 2.47-2.48 (m, 3H), 0.82- 0.88 (m, 1H), 0.09-0.15 (m, 2H), −0.25- −0.18 (m, 2H). |
| 55 | 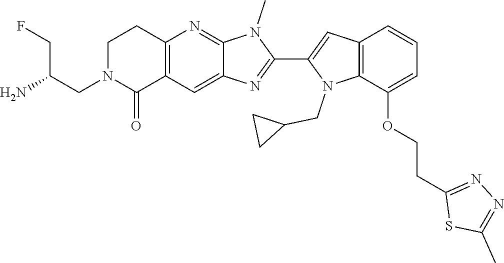 [SMILES: Cc1nnc(CCOc2cccc3cc(-c4nc5cc6c(nc5n4C)CCN(C[C@H](N)CF)C6=O)n(CC4CC4)c23)s1] | (S)-7-(2-amino-3-fluoropropyl)-2-(1- (cyclopropylmethyl)-7-(2-(5-methyl-1,3,4- thiadiazol-2-yl)ethoxy)-1H-indol-2-yl)-3- methyl-3,5,6,7-tetrahydro-8H- imidazo[4,5-b][1,6]naphthyridin-8-one LCMS (ESI, m/z): 589 [M + H]+; LCMS RT: 1.19 min (Method K). |
| 56 | 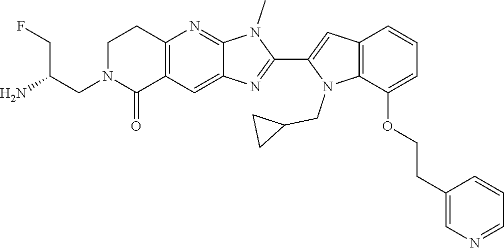 [SMILES: Cn1c(-c2cc3cccc(OCCc4cccnc4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(pyridin-3- yl)ethoxy)-1H-indol-2-yl)-3-methyl- 3,5,6,7-tetrahydro-8H-imidazo[4,5- b][1,6]naphthyridin-8-one LCMS (ESI, m/z): 568 [M + H]+; LCMS RT: 1.05 min (Method K) ¹H NMR (400 MHz, DMSO-d₆) δ 8.75- 8.81 (m, 1H), 8.59-8.65 (m, 1H), 8.50 (s, 1H), 8.24-8.34 (m, 3H), 8.14-8.21 (m, 1H), 7.61-7.69 (m, 1H), 7.26-7.31 (m, 1H), 7.14-7.18 (m, 1H), 7.04-7.10 (m, 1H), 6.88-6.93 (m, 1H), 4.59-4.82 (m, 3H), 4.51-4.58 (m, 4H), 3.94-3.95 (m, 3H), 3.84-3.88 (m, 3H), 3.29-3.35 (m, 4H), 0.79-0.88 (m, 1H), 0.08-0.14 (m, 2H), −0.33-−0.27 (m, 2H). |
| 57 | 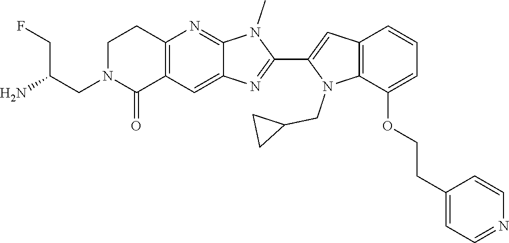 [SMILES: Cn1c(-c2cc3cccc(OCCc4ccncc4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(pyridin-4- yl)ethoxy)-1H-indol-2-yl)-3-methyl- 3,5,6,7-tetrahydro-8H-imidazo[4,5- b][1,6]naphthyridin-8-one LCMS (ESI, m/z): 568 [M + H]+; LCMS RT: 1.05 min (Method K) ¹H NMR (400 MHz, DMSO-d₆) δ 8.74- 8.81 (m, 2H), 8.47-8.51 (m, 1H), 8.25- 8.36 (m, 3H), 7.89-7.95 (m, 2H), 7.27- 7.33 (m, 1H), 7.14-7.17 (m, 1H), 7.04- 7.12 (m, 1H), 6.88-6.96 (m, 1H), 4.65- 4.83 (m, 2H), 4.59-4.63 (m, 2H), 4.46- 4.55 (m, 2H), 3.91-3.97 (m, 3H), 3.78- 3.87 (m, 2H), 3.66-3.77 (m, 3H), 3.39- 3.45 (m, 2H), 3.28-3.36 (m, 2H), 0.70- 0.82 (m, 1H), 0.02-0.11 (m, 2H), −0.40- −0.30 (m, 2H). |
| 58 | 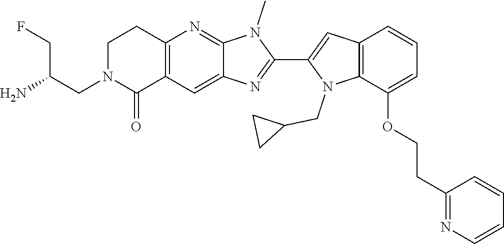 [SMILES: Cn1c(-c2cc3cccc(OCCc4ccccn4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(pyridin-2- yl)ethoxy)-1H-indol-2-yl)-3-methyl- 3,5,6,7-tetrahydro-8H-imidazo[4,5- b][1,6]naphthyridin-8-one LCMS (ESI, m/z): 568 [M + H]+; LCMS RT: 1.08 min (Method K) ¹H NMR (400 MHz, DMSO-d₆) δ 8.63- 8.70 (m, 1H), 8.47-8.52 (m, 1H), 8.21- 8.36 (m, 3H), 7.98-8.07 (m, 1H), 7.64- 7.73 (m, 1H), 7.43-7.55 (m, 1H), 7.25- 7.31 (m, 1H), 7.14-7.17 (m, 1H), 7.03- 7.11 (m, 1H), 6.89-6.95 (m, 1H), 4.65- 4.83 (m, 2H), 4.61-4.65 (m, 2H), 4.47- 4.57 (m, 2H), 3.90-3.96 (m, 3H), 3.79- 3.90 (m, 2H), 3.67-3.78 (m, 3H), 3.38- 3.46 (m, 2H), 3.28-3.36 (m, 2H), 0.67- 0.80 (m, 1H), 0.01-0.11 (m, 2H), −0.36- −0.24 (m, 2H). |

### Example 59. 7-((S)-2-amino-3-fluoropropyl)-2-(1-(cyclopropylmethyl)-7-(((S)-5-oxopyrrolidin-3-yl)methoxy)-1H-indol-2-yl)-3-methyl-3,5,6,7-tetrahydro-8H-imidazo[4,5-b][1,6]naphthyridin-8-one

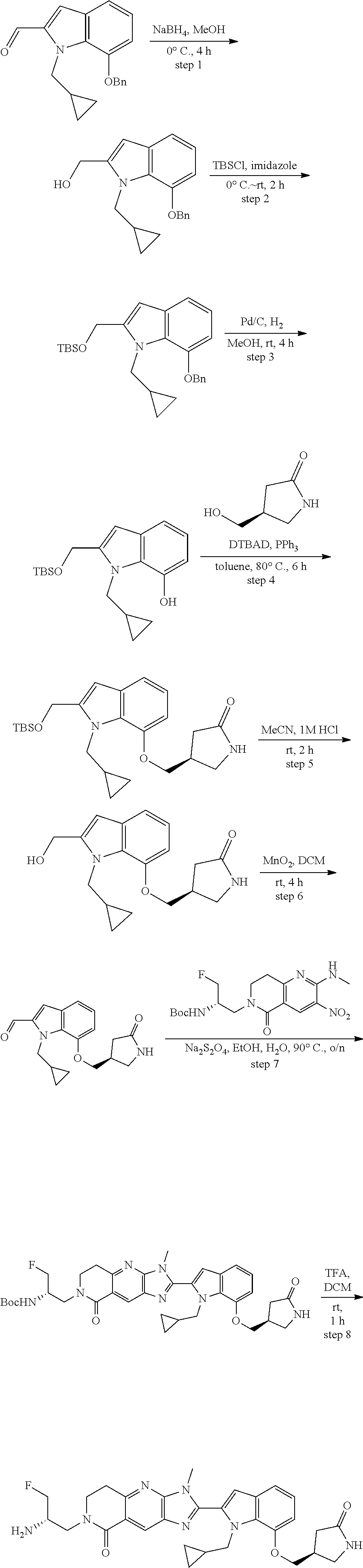

### Step 1: Synthesis of [7-benzyloxy-1-(cyclopropylmethyl)indol-2-yl]methanol

To a solution of 7-benzyloxy-1-(cyclopropylmethyl)indole-2-carbaldehyde (5.0 g, 16.37 mmol) in methanol (150 mL), was added sodium borohydride (0.62 g, 16.37 mmol) in portions at 0° C. under nitrogen atmosphere. The resulting mixture was stirred at 0° C. for 4 h. The reaction was monitored by TLC and LCMS. The reaction was quenched by adding water (100 mL) and extracted with ethyl acetate (2×80 mL). The combined organic extracts were washed with water (2×100 mL) and brine (100 mL), dried over anhydrous sodium sulfate, and concentrated under vacuum to afford the title compound (4.8 g, 95%) as brown oil. LCMS (ESI, m/z): 308 [M+H]+

### Step 2: Synthesis of [7-benzyloxy-1-(cyclopropylmethyl)indol-2-yl]methoxy-tert-butyl-dimethyl-silane

To a solution of [7-benzyloxy-1-(cyclopropylmethyl)indol-2-yl]methanol (4.8 g, 15.62 mmol) in DCM (100 mL), was added imidazole (3.19 g, 46.85 mmol) at room temperature. To the above mixture was added tert-butylchlorodimethylsilane (2.82 g, 18.74 mmol) at 0° C. under nitrogen atmosphere. The resulting mixture was stirred at room temperature for 2 h. The reaction was monitored by TLC and LCMS. The reaction was quenched by adding water (100 mL) and extracted with DCM (2×70 mL). The combined organic extracts were washed with water (100 mL) and brine (100 mL), dried over anhydrous sodium sulfate, and concentrated under vacuum to afford the title compound (6.0 g, 91%) as brown oil. LCMS (ESI, m/z): 422 [M+H]+

### Step 3: Synthesis of 2-[[tert-butyl(dimethyl)silyl]oxymethyl]-1-(cyclopropylmethyl)indol-7-ol

[7-benzyloxy-1-(cyclopropylmethyl)indol-2-yl]methoxy-tert-butyl-dimethylsilane (3.5 g, 8.3 mmol) in ethyl acetate (30 mL) and methanol (70 mL) was reacted according to General Procedure 9. The residue was purified by flash column chromatography to afford the title compound (2.5 g, 91%) as brown oil. LCMS (ESI, m/z): 332 [M+H]+

### Step 4 Synthesis of (4S)-4-[[2-[[tert-butyl(dimethyl)silyl]oxymethyl]-1-(cyclopropylmethyl)indol-7-yl]oxymethyl]pyrrolidin-2-one

2-[[tert-butyl(dimethyl)silyl]oxymethyl]-1-(cyclopropylmethyl)indol-7-ol (2.1 g, 6.33 mmol) and (S)-4-(hydroxymethyl)pyrrolidin-2-one (2.19 g, 19 mmol) were reacted according to General Procedure 10. The crude product was purified by flash column chromatography to afford the title compound (2.1 g, 77%) as brown oil. LCMS (ESI, m/z): 429 [M+H]+.

### Step 5 Synthesis of (4S)-4-[[1-(cyclopropylmethyl)-2-(hydroxymethyl)indol-7-yl]oxymethyl]pyrrolidin-2-one

To a stirred solution of (4S)-4-[[2-[[tert-butyl(dimethyl)silyl]oxymethyl]-1-(cyclopropylmethyl)indol-7-yl]oxymethyl]pyrrolidin-2-one (2.1 g, 4.9 mmol) in acetonitrile (20 mL), was added aqueous HCl (1M, 10 mL, 4.9 mmol) at room temperature. The resulting mixture was stirred at room temperature for 2 h. The reaction was monitored by LCMS. The mixture was quenched with saturated aqueous NaHCO₃ (100 mL), extracted with ethyl acetate (2×60 mL). The organic layer was washed with brine (2×70 mL), dried over anhydrous sodium sulfate and concentrated under vacuum to afford the title compound (1.25 g, 81%) as brown solid. LCMS (ESI, m/z): 315 [M+H]+

### Step 6: Synthesis of 1-(cyclopropylmethyl)-7-[[(3S)-5-oxopyrrolidin-3-yl]methoxy]indole-2-carbaldehyde

(4S)-4-[[1-(cyclopropylmethyl)-2-(hydroxymethyl)indol-7-yl]oxymethyl]pyrrolidin-2-one (1.25 g, 3.98 mmol) was reacted according to General Procedure 4. The crude product was purified by Prep-TLC (DCM/MeOH=15:1) to afford the title compound (0.80 g, 64%) as yellow oil. LCMS (ESI, m/z): 313 [M+H]+

### Step 7: Synthesis of tert-butyl ((S)-1-(2-(1-(cyclopropylmethyl)-7-(((S)-5-oxopyrrolidin-3-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 2 (550 mg, 1.38 mmol) and 1-(cyclopropylmethyl)-7-[[(3S)-5-oxopyrrolidin-3-yl]methoxy]indole-2-carbaldehyde (475 mg, 1.52 mmol) were reacted according to General Procedure 6. The crude product was purified by Prep-TLC (DCM/MeOH=12:1) to afford the title compound (450 mg, 49%) as yellow solid. LCMS (ESI, m/z): 660 [M+H]+

### Step 8: Synthesis of 7-((S)-2-amino-3-fluoropropyl)-2-(1-(cyclopropylmethyl)-7-(((S)-5-oxopyrrolidin-3-yl)methoxy)-1H-indol-2-yl)-3-methyl-3,5,6,7-tetrahydro-8H-imidazo[4,5-b][1,6]naphthyridin-8-one

Tert-butyl ((S)-1-(2-(1-(cyclopropylmethyl)-7-(((S)-5-oxopyrrolidin-3-yl)methoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (450 mg, 0.68 mmol) was reacted according to General Procedure 2. The crude product was purified by Prep HPLC (Method A) to afford the title compound (191 mg, 48.2%) as light-yellow solid. ¹H NMR (400 MHz, DMSO-d₆) δ 8.48 (s, 1H), 7.29 (d, J=7.9 Hz, 1H), 7.16 (s, 1H), 7.06 (t, J=7.8 Hz, 1H), 6.86 (d, J=7.8 Hz, 1H), 4.72 (d, J=6.9 Hz, 2H), 4.44-4.37 (m, 1H), 4.33-4.26 (m, 1H), 4.18 (d, J=6.5 Hz, 2H), 3.95 (s, 3H), 3.82-3.70 (m, 2H), 3.64-3.57 (m, 1H), 3.55-3.43 (m, 2H), 3.28 (d, J=13.3 Hz, 2H), 3.26-3.18 (m, 2H), 2.04-1.94 (m, 1H), 2.46-2.37 (m, 1H), 2.22-2.12 (m, 1H), 1.04-0.91 (m, 1H), 0.25-0.14 (m, 2H), −0.09-−0.20 (m, 2H). LCMS (ESI, m/z): 560 [M+H]+. LCMS RT: 1.320 min (Method D).

### Example 60. 7-((S)-2-amino-3-fluoropropyl)-2-(1-(cyclopropylmethyl)-7-(((R)-5-oxopyrrolidin-3-yl)methoxy)-1H-indol-2-yl)-3-methyl-3,5,6,7-tetrahydro-8H-imidazo[4,5-b][1,6]naphthyridin-8-one

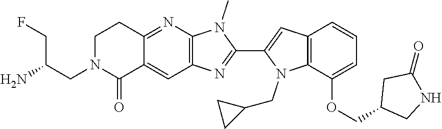 [SMILES: Cn1c(-c2cc3cccc(OC[C@H]4CNC(=O)C4)c3n2CC2CC2)nc2cc3c(nc21)CCN(C[C@H](N)CF)C3=O]

Example 60 was synthesized using a similar synthetic route to Example 59 using (R)-4-(hydroxymethyl)pyrrolidin-2-one in step 4 to afford the title compound (17.7 mg, 19.77%) as white solid. LCMS (ESI, m/z): 560 [M+H]+. LCMS RT: 1.628 min (Method D).

### Examples 61a and 61b. 7-((S)-2-amino-3-fluoropropyl)-2-(1-(cyclopropylmethyl)-7-((S)-2-hydroxy-2-(oxazol-5-yl)ethoxy)-1H-indol-2-yl)-3-methyl-3,5,6,7-tetrahydro-8H-imidazo[4,5-b][1,6]naphthyridin-8-one and 7-((S)-2-amino-3-fluoropropyl)-2-(1-(cyclopropylmethyl)-7-((R)-2-hydroxy-2-(oxazol-5-yl)ethoxy)-1H-indol-2-yl)-3-methyl-3,5,6,7-tetrahydro-8H-imidazo[4,5-b][1,6]naphthyridin-8-one

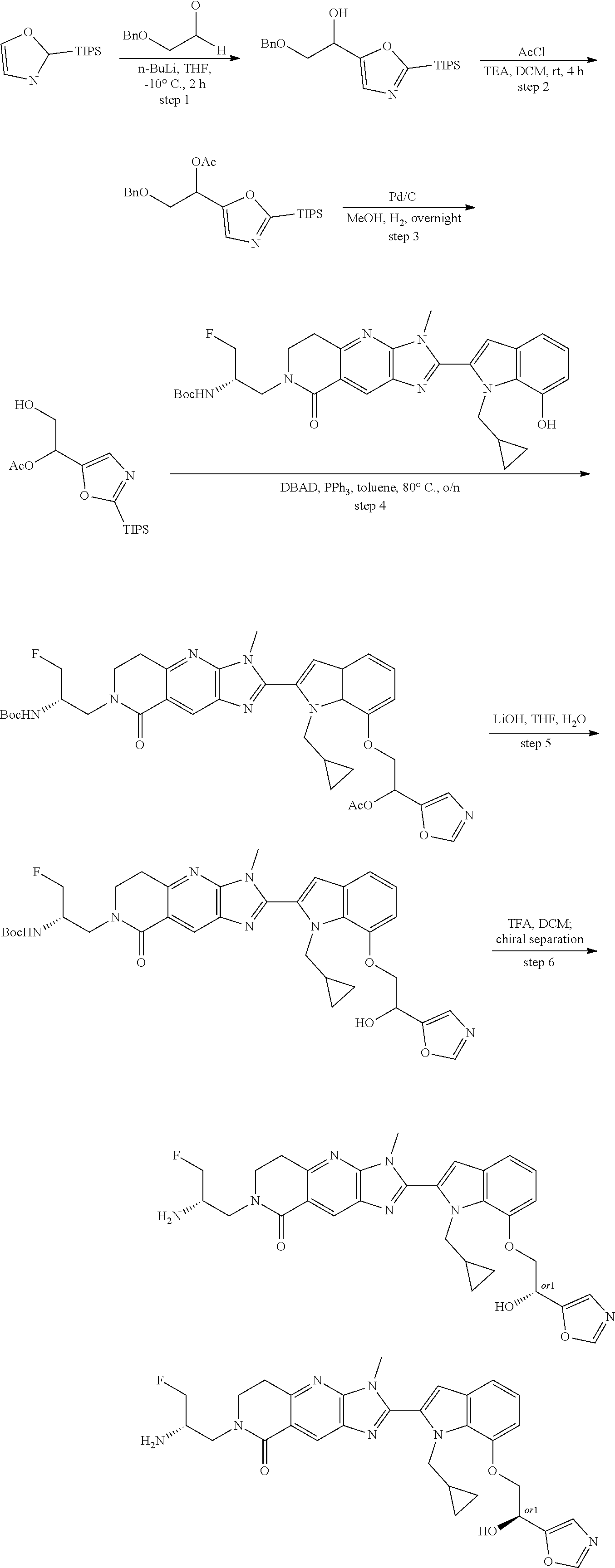

### Step 1: Synthesis of 2-benzyloxy-1-(2-triisopropylsilyloxazol-5-yl)ethanol

To a solution of triisopropyl(oxazol-2-yl)silane (1 g, 4.4 mmol) and 2-benzyloxyacetaldehyde (1 g, 6.6 mmol) in THF (50 mL), was added n-butyllithium (2.5 M in n-hexane, 4.4 mL, 11.1 mmol) dropwise at −10° C. under nitrogen atmosphere. The mixture was stirred at room temperature for 1 h under nitrogen atmosphere. The reaction was monitored by LCMS. The reaction was then quenched by adding saturated aqueous NH₄Cl (150 mL) at 0° C. and extracted with ethyl acetate (2×150 mL). The combined organic extracts were washed with brine (300 mL), dried over anhydrous sodium sulfate and concentrated under vacuum. The crude product was purified by column chromatography (petroleum ether/ethyl acetate=1:1) to afford the title compound (747 mg, 44.8%) as a light yellow solid. LCMS (ESI, m/z): 376 [M+H]+.

### Step 2: Synthesis of 2-(benzyloxy)-1-(2-(triisopropylsilyl)oxazol-5-yl)ethyl acetate

To a solution of 2-benzyloxy-1-(2-triisopropylsilyloxazol-5-yl)ethanol (670 mg, 1.8 mmol) in DCM (2 mL) was added TEA (1 mL, 5.4 mmol). To the above mixture was added a solution of acetyl chloride (168 mg, 2.1 mmol) in DCM (0.5 mL) dropwise at 0° C. under nitrogen atmosphere. The resulting mixture was stirred at room temperature for 2 h under nitrogen atmosphere. The reaction was monitored by LCMS. The reaction was quenched by adding saturated aqueous NH₄Cl (5 mL) at 0° C., extracted with DCM (2×5 mL). The combined organic extracts were washed with brine (10 mL), dried over anhydrous sodium sulfate and concentrated under vacuum. The crude product was purified by column chromatography (petroleum ether/ethyl acetate=3:1) to afford the title compound (450 mg, 60.4%) as a light yellow oil. LCMS (ESI, m/z): 418 [M+H]+.

### Step 3: Synthesis of 2-(hydroxy)-1-(2-(triisopropylsilyl)oxazol-5-yl)ethyl acetate

2-(Benzyloxy)-1-(2-(triisopropylsilyl)oxazol-5-yl)ethyl acetate (400 mg, 1.0 mmol) in methanol (10 mL) was reacted according to General Procedure 9. The crude product was purified by flash column chromatography on C18 silica to afford the title compound (160 mg, 51.0%) as a light yellow solid. LCMS (ESI, m/z): 328 [M+H]+.

### Step 4: Synthesis of 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-1-(oxazol-5-yl)ethyl acetate

2-(hydroxy)-1-(2-(triisopropylsilyl)oxazol-5-yl)ethyl acetate (142 mg, 0.4 mmol) was reacted with Intermediate 5 (163 mg, 0.3 mmol) according to General Procedure 10. The crude product was purified by column chromatography (petroleum ether/ethyl acetate=1:1) to afford the title compound (80 mg, 38.6%) as a mixture of diastereomers as a light yellow solid. LCMS (ESI, m/z): 716 [M+H]+.

### Step 5: Synthesis of tert-butyl ((2S)-1-(2-(1-(cyclopropylmethyl)-7-(2-hydroxy-2-(oxazol-5-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-1-(oxazol-5-yl)ethyl acetate (75 mg, 0.1 mmol) in THF (1 mL) and water (0.2 mL), was added LiOH (12.5 mg, 0.5 mmol). The resulting solution was stirred at 50° C. for 16 h. The reaction was monitored by LCMS. The mixture product was adjusted to pH 2 with aqueous HCl (1M). The resulting solution was stirred for 0.5 h at room temperature. The mixture was extracted with ethyl acetate (2×10 mL). The combined organic extracts were washed with brine (10 mL), dried over anhydrous sodium sulfate and concentrated under vacuum. The crude product was purified by flash column chromatography on C18 silica to afford the title compound (70 mg, 99.1%) as a mixture of diastereomers as a light yellow solid. LCMS (ESI, m/z): 674 [M+H]+.

### Step 6: Synthesis of 7-((S)-2-amino-3-fluoropropyl)-2-(1-(cyclopropylmethyl)-7-(2-hydroxy-2-(oxazol-5-yl)ethoxy)-1H-indol-2-yl)-3-methyl-3,5,6,7-tetrahydro-8H-imidazo[4,5-b][1,6]naphthyridin-8-one

Tert-butyl ((2S)-1-(2-(1-(cyclopropylmethyl)-7-(2-hydroxy-2-(oxazol-5-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (65 mg, 0.01 mmol) was reacted according to General Procedure 2. The crude product was purified by Chiral Prep HPLC [Column: CHIRALPAK IF, 2×25 cm, 5 μm; Mobile Phase A: MTBE (0.5% 2M NH₃ in MeOH), Mobile Phase B: MeOH: DCM (1:1) (0.1% 2M NH₃ in MeOH); Flow rate: 14 mL/min; Gradient: isocratic 30% B in 32 min.; Detection: UV (220/254 nm)] to afford single stereoisomers of unknown configuration at the alcohol stereocenter.

Example 61a. Isomer 1: Chiral HPLC RT: 19.2 min. ¹H NMR (400 MHz, DMSO-d₆) δ 8.48 (d, J=1.6 Hz, 1H), 8.38 (s, 1H), 7.37 (s, 1H), 7.28 (d, J=7.2 Hz, 1H), 7.14 (s, 1H), 7.01 (d, J=7.6 Hz, 2H), 5.74 (t, J=6.4 Hz, 1H), 5.29 (t, J=6.4 Hz, 1H), 4.83-4.82 (m, 1H), 4.70-4.68 (m, 1H), 4.43-4.42 (m, 1H), 4.33-4.30 (m, 1H), 4.07-4.03 (m, 1H), 3.96-3.94 (m, 4H), 3.81-3.72 (m, 2H), 3.64-3.46 (m, 5H), 1.02-1.0 (m, 1H), 0.19-0.17 (m, 2H), −0.04-−0.13 (m, 2H). LCMS (ESI, m/z): 574 [M+H]+; LCMS RT: 0.803 min. (Method D).

Example 61b. Isomer 2: Chiral HPLC RT: 28.11 min. ¹H NMR (400 MHz, DMSO-d₆): δ 8.48 (s, 1H), 8.38 (s, 1H), 7.37 (s, 1H), 7.28 (d, J=7.2 Hz, 1H), 7.14 (s, 1H), 7.01 (d, J=7.6 Hz, 2H), 5.74 (t, J=6.4 Hz, 1H), 5.29 (t, J=6.4 Hz, 1H), 4.83-4.82 (m, 1H), 4.70-4.68 (m, 1H), 4.43-4.42 (m, 1H), 4.33-4.30 (m, 1H), 4.07-4.03 (m, 1H), 3.96-3.94 (m, 4H), 3.81-3.72 (m, 2H), 3.64-3.46 (m, 5H), 1.02-1.0 (m, 1H), 0.19-0.17 (m, 2H), −0.04-−0.13 (m, 2H). LCMS (ESI, m/z): 574 [M+H]+; LCMS RT: 1.033 min. (Method D).

### Example 62. 2-(7-(((R)-1-(1H-imidazol-1-yl)propan-2-yl)oxy)-1-(cyclopropylmethyl)-1H-indol-2-yl)-7-((S)-2-amino-3-fluoropropyl)-3-methyl-3,5,6,7-tetrahydro-8H-imidazo[4,5-b][1,6]naphthyridin-8-one

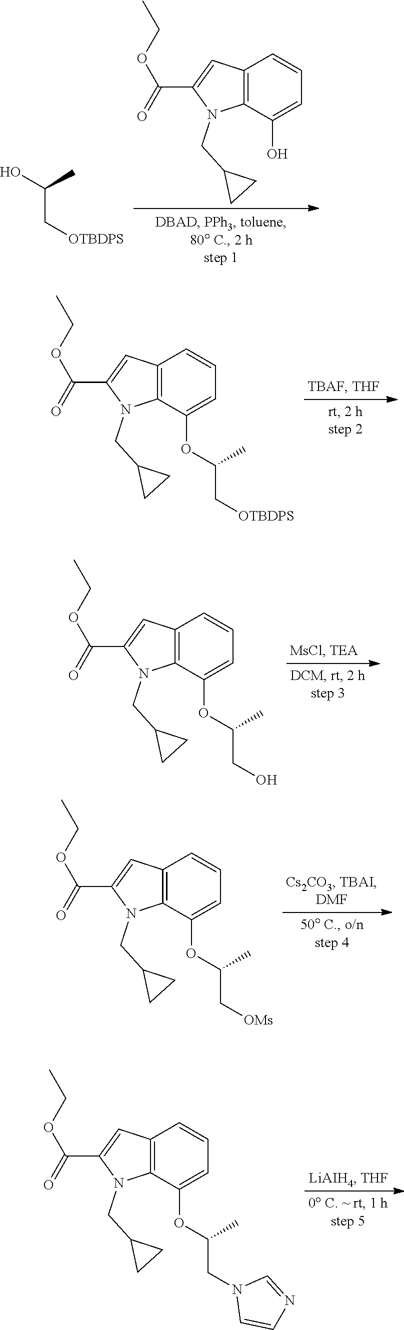

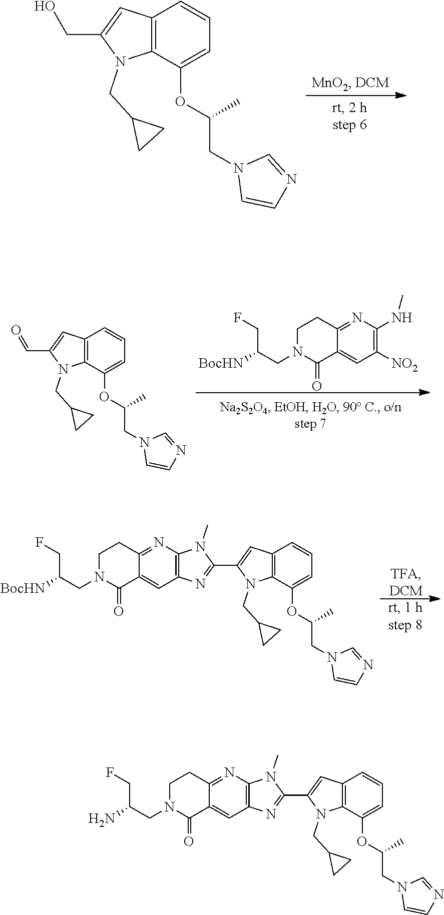 [SMILES: C[C@H](Cn1ccnc1)Oc1cccc2cc(CO)n(CC3CC3)c12 ; C[C@H](Cn1ccnc1)Oc1cccc2cc(C=O)n(CC3CC3)c12 ; CNc1nc2c(cc1[N+](=O)[O-])C(=O)N(C[C@@H](CF)NC(=O)OC(C)(C)C)CC2 ; C[C@H](Cn1ccnc1)Oc1cccc2cc(-c3nc4cc5c(nc4n3C)CCN(C[C@@H](CF)NC(=O)OC(C)(C)C)C5=O)n(CC3CC3)c12 ; C[C@H](Cn1ccnc1)Oc1cccc2cc(-c3nc4cc5c(nc4n3C)CCN(C[C@H](N)CF)C5=O)n(CC3CC3)c12]

### Step 1: Synthesis of ethyl (R)-7-((1-((tert-butyldiphenylsilyl)oxy)propan-2-yl)oxy)-1-(cyclopropylmethyl)-1H-indole-2-carboxylate

(S)-1-((tert-butyldiphenylsilyl)oxy)propan-2-ol (1.8 g, 5.72 mmol) was reacted with ethyl 1-(cyclopropylmethyl)-7-hydroxy-indole-2-carboxylate (300 mg, 1.16 mmol) according to General Procedure 10. The crude product was purified by flash column chromatography (DCM/MeOH=10:1) to afford the title compound (500 mg, 16%) as a yellow oil. LCMS (ESI, m/z): 556 [M+H]+.

### Step 2: Synthesis of ethyl (R)-1-(cyclopropylmethyl)-7-((1-hydroxypropan-2-yl)oxy)-1H-indole-2-carboxylate

To a solution of ethyl (R)-7-((1-((tert-butyldiphenylsilyl)oxy)propan-2-yl)oxy)-1-(cyclopropylmethyl)-1H-indole-2-carboxylate (550 mg, 0.99 mmol) in THE (10 mL), was added TBAF (10 mL) at 0° C. The resulting solution was stirred at room temperature for 2 h. The reaction was monitored by LCMS. The reaction was then quenched by adding water (20 mL) and extracted with ethyl acetate (20 mL), washed with water (5×20 ml) and brine (5×20 mL), dried over anhydrous sodium sulfate and concentrated under vacuum. The crude product was purified by column chromatography (DCM/MeOH=10:1) to afford the title compound (300 mg, 80%) as a yellow oil. LCMS (ESI, m/z): 318 [M+H]+.

### Step 3: Synthesis of ethyl (R)-1-(cyclopropylmethyl)-7-((1-((methylsulfonyl)oxy)propan-2-yl)oxy)-1H-indole-2-carboxylate

Ethyl (R)-1-(cyclopropylmethyl)-7-((1-hydroxypropan-2-yl)oxy)-1H-indole-2-carboxylate (200 mg, 0.63 mmol) in DCM (6 mL) was reacted with TEA (0.16 mL, 1.15 mmol) and MsCl (88 mg, 0.77 mmol) according to General Procedure 13 to afford the title compound (200 mg, 80%) as a light yellow oil. LCMS (ESI, m/z): 396 [M+H]+.

### Step 4: Synthesis of ethyl (R)-7-((1-(1H-imidazol-1-yl)propan-2-yl)oxy)-1-(cyclopropylmethyl)-1H-indole-2-carboxylate

Ethyl (R)-1-(cyclopropylmethyl)-7-((1-((methylsulfonyl)oxy)propan-2-yl)oxy)-1H-indole-2-carboxylate (254 mg, 0.64 mmol) was reacted with 1H-imidazole (435 mg, 6.39 mmol) according to General Procedure 14. The crude product was purified by column chromatography (DCM/MeOH=10:1) to afford the title compound (165 mg, 70%) as a yellow oil. LCMS (ESI, m/z): 368 [M+H]+

### Step 5: Synthesis of (R)-(7-((1-(1H-imidazol-1-yl)propan-2-yl)oxy)-1-(cyclopropylmethyl)-1H-indol-2-yl)methanol

Ethyl (R)-7-((1-(1H-imidazol-1-yl)propan-2-yl)oxy)-1-(cyclopropylmethyl)-1H-indole-2-carboxylate (80 mg, 0.36 mmol) was reacted according to General Procedure 3. The crude product was purified by column chromatography (DCM/MeOH=10:1) to afford the title compound (60 mg, 85%) as a yellow oil. LCMS (ESI, m/z): 326 [M+H]+.

### Step 6: Synthesis of (R)-7-((1-(1H-imidazol-1-yl)propan-2-yl)oxy)-1-(cyclopropylmethyl)-1H-indole-2-carbaldehyde

(R)-(7-((1-(1H-imidazol-1-yl)propan-2-yl)oxy)-1-(cyclopropylmethyl)-1H-indol-2-yl)methanol (80 mg, 0.25 mmol) was reacted according to General Procedure 4. The crude product was purified by column chromatography (DCM/MeOH=20:1) to afford the title compound (45 mg, 57%) as a yellow oil. LCMS (ESI, m/z): 324 [M+H]+.

### Step 7: Synthesis of tert-butyl ((S)-1-(2-(7-(((R)-1-(1H-imidazol-1-yl)propan-2-yl)oxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

(R)-7-((1-(1H-imidazol-1-yl)propan-2-yl)oxy)-1-(cyclopropylmethyl)-1H-indole-2-carbaldehyde (45 mg, 0.14 mmol) was reacted with Intermediate 2 (66 mg, 0.17 mmol) according to General Procedure 6. The crude product was purified by column chromatography (DCM/MeOH=10:1) to afford the title compound (35 mg, 37%) as a yellow oil. LCMS (ESI, m/z): 671 [M+H]+.

### Step 8: Synthesis of 2-(7-(((R)-1-(1H-imidazol-1-yl)propan-2-yl)oxy)-1-(cyclopropylmethyl)-1H-indol-2-yl)-7-((S)-2-amino-3-fluoropropyl)-3-methyl-3,5,6,7-tetrahydro-8H-imidazo[4,5-b][1,6]naphthyridin-8-one

Tert-butyl ((S)-1-(2-(7-(((R)-1-(1H-imidazol-1-yl)propan-2-yl)oxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (35 mg, 0.05 mmol) was reacted according to General Procedure 2. The crude product was purified by Prep-HPLC (Method G) to afford the title compound (21.2 mg, 73%) as a light yellow solid. ¹H NMR (400 MHz, DMSO-d₆): δ 9.24 (s, 1H), 8.50 (s, 1H), 8.36 (s, 2H), 7.88 (t, J=1.7 Hz, 1H), 7.73 (t, J=1.6 Hz, 1H), 7.31 (d, J=7.8 Hz, 1H), 7.19 (s, 1H), 7.09 (t, J=7.9 Hz, 1H), 6.90 (d, J=7.9 Hz, 1H), 5.32-5.24 (m, 1H), 4.84-4.58 (m, 6H), 3.96 (s, 3H), 3.91-3.71 (m, 5H), 3.34 (t, J=6.5 Hz, 2H), 1.39 (d, J=6.4 Hz, 3H), 0.87-0.74 (m, 1H), 0.22-0.10 (m, 2H), −0.21-−0.34 (m, 2H). LCMS (ESI, m/z): 571 [M+H]+. LCMS RT: 1.258 min (Method B).

### Example 63. 2-(7-(((S)-1-(1H-imidazol-1-yl)propan-2-yl)oxy)-1-(cyclopropylmethyl)-1H-indol-2-yl)-7-((S)-2-amino-3-fluoropropyl)-3-methyl-3,5,6,7-tetrahydro-8H-imidazo[4,5-b][1,6]naphthyridin-8-one

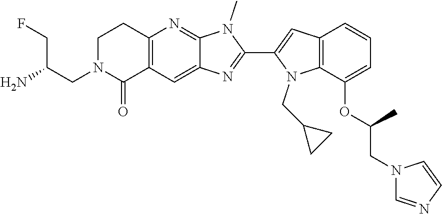 [SMILES: C[C@@H](Cn1ccnc1)Oc1cccc2cc(-c3nc4cc5c(nc4n3C)CCN(C[C@H](N)CF)C5=O)n(CC3CC3)c12]

Example 63 was synthesized according to a similar synthetic route to Example 62 starting from (R)-1-((tert-butyldiphenylsilyl)oxy)propan-2-ol. ¹H NMR (400 MHz, DMSO-d₆) δ 9.22 (s, 1H), 8.50 (s, 1H), 8.34 (s, 3H), 7.87 (s, 1H), 7.72 (s, 1H), 7.30 (d, J=8.0 Hz, 1H), 7.18-76.9 (m, 2H), 6.88 (s, 1H), 5.28 (d, J=4.0 Hz, 1H), 4.78-4.61 (m, 6H), 3.95 (s, 3H), 3.89-3.74 (m, 5H), 3.34 (t, J=6.4 Hz, 2H), 1.39 (d, J=6.0 Hz, 3H), 0.80 (s, 1H), 0.19-0.03 (m, 2H), −0.23-−0.3 (m, 2H). LCMS (ESI, m/z): 571 [M+H]+. LCMS RT: 1.256 min. (Method D).

### Example 64. 7-((S)-2-amino-3-fluoropropyl)-2-(1-(cyclopropylmethyl)-5-fluoro-7-((1-(oxazol-5-yl)propan-2-yl)oxy)-1H-indol-2-yl)-3-methyl-3,5,6,7-tetrahydro-8H-imidazo[4,5-b][1,6]naphthyridin-8-one

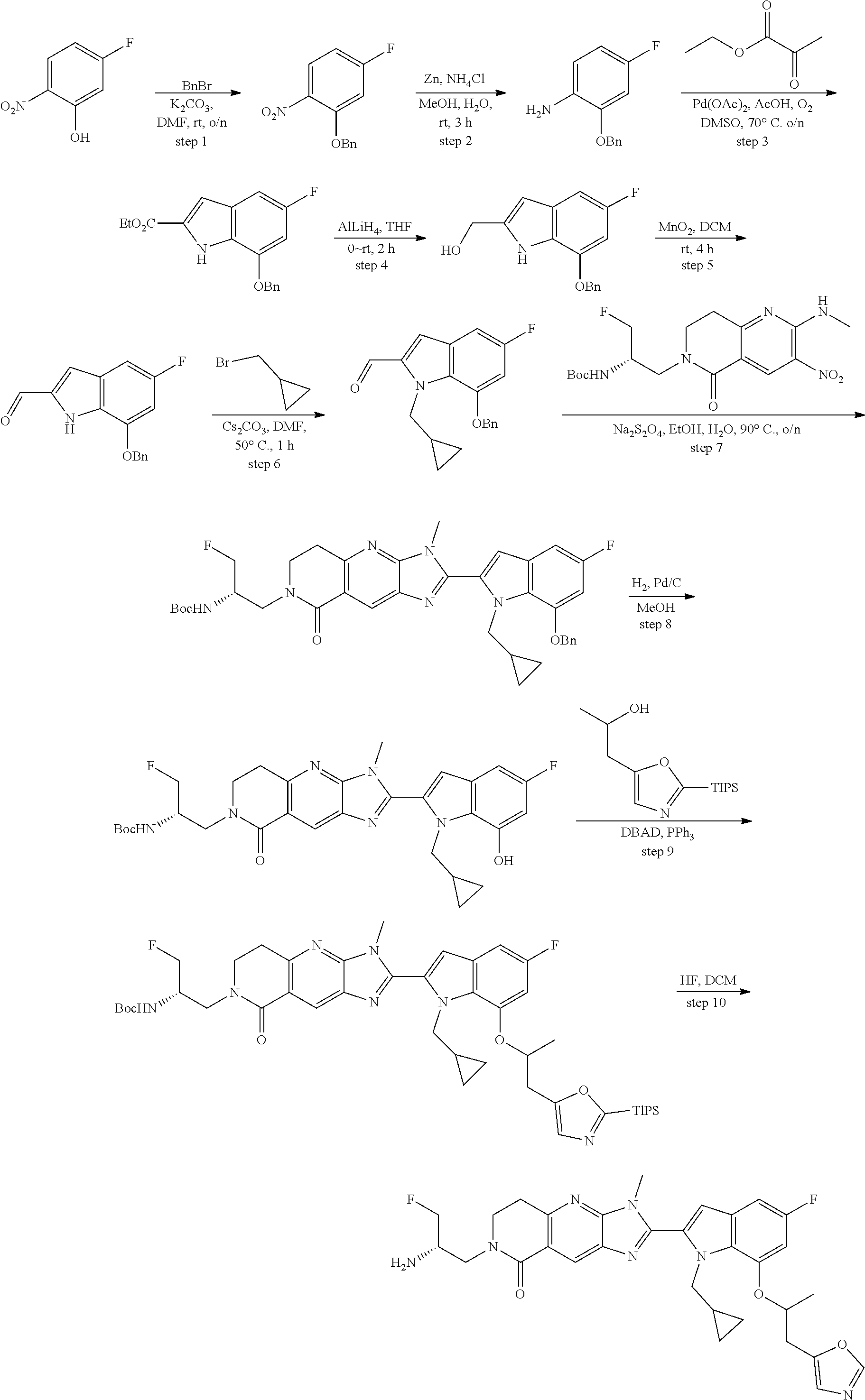

### Step 1: Synthesis of 2-(benzyloxy)-4-fluoro-1-nitrobenzene

To a solution of 5-fluoro-2-nitro-phenol (15.7 g, 99.9 mmol) in DMF (500 mL) was added (bromomethyl)benzene (25.6 g, 150 mmol) and K₂CO₃ (41.4 g, 299 mmol) at room temperature under nitrogen atmosphere. The mixture was stirred at room temperature overnight under nitrogen atmosphere. The reaction was monitored by LCMS. The reaction was then quenched by adding water (1.5 L) and extracted with ethyl acetate (1.5 L), washed with water (2×500 mL) and brine (500 mL), dried over anhydrous sodium sulfate and concentrated under vacuum. The crude product was purified by column chromatography (petroleum ether/ethyl acetate=5:1) to afford the title compound (24.5 g, 99.1%) as a light yellow solid. LCMS (ESI, m/z): 248 [M+H]+

### Step 2: Synthesis of 2-(benzyloxy)-4-fluoroaniline

To a solution of 2-(benzyloxy)-4-fluoro-1-nitrobenzene (24.4 g, 98.7 mmol) in methanol (600 mL), was added NH₄Cl (53.3 g, 987 mmol) in water (100 mL) and Zn (64.2 g, 987 mmol) at room temperature under nitrogen atmosphere. The mixture was stirred at room temperature for 3 h. The reaction was monitored by LCMS. Solids were filtered out and the solvent was evaporated under vacuum. The crude product was diluted with ethyl acetate (600 mL). The combined organic extracts were washed with water (2×500 mL) and brine (500 mL), dried over anhydrous sodium sulfate and concentrated under vacuum. The crude product was purified by column chromatography (petroleum ether/ethyl acetate=3:1) to afford the title compound (16.3 g, 76%) as a light yellow oil. LCMS (ESI, m/z): 218 [M+H]+

### Step 3: Synthesis of ethyl 7-(benzyloxy)-5-fluoro-1H-indole-2-carboxylate

To a solution of 2-(benzyloxy)-4-fluoroaniline (8 g, 36.8 mmol) in DMSO (100 mL) was added Pd(OAc)₂ (1.7 g, 7.3 mmol) and AcOH (2.1 mL, 36.8 mmol) at room temperature under nitrogen atmosphere. The reaction mixture was recharged with nitrogen gas three times and then recharged with oxygen gas three times. The mixture was stirred at 70° C. overnight under oxygen atmosphere. The reaction was monitored by LCMS. The reaction was then quenched by adding water (300 mL) and extracted with ethyl acetate (500 mL). The combined organic extracts were washed with water (2×300 mL) and brine (300 mL), dried over anhydrous sodium sulfate and concentrated under vacuum. The crude product was purified by column chromatography (petroleum ether/ethyl acetate=5:1) to afford the title compound (3.8 g, 33%) as a light yellow solid. LCMS (ESI, m/z): 314 [M+H]+

### Step 4: Synthesis of (7-(benzyloxy)-5-fluoro-1H-indol-2-yl)methanol

Ethyl 7-(benzyloxy)-5-fluoro-1H-indole-2-carboxylate (3.5 g, 11.2 mmol) was reacted according to General Procedure 3. The crude product was purified by column chromatography (petroleum ether/ethyl acetate=2:1) to afford the title compound (2.9 g, 95.6%) as a light yellow solid. LCMS (ESI, m/z): 272 [M+H]+

### Step 5: Synthesis of 7-(benzyloxy)-5-fluoro-1H-indole-2-carbaldehyde

(7-(benzyloxy)-5-fluoro-1H-indol-2-yl)methanol (3.4 g, 12.5 mmol) was reacted according to General Procedure 4. The crude product was purified by column chromatography (petroleum ether/ethyl acetate=3:1) to afford the title compound (1.9 g, 56%) as a light yellow solid. LCMS (ESI, m/z): 270 [M+H]+

### Step 6: Synthesis of 7-(benzyloxy)-1-(cyclopropylmethyl)-5-fluoro-1H-indole-2-carbaldehyde

7-(Benzyloxy)-5-fluoro-1H-indole-2-carbaldehyde (1 g, 3.7 mmol) was reacted with bromomethylcyclopropane (602 mg, 4.4 mmol) according to General Procedure 5. The crude product was purified by column chromatography (petroleum ether/ethyl acetate=4:1) to afford the title compound (1.0 g, 83%) as a light yellow solid. LCMS (ESI, m/z): 324 [M+H]+

### Step 7: Synthesis of tert-butyl (S)-(1-(2-(7-(benzyloxy)-1-(cyclopropylmethyl)-5-fluoro-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

7-(benzyloxy)-1-(cyclopropylmethyl)-5-fluoro-1H-indole-2-carbaldehyde (388 mg, 1.2 mmol) was reacted with Intermediate 2 (476 mg, 1.2 mmol) according to General Procedure 6. The crude product was purified by column chromatography (petroleum ether/ethyl acetate=1:1) to afford the title compound (370 mg, 45.9%) as a light yellow solid. LCMS (ESI, m/z): 671 [M+H]+

### Step 8: Synthesis of tert-butyl (S)-(1-(2-(1-(cyclopropylmethyl)-5-fluoro-7-hydroxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Tert-butyl (S)-(1-(2-(7-(benzyloxy)-1-(cyclopropylmethyl)-5-fluoro-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (370 mg, 0.5 mmol) was reacted according to General Procedure 9. The crude product was purified by column chromatography (petroleum ether/ethyl acetate=1:2) to afford the title compound (270 mg, 84.3%) as a light yellow solid. LCMS (ESI, m/z): 581 [M+H]+

### Step 9: Synthesis of 2-(triisopropylsilyl)oxazole

To a stirred solution of oxazole (6 g, 86.9 mmol) in THF (40 mL) was added n-butyllithium (21.8 g, 130.3 mmol) in portions at −10° C. under a nitrogen atmosphere. The mixture was stirred for 30 min at −10° C. under a nitrogen atmosphere. To the above mixture was added triisopropylsilyl trifluoromethanesulfonate (31.9 g, 104.3 mmol) dropwise at −10° C. under a nitrogen atmosphere. The mixture was stirred for 2 h at room temperature under nitrogen atmosphere. The reaction was monitored by LCMS. The reaction was then quenched by adding water (120 mL) at 0° C., extracted with ethyl acetate (2×200 mL). The combined organic extracts were washed with brine (120 mL), dried over anhydrous sodium sulfate, concentrated under vacuum. The crude product was purified by column chromatography (petroleum ether/ethyl acetate=1:2) to afford the title compound (5 g, 25.5%) as a light yellow solid. LCMS (ESI, m/z): 226 [M+H]+

### Step 10: Synthesis of 1-(2-(triisopropylsilyl)oxazol-5-yl)propan-2-ol

To a stirred solution of 2-(triisopropylsilyl)oxazole (1 g, 4.4 mmol) in THE (40 mL) was added n-butyllithium (1.9 g, 11 mmol) in portions at −10° C. under a nitrogen atmosphere. The mixture was stirred for 30 min at −10° C. under a nitrogen atmosphere. To the above mixture was added 2-methyloxirane (515 mg, 8.8 mmol) dropwise at −10° C. under a nitrogen atmosphere. The mixture was stirred for 2 h at room temperature under nitrogen atmosphere. The reaction was monitored by LCMS. The reaction was then quenched by adding water (120 mL) at 0° C., extracted with ethyl acetate (2×200 mL). The combined organic extracts were washed with brine (120 mL), dried over anhydrous sodium sulfate, concentrated under vacuum. The crude product was purified by column chromatography (petroleum ether/ethyl acetate=1:2) to afford the title compound (800 mg, 63.6%) as a light yellow oil. LCMS (ESI, m/z): 284 [M+H]+

### Step 11: Synthesis of tert-butyl ((2S)-1-(2-(1-(cyclopropylmethyl)-5-fluoro-7-((1-(2-(triisopropylsilyl)oxazol-5-yl)propan-2-yl)oxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Tert-butyl (S)-(1-(2-(1-(cyclopropylmethyl)-5-fluoro-7-hydroxy-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (200 mg, 0.3 mmol) and 1-(2-triisopropylsilyloxazol-5-yl)propan-2-ol (292 mg, 1 mmol) were reacted according to General Procedure 10. The crude product was purified by flash column chromatography on C18 silica to afford the title compound (140 mg, 48%) as a light yellow solid. LCMS (ESI, m/z): 847 [M+H]+

### Step 12: Synthesis of 7-((S)-2-amino-3-fluoropropyl)-2-(1-(cyclopropylmethyl)-5-fluoro-7-((1-(oxazol-5-yl)propan-2-yl)oxy)-1H-indol-2-yl)-3-methyl-3,5,6,7-tetrahydro-8H-imidazo[4,5-b][1,6]naphthyridin-8-one

To a solution of tert-butyl ((2S)-1-(2-(1-(cyclopropylmethyl)-5-fluoro-7-((1-(2-(triisopropylsilyl)oxazol-5-yl)propan-2-yl)oxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (140 mg, 0.17 mmol) in DCM (5 mL), was added HF (2 mL). The mixture was stirred at room temperature for 1 h. The reaction was monitored by LCMS. The mixture was concentrated under vacuum. The crude product was purified by Prep-HPLC (Method F) to afford the title compound (23.6 mg, 23.3%) as an off-white solid. ¹H NMR (400 MHz, DMSO-d₆) δ 8.48 (s, 1H), 8.28 (s, 1H), 7.12 (s, 1H), 7.04-7.01 (m, 2H), 6.89-6.86 (m, 1H), 5.08-5.07 (m, 1H), 4.64-4.62 (m, 2H), 4.42 (m, 1H), 4.30 (m, 1H), 3.93 (s, 3H), 3.85-3.58 (m, 4H), 3.28-3.2 (m, 5H), 1.42 (d, J=8.0 Hz, 3H), 0.92-0.89 (m, 1H), 0.18-0.16 (m, 2H), −0.18-−0.01 (m, 2H). LCMS (ESI, m/z): 590 [M+H]+. LCMS RT: 0.909 min. (Method B).

Compounds of Examples 65a and 65b in Table 6 were obtained following chiral separation of Example 64 [Column: CHIRALPAK IF, 2×25 cm, 5 μm; Mobile Phase A: hexane:DCM (3:1) (0.5% 2M NH₃ in MeOH); Mobile Phase B: MeOH; Flow rate: 20 mL/min.; Gradient: ioscratic 50% B for 17 min.; Detection: UV (220/254 nm)] to afford single isomers of unknown configuration at the stereogenic methyl group.

TABLE 6

| Example | | LCMS m/z [M + H]+; LCMS RT (LCMS |
|---|---|---|
| Number | Structure | method); ¹H NMR (MHz, solvent) |
| 65a earlier eluting isomer | 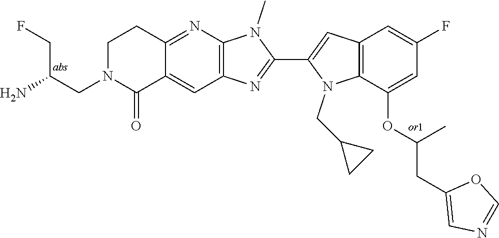 [SMILES: CC(Cc1cnco1)Oc1cc(F)cc2cc(-c3nc4cc5c(nc4n3C)CCN(C[C@H](N)CF)C5=O)n(CC3CC3)c12] | 7-((S)-2-amino-3-fluoropropyl)-2-(1- (cyclopropylmethyl)-5-fluoro-7-((1-(oxazol-5- yl)propan-2-yl)oxy)-1H-indol-2-yl)-3-methyl- 3,5,6,7-tetrahydro-8H-imidazo[4,5- b][1,6]naphthyridin-8-one LCMS (ESI, m/z): 590 [M + H]+; LCMS RT: 0.914 min. (Method B) |
| 65b later eluting isomer | 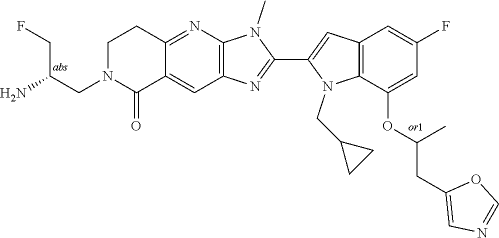 [SMILES: CC(Cc1cnco1)Oc1cc(F)cc2cc(-c3nc4cc5c(nc4n3C)CCN(C[C@H](N)CF)C5=O)n(CC3CC3)c12] | 7-((S)-2-amino-3-fluoropropyl)-2-(1- (cyclopropylmethyl)-5-fluoro-7-((1-(oxazol-5- yl)propan-2-yl)oxy)-1H-indol-2-yl)-3-methyl- 3,5,6,7-tetrahydro-8H-imidazo[4,5- b][1,6]naphthyridin-8-one LCMS (ESI, m/z): 590 [M + H]+; LCMS RT: 0.914 min (Method B). |

### Example 66. 7—((S)-2-amino-3-fluoropropyl)-2-(1-(cyclopropylmethyl)-7-(((S)-1-(4-fluoro-1H-imidazol-1-yl)propan-2-yl)oxy)-1H-indol-2-yl)-3-methyl-3,5,6,7-tetrahydro-8H-imidazo[4,5-b][1,6]naphthyridin-8-one

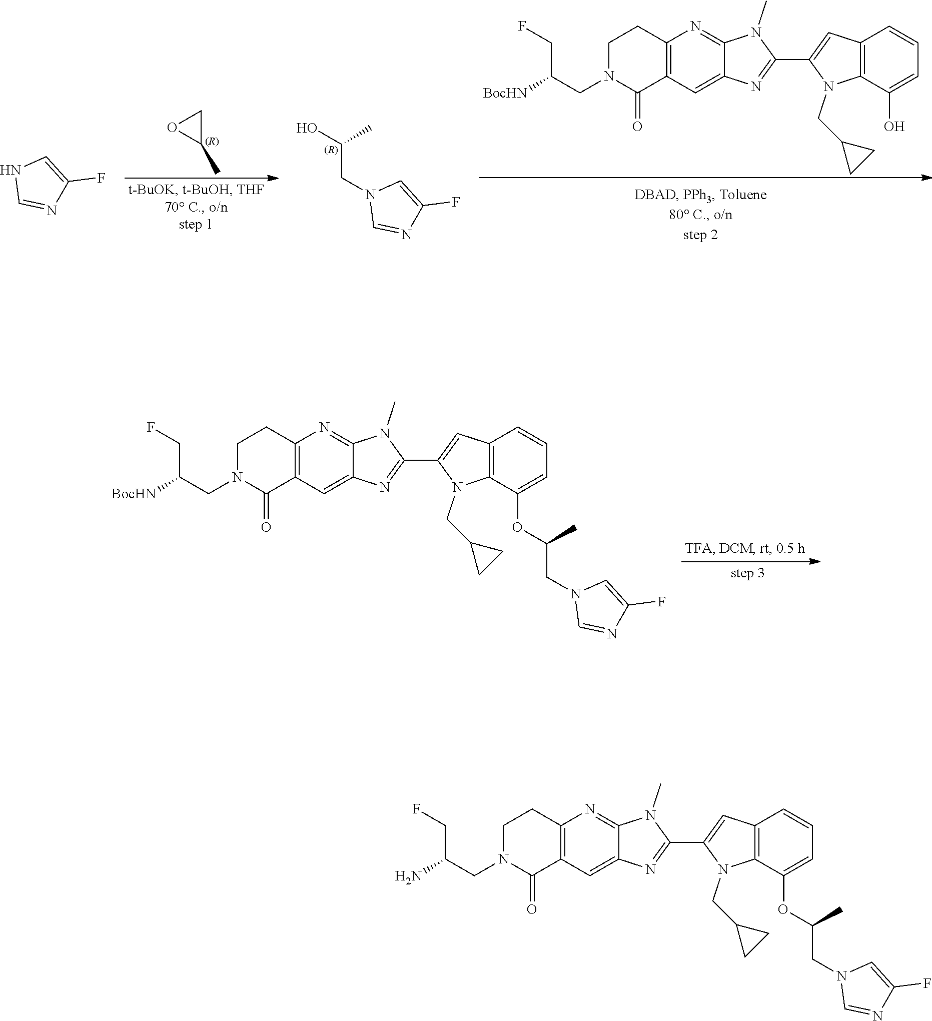

### Step 1: Synthesis of (R)-1-(4-fluoro-1H-imidazol-1-yl)propan-2-ol

4-Fluoro-1H-imidazole (600 mg, 6.97 mmol), (R)-2-methyloxirane (485 mg, 8.37 mmol) and potassium tert-butoxide (2.15 mL, 13.9 mmol) were dissolved in THF (4 mL) and tert-butanol (4 mL). The mixture was stirred at 70° C. for 1 h. The reaction was monitored by LCMS. The mixture was concentrated under vacuum. The crude product was purified by flash column chromatography on C18 silica to afford the title compound (430 mg, 42.6%) and as a colorless oil. LCMS (ESI, m/z): 145 [M+H]+.

### Step 2: Synthesis of tert-butyl ((S)-1-(2-(1-(cyclopropylmethyl)-7-(((S)-1-(4-fluoro-1H-imidazol-1-yl)propan-2-yl)oxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 5 (50 mg, 0.09 mmol) was reacted with (R)-1-(4-fluoro-1H-imidazol-1-yl)propan-2-ol (64 mg, 0.44 mmol) according to General Procedure 10. The crude product was purified by flash column chromatography on C18 silica to afford the title compound (50 mg, 81.7%) as a yellow solid. LCMS (ESI, m/z): 689 [M+H]+.

### Step 3: Synthesis of 7-((S)-2-amino-3-fluoropropyl)-2-(1-(cyclopropylmethyl)-7-(((S)-1-(4-fluoro-1H-imidazol-1-yl)propan-2-yl)oxy)-1H-indol-2-yl)-3-methyl-3,5,6,7-tetrahydro-8H-imidazo[4,5-b][1,6]naphthyridin-8-one

Tert-butyl ((S)-1-(2-(1-(cyclopropylmethyl)-7-(((S)-1-(4-fluoro-1H-imidazol-1-yl)propan-2-yl)oxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (60 mg. 0.09 mmol) was reacted according to General Procedure 2. The crude product was purified by (Method G) to afford the title compound (29.8 mg, 57.8%) as a yellow solid. ¹H NMR (400 MHz, DMSO-d₆) δ 8.52 (s, 1H), 8.41-8.29 (m, 3H), 7.42 (s, 1H), 7.17-7.07 (m, 1H), 7.05 (s, 1H), 7.03 (t, J=14 Hz, 1H), 6.99 (d, J=6.8 Hz, 1H), 6.89-6.88 (m, 1H), 5.11-5.10 (m, 1H), 4.75-4.63 (m, 4H), 4.36-4.31 (m, 2H), 3.95 (s, 3H), 3.87-3.72 (m, 2H), 3.54-3.35 (m, 3H), 3.33-3.32 (m, 2H), 1.33 (s, 3H), 0.95-0.75 (m, 1H), 0.25-0.15 (m, 2H), −0.15-−0.35 (m, 2H). LCMS (ESI, m/z): 589 [M+H]+. LCMS RT: 0.866 min (Method D).

Compounds of Examples 67 to 77 in Table 7 were obtained following a procedure similar to the preparation of a compound of Example 66 using the appropriate starting reagents.

TABLE 7

| | | LCMS m/z [M + H]+; LCMS RT |
|---|---|---|
| Example | | (LCMS method); |
| Number | Structure | ¹H NMR (MHz, solvent) |
| 67 | 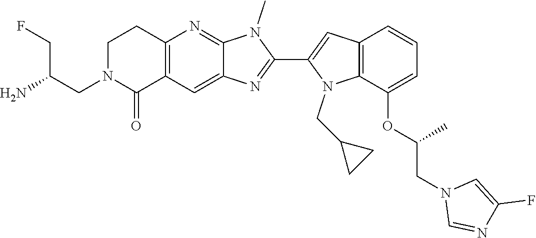 [SMILES: C[C@H](Cn1cnc(F)c1)Oc1cccc2cc(-c3nc4cc5c(nc4n3C)CCN(C[C@H](N)CF)C5=O)n(CC3CC3)c12] | 7-((S)-2-amino-3-fluoropropyl)-2-(1- (cyclopropylmethyl)-7-(((R)-1-(4- fluoro-1H-imidazol-1-yl)propan-2- yl)oxy)-1H-indol-2-yl)-3-methyl- 3,5,6,7-tetrahydro-8H-imidazo[4,5- b][1,6]naphthyridin-8-one LCMS (ESI, m/z): 589 [M + H]+; LCMS RT: 0.874 min (Method D) ¹H NMR (400 MHz, DMSO-d₆): δ 8.52 (s, 1H), 8.28 (s, 3H), 7.41-7.40 (m, 1H), 7.28 (d, J = 8 Hz, 1H), 7.17 (s, 1H), 7.07-6.97 (m, 2H), 6.88 (d, J = 8 Hz, 1H), 5.10 (d, J = 4 Hz, 1H), 4.78- 4.61 (m, 4H), 4.35-4.31 (m, 2H), 3.96 (s, 3H), 3.88-3.64 (m, 5H), 3.51-3.31 |
| | | (m, 2H), 1.34-1.33 (m, 3H), 0.87- |
| | | 0.84 (m, 1H), 0.19-0.16 (m, 2H), |
| | | −0.05-−0.22 (m, 2H). |
| 68 | 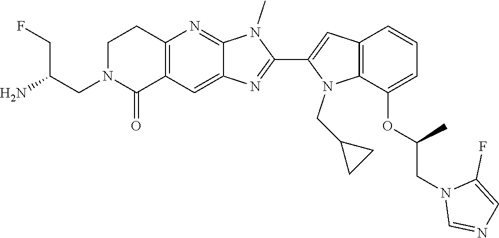 [SMILES: C[C@@H](Cn1cncc1F)Oc1cccc2cc(-c3nc4cc5c(nc4n3C)CCN(C[C@H](N)CF)C5=O)n(CC3CC3)c12] | 7-((S)-2-amino-3-fluoropropyl)-2-(1- (cyclopropylmethyl)-7-(((S)-1-(5- fluoro-1H-imidazol-1-yl)propan-2- yl)oxy)-1H-indol-2-yl)-3-methyl- 3,5,6,7-tetrahydro-8H-imidazo[4,5- b][1,6]naphthyridin-8-one LCMS (ESI, m/z): 589 [M + H]+; LCMS RT: 0.761 min (Method D) ¹H NMR (400 MHz, DMSO-d6) δ 8.52 (s, 1H), 8.31 (s, 3H), 8.21 (s, 1H), 7.31 (d, J = 8 Hz, 1H), 7.05 (s, 1H), 7.03- 6.97 (m, 2H), 6.89-6.88 (m, 1H), 5.11- 5.10 (m, 1H), 4.75-4.63 (m, 5H), 4.36-4.31 (m, 1H), 3.95 (s, 3H), 3.87- 3.72 (m, 2H), 3.54-3.35 (m, 3H), |
| | | 3.35-3.32 (m, 2H), 1.45-1.39 (m, |
| | | 3H), 0.90-0.85 (m, 1H), 0.20-0.10 |
| | | (m, 2H), −0.15-−0.24 (m, 1H), |
| | | −0.25-−0.35 (m, 1H). |
| 69 | 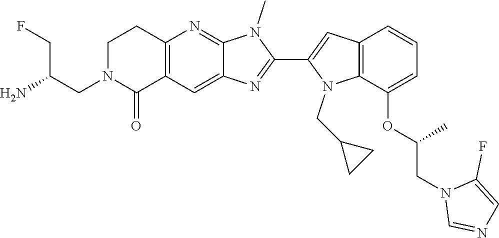 [SMILES: C[C@H](Cn1cncc1F)Oc1cccc2cc(-c3nc4cc5c(nc4n3C)CCN(C[C@H](N)CF)C5=O)n(CC3CC3)c12] | 7-((S)-2-amino-3-fluoropropyl)-2-(1- (cyclopropylmethyl)-7-(((R)-1-(5- fluoro-1H-imidazol-1-yl)propan-2- yl)oxy)-1H-indol-2-yl)-3-methyl- 3,5,6,7-tetrahydro-8H-imidazo[4,5- b][1,6]naphthyridin-8-one LCMS (ESI, m/z): 589 [M + H]+; LCMS RT: 1.270 min (Method D) ¹H NMR (400 MHz, DMSO-d₆) δ 8.51 (s, 1H), 8.30 (s, 3H), 8.17 (s, 1H), 7.29 (d, J = 8 Hz, 1H), 7.18 (d, J = 8 Hz, 1H), 7.07-7.03 (m, 2H), 6.90 (d, J = 7.6 Hz, 1H), 5.17-5.16 (m, 1H), 4.78- 4.38 (m, 6H), 3.96 (s, 3H), 3.85-3.51 (m, 5H), 3.34-3.32 (m, 2H), 1.38 (s, |
| | | 3H), 0.86-0.83 (m, 1H), 0.16 (d, J = 8 |
| | | Hz, 2H), −0.20-−0.22 (m, 1H), |
| | | −0.27-−0.29 (m, 1H). |
| 70 | 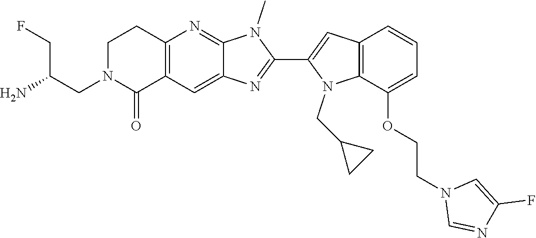 [SMILES: Cn1c(-c2cc3cccc(OCCn4cnc(F)c4)c3n2CC2CC2)nc2cc3c(nc21)CCN(C[C@H](N)CF)C3=O] | (S)-7-(2-amino-3-fluoropropyl)-2-(1- (cyclopropylmethyl)-7-(2-(4-fluoro- 1H-imidazol-1-yl)ethoxy)-1H-indol-2- yl)-3-methyl-3,5,6,7-tetrahydro-8H- imidazo[4,5-b][1,6]naphthyridin-8-one LCMS (ESI, m/z): 575 [M + H]+; LCMS RT: 1.446 min (Method B) ¹H NMR (400 MHz, DMSO-d₆): δ 8.52 (s, 1H), 8.31 (s, 3H), 7.48 (s, 1H), 7.31 (d, J = 7.9 Hz, 1H), 7.17 (s, 1H), 7.11-6.98 (m, 2H), 6.89 (d, J = 7.8 Hz, 1H), 4.83-4.59 (m, 2H), 4.62-4.50 (m, 4H), 4.50-4.42 (m, 2H), 3.97-3.90 (m, 5H), 3.90-3.70 (m, 3H), 3.33 |
| | | (t, J = 6.6 Hz, 2H), 0.84-0.79 (m, |
| | | 1H), 0.19-0.11 (m, 2H), −0.24-−0.33 |
| | | (m, 2H). |
| 71 | 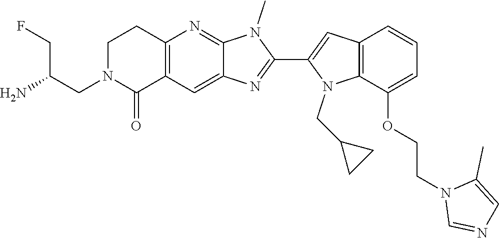 [SMILES: Cc1cncn1CCOc1cccc2cc(-c3nc4cc5c(nc4n3C)CCN(C[C@H](N)CF)C5=O)n(CC3CC3)c12] | (S)-7-(2-amino-3-fluoropropyl)-2-(1- (cyclopropylmethyl)-7-(2-(5-methyl- 1H-imidazol-1-yl)ethoxy)-1H-indol-2- yl)-3-methyl-3,5,6,7-tetrahydro-8H- imidazo[4,5-b][1,6]naphthyridin-8-one LCMS (ESI, m/z): 571 [M + H]+; LCMS RT: 0.741 min (Method D) ¹H NMR (400 MHz, Methanol-d4) δ 8.56 (s, 1H), 7.86 (s, 1H), 7.32-7.30 (m, 1H), 7.07-7.02 (m, 2H), 6.84- 6.80 (m, 2H), 4.49-4.40 (m, 8H), 3.96 (s, 3H), 3.85-3.72 (m, 2H), 3.69-3.62 (m, 2H), 3.45-3.32 (m, 3H), 2.29 (s, 3H), 0.83-0.80 (m, 1H), 0.16-0.12 (m, 2H), −0.31-−0.35 (m, 2H). |
| 72 | 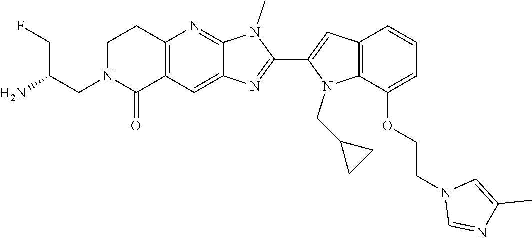 [SMILES: Cc1cn(CCOc2cccc3cc(-c4nc5cc6c(nc5n4C)CCN(C[C@H](N)CF)C6=O)n(CC4CC4)c23)cn1] | (S)-7-(2-amino-3-fluoropropyl)-2-(1- (cyclopropylmethyl)-7-(2-(4-methyl- 1H-imidazol-1-yl)ethoxy)-1H-indol-2- yl)-3-methyl-3,5,6,7-tetrahydro-8H- imidazo[4,5-b][1,6]naphthyridin-8-one LCMS (ESI, m/z): 571 [M + H]+; LCMS RT: 0.738 min (Method D) ¹H NMR (400 MHz, Methanol-d4) δ 8.58 (s, 1H), 7.75 (s, 1H), 7.30 (d, J = 8 Hz, 1H), 7.08-7.03 (m, 3H), 6.85- 6.83 (d, J = 7.6 Hz, 1H), 4.48-4.42 (m, 8H), 4.10 (s, 3H), 3.85-3.82 (m, 2H), 3.68-3.65 (m, 2H), 3.62-3.46 (m, 1H), 3.41-3.32 (m, 2H), 2.17 (s, 3H), 0.86-0.83 (m, 1H), 0.20-0.12 (m, 2H), −0.28-−0.31 (m, 2H). |

### Example 73a and 73b. 7-((S)-2-amino-3-fluoropropyl)-2-(1-(cyclopropylmethyl)-7-(1-fluoro-2-(4-fluoro-1H-imidazol-1-yl)ethoxy)-1H-indol-2-yl)-3-methyl-3,5,6,7-tetrahydro-8H-imidazo[4,5-b][1,6]naphthyridin-8-one

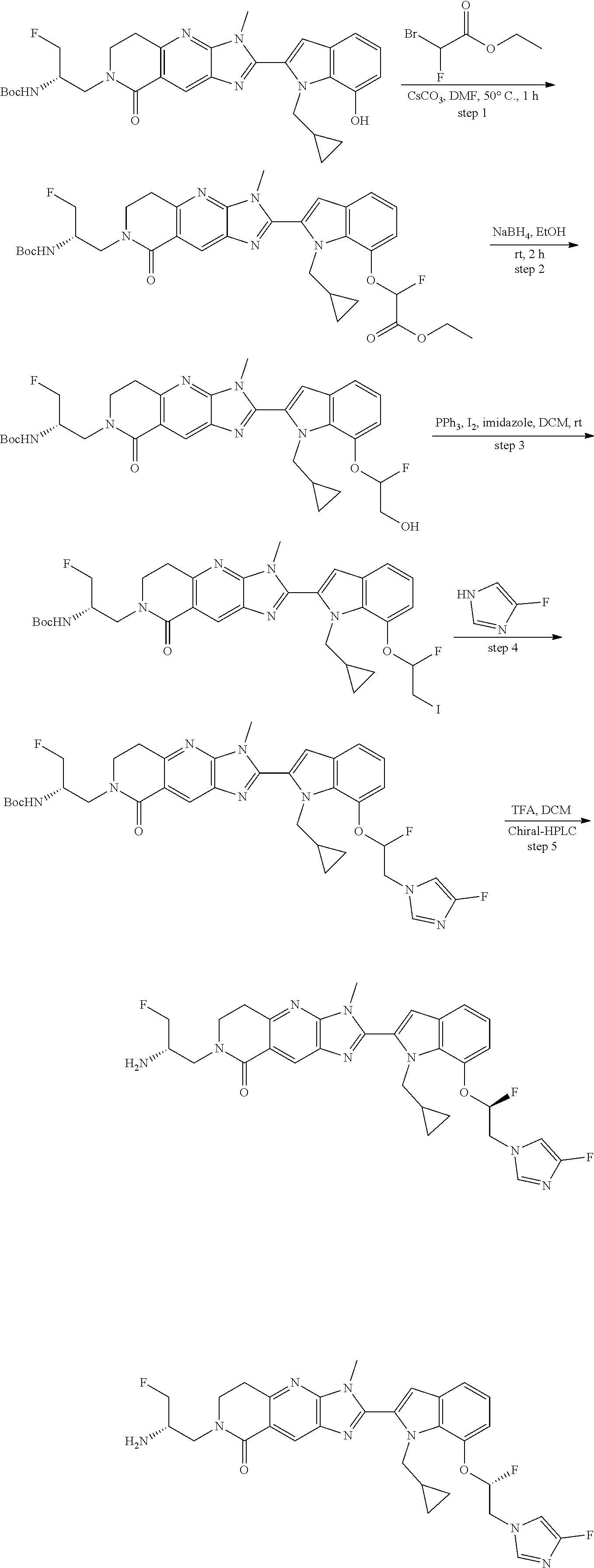

### Step 1: Synthesis of ethyl 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-2-fluoroacetate

To a solution of Intermediate 5 (300 mg, 0.53 mmol) in DMF (5 mL) was added ethyl 2-bromo-2-fluoro-acetate (148 mg, 0.8 mmol) and Cs₂CO₃ (520 mg, 1.6 mmol). The reaction was stirred at 50° C. for 2 h. The reaction was monitored by LCMS. The mixture was quenched with water (15 mL) and extracted with ethyl acetate (15 mL). The organic layer was washed with water (15 mL) and brine (2×15 mL), dried over anhydrous sodium sulfate and concentrated under vacuum. The crude product was purified by column chromatography (petroleum ether/ethyl acetate=2:1) to afford the title compound (280 mg, 78.7%) as a mixture of diastereomers as a light yellow solid. LCMS (ESI, m/z): 667 [M+H]+.

### Step 2: Synthesis of tert-butyl ((2S)-1-(2-(1-(cyclopropylmethyl)-7-(1-fluoro-2-hydroxyethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of ethyl 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-2-fluoroacetate (238 mg, 0.36 mmol) in ethanol (5 mL) was added NaBH₄ (40.5 mg, 1.07 mmol) at 0° C. The reaction was stirred at room temperature for 2 h. The reaction was monitored by LCMS. The solvent was evaporated under vacuum. The residue was diluted with water (10 mL) and extracted with DCM (3×10 mL), the organic layers combined and dried over anhydrous sodium sulfate and concentrated under vacuum. The crude product was purified by column chromatography (petroleum ether/ethyl acetate=1:1) to afford the title compound (200 mg, 89.6%) as a mixture of diastereomers as a light yellow solid. LCMS (ESI, m/z): 625 [M+H]+.

### Step 3: Synthesis of tert-butyl ((2S)-1-(2-(1-(cyclopropylmethyl)-7-(1-fluoro-2-iodoethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of tert-butyl ((2S)-1-(2-(1-(cyclopropylmethyl)-7-(1-fluoro-2-hydroxyethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (100 mg, 0.02 mmol) in DCM (3 mL) was added iodine (81.3 mg, 0.03 mmol), imidazole (27.2 mg, 0.04 mmol), and PPh₃ (84 mg, 0.03 mmol). The reaction was stirred at room temperature for 2 h under a nitrogen atmosphere. The reaction was monitored by LCMS. The mixture was quenched by adding water (30 mL), extracted with DCM (3×30 mL). The organic layers were combined and dried over anhydrous sodium sulfate and concentrated under vacuum. The crude product was purified by column chromatography (petroleum ether/ethyl acetate=1:1) to afford the title compound (30 mg, 28.9%) as a mixture of diastereomers as a light yellow solid. LCMS (ESI, m/z): 735 [M+H]+.

### Step 4: Synthesis of tert-butyl ((2S)-1-(2-(1-(cyclopropylmethyl)-7-(1-fluoro-2-(4-fluoro-1H-imidazol-1-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Tert-butyl ((2S)-1-(2-(1-(cyclopropylmethyl)-7-(1-fluoro-2-iodoethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.04 mmol) in DMF (3 mL) was reacted with 4-fluoro-1H-imidazole (3.87 mg, 0.04 mmol) and Cs₂CO₃ (39.8 mg, 0.12 mmol) according to General Procedure 14. The crude product was purified by column chromatography (petroleum ether/ethyl acetate=1:1) to afford the title compound (10 mg, 35.3%) as a mixture of diastereomers as a light yellow solid. LCMS (ESI, m/z): 693 [M+H]+.

### Step 5: Synthesis of 7-((S)-2-amino-3-fluoropropyl)-2-(1-(cyclopropylmethyl)-7-(1-fluoro-2-(4-fluoro-1H-imidazol-1-yl)ethoxy)-1H-indol-2-yl)-3-methyl-3,5,6,7-tetrahydro-8H-imidazo[4,5-b][1,6]naphthyridin-8-one

Tert-butyl ((2S)-1-(2-(1-(cyclopropylmethyl)-7-(1-fluoro-2-(4-fluoro-1H-imidazol-1-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.04 mmol) was reacted according to General Procedure 2. The crude material was purified by Prep-HPLC (Method F) to afford the title compound as a mixture of diastereomers which were separated by Chiral Prep HPLC [Column: CHIRALPAK ID, 2×25 cm, 5 m; Mobile Phase A: MTBE (0.5% 2M NH₃ in MeOH), Mobile Phase B: MeOH; Flow rate: 20 mL/min.; Gradient: ioscratic 50% B for 11 min.; Detection: UV (254/220 nm)] to afford single stereoisomers of unknown configuration at the fluorine center.

Example 73a. Isomer 1: Chiral HPLC RT: 5.85 min. ¹H NMR (400 MHz, DMSO-d₆) δ 8.50 (s, 1H), 7.52 (s, 1H), 7.48 (d, J=8.0 Hz, 1H), 7.22 (s, 1H), 7.13 (d, J=8.0 Hz, 1H), 7.05 (s, 1H), 7.04 (d, J=8.0 Hz, 1H) 6.61 (d, J=60 Hz, 1H), 4.67-4.31 (m, 6H), 3.95 (s, 3H), 3.78-3.29 (m, 4H), 3.25-3.21 (m, 3H), 0.93-0.91 (m, 1H), 0.25-0.17 (m, 2H), −0.18-−0.28 (m, 2H). LCMS (ESI, m/z): 593 [M+H]+. LCMS RT: 0.878 min. (Method D).

Example 73b. Isomer 2: Chiral HPLC RT: 8.44 min. ¹H NMR (400 MHz, DMSO-d₆): δ 8.49 (s, 1H), 7.52 (s, 1H), 7.49 (d, J=8.0 Hz, 1H), 7.22 (s, 1H), 7.13 (d, J=8.0 Hz, 1H), 7.05 (s, 1H), 7.04 (d, J=8.0 Hz, 1H) 6.61 (d, J=60 Hz, 1H), 4.67-4.31 (m, 6H), 3.95 (s, 3H), 3.78-3.29 (in 4H), 3.25-3.21 (m, 3H), 0.92-0.89 (m, 1H), 0.21-0.17 (in, 2H), −0.18-−0.23 (in, 2H). LCMS (ESI, m/z): 593 [M+H]+; LCMS RT: 0.878 min (Method D).

Compounds of Examples 74 to 77 in Table 8 were obtained following a procedure similar to the preparation of a compound of Example 73 using the appropriate starting reagents.

TABLE 8

| | | LCMS m/z [M + H]+; LCMS RT |
|---|---|---|
| Example | | (LCMS method); ¹H NMR (MHz, |
| Number | Structure | solvent) |
| 74a earlier eluting isomer | 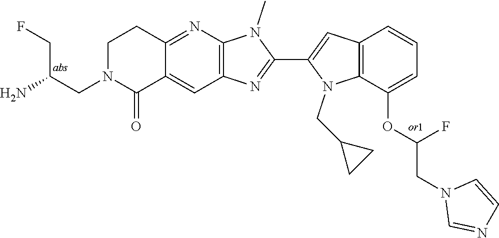 [SMILES: Cn1c(-c2cc3cccc(OC(F)Cn4ccnc4)c3n2CC2CC2)nc2cc3c(nc21)CCN(C[C@H](N)CF)C3=O] | 7-((S)-2-amino-3-fluoropropyl)-2-(1- (cyclopropylmethyl)-7-(1-fluoro-2-(1H- imidazol-1-yl)ethoxy)-1H-indol-2-yl)-3- methyl-3,5,6,7-tetrahydro-8H- imidazo[4,5-b][1,6]naphthyridin-8-one LCMS (ESI, m/z): 575 [M + H]+; LCMS RT: 0.729 min (Method D) ¹H NMR (400 MHz, DMSO-d₆) δ 8.52 (s, 1H), 7.79 (s, 1H), 7.48 (d, J = 8.0 Hz, 1H), 7.34 (s, 1H), 7.24 (s, 1H), 7.11-7.16 (m, , 2H), 6.98 (s, 1H), 6.61 (d, J = 68.0 Hz, 1H), 4.72-4.47 (m, 6H), 3.96 (s, 3H), 3.80-3.70 (m, 5H), 3.52-3.45 (m, 2H), 0.93-0.91 (m, 1H), 0.29-0.15 (m, 2H), −0.18-−0.28 (m, 2H). |
| | | Prep-Chiral HPLC RT: 1.47 min. |
| | | (Column: CHIRALPAK IF-3, |
| | | 4.6x50 mm, 3.0 um; mobile phase |
| | | A: MTBE + 0.1% DEA:MeOH (1:1); flow |
| | | rate: 1 mL/min.; isocratic 0% B. |
| 74b later eluting isomer | 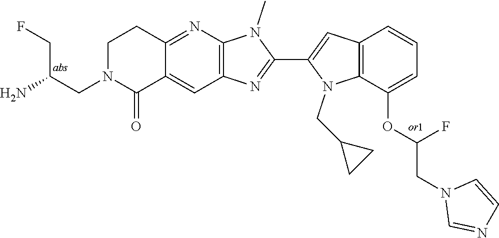 [SMILES: Cn1c(-c2cc3cccc(OC(F)Cn4ccnc4)c3n2CC2CC2)nc2cc3c(nc21)CCN(C[C@H](N)CF)C3=O] | 7-((S)-2-amino-3-fluoropropyl)-2-(1- (cyclopropylmethyl)-7-(1-fluoro-2-(1H- imidazol-1-yl)ethoxy)-1H-indol-2-yl)-3- methyl-3,5,6,7-tetrahydro-8H- imidazo[4,5-b][1,6]naphthyridin-8-one LCMS (ESI, m/z): 575 [M + H]+; LCMS RT: 0.729 min (Method D) ¹H NMR (400 MHz, DMSO-d₆) δ 8.52 (s, 1H), 7.79 (s, 1H), 7.48 (d, J = 8.0 Hz, 1H), 7.34 (s, 1H), 7.24 (s, 1H), 7.11-7.16 (m, 2H), 6.98 (s, 1H), 6.61 (d, J = 68.0 Hz, 1H), 4.72-4.47 (m, 6H), 3.96 (s, 3H), 3.80-3.70 (m, 5H), 3.52-3.45 (m, 2H), 0.93-0.91 (m, 1H), 0.29-0.15 (m, 2H), −0.18-−0.28 (m, 2H). Prep-Chiral- HPLC RT: 1.91 min. (Method see example 74a). |
| 75a earlier eluting isomer | 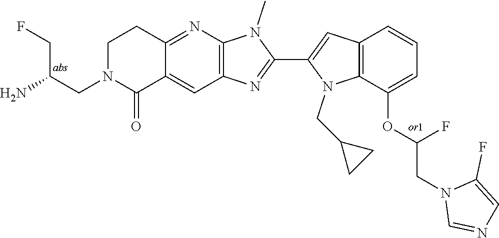 [SMILES: Cn1c(-c2cc3cccc(OC(F)Cn4cncc4F)c3n2CC2CC2)nc2cc3c(nc21)CCN(C[C@H](N)CF)C3=O] | 7-((S)-2-amino-3-fluoropropyl)-2-(1- (cyclopropylmethyl)-7-(1-fluoro-2-(5- fluoro-1H-imidazol-1-yl)ethoxy)-1H- indol-2-yl)-3-methyl-3,5,6,7-tetrahydro- 8H-imidazo[4,5-b][1,6]naphthyridin-8- one LCMS (ESI, m/z): 593 [M + H]+; LCMS RT: 1.534 min (Method B) ¹H NMR (400 MHz, DMSO-d₆) δ 8.50 (s, 1H), 7.52 (s, 1H), 7.49 (d, J = 8.0 Hz, 1H), 7.24 (s, 1H), 7.15 (d, J = 8.0 Hz, 1H), 7.13 (s, 1H), 7.04 (d, J = 8.0 Hz, 1H), 6.69- 6.54 (m, 1H), 4.67-4.32 (m, 6H), 3.96 (s, 3H), 3.79-3.48 (m, 4H), 3.33-3.28 (m, 3H), 0.91-0.89 (m, 1H), 0.2-0.18 |
| | | (m, 2H), −0.18-−0.22 (m, 2H). |
| | | Prep-Chiral HPLC RT: 12.67 min. |
| | | [Column: CHIRALPAK IE, 2 x 25 cm, 5 |
| | | μm; Mobile Phase A: MTBE (0.5% 2M |
| | | NH₃ in MeOH), Mobile Phase B: EtOH; |
| | | flow rate: 16 mL/min.; Gradient: isocratic |
| | | 50% B for 22 min.; Detection UV |
| | | (220/254 nm)]. |
| 75b later eluting isomer | 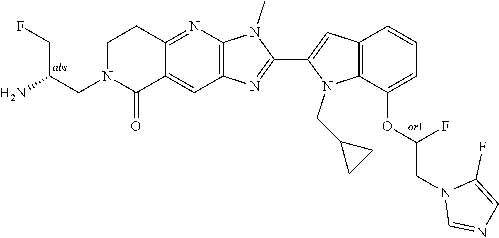 [SMILES: Cn1c(-c2cc3cccc(OC(F)Cn4cncc4F)c3n2CC2CC2)nc2cc3c(nc21)CCN(C[C@H](N)CF)C3=O] | 7-((S)-2-amino-3-fluoropropyl)-2-(1- (cyclopropylmethyl)-7-(1-fluoro-2-(5- fluoro-1H-imidazol-1-yl)ethoxy)-1H- indol-2-yl)-3-methyl-3,5,6,7-tetrahydro- 8H-imidazo[4,5-b][1,6]naphthyridin-8- one LCMS (ESI, m/z): 593 [M + H]+; LCMS RT: 0.869 min (Method D) ¹H NMR (400 MHz, DMSO-d₆) δ 8.50 (s, 1H), 7.52 (s, 1H), 7.49 (d, J = 8.0 Hz, 1H), 7.24 (s, 1H), 7.15 (d, J = 8.0 Hz, 1H), 7.13 (s, 1H), 7.04 (d, J = 8.0 Hz, 1H), 6.69- 6.54 (m, 1H), 4.67-4.32 (m, 6H), 3.96 (s, 3H), 3.79-3.48 (m, 4H), 3.33-3.28 (m, 3H), 0.91-0.89 (m, 1H), 0.2-0.18 |
| | | (m, 2H), −0.18-−0.22 (m, 2H) |
| | | Prep Chiral HPLC RT: 19.01 min. |
| | | (Method see Example 75a). |
| 76a earlier eluting isomer | 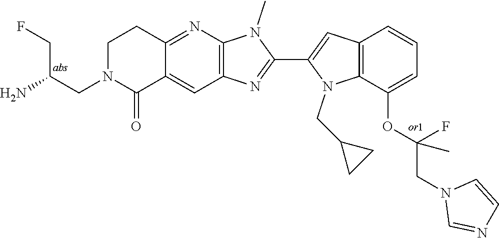 [SMILES: Cn1c(-c2cc3cccc(OC(C)(F)Cn4ccnc4)c3n2CC2CC2)nc2cc3c(nc21)CCN(C[C@H](N)CF)C3=O] | 7-((S)-2-amino-3-fluoropropyl)-2-(1- (cyclopropylmethyl)-7-((2-fluoro-1-(1H- imidazol-1-yl)propan-2-yl)oxy)-1H- indol-2-yl)-3-methyl-3,5,6,7-tetrahydro- 8H-imidazo[4,5-b][1,6]naphthyridin-8- one LCMS (ESI, m/z): 589 [M + H]+; LCMS RT: 0.784 min. (Method H) ¹H NMR (400 MHz, Methanol-d₄) δ 8.66 (s, 1H), 8.51 (s, 1H), 7.64-7.54 (m, 2H), 7.39 (s, 1H), 7.24-7.13 (m, 3H), 4.92- 4.88 (m, 1H), 4.86- 4.81 (m, 2H), 4.77-4.66 (m, 1H), 4.60- 4.50 (m, 1H), 4.42-4.32 (m, 1H), 4.13-4.03 (m, 4H), 4.03-3.77 (m, 4H), |
| | | 3.44 (t, J = 6.8 Hz, 2H), 1.67 |
| | | (d, J = 18 Hz, 3H), 0.93-0.81 (m, 1H), |
| | | 0.36-0.17 (m, 2H), −0.08-−0.19 (m, |
| | | 1H), −0.27-−0.38 (m, 1H) |
| | | Prep Chiral HPLC RT: 21.7 min. |
| | | [CHIRALPAK IF, 2 x 25 cm, 5 μm; |
| | | Mobile Phase A: MTBE (0.5% 2M NH₃ |
| | | in MeOH), Mobile Phase B: MeOH; |
| | | Flow rate: 16 mL/min.; Gradient: |
| | | isocratic 20% B for 30 min.; Detection: |
| | | UV (220/254 nm)]. |
| 76b later eluting siomer | 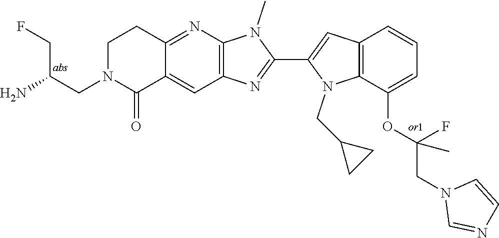 [SMILES: Cn1c(-c2cc3cccc(OC(C)(F)Cn4ccnc4)c3n2CC2CC2)nc2cc3c(nc21)CCN(C[C@H](N)CF)C3=O] | 7-((S)-2-amino-3-fluoropropyl)-2-(1- (cyclopropylmethyl)-7-((2-fluoro-1-(1H- imidazol-1-yl)propan-2-yl)oxy)-1H- indol-2-yl)-3-methyl-3,5,6,7-tetrahydro- 8H-imidazo[4,5-b][1,6]naphthyridin-8- one LCMS (ESI, m/z): 589 [M + H]+; LCMS RT: 0.777 min (Method H) ¹H NMR (400 MHz, Methanol-d₄) δ 8.64 (s, 1H), 7.80 (s, 1H), 7.61-7.54 (m, 1H), 7.30 (s, 1H), 7.24-7.10 (m, 3H), 7.05 (s, 1H), 4.70 (d, J = 15.2 Hz, 2H), 4.62- 4.49 (m, 2H), 4.49-4.31 (m, 2H), 4.04 (s, 3H), 3.87 (t, J = 6.6 Hz, 2H), 3.78- 3.64 (m, 2H), 3.41 (t, J = 6.8 Hz, 3H), |
| | | 1.61 (d, J = 17.6 Hz, 3H), 0.93-0.81 (m, |
| | | 1H), 0.36-0.17 (m, 2H), −0.08-−0.19 |
| | | (m, 1H), −0.27-−0.38 (m, 1H). |
| | | Prep Chiral HPLC RT: 26.4 min. |
| | | (Method see Example 76a). |
| 77a earlier eluting isomer | 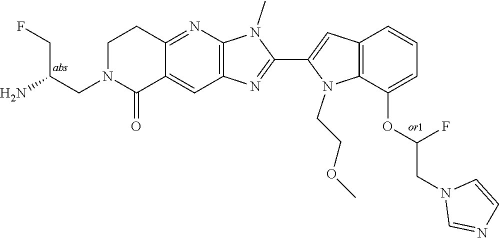 [SMILES: COCCn1c(-c2nc3cc4c(nc3n2C)CCN(C[C@H](N)CF)C4=O)cc2cccc(OC(F)Cn3ccnc3)c21] | 7-((S)-2-amino-3-fluoropropyl)-2-(7-(1- fluoro-2-(1H-imidazol-1-yl)ethoxy)-1- (2-methoxyethyl)-1H-indol-2-yl)-3- methyl-3,5,6,7-tetrahydro-8H- imidazo[4,5-b][1,6]naphthyridin-8-one LCMS (ESI, m/z): 579 [M + H]+; LCMS RT: 1.124 min (Method D) ¹H NMR (400 MHz, DMSO-d₆ + D₂O) δ 8.51 (s, 1H), 7.78 (s, 1H), 7.47 (d, J = 9.2 Hz, 1H), 7.32 (s, 1H), 7.12 (d, J = 12 Hz, 3H), 6.98 (s, 1H), 6.67-6.47 (m, 1H), 4.70-4.39 (m, 6H), 3.89 (s, 3H), 3.77-3.73 (m, 2H), 3.67-3.65 (m, 2H), 3.56-3.51 (m, 1H), 3.30 (s, 4H), 2.78 (s, 3H). |
| | | Chiral HPLC RT: 1.60 min. |
| | Key phenol intermediate was synthesized | (Method:CHIRALPAK IF-3, 4.6 x 50 |
| | via a similar synthetic route to Intermediate | mm, 3 μm; mobile phase |
| | 5 alkylating the indole NH with 1-bromo-2- | A: (MTBE + 0.1% DEA):MeOH (1:1), |
| | methoxyethane. | flow rate: 1 mL/min; gradient: isocratic |
| | | 0% B. |
| 77b later eluting isomer | 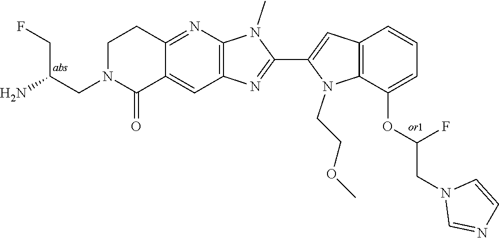 [SMILES: COCCn1c(-c2nc3cc4c(nc3n2C)CCN(C[C@H](N)CF)C4=O)cc2cccc(OC(F)Cn3ccnc3)c21] | 7-((S)-2-amino-3-fluoropropyl)-2-(7-(1- fluoro-2-(1H-imidazol-1-yl)ethoxy)-1- (2-methoxyethyl)-1H-indol-2-yl)-3- methyl-3,5,6,7-tetrahydro-8H- imidazo[4,5-b][1,6]naphthyridin-8-one LCMS (ESI, m/z): 579 [M + H]+; LCMS RT: 1.120 min (Method D) ¹H NMR (400 MHz, DMSO-d₆ + D2O) δ 8.51 (s, 1H), 7.78 (s, 1H), 7.47 (d, J = 9.2 Hz,1H), 7.32 (s, 1H), 7.12 (d, J = 12 Hz, 3H), 6.98 (s, 1H), 6.67-6.47 (m, 1H), 4.70-4.39 (m, 6H), 3.89 (s, 3H), 3.77-3.73 (m, 3H), 3.67- 3.65 (m, 2H), 3.34-3.30 (m, 4H), 2.78 (s, 3H). Chiral HPLC RT: 2.07 min. (Method see Example 77a) |

### Examples 78a, 78b, 78c, and 78d. 7-((S)-2-amino-3-fluoropropyl)-2-(1-(cyclopropylmethyl)-7-(1-fluoro-2-(1H-imidazol-1-yl)propoxy)-1H-indol-2-yl)-3-methyl-3,5,6,7-tetrahydro-8H-imidazo[4,5-b][1,6]naphthyridin-8-one

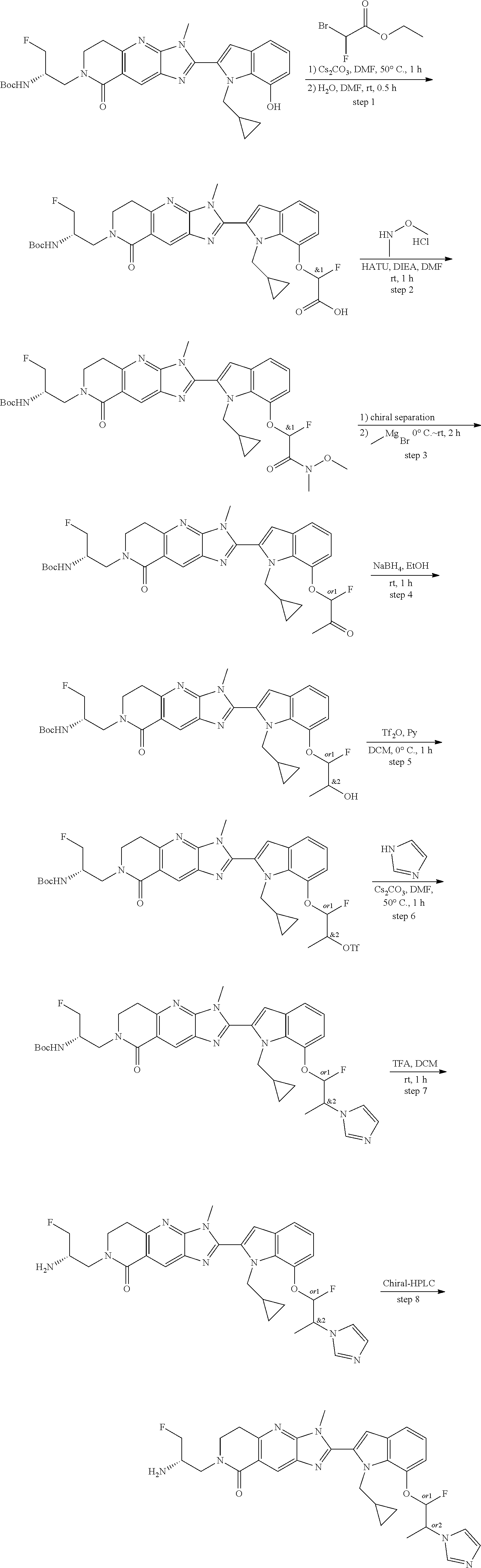

### Step 1: Synthesis of 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-2-fluoroacetic acid

To a solution of Intermediate 5 (1.5 g, 2.67 mmol) in DMF (26 mL), was added ethyl 2-bromo-2-fluoro-acetate (740 mg, 4.00 mmol) and Cs₂CO₃ (2.61 g, 8.00 mmol) under nitrogen atmosphere. The resulting solution was stirred at 50° C. for 1 h under nitrogen atmosphere. The reaction was monitored by LCMS. To the above mixture was added water (50 mL). The resulting solution was stirred at room temperature for 0.5 h. The reaction was monitored by LCMS. The mixture was extracted with ethyl acetate (80 mL), washed with water (2×80 mL) and brine (2×80 mL), dried over anhydrous sodium sulfate and concentrated under vacuum. The crude product was purified by column chromatography (ethyl acetate) to afford the title compound (1 g, 58%) as a mixture of diastereomers as a yellow solid. LCMS (ESI, m/z): 639 [M+H]+.

### Step 2: Synthesis of tert-butyl ((2S)-1-(2-(1-(cyclopropylmethyl)-7-(1-fluoro-2-(methoxy(methyl)amino)-2-oxoethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of 2-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-2-fluoroacetic acid (1 g, 1.57 mmol) and DIEA (1.02 g, 7.83 mmol) in DMF (15 mL), was added HATU (1.19 g, 3.13 mmol) at room temperature under nitrogen atmosphere. The mixture was stirred at room temperature for 10 min under nitrogen atmosphere. Then to the above resulting mixture was added dropwise a solution of N,O-dimethylhydroxylamine hydrochloride (764 mg, 7.83 mmol) in DMF (1 mL) at room temperature under nitrogen atmosphere. The resulting mixture was stirred at room temperature for 1 h under nitrogen atmosphere. The reaction was monitored by LCMS. The reaction was then quenched by adding water (20 mL) and extracted with ethyl acetate (60 mL). The combined organic extracts were washed with water (2×60 mL) and brine (2×60 mL), dried over anhydrous sodium sulfate and concentrated under vacuum. The crude product was purified by flash column chromatography on C18 silica to afford the title compound (1 g, 93%) as a mixture of diastereomers as a brown solid. LCMS (ESI, m/z): 682 [M+H]+.

### Step 3: Synthesis of tert-butyl ((2S)-1-(2-(1-(cyclopropylmethyl)-7-(1-fluoro-2-oxopropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

The diastereomers of tert-butyl ((2S)-1-(2-(1-(cyclopropylmethyl)-7-(1-fluoro-2-(methoxy(methyl)amino)-2-oxoethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (1 g) were separated by Prep-SFC [(Column: CHIRALPAK 1H, 5×25 cm, 5 mm; mobile phase A: CO₂, mobile phase B: IPA (0.5% 2M NH₃ in MeOH); flow rate: 200 mL/min.; gradient: isocratic 35% B; column temperature: 35° C.; back pressure: 100 bar; detection: UV (220 nm)] to afford single stereoisomers of unknown configuration at the fluorine center. Isomer 1 RT: 5.84 min.; Isomer 2 RT: 8.08 min.

To a solution of Isomer 1 (250 mg, 0.37 mmol) in THE (4 mL) was added methylmagnesium bromide (0.73 mL, 2.19 mmol) at 0° C. under nitrogen atmosphere. The resulting mixture was stirred at room temperature for 2 h under nitrogen atmosphere. The reaction was monitored by LCMS. The reaction was then quenched by adding water (10 mL), extracted with ethyl acetate (3×10 mL). The combined organic extracts were washed with brine (3×10 mL), dried over anhydrous sodium sulfate and concentrated under vacuum to afford the title compound (230 mg, 98%). LCMS (ESI, m/z): 637 [M+H]+

### Step 4 Synthesis of tert-butyl ((2S)-1-(2-(1-(cyclopropylmethyl)-7-(1-fluoro-2-hydroxypropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of tert-butyl ((2S)-1-(2-(1-(cyclopropylmethyl)-7-(1-fluoro-2-oxopropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (230 mg, 0.37 mmol) in ethanol (4 mL), was added NaBH₄ (138 mg, 3.66 mmol) at 0° C. under nitrogen atmosphere. The mixture was stirred at room temperature for 1 h under nitrogen atmosphere. The reaction was monitored by LCMS. The reaction was then quenched by adding acetone (5 mL). The mixture solution was concentrated under vacuum. The crude product was purified by flash column chromatography on C18 silica to afford the title compound (180 mg, 77%) as a mixture of diastereomers at the alcohol center as a yellow oil. LCMS (ESI, m/z): 639 [M+H]+

### Step 5: Synthesis of 1-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-1-fluoropropan-2-yl trifluoromethanesulfonate

To a solution of tert-butyl ((2S)-1-(2-(1-(cyclopropylmethyl)-7-(1-fluoro-2-hydroxypropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (160 mg, 0.25 mmol) in DCM (2.5 mL), was added Tf₂O (0.06 mL, 0.38 mmol) and pyridine (0.06 mL, 0.75 mmol) at 0° C. under nitrogen atmosphere. The resulting solution was stirred for 1 h at 0° C. under nitrogen atmosphere. The reaction was monitored by LCMS. The reaction was then quenched by adding saturated aqueous NH₄Cl (1 mL) at 0° C., extracted with DCM (3×10 mL). The combined organic extracts were washed with brine (3×10 mL), dried over anhydrous sodium sulfate and concentrated under vacuum and purified by column chromatography (DCM/MeOH=20:1) to afford the title compound (120 mg, 62%) as a mixture of diastereomers at the triflate center as a yellow oil. LCMS (ESI, m/z): 771 [M+H]+.

### Step 6 Synthesis of tert-butyl ((2S)-1-(2-(1-(cyclopropylmethyl)-7-(1-fluoro-2-(1H-imidazol-1-yl)propoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

1-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)-1-fluoropropan-2-yl trifluoromethanesulfonate (120 mg, 0.16 mmol) in DMF (2 mL) was reacted with 1H-imidazole (104 mg, 1.53 mmol) and Cs₂CO₃ (152 mg, 0.47 mmol) according to General Procedure 14. The reaction mixture was purified by flash column chromatography on C18 silica to afford the title compound (100 mg, 93%) as a mixture of diastereomers at the imidazolyl center as a yellow oil. LCMS (ESI, m/z): 689 [M+H]+.

### Step 7: Synthesis of 7-((S)-2-amino-3-fluoropropyl)-2-(1-(cyclopropylmethyl)-7-(1-fluoro-2-(1H-imidazol-1-yl)propoxy)-1H-indol-2-yl)-3-methyl-3,5,6,7-tetrahydro-8H-imidazo[4,5-b][1,6]naphthyridin-8-one

Tert-butyl ((2S)-1-(2-(1-(cyclopropylmethyl)-7-(1-fluoro-2-(1H-imidazol-1-yl)propoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (100 mg, 0.14 mmol) was reacted according to General Procedure 2. The crude product was purified by Prep-HPLC (Method F) to afford the title compound (70 mg, 82%) as a mixture of diastereomers at the imidazolyl center as a white solid. LCMS (ESI, m/z): 589 [M+H]+. The diastereomers were separated by Prep Chiral HPLC (Column: CHIRAL ART Cellulose-SB, 4.6×100 mm, 3 m; mobile phase A: MTBE (0.1% DEA): EtOH (1:1); flow rate: 1 mL/min; gradient: isocratic 0% B) to afford single stereoisomers of unknown configuration at the fluorine and imidazolyl centers.

Example 78a. Isomer 1: Chiral HPLC RT: 2.23 min. ¹H NMR (400 MHz, DMSO-d₆) δ 8.50 (s, 1H), 7.85 (s, 1H), 7.48 (dd, J=2.0, 6.8 Hz, 1H), 7.39 (s, 1H), 7.23 (s, 1H), 7.15-7.12 (m, 2H), 6.98 (s, 1H), 6.64 (dd, J=3.2, 57.2 Hz, 1H), 5.05-4.90 (m, 1H), 4.55-4.38 (m, 4H), 3.95 (s, 3H), 3.77-3.65 (m, 2H), 3.63-3.58 (m, 2H), 3.41-3.31 (m, 1H), 3.29-3.08 (m, 2H), 1.66 (d, J=7.1 Hz, 3H), 0.84-0.80 (m, 1H), 0.19-0.14 (m, 2H), −0.26-−0.31 (m, 2H). LCMS (ESI, m/z): 589 [M+H]+. LCMS RT: 1.219 min (Method D).

Example 78b. Isomer 2: Chiral HPLC RT: 2.60 min. ¹H NMR (400 MHz, DMSO-d₆): δ 8.49 (s, 1H), 7.84 (s, 1H), 7.48 (dd, J=1.2, 7.2 Hz, 1H), 7.40 (s, 1H), 7.22 (s, 1H), 7.14-7.08 (m, 2H), 6.97 (s, 1H), 6.45 (dd, J=4.0, 56.8 Hz, 1H), 5.05-4.95 (m, 1H), 4.57-4.49 (m, 1H), 4.47-4.44 (m, 2H), 4.34-4.31 (m, 1H), 3.95 (s, 3H), 3.79-3.74 (m, 2H), 3.63-3.59 (m, 1H), 3.53-3.51 (m, 1H), 3.32-3.28 (m, 3H), 1.69 (d, J=7.2 Hz, 3H), 0.94-0.92 (m, 1H), 0.21-0.19 (m, 2H), −0.18-−0.22 (m, 2H). LCMS (ESI, m/z): 589 [M+H]+; LCMS RT: 0.766 min (Method D).

Compounds of Examples 78c and 78d in Table 9 were obtained via a similar procedure to a compound of Examples 78a and 78b starting from Isomer 2 in step 3.

TABLE 9

| Example | | LCMS m/z [M + H]+; LCMS RT (LCMS |
|---|---|---|
| Number | Structure | method); ¹H NMR (MHz, solvent) |
| 78c | 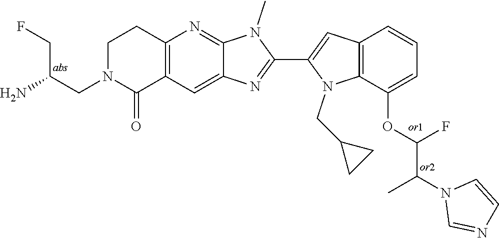 [SMILES: CC(C(F)Oc1cccc2cc(-c3nc4cc5c(nc4n3C)CCN(C[C@H](N)CF)C5=O)n(CC3CC3)c12)n1ccnc1] | 7-((S)-2-amino-3-fluoropropyl)-2-(1- (cyclopropylmethyl)-7-(1-fluoro-2-(1H- imidazol-1-yl)propoxy)-1H-indol-2-yl)-3- methyl-3,5,6,7-tetrahydro-8H-imidazo[4,5- b][1,6]naphthyridin-8-one LCMS (ESI, m/z): 589 [M + H]+; LCMS RT: 1.236 min (Method D). Prep Chiral HPLC RT: 7.14 min. [Column: CHIRAL ART Cellulose-SB, 2 x 25 cm, 5 μm; mobile phase A: MTBE (0.5% 2M NH₃ in MeOH), mobile phase B: MeOH; flow rate: 20 mL/min.; gradient: isocratic 50% B in 10 min.; detection: UV (220/254 nm)]. |
| 78d | 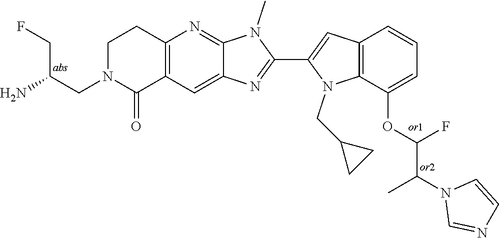 [SMILES: CC(C(F)Oc1cccc2cc(-c3nc4cc5c(nc4n3C)CCN(C[C@H](N)CF)C5=O)n(CC3CC3)c12)n1ccnc1] | 7-((S)-2-amino-3-fluoropropyl)-2-(1- (cyclopropylmethyl)-7-(1-fluoro-2-(1H- imidazol-1-yl)propoxy)-1H-indol-2-yl)-3- methyl-3,5,6,7-tetrahydro-8H-imidazo[4,5- b][1,6]naphthyridin-8-one LCMS (ESI, m/z): 589 [M + H]+; LCMS RT: 1.235 min (Method D) ¹H NMR (400 MHz, DMSO-d₆) δ 8.52 (s, 1H), 8.25 (s, 4H), 7.54-7.48 (m, 2H), 7.25 (s, 1H), 7.18-7.10 (m, 3H), 6.64 (dd, J = 3.6, 57.2 Hz, 1H), 5.06 (s, 1H), 4.78-4.46 (m, 4H), 3.96 (s, 3H), 3.88-3.84 (m, 2H), 3.77-3.71 (m, 3H), 3.35-3.32 (m, 2H), 1.73 (d, J = 6.8 Hz, 3H), 0.90-0.81 (m, 1H), 0.20-0.01 (m, 2H), −0.19-−0.31 (m, 2H) Prep Chiral HPLC RT: 8.49 min (Method see example 78c). |

### Example 79a and 79b. 7-((S)-2-amino-3-fluoropropyl)-2-(1-(cyclopropylmethyl)-7-(2-fluoro-2-(1H-imidazol-1-yl)ethoxy)-1H-indol-2-yl)-3-methyl-3,5,6,7-tetrahydro-8H-imidazo[4,5-b][1,6]naphthyridin-8-one

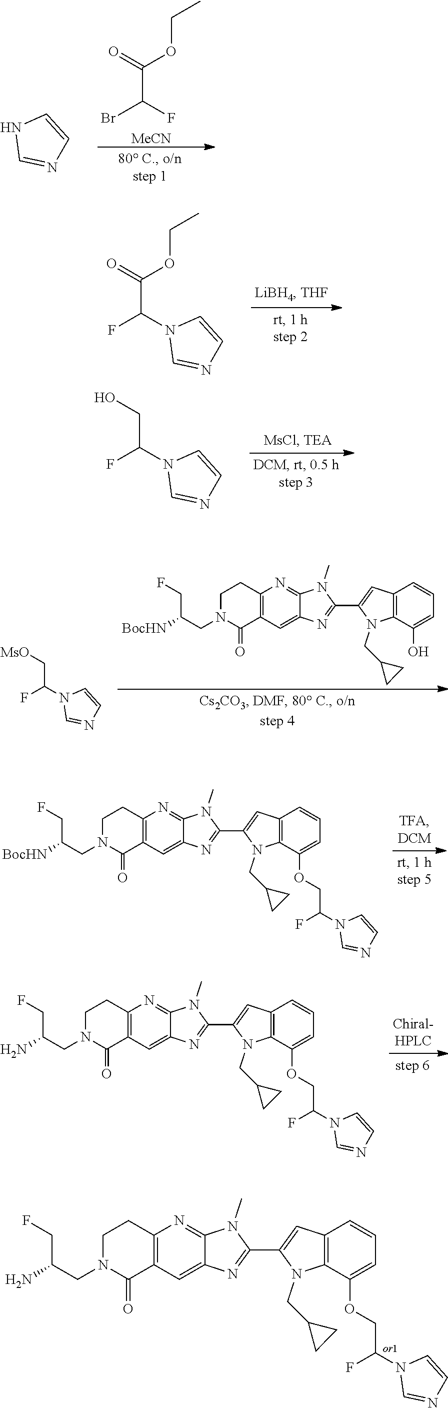

### Step 1: Synthesis of ethyl 2-fluoro-2-imidazol-1-yl-acetate

To a solution of 1H-imidazole (1 g, 14.7 mmol) in MeCN (20 mL) was added ethyl 2-bromo-2-fluoro-acetate (1.36 g, 7.34 mmol) under nitrogen atmosphere. The resulting solution was stirred at 80° C. overnight under nitrogen atmosphere. The reaction was monitored by LCMS. The resulting solution was concentrated under vacuum. The crude product was purified by column chromatography (ethyl acetate) to afford the title compound (880 mg, 34.7%) as a light yellow oil. LCMS (ESI, m/z): 173 [M+H]+.

### Step 2: Synthesis of 2-fluoro-2-imidazol-1-yl-ethanol

To a solution of ethyl 2-fluoro-2-imidazol-1-yl-acetate (4.9 g, 28.5 mmol) in THF (230 mL), was added LiBH₄ (46 mL, 113 mmol) dropwise at 0° C. under nitrogen atmosphere. The resulting solution was stirred at room temperature for 1 h under nitrogen atmosphere. The reaction was monitored by LCMS. The reaction was then quenched by adding acetone (60 mL). The mixture solution was concentrated under vacuum. The crude product was purified by column chromatography (petroleum ether/ethyl acetate=1:2) to afford the title compound (2.2 g, 59.4%) as a colorless oil. LCMS (ESI, m/z): 131 [M+H]+.

### Step 3: Synthesis of (2-fluoro-2-imidazol-1-yl-ethyl) methanesulfonate

2-Fluoro-2-imidazol-1-yl-ethanol (1 g, 7.69 mmol) in DCM (70 mL) was reacted with TEA (3.14 mL, 23.1 mmol) and MsCl (0.89 mL, 11.5 mmol) according to General Procedure 13 to afford the title compound (1.14 g, 71%) as a yellow oil. LCMS (ESI, m/z): 209 [M+H]+.

### Step 4: Synthesis of tert-butyl ((2S)-1-(2-(1-(cyclopropylmethyl)-7-(2-fluoro-2-(1H-imidazol-1-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 5 (150 mg, 0.27 mmol) in DMF (3 mL), was reacted with (2-fluoro-2-imidazol-1-yl-ethyl) methanesulfonate (370 mg, 1.78 mmol) according to General Procedure 5. The crude product was purified by flash column chromatography on C18 silica to afford the title compound (110 mg, 62%) as a mixture of diastereomers as a brown oil. LCMS (ESI, m/z): 675 [M+H]+.

### Step 5: Synthesis of 7-((S)-2-amino-3-fluoropropyl)-2-(1-(cyclopropylmethyl)-7-(2-fluoro-2-(1H-imidazol-1-yl)ethoxy)-1H-indol-2-yl)-3-methyl-3,5,6,7-tetrahydro-8H-imidazo[4,5-b][1,6]naphthyridin-8-one

Tert-butyl ((2S)-1-(2-(1-(cyclopropylmethyl)-7-(2-fluoro-2-(1H-imidazol-1-yl)ethoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (110 mg, 0.16 mmol) was reacted according to General Procedure 2. The crude product was purified by Prep-HPLC (Method F) to afford the title compound (75 mg, 80%) as a mixture of diastereomers as a white solid. LCMS (ESI, m/z): 575 [M+H]+. The diastereomers were separated by Chiral Prep HPLC [Column: CHIRAL ART Cellulose-SB, 2×25 cm, 5 μm; mobile phase A: MTBE (0.5% 2M NH3 in MeOH), mobile phase B: MeOH; flow rate: 20 mL/min; gradient: isocratic 30% B for 15 min.; detection: UV (220/254 nm)] to afford single stereoisomers of unknown configuration at the fluorine center.

Example 79a. Isomer 1: Chiral HPLC RT: 8.5 min. 1H NMR (400 MHz, DMSO-d6) δ 8.48 (s, 1H), 8.16 (s, 1H), 7.70 (s, 1H), 7.34 (d, J=7.6 Hz, 1H), 7.17 (s, 1H), 7.11-7.07 (m, 2H), 7.01-6.85 (m, 2H), 4.99-4.97 (m, 1H), 4.79-4.59 (m, 1H), 4.59-4.55 (m, 1H), 4.55-4.42 (m, 2H), 4.33-4.30 (m, 1H), 3.93 (s, 3H), 3.78-3.73 (m, 2H), 3.63-3.60 (m, 1H), 3.58-3.46 (m, 1H), 3.32-3.27 (m, 3H), 0.89-0.80 (m, 1H), 0.14-0.01 (m, 2H), −0.18-−0.22 (m, 2H). LCMS (ESI, m/z): 575 [M+H]+. LCMS RT: 1.179 min (Method D).

Example 79b. Isomer 2: Chiral HPLC RT: 11.33 min. ¹H NMR (400 MHz, DMSO-d₆): δ 8.48 (s, 1H), 8.16 (s, 1H), 7.70 (s, 1H), 7.34 (d, J=7.6 Hz, 1H), 7.17-6.85 (m, 5H), 5.02-4.98 (m, 1H), 4.95-4.81 (m, 1H), 4.79-4.41 (m, 3H), 4.31-4.29 (m, 1H), 3.93 (s, 3H), 3.80-3.71 (m, 2H), 3.63-3.45 (m, 2H), 3.32-3.28 (m, 3H), 2.15 (s, 2H), 0.87 (s, 1H), 0.15-0.01 (m, 2H), −0.20-−0.32 (m, 2H). LCMS (ESI, m/z): 575 [M+H]+; LCMS RT: 1.174 min (Method D).

### Example 80. 2-(7-((S)-2-(1H-imidazol-1-yl)propoxy)-1-(cyclopropylmethyl)-1H-indol-2-yl)-7-((S)-2-amino-3-fluoropropyl)-3-methyl-3,5,6,7-tetrahydro-8H-imidazo[4,5-b][1,6]naphthyridin-8-one

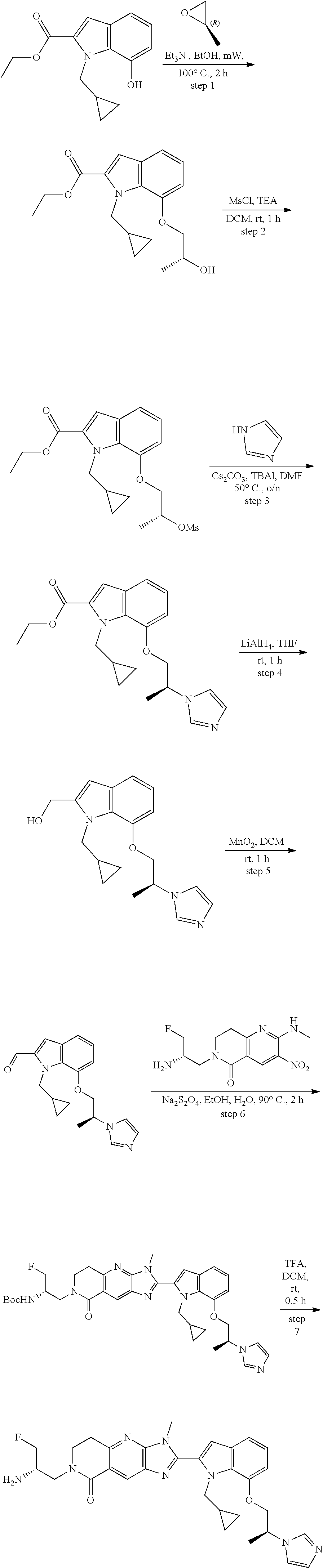

### Step 1 Synthesis of ethyl (R)-1-(cyclopropylmethyl)-7-(2-hydroxypropoxy)-1H-indole-2-carboxylate

Ethyl 1-(cyclopropylmethyl)-7-hydroxy-indole-2-carboxylate (100 mg, 0.39 mmol) in ethanol (2 mL) was reacted with (2R)-2-methyloxirane (224 mg, 3.86 mmol) and TEA (0.34 mL, 1.93 mmol) according to General Procedure 12. The crude product was purified by column chromatography (petroleum ether/ethyl acetate=1:1) to afford the title compound (90 mg, 73.5%) as a light-yellow solid. LCMS (ESI, m/z): 318 [M+H]+

### Step 2: Synthesis of ethyl (R)-1-(cyclopropylmethyl)-7-(2-((methylsulfonyl)oxy)propoxy)-1H-indole-2-carboxylate

Ethyl (R)-1-(cyclopropylmethyl)-7-(2-hydroxypropoxy)-1H-indole-2-carboxylate (90 mg, 0.28 mmol) in DCM (4 mL) was reacted with TEA (0.2 mL, 1.13 mmol) and MsCl (0.04 mL, 0.57 mmol) according to General Procedure 13 to afford the title compound (90 mg, 80%) as a light yellow oil. LCMS (ESI, m/z): 396 [M+H]+

### Step 3: Synthesis of ethyl (S)-7-(2-(1H-imidazol-1-yl)propoxy)-1-(cyclopropylmethyl)-1H-indole-2-carboxylate

To a solution of ethyl (R)-1-(cyclopropylmethyl)-7-(2-((methylsulfonyl)oxy)propoxy)-1H-indole-2-carboxylate (90 mg, 0.23 mmol) in DMF (3 mL), was added 1H-imidazole (23.3 mg, 0.34 mmol) and Cs₂CO₃ (222 mg, 0.68 mmol). The reaction was stirred at 50° C. for overnight. The reaction was monitored by LCMS. The reaction was then quenched by adding water (10 mL) and extracted with ethyl acetate (10 mL), washed with water (5×10 mL) and brine (5×10 mL), dried over anhydrous sodium sulfate and concentrated under vacuum. The crude product was purified by column chromatography (petroleum ether/ethyl acetate=1:1) to afford the title compound (70 mg, 83.7%) as a light yellow solid. LCMS (ESI, m/z): 368 [M+H]+

### Step 4: Synthesis of (S)-(7-(2-(1H-imidazol-1-yl)propoxy)-1-(cyclopropylmethyl)-1H-indol-2-yl)methanol

Ethyl (S)-7-(2-(1H-imidazol-1-yl)propoxy)-1-(cyclopropylmethyl)-1H-indole-2-carboxylate (70 mg, 0.19 mmol) was reacted according to General Procedure 3. The crude product was purified by column chromatography (petroleum ether/ethyl acetate=1:1) to afford the title compound (50 mg, 81%) as a light yellow solid. LCMS (ESI, m/z): 326 [M+H]+.

### Step 5: Synthesis of (S)-7-(2-(1H-imidazol-1-yl)propoxy)-1-(cyclopropylmethyl)-1H-indole-2-carbaldehyde

(S)-(7-(2-(1H-imidazol-1-yl)propoxy)-1-(cyclopropylmethyl)-1H-indol-2-yl)methanol (50 mg, 0.15 mmol) as reacted according to General Procedure 4. The crude product was purified by column chromatography (petroleum ether/ethyl acetate=1:1) to afford the title compound (40 mg, 80%) as a light yellow solid. LCMS (ESI, m/z): 324 [M+H]+.

### Step 6 Synthesis of tert-butyl ((S)-1-(2-(7-((S)-2-(1H-imidazol-1-yl)propoxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

(S)-7-(2-(1H-imidazol-1-yl)propoxy)-1-(cyclopropylmethyl)-1H-indole-2-carbaldehyde (40 mg, 0.12 mmol) and Intermediate 2 (49.2 mg, 0.12 mmol) was reacted according to General Procedure 6. The crude product was purified by column chromatography (DCM/MeOH=15:1) to afford the title compound (30 mg, 36%) as a light yellow solid. LCMS (ESI, m/z): 671 [M+H]+.

### Step 7: Synthesis of 2-(7-((S)-2-(1H-imidazol-1-yl)propoxy)-1-(cyclopropylmethyl)-1H-indol-2-yl)-7-((S)-2-amino-3-fluoropropyl)-3-methyl-3,5,6,7-tetrahydro-8H-imidazo[4,5-b][1,6]naphthyridin-8-one

Tert-butyl ((S)-1-(2-(7-((S)-2-(1H-imidazol-1-yl)propoxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.04 mmol) was reacted according to General Procedure 2. The crude product was purified by Prep-HPLC (Method F) to afford the title compound (15 mg, 58%) as a white solid. ¹H NMR (400 MHz, DMSO-d₆) δ 8.51 (s, 1H), 8.15 (s, 1H), 7.83 (s, 1H), 7.41 (s, 1H), 7.30 (d, J=7.9 Hz, 1H), 7.16 (s, 1H), 7.07 (t, J=7.9 Hz, 1H), 6.95 (s, 1H), 6.88 (d, J=7.8 Hz, 1H), 4.86-4.83 (m, 1H), 4.82-4.70 (m, 1H), 4.66-4.55 (m, 1H), 4.53-4.41 (m, 4H), 3.94 (s, 3H), 3.81-3.73 (m, 4H), 3.32 (s, 3H), 1.58 (d, J=6.9 Hz, 3H), 0.81-0.79 (m, 1H), 0.18-0.12 (m, 2H), −0.24-−0.33 (m, 2H). LCMS (ESI, m/z): 571 [M+H]+. LCMS RT: 1.202 min (Method B).

### Example 81. 7-((R)-2-aminopropyl)-2-(1-(cyclopropylmethyl)-7-((R)-2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one

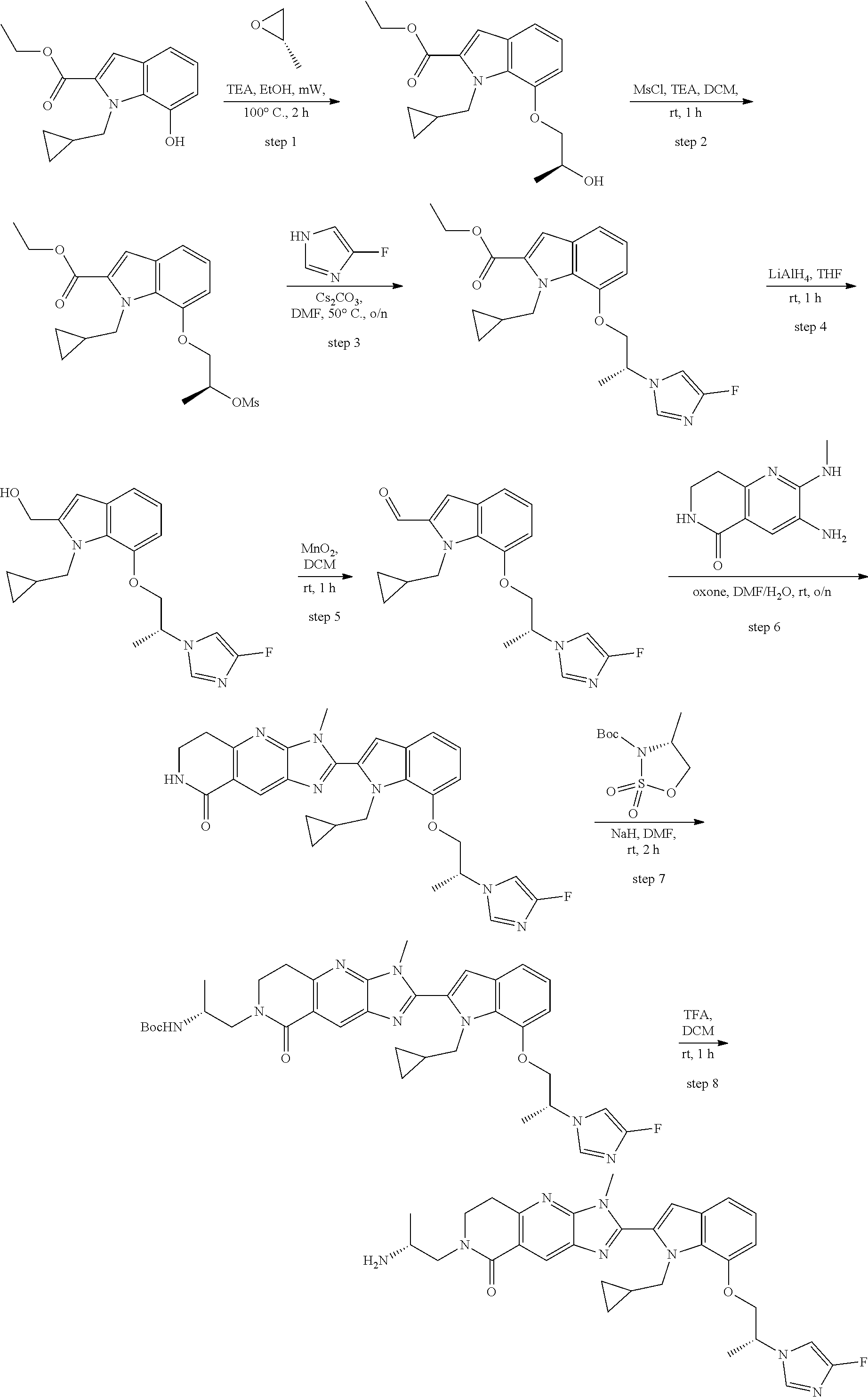

### Step 1 Synthesis of ethyl (S)-1-(cyclopropylmethyl)-7-(2-hydroxypropoxy)-1H-indole-2-carboxylate

Ethyl 1-(cyclopropylmethyl)-7-hydroxy-indole-2-carboxylate (1 g, 3.86 mmol) in ethanol (12.5 mL) was reacted with (2S)-2-methyloxirane (672 mg, 11.5 mmol) and TEA (3.36 mL, 19.3 mmol) according to General Procedure 12. The crude product was purified by column chromatography (petroleum ether/ethyl acetate=5:1) to afford the title compound (1 g, 81%) as a pink solid. LCMS (ESI, m/z): 318[M+H]+

### Step 2: Synthesis of ethyl (S)-1-(cyclopropylmethyl)-7-(2-((methylsulfonyl)oxy)propoxy)-1H-indole-2-carboxylate

Ethyl (S)-1-(cyclopropylmethyl)-7-(2-hydroxypropoxy)-1H-indole-2-carboxylate (2 g, 6.3 mmol) in DCM (50 mL) was reacted with TEA (3.3 mL, 18.9 mmol) and MsC1 (866 mg, 7.56 mmol) according to General Procedure 13 to afford the title compound (2 g, 80%) as a yellow oil. LCMS (ESI, m/z): 396 [M+H]+

### Step 3: Synthesis of ethyl (R)-1-(cyclopropylmethyl)-7-(2-(4-fluoro-1H-imidazol-1-yl)propoxy)-1H-indole-2-carboxylate

Ethyl (S)-1-(cyclopropylmethyl)-7-(2-((methylsulfonyl)oxy)propoxy)-1H-indole-2-carboxylate (2 g, 5.06 mmol) in DMF (50 mL) was reacted with 4-fluoro-1H-imidazole (522 mg, 6.07 mmol) and Cs₂CO₃ (4.95 g, 15.2 mmol) according to General Procedure 14. The crude product was purified by column chromatography (petroleum ether/ethyl acetate=1:1) to afford the title compound (1.7 g, 87%) as a yellow solid. LCMS (ESI, m/z): 386 [M+H]+

### Step 4: Synthesis of (R)-(1-(cyclopropylmethyl)-7-(2-(4-fluoro-1H-imidazol-1-yl)propoxy)-1H-indol-2-yl)methanol

Ethyl (R)-1-(cyclopropylmethyl)-7-(2-(4-fluoro-1H-imidazol-1-yl)propoxy)-1H-indole-2-carboxylate (2 g, 5.19 mmol) was reacted according to General Procedure 3. The crude product was purified by column chromatography (petroleum ether/ethyl acetate=1:1) to afford the title compound (1.5 g, 84%) as a yellow oil. LCMS (ESI, m/z): 344 [M+H]+

### Step 5: Synthesis of (R)-1-(cyclopropylmethyl)-7-(2-(4-fluoro-1H-imidazol-1-yl)propoxy)-1H-indole-2-carbaldehyde

(R)-(1-(cyclopropylmethyl)-7-(2-(4-fluoro-1H-imidazol-1-yl)propoxy)-1H-indol-2-yl)methanol (2 g, 5.82 mmol) was reacted according to General Procedure 4. The crude product was purified by column chromatography (petroleum ether/ethyl acetate=1:1) to afford the title compound (1.4 g, 70%) as a light green solid. LCMS (ESI, m/z): 342 [M+H]+

### Step 6 Synthesis of (R)-2-(1-(cyclopropylmethyl)-7-(2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one

Intermediate 6 (212 mg, 1.1 mmol) and (R)-1-(cyclopropylmethyl)-7-(2-(4-fluoro-1H-imidazol-1-yl)propoxy)-1H-indole-2-carbaldehyde (342 mg, 1.1 mmol) were reacted according to General Procedure 11. The reaction mixture was then purified by flash column chromatography on C18 silica to afford the title compound (160 mg, 31%) as a yellow solid. LCMS (ESI, m/z): 514 [M+H]+

### Step 7: Synthesis of tert-butyl ((R)-1-(2-(1-(cyclopropylmethyl)-7-((R)-2-(4-fluoro-1H-imidazol-1-yl)propoxy)-1H-indol-2-yl)-3-methyl-8-oxo-3,5,6,8-tetrahydro-7H-imidazo[4,5-b][1,6]naphthyridin-7-yl)propan-2-yl)carbamate

(R)-2-(1-(cyclopropylmethyl)-7-(2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one (200 mg, 0.39 mmol) was reacted with tert-butyl (R)-4-methyl-1,2,3-oxathiazolidine-3-carboxylate 2,2-dioxide (277 mg, 1.17 mmol) according to General Procedure 1. The crude product was purified by column chromatography (DCM/MeOH=10:1) to afford the title compound (110 mg, 42%) as a yellow solid. LCMS (ESI, m/z): 671 [M+H]+.

### Step 8: Synthesis of 7-((R)-2-aminopropyl)-2-(1-(cyclopropylmethyl)-7-((R)-2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one

tert-butyl ((R)-1-(2-(1-(cyclopropylmethyl)-7-((R)-2-(4-fluoro-1H-imidazol-1-yl)propoxy)-1H-indol-2-yl)-3-methyl-8-oxo-3,5,6,8-tetrahydro-7H-imidazo[4,5-b][1,6]naphthyridin-7-yl)propan-2-yl)carbamate (100 mg, 0.15 mmol) was reacted according to General Procedure 2. The crude product was purified by Prep-HPLC (Method F) to afford the title compound (33.6 mg, 39%) as a light yellow solid. ¹H NMR (400 MHz, DMSO-d₆): δ 8.52 (s, 1H), 7.87 (s, 3H), 7.53 (s, 1H), 7.29 (d, J=7.6 Hz, 1H), 7.17-7.12 (m, 2H), 7.07 (t, J=7.6 Hz, 1H), 6.88 (d, J=7.6 Hz, 1H), 4.79-4.78 (m, 1H), 4.57-4.40 (m, 4H), 3.96 (s, 3H), 3.82-3.71 (m, 3H), 3.59-3.53 (m, 2H), 3.32 (t, J=6.8 Hz, 2H), 1.55 (d, J=6.8 Hz, 3H), 1.26 (d, J=6.4 Hz, 3H), 0.79 (s, 1H), 0.15-0.12 (m, 2H), −0.29-−0.31 (m, 2H). LCMS (ESI, m/z): 571 [M+H]+. LCMS RT: 1.390 min (Method D).

### Example 82. 7—((S)-2-amino-3-fluoropropyl)-2-(1-(cyclopropylmethyl)-7-(((S)-5-oxopyrrolidin-3-yl)methoxy)-1H-pyrrolo[2,3-c]pyridin-2-yl)-3-methyl-3,5,6,7-tetrahydro-8H-imidazo[4,5-b][1,6]naphthyridin-8-one

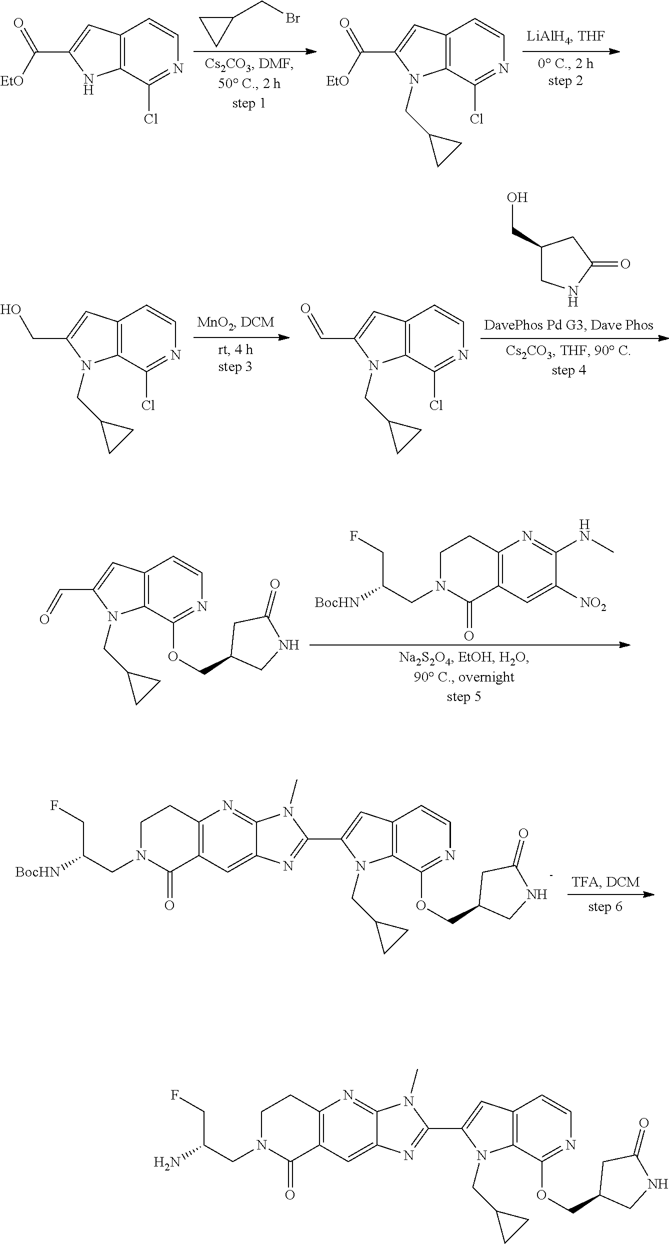

### Step 1 Synthesis of ethyl 7-chloro-1-(cyclopropylmethyl)pyrrolo[2,3-c]pyridine-2-carboxylate

Ethyl 7-chloro-1H-pyrrolo[2,3-c]pyridine-2-carboxylate (250 mg, 1.11 mmol) was reacted with bromomethylcyclopropane (225 mg, 1.67 mmol) according to General Procedure 5. The crude product was purified by Prep-TLC (petroleum ether/ethyl acetate=1:2) to afford the title compound (240 mg, 77.37%) as white solid. LCMS (ESI, m/z): 279 [M+H]+.

### Step 2: Synthesis of [7-chloro-1-(cyclopropylmethyl)pyrrolo[2,3-c]pyridin-2-yl]methanol

Ethyl 7-chloro-1-(cyclopropylmethyl)pyrrolo[2,3-c]pyridine-2-carboxylate (240 mg, 0.86 mmol) was reacted according to General Procedure 3. The crude product was purified by Prep-TLC (DCM/MeOH=15:1) to afford the title compound (80 mg, 39.25%) as yellow solid. LCMS (ESI, m/z): 237 [M+H]+.

### Step 3: Synthesis of 7-chloro-1-(cyclopropylmethyl)pyrrolo[2,3-c]pyridine-2-carbaldehyde

[7-chloro-1-(cyclopropylmethyl)pyrrolo[2,3-c]pyridin-2-yl]methanol (80 mg, 0.34 mmol) was reacted according to General Procedure 4. The crude product was purified by Prep-TLC (petroleum ether/ethyl acetate=1:1) to afford the title compound (50 mg, 63.04%) as yellow oil. LCMS (ESI, m/z): 235 [M+H]+.

### Step 4: Synthesis of (S)-1-(cyclopropylmethyl)-7-((5-oxopyrrolidin-3-yl)methoxy)-1H-pyrrolo[2,3-c]pyridine-2-carbaldehyde

To a solution of 7-chloro-1-(cyclopropylmethyl)pyrrolo[2,3-c]pyridine-2-carbaldehyde (50 mg, 0.21 mmol) in THF (2 mL), was added (4S)-4-(hydroxymethyl)pyrrolidin-2-one (73 mg, 0.64 mmol), Cs₂CO₃ (208 mg, 0.64 mmol), Dave Phos (14 mg, 0.04 mmol) and DavePhos Pd G3 (16 mg, 0.02 mmol) at room temperature under nitrogen atmosphere. The resulting mixture was stirred at 90° C. for 6 h. The reaction was monitored by TLC and LCMS. The reaction was quenched by adding water (15 mL) and extracted with ethyl acetate (2×30 mL). The combined organic extracts were washed with water (50 mL) and brine (50 mL), dried over anhydrous sodium sulfate, and concentrated under vacuum. The crude product was purified by Prep-TLC (DCM/MeOH=15:1) to afford the title compound (20 mg, 29.96%) as yellow oil. LCMS (ESI, m/z): 314 [M+H]+.

### Step 5: Synthesis of tert-butyl ((S)-1-(2-(1-(cyclopropylmethyl)-7-(((S)-5-oxopyrrolidin-3-yl)methoxy)-1H-pyrrolo[2,3-c]pyridin-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 2 (28 mg, 0.07 mmol) and (S)-1-(cyclopropylmethyl)-7-((5-oxopyrrolidin-3-yl)methoxy)-1H-pyrrolo[2,3-c]pyridine-2-carbaldehyde (19 mg, 0.06 mmol) were reacted according to General Procedure 6. The crude product was purified by Prep-TLC (DCM/MeOH=10:1) to afford the title compound (15 mg, 32.22%) as yellow solid. LCMS (ESI, m/z): 661 [M+H]+.

### Step 6: Synthesis of 7-((S)-2-amino-3-fluoropropyl)-2-(1-(cyclopropylmethyl)-7-(((S)-5-oxopyrrolidin-3-yl)methoxy)-1H-pyrrolo[2,3-c]pyridin-2-yl)-3-methyl-3,5,6,7-tetrahydro-8H-imidazo[4,5-b][1,6]naphthyridin-8-one

Tert-butyl ((S)-1-(2-(1-(cyclopropylmethyl)-7-(((S)-5-oxopyrrolidin-3-yl)methoxy)-1H-pyrrolo[2,3-c]pyridin-2-yl)-3-methyl-8-oxo-3,5,6,8-tetrahydro-7H-imidazo[4,5-b][1,6]naphthyridin-7-yl)-3-fluoropropan-2-yl)carbamate (15 mg, 0.02 mmol) was reacted according to General Procedure 2. The obtained crude orange sticky oil was purified by Prep HPLC (Method F) to afford the title compound (10.2 mg, 78.2%) as light-yellow solid. ¹H NMR (400 MHz, DMSO-d₆) δ 8.55 (s, 1H), 8.27 (s, 3H), 7.76 (d, J=5.6 Hz, 1H), 7.61 (s, 1H), 7.32 (d, J=5.6 Hz, 1H), 7.23 (s, 1H), 4.82-4.74 (m, 1H), 4.71 (d, J=7.1 Hz, 2H), 4.68-4.61 (m, 1H), 4.55-4.47 (m, 2H), 3.96 (s, 3H), 3.90-3.81 (m, 2H), 3.80-3.70 (m, 3H), 3.50 (t, J=8.9 Hz, 1H), 3.35 (t, J=6.6 Hz, 2H), 3.25-3.17 (m, 1H), 3.02-2.92 (m, 1H), 2.45-2.36 (m, 1H), 2.21-2.12 (m, 1H), 1.12-1.01 (m, 1H), 0.31-0.19 (m, 2H), −0.03-−0.13 (m, 2H). LCMS (ESI, m/z): 561 [M+H]+. LCMS RT: 1.608 min (Method D).

Example 83 in Table 10 was obtained following a procedure similar to the preparation of Example 82

TABLE 10

| Exam- | | |
|---|---|---|
| ple | | LCMS m/z [M + H]+; LCMS RT (LCMS |
| Num- | | method); |
| ber | Structure | ¹H NMR (MHz, solvent) |
| 83 | 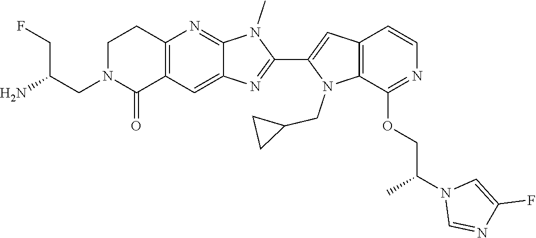 [SMILES: C[C@H](COc1nccc2cc(-c3nc4cc5c(nc4n3C)CCN(C[C@H](N)CF)C5=O)n(CC3CC3)c12)n1cnc(F)c1] | 7-((S)-2-amino-3-fluoropropyl)-2-(1- (cyclopropylmethyl)-7-((R)-2-(4-fluoro-1H- imidazol-1-yl)propoxy)-1H-pyrrolo[2,3-c] pyridin-2-yl)-3-methyl-3,5,6,7- tetrahydro-8H-imidazo[4,5- b][1,6]naphthyridin-8-one LCMS (ESI, m/z): 590 [M + H]+; LCMS RT: 0.825 min. (Method B) ¹H NMR (400 MHz, DMSO-d₆): δ 8.55 (s, 1H), 8.30 (s, 3H), 7.76 (d, J = 6.4 Hz, 1H), 7.50 (t, J = 1.6 Hz, 1H), 7.33 (d, J = 6.4 Hz, 1H), 7.22 (s, 1H), 7.16-7.13 (m, 1H), 4.90-4.89 (m, 1H), 4.86-4.73 (m, 2H), 4.69-4.61 (m, 2H), 4.49 (d, J = 7.2 Hz, 2H), 3.94-3.84 (m, 5H), 3.78-3.71 (m, |
| | | 3H), 3.34 (t, J = 6.4 Hz, 2H), 1.54 (d, J = 6.8 Hz, |
| | | 3H), 0.82-0.78 (m, 1H), 0.19-0.15 (m, 2H), |
| | | −0.24-−0.25 (m, 2H). |

### Example 84. (S)-2-(7-(2-(1H-imidazol-1-yl)ethoxy)-1-(cyclopropylmethyl)-iTI-pyrrolo[2,3-c]pyridin-2-yl)-7-(2-amino-3-fluoropropyl)-3-methyl-3,5,6,7-tetrahydro-8H-imidazo[4,5-b][1,6]naphthyridin-8-one

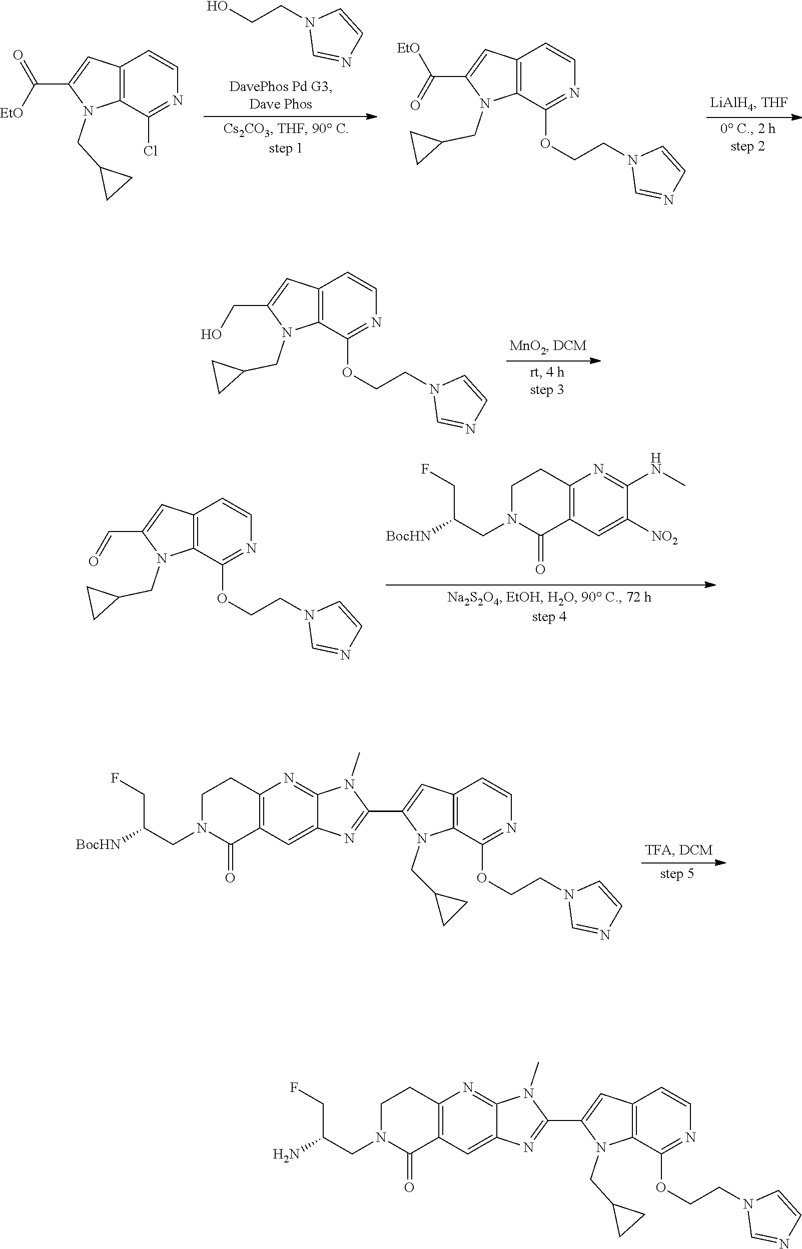

### Step 1: Synthesis of ethyl 1-(cyclopropylmethyl)-7-(2-imidazol-1-ylethoxy)pyrrolo[2,3-c]pyridine-2-carboxylate

To a solution of ethyl 7-chloro-1-(cyclopropylmethyl)pyrrolo[2,3-c]pyridine-2-carboxylate (100 mg, 0.36 mmol) in THF (4 mL) was added Cs₂CO₃ (350 mg, 1.08 mmol), 2-imidazol-1-ylethanol (80 mg, 0.72 mmol), DavePhos (28 mg, 0.07 mmol) and DavePhos Pd G3 (27 mg, 0.04 mmol) at room temperature under nitrogen atmosphere. The resulting mixture was stirred at 90° C. for 1 h. The reaction was monitored by TLC and LCMS. The reaction was quenched by adding water (10 mL) and extracted with EtOAc (2×20 ml). The combined organic extracts were dried over anhydrous sodium sulfate and concentrated under vacuum. The crude product was purified by Prep-TLC (petroleum ether/EtOAc=5:1) to afford the title compound (110 mg, 86.5%) as a yellow oil. LCMS (ESI, m/z): 355 [M+H]+.

### Step 2: Synthesis of [1-(cyclopropylmethyl)-7-(2-imidazol-1-ylethoxy)pyrrolo[2,3-c]pyridin-2-yl]methanol

Ethyl 1-(cyclopropylmethyl)-7-(2-imidazol-1-ylethoxy)pyrrolo[2,3-c]pyridine-2-carboxylate (100 mg, 0.28 mmol) was reacted according to General Procedure 3. The resulting mixture was purified by flash column chromatography on C18 silica (Mobile Phase A: water (0.5% TFA), Mobile Phase B: ACN; Flow rate: 20 mL/min; Gradient: 45% B to 45% B in 7 min; 254/210 nm) to afford the title compound (80 mg, 90.7%) as a white solid. LCMS (ESI, m/z): 313 [M+H]+.

### Step 3: Synthesis of 1-(cyclopropylmethyl)-7-(2-imidazol-1-ylethoxy)pyrrolo[2,3-c]pyridine-2-carbaldehyde

[1-(cyclopropylmethyl)-7-(2-imidazol-1-ylethoxy)pyrrolo[2,3-c]pyridin-2-yl]methanol (80 mg, 0.26 mmol) was reacted according to General Procedure 4. The filtrate was concentrated under reduced pressure to afford the title compound (60 mg, 75.4%) as a yellow oil. LCMS (ESI, m/z): 311 [M+H]+.

### Step 4 Synthesis of tert-butyl (S)-(1-(2-(7-(2-(1H-imidazol-1-yl)ethoxy)-1-(cyclopropylmethyl)-1H-pyrrolo[2,3-c]pyridin-2-yl)-3-methyl-8-oxo-3,5,6,8-tetrahydro-7H-imidazo[4,5-b][1,6]naphthyridin-7-yl)-3-fluoropropan-2-yl)carbamate

1-(cyclopropylmethyl)-7-(2-imidazol-1-ylethoxy)pyrrolo[2,3-c]pyridine-2-carbaldehyde (11 mg, 0.04 mmol) and Intermediate 2 (50 mg, 0.13 mmol) were reacted according to General Procedure 6. The crude was purified by flash column chromatography on C18 silica to afford the title compound (60 mg, 72.5%) as a yellow solid. LCMS (ESI, m/z): 658 [M+H]+.

### Step 5: Synthesis of (S)-2-(7-(2-(1H-imidazol-1-yl)ethoxy)-1-(cyclopropylmethyl)-1H-pyrrolo[2,3-c]pyridin-2-yl)-7-(2-amino-3-fluoropropyl)-3-methyl-3,5,6,7-tetrahydro-8H-imidazo[4,5-b][1,6]naphthyridin-8-one

Tert-butyl (S)-(1-(2-(7-(2-(1H-imidazol-1-yl)ethoxy)-1-(cyclopropylmethyl)-1H-pyrrolo[2,3-c]pyridin-2-yl)-3-methyl-8-oxo-3,5,6,8-tetrahydro-7H-imidazo[4,5-b][1,6]naphthyridin-7-yl)-3-fluoropropan-2-yl)carbamate (50 mg, 0.08 mmol) was reacted according to General Procedure 2. The crude was purified by Prep HPLC (Method F) to afford the title compound (14.4 mg, 33.6%) as a yellow solid. ¹H NMR (400 MHz, DMSO-d₆+D₂O): δ 9.26 (s, 1H), 8.54 (s, 1H), 7.93 (t, J=1.8 Hz, 1H), 7.77 (d, J=5.6 Hz, 1H), 7.72 (t, J=1.8 Hz, 1H), 7.36 (d, J=5.6 Hz, 1H), 7.23 (s, 1H), 4.98 (t, J=5.0 Hz, 2H), 4.77 (d, J=9.4, 4.6 Hz, 2H), 4.75-4.58 (m, 2H), 4.51 (d, J=7.0 Hz, 2H), 3.93 (s, 3H), 3.91-3.78 (m, 3H), 3.76 (t, J=6.7 Hz, 2H), 3.34 (t, J=6.6 Hz, 2H), 0.82-0.67 (m, 1H), 0.24-0.09 (m, 2H), −0.23-−0.39 (m, 2H). LCMS (ESI, m/z): 558 [M+H]+. LCMS RT: 0.672 min (Method D).

### Example 85. 7—((S)-2-amino-3-fluoropropyl)-2-(1-(cyclopropylmethyl)-7-((R)-2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one

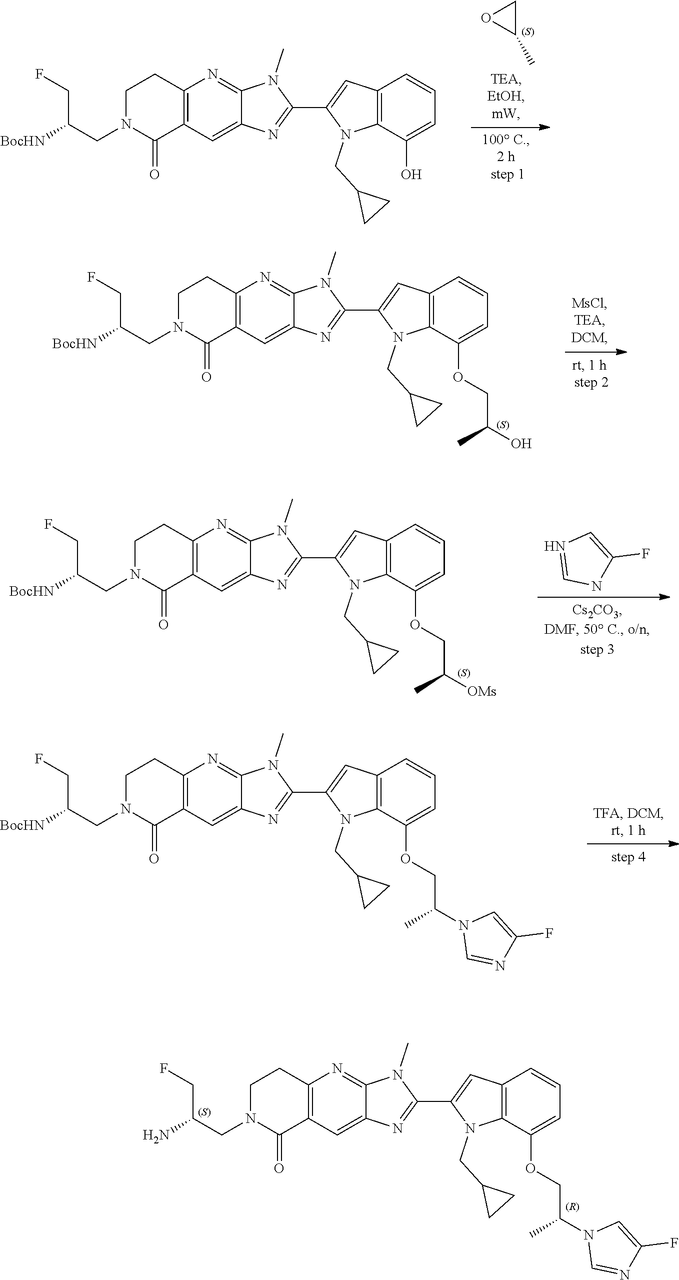

### Step 1: Synthesis of tert-butyl ((S)-1-(2-(1-(cyclopropylmethyl)-7-((S)-2-hydroxypropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 5 (50 mg, 0.09 mmol) in ethanol (2 mL) was reacted with (2S)-2-methyloxirane (51.6 mg, 0.89 mmol) and TEA (0.06 mL, 0.44 mmol) according to General Procedure 12. The crude product was purified by column chromatography (petroleum ether/ethyl acetate=1:5) to afford the title compound (45 mg, 81.8%) as a yellow solid. LCMS (ESI, m/z): 419 [M+H]⁺.

### Step 2: Synthesis of (S)-1-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)propan-2-yl methanesulfonate

Tert-butyl ((S)-1-(2-(1-(cyclopropylmethyl)-7-((S)-2-hydroxypropoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (45 mg, 0.07 mmol) in DCM (5 mL) was reacted with TEA (0.03 mL, 0.22 mmol) and MsCl (12.5 mg, 0.11 mmol) according to General Procedure 13 to afford the title compound (50 mg, 98.8%) as a light yellow oil. LCMS (ESI, m/z): 699 [M+H]+.

### Step 3: Synthesis of tert-butyl ((S)-1-(2-(1-(cyclopropylmethyl)-7-((R)-2-(4-fluoro-1H-imidazol-1-yl)propoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

(S)-1-((2-(7-((S)-2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)propan-2-yl methanesulfonate (60 mg, 0.09 mmol) in DMF (1 mL) was reacted with 4-fluoro-1H-imidazole (50 mg, 0.58 mmol) and Cs₂CO₃ (110 mg, 0.34 mmol) according to General Procedure 14. The crude product was purified by column chromatography (petroleum ether/ethyl acetate=1:3) to afford the title compound (25 mg, 58.9%) as a yellow solid. LCMS (ESI, m/z): 689 [M+H]⁺.

### Step 4: Synthesis of 7-((S)-2-amino-3-fluoropropyl)-2-(1-(cyclopropylmethyl)-7-((R)-2-(4-fluoro-1H-imidazol-1-yl)propoxy)-1H-indol-2-yl)-3-methyl-3,5,6,7-tetrahydro-8H-imidazo[4,5-b][1,6]naphthyridin-8-one

Tert-butyl ((S)-1-(2-(1-(cyclopropylmethyl)-7-((R)-2-(4-fluoro-1H-imidazol-1-yl)propoxy)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (20 mg, 0.03 mmol) was reacted according to General Procedure 2. The crude product was purified by Prep-HPLC (Method F) to afford the title compound (10.3 mg, 60.5%) as a white solid. ¹H NMR (400 MHz, Methanol-d4) δ 8.65 (s, 1H), 7.54 (s, 1H), 7.34 (d, J=8 Hz, 1H), 7.12 (t, J=8 Hz, 1H), 7.09 (s, 1H), 6.99 (d, J=2 Hz, 1H), 6.91 (d, J=7.6 Hz, 1H), 4.88-4.82 (m, 3H), 4.75-4.44 (m, 4H), 4.41-3.42 (m, 8H), 3.43 (d, J=6.8 Hz, 2H), 1.67 (s, 3H), 0.81-0.80 (m, 1H), 0.21-0.19 (m, 2H), −0.33-−0.34 (m, 2H). LCMS (ESI, m/z): 589 [M+H]⁺. LCMS RT: 0.881 min (Method D).

Compounds of Examples 86 to 94 in Table 11 were obtained following a procedure similar to the preparation of a compound of Example 85 using the appropriate starting reagents.

TABLE 11

| | | LCMS m/z [M + H]+; LCMS RT |
|---|---|---|
| Example | | (LCMS method); ¹H NMR (MHz, |
| Number | Structure | solvent) |
| 86 | 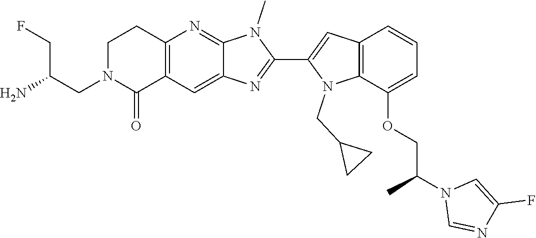 [SMILES: C[C@@H](COc1cccc2cc(-c3nc4cc5c(nc4n3C)CCN(C[C@H](N)CF)C5=O)n(CC3CC3)c12)n1cnc(F)c1] | 7-((S)-2-amino-3-fluoropropyl)-2-(1- (cyclopropylmethyl)-7-((S)-2-(4-fluoro- 1H-imidazol-1-yl)propoxy)-1H-indol-2- yl)-3-methyl-3,5,6,7-tetrahydro-8H- imidazo[4,5-b][1,6]naphthyridin-8-one LCMS (ESI, m/z): 589 [M + H]+; LCMS RT: 0.878 min (Method D) ¹H NMR (400 MHz, Methanol-d₄) δ 8.65 (s, 1H), 7.54 (s, 1H), 7.34 (d, J = 7.6 Hz, 1H), 7.12 (t, J = 8 Hz, 1H), 7.09 (s, 1H), 7.03 (d, J = 18 Hz, 1H), 6.99 (d, J = 1.6 Hz, 1H), 4.88-4.82 (m, 3H), 4.76-4.41 (m, 4H), 4.08-4.00 (m, 5H), 3.89-3.78 (m, 3H), 3.43 (t, J = 6.8 Hz, 2H), 1.67 (s, 3H), 0.90-0.70 (m, 1H), 0.21-0.12 (m, 2H), −0.33-−0.34 (m, 2H). |
| 87 | 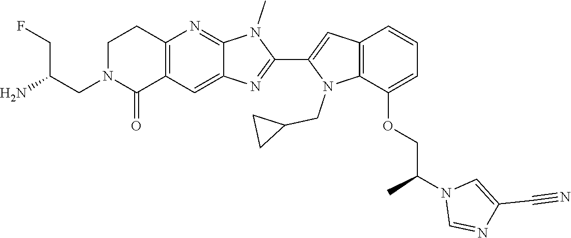 [SMILES: C[C@@H](COc1cccc2cc(-c3nc4cc5c(nc4n3C)CCN(C[C@H](N)CF)C5=O)n(CC3CC3)c12)n1cnc(C#N)c1] | 1-((S)-1-((2-(7-((S)-2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)propan-2-yl)-1H-imidazole-4- carbonitrile LCMS (ESI, m/z): 596 [M + H]+; LCMS RT: 0.913 min (Method D) ¹H NMR (400 MHz, DMSO-d₆) δ 8.48 (s, 1H), 8.43 (s, 1H), 8.14 (s, 1H), 7.31 (d, J = 7.6 Hz, 1H), 7.15 (s, 1H), 7.07 (t, J = 8.0 Hz, 1H), 6.89 (d, J = 8.0 Hz, 1H), 4.98-4.96 (m, 1H), 4.57-4.35 (m, 6H), 3.93 (s, 3H), 3.79-3.74 (m, 2H), |
| | | 3.65-3.28 (m, 5H), 1.60 (s, 3H), 0.72- |
| | | 0.70 (m, 1H), 0.15-0.12 (m, 2H), |
| | | −0.30-−0.32 (m, 2H). |
| 88 | 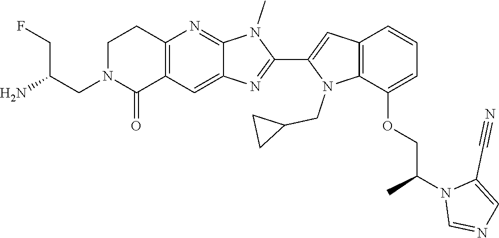 [SMILES: C[C@@H](COc1cccc2cc(-c3nc4cc5c(nc4n3C)CCN(C[C@H](N)CF)C5=O)n(CC3CC3)c12)n1cncc1C#N] | 1-((S)-1-((2-(7-((S)-2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)propan-2-yl)-1H-imidazole-5- carbonitrile LCMS (ESI, m/z): 596 [M + H]+; LCMS RT: 1.744 min (Method D) ¹H NMR (400 MHz, DMSO-d₆) δ 8.50 (s, 1H), 8.37 (s, 1H), 7.92 (s, 1H), 7.32 (d, J = 8.0 Hz, 1H), 7.16 (s, 1H), 7.09 (d, J = 8.0 Hz, 1H), 6.94 (d, J = 8.0 Hz, 1H), 5.03-4.98 (m, 1H), 4.63-4.32 (m, 6H), 3.94 (s, 3H), 3.78-3.65 (m, 5H), |
| | | 3.31-3.29 (m, 2H), 1.67 (s, 3H), 0.82- |
| | | 0.80 (m, 1H), 0.14-0.11 (m, 2H), |
| | | −0.30-−0.32 (m, 2H). |
| 89 | 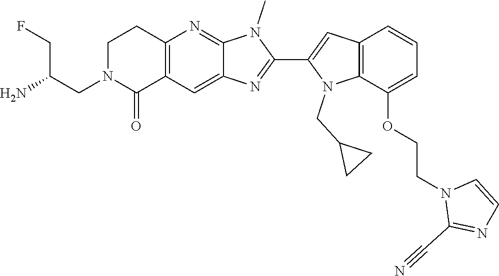 [SMILES: Cn1c(-c2cc3cccc(OCCn4ccnc4C#N)c3n2CC2CC2)nc2cc3c(nc21)CCN(C[C@H](N)CF)C3=O] | (S)-1-(2-((2-(7-(2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)ethyl)-1H-imidazole-2- carbonitrile LCMS (ESI, m/z): 582 [M + H]+; LCMS RT 0.901 min (Method D) ¹H NMR (400 MHz, DMSO-d₆) δ 8.53 (s, 1H), 8.31 (s, 3H), 7.87 (s, 1H), 7.36- 7.31 (m, 2H), 7.28 (s, 1H), 7.17-7.10 (m, 1H), 6.92 (d, J = 8.0 Hz, 1H), 4.78- 4.62 (m, 6H), 4.52 (d, J = 7.2 Hz, 2H), 4.12 (s, 3H), 3.98-3.71 (m, 5H), 3.34- 3.31 (m, 2H), 0.79-0.71 (m, 1H), 0.15- 0.09 (m, 2H) −0.33-−0.34 (m, 2H). |
| 90 | 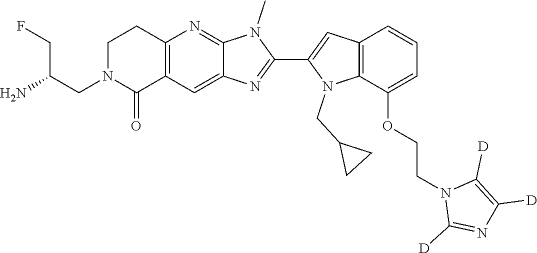 [SMILES: [2H]c1nc([2H])n(CCOc2cccc3cc(-c4nc5cc6c(nc5n4C)CCN(C[C@H](N)CF)C6=O)n(CC4CC4)c23)c1[2H]] | (S)-2-(7-(2-(1H-imidazol-1-yl- d3)ethoxy)-1-(cyclopropylmethyl)-1H- indol-2-yl)-7-(2-amino-3-fluoropropyl)- 3-methyl-3,5,6,7-tetrahydro-8H- imidazo[4,5-b][1,6]naphthyridin-8-one LCMS (ESI, m/z): 560 [M + H] +; LCMS RT: 0.725 min (Method D) ¹H NMR (400 MHz, DMSO-d₆) δ 8.48 (s, 1H), 8.36 (s, 3H), 7.32 (d, J = 7.6 Hz, 1H), 7.18 (s, 1H), 7.08 (d, J = 8 Hz, 1H), 6.92 (d, J = 7.6 Hz, 1H),4.78-4.65 (m, 6H), 4.59-4.50 (m, 2H), 4.00 (s, 3H), 3.94-3.80 (m, 5H), 3.34-3.31 (m, 2H), 0.71-0.70 (m, 1H), 0.28-0.20 (m, 2H), −0.30-−0.04 (m, 2H). |
| 91 | 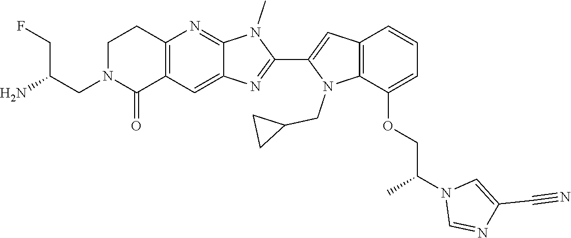 [SMILES: C[C@H](COc1cccc2cc(-c3nc4cc5c(nc4n3C)CCN(C[C@H](N)CF)C5=O)n(CC3CC3)c12)n1cnc(C#N)c1] | 1-((R)-1-((2-(7-((S)-2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)propan-2-yl)-1H-imidazole-4- carbonitrile LCMS (ESI, m/z): 596 [M + H]+; LCMS RT: 1.451 min (Method D) ¹H NMR (400 MHz, DMSO-d6) δ 8.51 (s, 1H), 8.42 (s, 1H), 8.13 (s, 1H), 7.31 (d, J = 7.6 Hz, 1H), 7.16 (s, 1H), 7.07 (t, J = 8.0 Hz, 1H), 6.90 (d, J = 6.8 Hz, 1H), 4.48 (s, 1H), 4.90-4.40 (m, 5H), 4.40-4.30 (m, 1H), 3.93 (s, 3H), 3.76- |
| | | 3.74 (m, 2H), 3.75-3.65 (m, 3H), 3.45- |
| | | 3.43 (m, 2H), 2.55 (s, 1H), 1.60 (s, 3H), |
| | | 0.90-0.60 (m, 1H), 0.20-0.10 (m, 2H), |
| | | −0.36-−0.40 (m, 2H). |
| 92 | 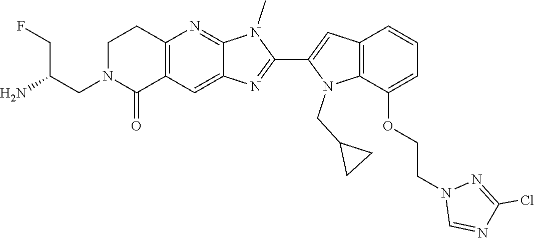 | (S)-7-(2-amino-3-fluoropropyl)-2-(7-(2- (3-chloro-1H-1,2,4-triazol-1-yl)ethoxy)- 1-(cyclopropylmethyl)-1H-indol-2-yl)- 3-methyl-3,5,6,7-tetrahydro-8H- imidazo[4,5-b][1,6]naphthyridin-8-one LCMS (ESI, m/z): 582 [M + H]+; LCMS RT: 0.933 min (Method D) ¹H NMR (400 MHz, DMSO-d₆) δ 8.76 (s, 1H), 8.52 (s, 1H), 8.42 (s, 3H), 7.32 (d, J = 10.8 Hz, 1H), 7.17 (s, 1H), 7.10- 7.05 (m, 1H), 6.96-6.90 (m, 1H), 4.84- 4.46 (m, 7H), 3.99 (s, 3H), 3.89- 3.72 (m, 5H), 3.35-3.31 (m, 2H), 0.91-0.85 (m, 1H), 0.17-0.05 (m, 2H), −0.25-−0.28 (m, 2H). |
| 93 | 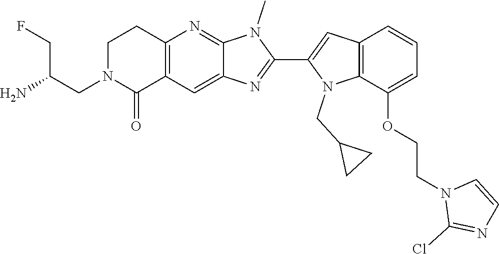 [SMILES: Cn1c(-c2cc3cccc(OCCn4ccnc4Cl)c3n2CC2CC2)nc2cc3c(nc21)CCN(C[C@H](N)CF)C3=O] | (S)-7-(2-amino-3-fluoropropyl)-2-(7-(2- (2-chloro-1H-imidazol-1-yl)ethoxy)-1- (cyclopropylmethyl)-1H-indol-2-yl)-3- methyl-3,5,6,7-tetrahydro-8H- imidazo[4,5-b][1,6]naphthyridin-8-one LCMS (ESI, m/z): 591[M + H]+; LCMS RT: 0.917 min (Method D) ¹H NMR (400 MHz, DMSO-d₆) δ 8.52 (s, 1H), 8.27 (s, 3H), 7.50 (d, J = 12 Hz, 1H), 7.31 (d, J = 8 Hz, 1H), 7.17 (s, 1H), 7.07 (d, J = 15.6 Hz, 1H), 6.95-6.90 (m, 1H), 4.78-4.60 (m, 6H), 4.51 (d, J = 6.8 Hz, 2H), 4.12 (s, 3H), 3.88-3.71 (m, 5H), 3.34-3.31 (m, 2H), 0.71-0.70 (m, 1H), 0.28-0.20 (m, 2H), −0.30-−0.04 (m, 2H). |
| 94 | 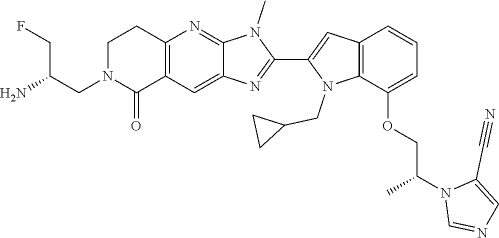 [SMILES: C[C@H](COc1cccc2cc(-c3nc4cc5c(nc4n3C)CCN(C[C@H](N)CF)C5=O)n(CC3CC3)c12)n1cncc1C#N] | 1-((R)-1-((2-(7-((S)-2-amino-3- fluoropropyl)-3-methyl-8-oxo-5,6,7,8- tetrahydro-3H-imidazo[4,5- b][1,6]naphthyridin-2-yl)-1- (cyclopropylmethyl)-1H-indol-7- yl)oxy)propan-2-yl)-1H-imidazole-5- carbonitrile LCMS (ESI, m/z): 596 [M + H]+; LCMS RT: 1.356 min (Method D) ¹H NMR (400 MHz, DMSO-d₆) δ 8.51 (s, 1H), 8.42 (s, 1H), 7.91 (s, 1H), 7.31 (d, J = 7.6 Hz, 1H), 7.16 (s, 1H), 7.08- 7.06 (m, 1H), 6.91-6.90 (m, 1H), 5.10- 4.90 (m, 1H), 4.90-4.40 (m, 4H), 4.40- 4.30 (m, 1H), 4.20-4.11 (m, 1H), 3.93 |
| | | (s, 6H), 3.76-3.74 (m, 2H), 3.45-3.43 |
| | | (m, 2H), 1.60 (s, 3H), 0.90-0.60 (m, |
| | | 1H), 0.20-0.10 (m, 2H), −0.36-−0.40 |
| | | (m, 2H). |

### Example 95. (S)-2-(7-(2-(1H-imidazol-1-yl)ethoxy-1,1,2,2-d4)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

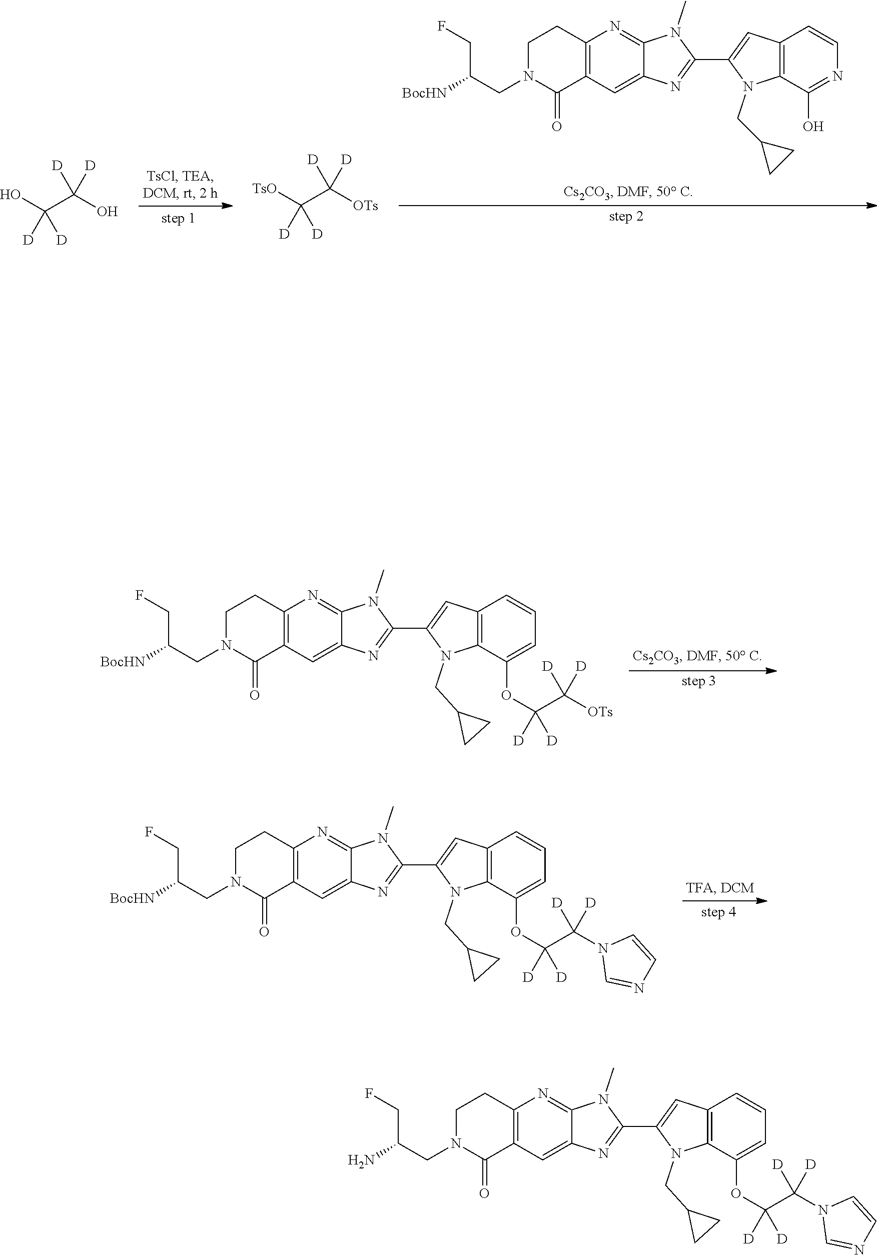

### Step 1: Synthesis of ethane-1,2-diyl-d4 bis(4-methylbenzenesulfonate)

Ethane-d4-1,2-diol (100 mg, 1.51 mmol) in DCM (10 mL) was reacted with TEA (459 mg, 4.54 mmol) and TsCl (433 mg, 2.27 mmol) according to General Procedure 13 to afford the title compound as a light yellow oil. LCMS (ESI, m/z): 375 [M+H]+.

### Step 2: Synthesis of (S)-2-((2-(7-(2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)ethyl-1,1,2,2-d4 4-methylbenzenesulfonate

Intermediate 5 (40 mg, 0.07 mmol) in DMF (2 mL) was reacted with ethane-1,2-diyl-d4 bis(4-methylbenzenesulfonate) (39.9 mg, 0.11 mmol) according to General Procedure 5. The crude product was purified by column chromatography (petroleum ether/ethyl acetate=2:1) to afford the title compound (30 mg, 55.1%) as a light yellow solid. LCMS (ESI, m/z): 765 [M+H]+

### Step 3: Synthesis of tert-butyl (S)-(1-(2-(7-(2-(1H-imidazol-1-yl)ethoxy-1,1,2,2-d4)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

(S)-2-((2-(7-(2-((tert-butoxycarbonyl)amino)-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)oxy)ethyl-1,1,2,2-d4 4-methylbenzenesulfonate (30 mg, 0.04 mmol) in DMF (2 mL) was reacted with 1H-imidazole (4.01 mg, 0.06 mmol) and Cs₂CO₃ (38.3 mg, 0.12 mmol) according to General Procedure 14. The crude product was purified by column chromatography (petroleum ether/ethyl acetate=4:1) to afford the title compound (20 mg, 70.7%) as a light-yellow solid. LCMS (ESI, m/z): 661 [M+H]+.

### Step 4 Synthesis of (S)-2-(7-(2-(1H-imidazol-1-yl)ethoxy-1,1,2,2-d4)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

Tert-butyl (S)-(1-(2-(7-(2-(1H-imidazol-1-yl)ethoxy-1,1,2,2-d4)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (20 mg, 0.03 mmol) was reacted according to General Procedure 2. The reaction system was purified by Prep-HPLC (Method F) to afford the title compound (7.2 mg, 42%) as a white solid. ¹H NMR (400 MHz, DMSO-d₆) δ 8.70 (s, 1H), 8.37 (s, 1H), 8.20 (s, 2H), 7.64 (s, 1H), 7.38 (s, 1H), 7.19 (d, J=8.0 Hz, 1H), 7.04 (s, 1H), 6.95 (d, J=8.0 Hz, 1H), 6.78 (d, J=8.0 Hz, 1H), 4.65-4.39 (m, 2H), 3.82 (s, 3H), 3.75-3.63 (m, 5H), 3.42-3.38 (m, 2H), 0.62-0.61 (s, 1H), 0.00-0.02 (m, 2H), −0.45-−0.46 (m, 2H). LCMS (ESI, m/z): 561 [M+H]+. LCMS RT: 0.719 min (Method D).

### Example 96. (S)-2-(7-(2-(1H-imidazol-1-yl)-2-methylpropoxy)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

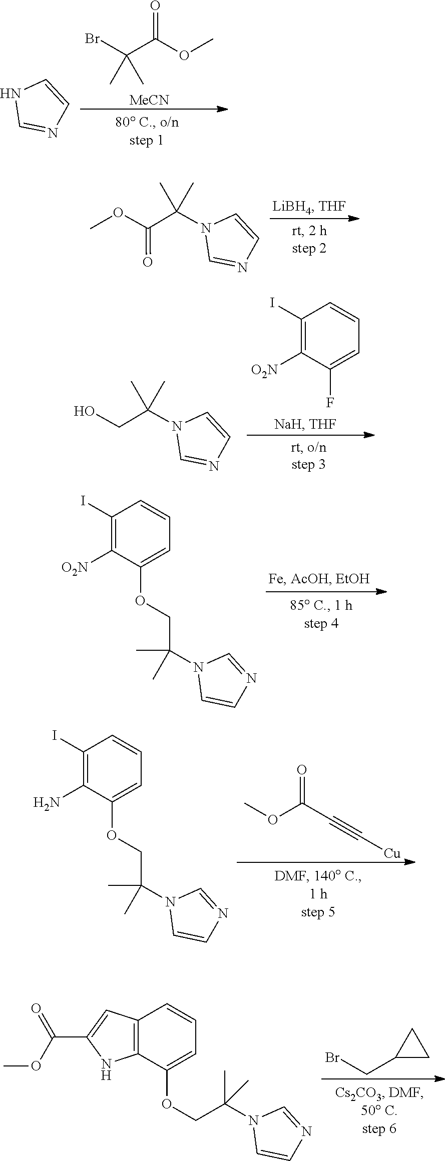

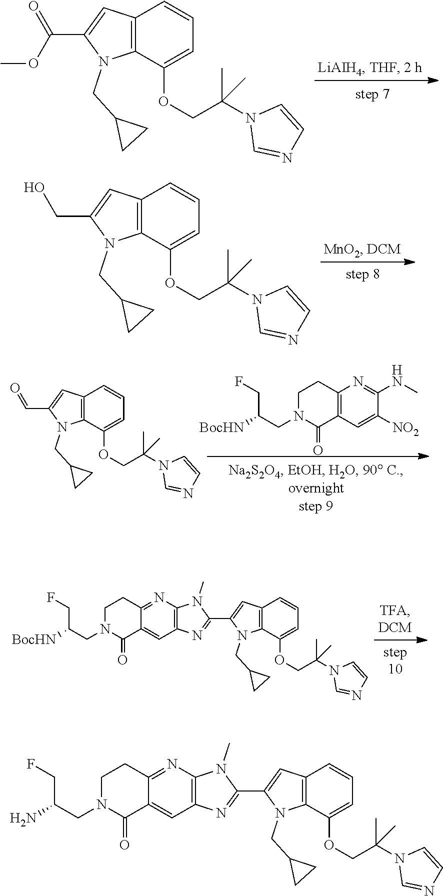 [SMILES: COC(=O)c1cc2cccc(OCC(C)(C)n3ccnc3)c2n1CC1CC1 ; CC(C)(COc1cccc2cc(CO)n(CC3CC3)c12)n1ccnc1 ; CC(C)(COc1cccc2cc(C=O)n(CC3CC3)c12)n1ccnc1 ; CNc1nc2c(cc1[N+](=O)[O-])C(=O)N(C[C@@H](CF)NC(=O)OC(C)(C)C)CC2 ; Cn1c(-c2cc3cccc(OCC(C)(C)n4ccnc4)c3n2CC2CC2)nc2cc3c(nc21)CCN(C[C@@H](CF)NC(=O)OC(C)(C)C)C3=O ; Cn1c(-c2cc3cccc(OCC(C)(C)n4ccnc4)c3n2CC2CC2)nc2cc3c(nc21)CCN(C[C@H](N)CF)C3=O]

### Step 1: Synthesis of methyl 2-(1H-imidazol-1-yl)-2-methylpropanoate

To a solution of 1H-imidazole (1 g, 14.6 mmol) in MeCN (20 mL) was added methyl 2-bromo-2-methylpropanoate (1.3 g, 7.34 mmol) at room temperature. The resulting mixture was stirred at 80° C. overnight. The reaction was monitored by LCMS. The mixture was concentrated under reduced pressure purified by flash column chromatography on C18 silica to afford the title compound (900 mg, 36.4%) as colorless oil. LCMS (ESI, m/z): 169 [M+H]+

### Step 2: Synthesis of 2-(1H-imidazol-1-yl)-2-methylpropan-1-ol

To a solution of methyl 2-imidazol-1-yl-2-methyl-propanoate (900 mg, 5.35 mmol) in THE (20 mL), was added LiBH₄ (2.5 M in THF) (10.8 mL, 21.4 mmol) dropwise at 0° C. under nitrogen atmosphere. The resulting solution was stirred at room temperature for 2 h under nitrogen atmosphere. The reaction was monitored by LCMS. The reaction was then quenched by adding MeOH and stirred at room temperature for 20 min and concentrated under vacuum. The crude product was purified by flash column chromatography on C18 silica to afford the title compound as a white solid. LCMS (ESI, m/z): 141 [M+H]+

### Step 3: Synthesis of 1-(1-(3-iodo-2-nitrophenoxy)-2-methylpropan-2-yl)-1H-imidazole

To a solution of 2-(1H-imidazol-1-yl)-2-methylpropan-1-ol (350 mg, 2.5 mmol) in DMF (15 mL), was added NaH (60%) (5.4 mL, 4.99 mmol) in portions at 0° C. was under nitrogen atmosphere. The mixture was stirred at room temperature for 0.5 h. The resulting mixture was stirred for 2 h at room temperature. The reaction was monitored by LCMS. The reaction was quenched by adding water (50 mL), extracted with ethyl acetate (2×50 mL). The combined organic extracts were washed with water (2×100 mL) and brine (100 mL), dried over anhydrous sodium sulfate and concentrated under vacuum. The crude product was purified by column chromatography (petroleum ether/ethyl acetate=10:1) to afford the title compound (600 mg, 62.1%) as a brown oil. LCMS (ESI, m/z): 388 [M+H]+

### Step 4: Synthesis of 2-(2-(1H-imidazol-1-yl)-2-methylpropoxy)-6-iodoaniline

To a solution of 1-(1-(3-iodo-2-nitrophenoxy)-2-methylpropan-2-yl)-1H-imidazole (600 mg, 1.55 mmol) in AcOH (4 mL, 69.8 mmol) and ethanol (8 mL) was added iron (865 mg, 15.5 mmol) at room temperature and was stirred for 1 h at 85° C. The reaction was monitored by TLC and LCMS. The mixture was filtered through a Celite pad. The filtrate was concentrated under reduced pressure and purified by flash column chromatography on C18 silica to afford the title compound (550 mg, 99.4%) as brown oil. LCMS (ESI, m/z): 358 [M+H]+

### Step 5: Synthesis of methyl 7-(2-(1H-imidazol-1-yl)-2-methylpropoxy)-1H-indole-2-carboxylate

To a solution of 2-(2-(1H-imidazol-1-yl)-2-methylpropoxy)-6-iodoaniline (550 mg, 1.54 mmol) in DMF (10 mL), was added (3-methoxy-3-oxo-prop-1-ynyl)copper (293 mg, 2 mmol) at room temperature. The mixture was stirred at 50° C. for 1.5 h. The reaction was monitored by TLC and LCMS. The reaction was then quenched by adding water (30 mL) and extracted with ethyl acetate (2×30 mL). The combined organic extracts were washed with water (2×60 mL) and brine (60 mL), dried over anhydrous sodium sulfate and concentrated under vacuum. The crude product was purified by column chromatography (petroleum ether/ethyl acetate=3:1) to afford the title compound (70 mg, 14.5%) as a brown oil. LCMS (ESI, m/z): 314 [M+H]+

### Step 6: Synthesis of methyl 7-(2-(1H-imidazol-1-yl)-2-methylpropoxy)-1-(cyclopropylmethyl)-1H-indole-2-carboxylate

methyl 7-(2-(1H-imidazol-1-yl)-2-methylpropoxy)-1H-indole-2-carboxylate (40 mg, 0.13 mmol) was reacted with bromomethylcyclopropane (34 mg, 0.26 mmol) according to General Procedure 5. The crude product was purified by column chromatography (petroleum ether/ethyl acetate=3:1) to afford the title compound (20 mg, 42.6%) as a yellow oil. LCMS (ESI, m/z): 368 [M+H]+

### Step 7: Synthesis of (7-(2-(1H-imidazol-1-yl)-2-methylpropoxy)-1-(cyclopropylmethyl)-1H-indol-2-yl)methanol

Methyl 7-(2-(1H-imidazol-1-yl)-2-methylpropoxy)-1-(cyclopropylmethyl)-1H-indole-2-carboxylate (20 mg, 0.05 mmol) was reacted according to General Procedure 3. The crude product was purified by column chromatography (petroleum ether/ethyl acetate=1:1) to afford the title compound (20 mg, 98.2%) as a yellow oil. The crude product was used to the next step directly. LCMS (ESI, m/z): 340 [M+H]+

### Step 8: Synthesis of 7-(2-(1H-imidazol-1-yl)-2-methylpropoxy)-1-(cyclopropylmethyl)-1H-indole-2-carbaldehyde

(7-(2-(1H-imidazol-1-yl)-2-methylpropoxy)-1-(cyclopropylmethyl)-1H-indol-2-yl)methanol (20 mg, 0.06 mmol) was reacted according to General Procedure 4. The crude product was purified by column chromatography (petroleum ether/ethyl acetate=3:1) to afford the title compound (18 mg, 90.5%) as a yellow oil. LCMS (ESI, m/z): 338 [M+H]+

### Step 9: Synthesis of tert-butyl (S)-(1-(2-(7-(2-(1H-imidazol-1-yl)-2-methylpropoxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

7-(2-(1H-imidazol-1-yl)-2-methylpropoxy)-1-(cyclopropylmethyl)-1H-indole-2-carbaldehyde (18 mg, 0.05 mmol) and Intermediate 2 (25 mg, 0.06 mmol) were reacted according to General Procedure 6. The crude material was purified by Prep-TLC (DCM/MeOH=20/1) to afford the title compound (25 mg, 68.4%) as a yellow oil. LCMS (ESI, m/z): 685 [M+H]⁺

### Step 10 Synthesis of (S)-2-(7-(2-(1H-imidazol-1-yl)-2-methylpropoxy)-1-(cyclopropylmethyl)-1H-indol-2-yl)-7-(2-amino-3-fluoropropyl)-3-methyl-3,5,6,7-tetrahydro-8H-imidazo[4,5-b][1,6]naphthyridin-8-one

Tert-butyl (S)-(1-(2-(7-(2-(1H-imidazol-1-yl)-2-methylpropoxy)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (25 mg, 0.04 mmol) was reacted according to General Procedure 2. The crude material was purified by Prep HPLC (Method F) to afford the title compound (6 mg, 27.7%) as an off-white solid. ¹H NMR (400 MHz, DMSO-d₆) δ 9.37 (s, 1H), 8.48 (s, 1H), 8.33 (s, 3H), 8.14 (s, 1H), 7.81 (s, 1H), 7.34 (d, J=7.9 Hz, 1H), 7.19 (s, 1H), 7.10 (t, J=7.9 Hz, 1H), 6.93 (d, J=7.9 Hz, 1H), 4.82-4.58 (m, 4H), 4.43 (d, J=6.8 Hz, 2H), 3.95 (s, 3H), 3.86-3.73 (m, 5H), 3.33 (t, J=6.6 Hz, 2H), 1.82 (s, 6H), 0.72-0.59 (m, 1H), 0.15-0.05 (m, 2H), −0.35-−0.47 (m, 2H). LCMS (ESI, m/z): 585 [M+H]+. LCMS RT: 0.711 min (Method D).

### Example 97. (S,E)-7-(2-amino-3-fluoropropyl)-2-(1-(cyclopropylmethyl)-7-(3-(4-fluoro-1H-imidazol-1-yl)prop-1-en-1-yl)-1H-indol-2-yl)-3-methyl-3,5,6,7-tetrahydro-8H-imidazo[4,5-b][1,6]naphthyridin-8-one

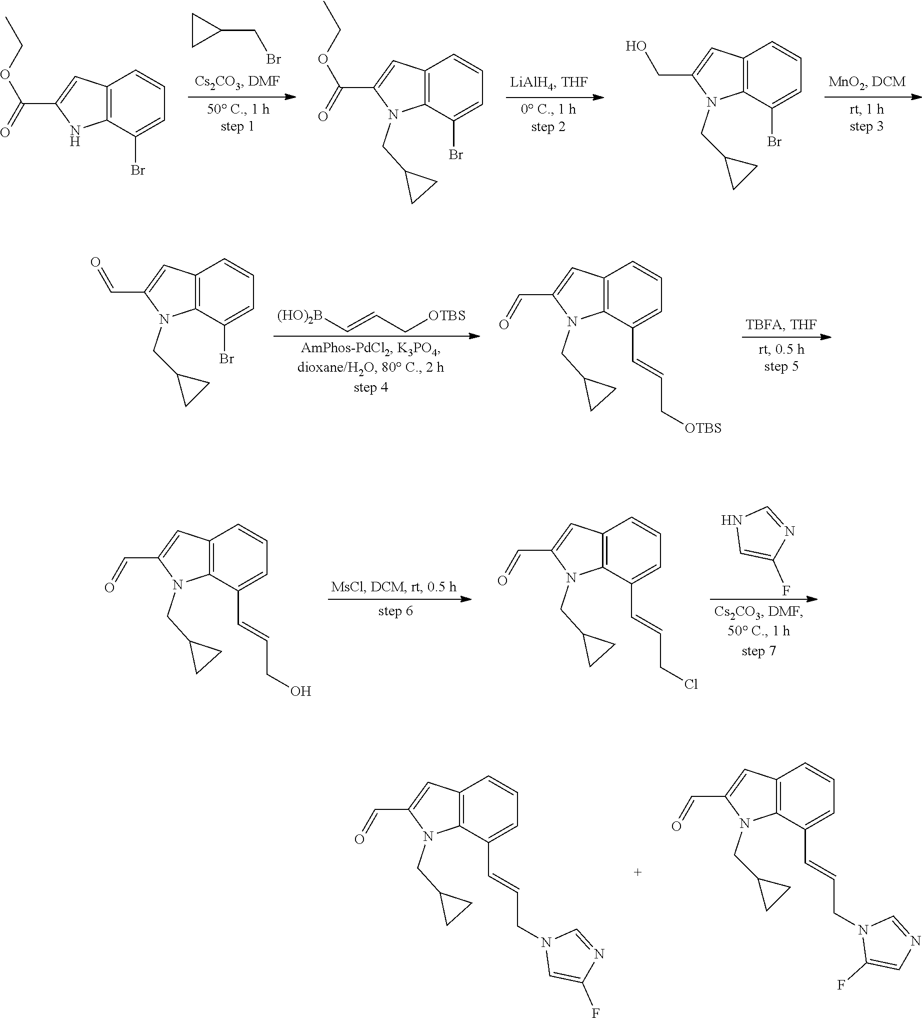

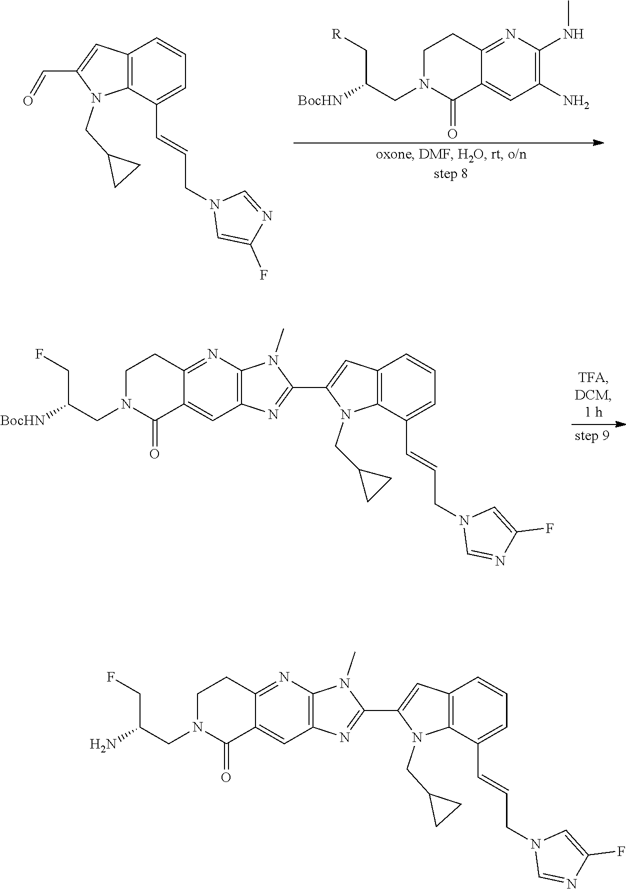

### Step 1: Synthesis of ethyl 7-bromo-1-(cyclopropylmethyl)-1H-indole-2-carboxylate

Ethyl 7-bromo-1H-indole-2-carboxylate (500 mg, 1.86 mmol) was reacted with (bromomethyl)cyclopropane (510 mg, 3.7 mmol) according to General Procedure 5. The crude product was purified by column chromatography (petroleum ether/ethyl acetate=6:1) to afford the title compound (480 mg, 81%) as a light yellow solid. LCMS (ESI, m/z): 322 [M+H]+

### Step 2: Synthesis of (7-bromo-1-(cyclopropylmethyl)-1H-indol-2-yl)methanol

Ethyl 7-bromo-1-(cyclopropylmethyl)-1H-indole-2-carboxylate (480 mg, 1.49 mmol) was reacted according to General Procedure 3. The crude product was purified by column chromatography (petroleum ether/ethyl acetate=3:1) to afford the title compound (400 mg, 91%) as a light yellow solid. LCMS (ESI, m/z): 294 [M+H]+

### Step 3: Synthesis of 7-bromo-1-(cyclopropylmethyl)-1H-indole-2-carbaldehyde

(7-bromo-1-(cyclopropylmethyl)-1H-indol-2-yl)methanol (400 mg, 1.36 mmol) was reacted according to General Procedure 4. The crude product was purified by column chromatography (petroleum ether/ethyl acetate=5:1) to afford the title compound (300 mg, 78%) as a light yellow solid. LCMS (ESI, m/z): 278 [M+H]+

### Step 4 Synthesis of (E)-7-(3-((tert-butyldimethylsilyl)oxy)prop-1-en-1-yl)-1-(cyclopropylmethyl)-1H-indole-2-carbaldehyde

To a solution of 7-bromo-1-(cyclopropylmethyl)-1H-indole-2-carbaldehyde (300 mg, 1.07 mmol) and (E)-(3-((tert-butyldimethylsilyl)oxy)prop-1-en-1-yl)boronic acid (350 mg, 1.60 mmol) in dioxane (10 mL) and water (2 mL), was added K₃PO₄ (580 mg, 3.21 mmol) and AmPhos-PdCl2 (85 mg, 0.1 mmol) under nitrogen atmosphere. The mixture was stirred at 80° C. for 2 h under nitrogen atmosphere. The reaction was monitored by LCMS. The reaction was then quenched by adding water (30 mL) and extracted with ethyl acetate (2×30 mL), washed with water (50 mL) and brine (50 mL), dried over anhydrous sodium sulfate and concentrated under vacuum. The crude product was purified by column chromatography (petroleum ether/ethyl acetate=10:1) to afford the title compound (320 mg, 81%) as a light yellow solid. LCMS (ESI, m/z): 370 [M+H]+

### Step 5 Synthesis of (E)-1-(cyclopropylmethyl)-7-(prop-1-en-1-yl)-1H-indole-2-carbaldehyde

To a solution of (E)-7-(3-((tert-butyldimethylsilyl)oxy)prop-1-en-1-yl)-1-(cyclopropylmethyl)-1H-indole-2-carbaldehyde (320 mg, 0.8 mmol) in THE (10 mL), was added TBAF (5 mL) under nitrogen atmosphere. The resulting solution was stirred at room temperature for 2 h under nitrogen atmosphere. The reaction was monitored by LCMS. The reaction was then quenched by adding water (30 mL) and extracted with ethyl acetate (30 mL), washed with water (5×30 mL) and brine (5×30 mL), dried over anhydrous sodium sulfate and concentrated under vacuum. The crude product was purified by column chromatography (petroleum ether/ethyl acetate=5:1) to afford the title compound (180 mg, 90%) as a light yellow solid. LCMS (ESI, m/z): 239 [M+H]+

### Step 6: Synthesis of (E)-7-(3-chloroprop-1-en-1-yl)-1-(cyclopropylmethyl)-1H-indole-2-carbaldehyde

(E)-1-(cyclopropylmethyl)-7-(prop-1-en-1-yl)-1H-indole-2-carbaldehyde (180 mg, 0.75 mmol) in DCM (10 mL) was reacted with TEA (228 mg, 2.1 mmol) and MsCl (165 mg, 1.5 mmol) according to General Procedure 13 to afford the title compound (180 mg, 90%) as a light yellow oil. LCMS (ESI, m/z): 274 [M+H]+

### Step 7: Synthesis of (E)-1-(cyclopropylmethyl)-7-(3-(4-fluoro-1H-imidazol-1-yl)prop-1-en-1-yl)-1H-indole-2-carbaldehyde

(E)-7-(3-chloroprop-1-en-1-yl)-1-(cyclopropylmethyl)-1H-indole-2-carbaldehyde (180 mg, 0.65 mmol) in DMF (10 mL) was reacted with 4-fluoro-1H-imidazole (111 mg, 1.3 mmol) and Cs₂CO₃ (635 mg, 1.95 mmol) according to General Procedure 14. The crude product was purified by column chromatography (petroleum ether/ethyl acetate=5:1) to afford the title compound (150 mg, 71%) as a light yellow solid. LCMS (ESI, m/z): 323 [M+H]+

### Step 8: Synthesis of tert-butyl (S,E)-(1-(2-(1-(cyclopropylmethyl)-7-(3-(4-fluoro-1H-imidazol-1-yl)prop-1-en-1-yl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

1-(isoxazol-5-ylmethyl)-7-methyl-1H-indole-2-carbaldehyde (100 mg, 0.22 mmol) and Intermediate 7 (60 mg, 0.15 mmol) were reacted according to General Procedure 11. The reaction mixture was purified by flash column chromatography on C18 silica to afford the title compound (70 mg, 79%) as a yellow solid. LCMS (ESI, m/z): 671[M+H]+

### Step 9: Synthesis of (S,E)-7-(2-amino-3-fluoropropyl)-2-(1-(cyclopropylmethyl)-7-(3-(4-fluoro-1H-imidazol-1-yl)prop-1-en-1-yl)-1H-indol-2-yl)-3-methyl-3,5,6,7-tetrahydro-8H-imidazo[4,5-b][1,6]naphthyridin-8-one

Tert-butyl (S,E)-(1-(2-(1-(cyclopropylmethyl)-7-(3-(4-fluoro-1H-imidazol-1-yl)prop-1-en-1-yl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (25 mg, 0.04 mmol) was reacted according to General Procedure 2. The crude product was purified by Prep-HPLC (Method E) to afford (S,E)-7-(2-amino-3-fluoropropyl)-2-(1-(cyclopropylmethyl)-7-(3-(4-fluoro-1H-imidazol-1-yl)prop-1-en-1-yl)-1H-indol-2-yl)-3-methyl-3,5,6,7-tetrahydro-8H-imidazo[4,5-b][1,6]naphthyridin-8-one (8.1 mg, 79%) as a yellow solid. ¹H NMR (400 MHz, DMSO-d₆) δ 8.52 (s, 1H), 8.28 (s, 3H), 7.67 (d, J=2.4 Hz, 1H), 7.59 (s, 1H), 7.14 (s, 3H), 7.24 (s, 1H), 7.09-7.02 (m, 1H), 6.32 (d, J=15.2 Hz, 1H), 4.78-4.62 (m, 2H), 4.43 (d, J=6.4 Hz, 2H), 4.45-3.72 (m, 10H), 3.35-3.32 (m, 2H), 0.72 (s, 1H), 0.16-0.13 (m, 2H), −0.31-−0.34 (m, 2H). LCMS (ESI, m/z): 571 [M+H]+. LCMS RT: 0.896 min (Method I).

Example 98 in Table 12 was obtained following a procedure similar to the preparation of a Example 97 using the regioisomeric imidazole intermediate from step 7.

TABLE 12

| Example | | LCMS m/z [M + H]+; LCMS RT (LCMS |
|---|---|---|
| Number | Structure | method); ¹H NMR (MHz, solvent) |
| 98 | 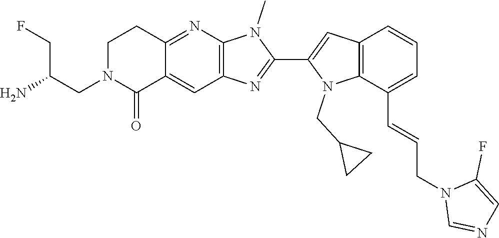 [SMILES: Cn1c(-c2cc3cccc(/C=C/Cn4cncc4F)c3n2CC2CC2)nc2cc3c(nc21)CCN(C[C@H](N)CF)C3=O] | (S,E)-7-(2-amino-3-fluoropropyl)-2-(1- (cyclopropylmethyl)-7-(3-(5-fluoro-1H-imidazol- 1-yl)prop-1-en-1-yl)-1H-indol-2-yl)-3-methyl- 3,5,6,7-tetrahydro-8H-imidazo[4,5- b][1,6]naphthyridin-8-one LCMS (ESI, m/z): 571 [M + H]+; LCMS RT: 0.929 min (Method I) ¹H NMR (400 MHz, DMSO-d₆) δ 8.50 (s, 1H), 8.28 (m, 3H), 7.65 (d, J = 7.6, 2H), 7.44 (d, J = 1.2, 1H), 7.27 (s, 1H), 7.23-7.14 (m, 2H), 6.92- 6.88 (m, 1H), 6.42-6.36 (m, 1H), 4.80-4.60 (m, 4H), 4.02 (d, J = 6.0 Hz, 2H), 3.98 (s, 3H), 3.87- 3.71 (m, 5H), 3.48-3.32 (m, 2H), 0.91-0.88 (m, 1H), 0.88-0.16 (m, 2H), −0.38-−0.41 (m, 2H). |

### Example 99. (S)-7-(2-amino-3-fluoropropyl)-2-(1-(cyclopropylmethyl)-7-(3-(4-fluoro-1H-imidazol-1-yl)propyl)-1H-indol-2-yl)-3-methyl-3,5,6,7-tetrahydro-8H-imidazo[4,5-b][1,6]naphthyridin-8-one

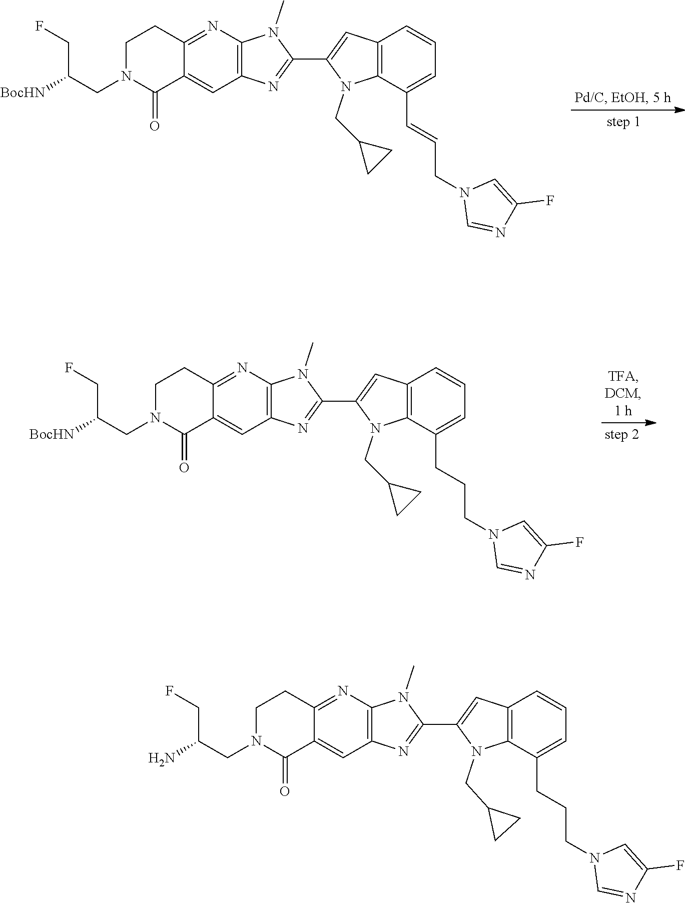 [SMILES: Cn1c(-c2cc3cccc(/C=C/Cn4cnc(F)c4)c3n2CC2CC2)nc2cc3c(nc21)CCN(C[C@@H](CF)NC(=O)OC(C)(C)C)C3=O ; Cn1c(-c2cc3cccc(CCCn4cnc(F)c4)c3n2CC2CC2)nc2cc3c(nc21)CCN(C[C@@H](CF)NC(=O)OC(C)(C)C)C3=O ; Cn1c(-c2cc3cccc(CCCn4cnc(F)c4)c3n2CC2CC2)nc2cc3c(nc21)CCN(C[C@H](N)CF)C3=O]

### Step 1: Synthesis of tert-butyl (S)-(1-(2-(1-(cyclopropylmethyl)-7-(3-(4-fluoro-1H-imidazol-1-yl)propyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Tert-butyl (S,E)-(1-(2-(1-(cyclopropylmethyl)-7-(3-(4-fluoro-1H-imidazol-1-yl)prop-1-en-1-yl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (65 mg, 0.77 mmol) in methanol (30 mL) was reacted according to General Procedure 9. The crude product was purified by column chromatography (petroleum ether/ethyl acetate=1:2) to afford the title compound (30 mg, 80.5%) as a light yellow solid. LCMS (ESI, m/z): 673 [M+H]+

### Step 2: Synthesis of (S)-7-(2-amino-3-fluoropropyl)-2-(1-(cyclopropylmethyl)-7-(3-(4-fluoro-1H-imidazol-1-yl)propyl)-1H-indol-2-yl)-3-methyl-3,5,6,7-tetrahydro-8H-imidazo[4,5-b][1,6]naphthyridin-8-one

Tert-butyl (S)-(1-(2-(1-(cyclopropylmethyl)-7-(3-(4-fluoro-1H-imidazol-1-yl)propyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.04 mmol) was reacted according to General Procedure 2. The crude product was purified by Prep-HPLC (Method E) to afford the title compound (19.3 mg, 79%) as a yellow solid. ¹H NMR (400 MHz, DMSO-d6) δ 8.51 (s, 1H), 8.30 (s, 3H), 7.60 (d, J=2.4 Hz, 1H), 7.59 (s, 1H), 7.14 (s, 1H), 7.24 (s, 2H), 7.09-7.02 (m, 1H), 4.78-4.62 (m, 2H), 4.43 (d, J=6.4 Hz, 2H), 4.45-3.72 (m, 10H), 3.35-3.32 (m, 2H), 3.06-3.02 (m, 2H), 2.17-2.13 (m, 2H), 0.72 (s, 1H), 0.16-0.13 (m, 2H), −0.43-−0.47 (m, 2H). LCMS (ESI, m/z): 573 [M+H]+. LCMS RT: 0.896 min (Method I).

### Example 100. (S)—N-(2-(7-(2-amino-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)-2-(1H-imidazol-1-yl)acetamide

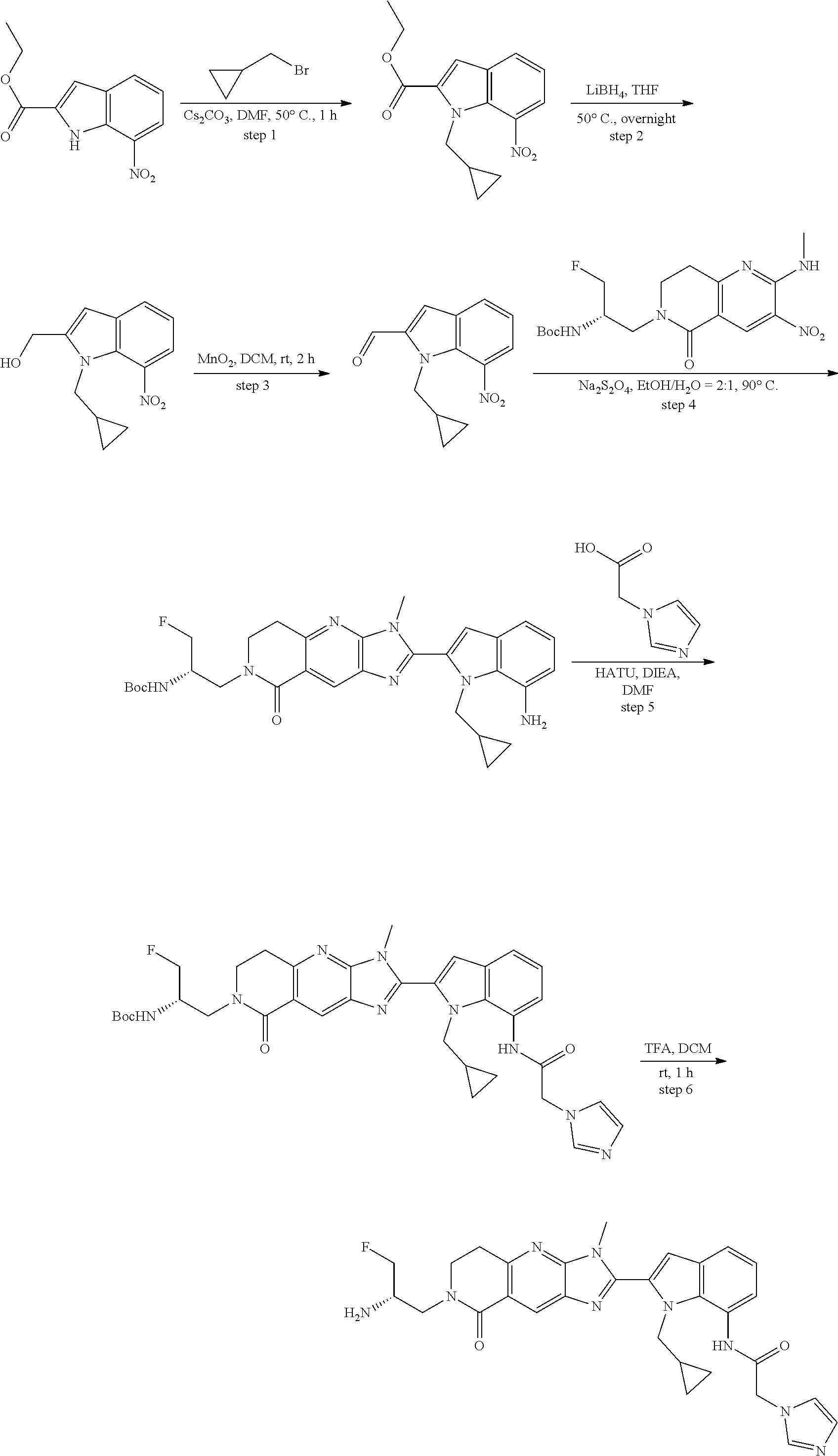

### Step 1: Synthesis of ethyl 1-(cyclopropylmethyl)-7-nitro-1H-indole-2-carboxylate

Ethyl 7-nitro-1H-indole-2-carboxylate (5 g, 21.4 mmol) in DMF (100 mL), was reacted with bromomethylcyclopropane (4.32 g, 32 mmol) according to General Procedure 5. The crude product was purified by column chromatography (petroleum ether/ethyl acetate=5:1) to afford the title compound (5 g, 81.2%) as a yellow solid. LCMS (ESI, m/z): 289 [M+H]+.

### Step 2: Synthesis of (1-(cyclopropylmethyl)-7-nitro-1H-indol-2-yl)methanol

To a solution of ethyl 1-(cyclopropylmethyl)-7-nitro-1H-indole-2-carboxylate (2 g, 6.94 mmol) in THF (1.5 mL), was added LiBH₄ (2 M in THF) (302 mg, 13.9 mmol) drop wise at 0° C. under nitrogen atmosphere. The resulting solution was stirred at room temperature for 2 h under nitrogen atmosphere. The reaction was monitored by LCMS. The reaction was then quenched by adding water (0.7 mL)/NaOH (aq) (2 mL)/water (0.7 mL) and stirred at room temperature for 20 minute, dried over anhydrous sodium sulfate. Solids were filtered out and the solvent was concentrated under vacuum. The crude product was purified by column chromatography (petroleum ether/ethyl acetate=3:1) to afford the title compound (647 mg, 38%) as a yellow solid. LCMS (ESI, m/z): 247 [M+H]+

### Step 3: Synthesis of 1-(cyclopropylmethyl)-7-nitro-1H-indole-2-carbaldehyde

(1-(cyclopropylmethyl)-7-nitro-1H-indol-2-yl)methanol (670 mg, 2.07 mmol) was reacted according to General Procedure 4. The crude product was purified by column chromatography (petroleum ether/ethyl acetate=10:1) to afford the title compound (460 mg, 69.1%) as a yellow solid. LCMS (ESI, m/z): 245 [M+H]+

### Step 4: Synthesis of tert-butyl (S)-(1-(2-(7-amino-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

Intermediate 2 (400 mg, 1 mmol) and 1-(cyclopropylmethyl)-7-nitro-1H-indole-2-carbaldehyde (367 mg, 1.5 mmol) was reacted according to General Procedure 6. The crude product was purified by column chromatography (petroleum ether/ethyl acetate=1:2) to afford the title compound (30 mg, 5%) yellow solid. LCMS (ESI, m/z): 562 [M+H]+

### Step 5 Synthesis of tert-butyl (S)-(1-(2-(7-(2-(1H-imidazol-1-yl)acetamido)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate

To a solution of 2-imidazol-1-ylacetic acid (82 mg, 0.65 mmol) and DIEA (0.06 mL, 0.65 mmol) in DMF (1 mL), was added HATU (74.1 mg, 0.19 mmol) at room temperature. The mixture was stirred at room temperature for 10 minute. Then the above resulting mixture was added into a solution of tert-butyl (S)-(1-(2-(7-amino-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (73 mg, 0.13 mmol) in DMF (1 mL) with drop wise at room temperature. The resulting mixture was stirred for 2 h at room temperature. The reaction was monitored by LCMS. The reaction was then quenched by adding water (10 mL) and extracted with ethyl acetate (10 mL). The combined organic extracts were washed with water (3×10 mL) and brine (3×10 mL), dried over anhydrous sodium sulfate and concentrated under vacuum. The crude product was purified by column chromatography (petroleum ether/ethyl acetate=1:2) to afford the title compound (30 mg, 35%) as a yellow solid. LCMS (ESI, m/z): 670 [M+H]+

### Step 6 Synthesis of (S)—N-(2-(7-(2-amino-3-fluoropropyl)-3-methyl-8-oxo-5,6,7,8-tetrahydro-3H-imidazo[4,5-b][1,6]naphthyridin-2-yl)-1-(cyclopropylmethyl)-1H-indol-7-yl)-2-(1H-imidazol-1-yl)acetamide

Tert-butyl (S)-(1-(2-(7-(2-(1H-imidazol-1-yl)acetamido)-1-(cyclopropylmethyl)-1H-indol-2-yl)-3-methyl-8-oxo-3,5,6,8-tetrahydro-7H-imidazo[4,5-b][1,6]naphthyridin-7-yl)-3-fluoropropan-2-yl)carbamate (30 mg, 0.04 mmol) was reacted according to General Procedure 2. The crude product was purified by Prep-HPLC (Method F) to afford the title compound (6.9 mg, 26%) as yellow solid. ¹HNMR (400 MHz, Methanol-d4) δ 9.10 (s, 1H), 8.66 (s, 1H), 7.76-7.75 (m, 2H), 7.66 (s, 1H), 7.30-7.28 (m, 3H), 5.45 (s, 2H), 4.90-4.82 (m, 2H), 4.53 (d, J=6.4 Hz, 2H), 4.10-4.08 (m, 1H), 4.04 (s, 3H), 3.93-3.80 (m, 4H), 3.46-3.43 (m, 2H), 0.90-0.88 (m, 1H), 0.25-0.23 (m, 2H), −0.24-−0.28 (m, 2H). LCMS (ESI, m/z): 570 [M+H]+. LCMS RT: 1.079 min. (Method D).

### Biological Assays

Compounds of the present disclosure were assayed as inhibitors of PAD4 using the assay protocols described below.

### RFMS Assay 1 Conditions

Compounds were solubilized in 100% DMSO to achieve a 10 mM compound concentration. Compound stock solutions were stored at RT. A series of dilutions were prepared in DMSO and mixed 8 times with 20 μL mixing volume. Final top concentration of compound in the assay is 50 μM. Final assay conditions were as follows:
Reaction volume: 26 μl
Assay buffer: 25 mM hepes, pH 7.5, 5 mM NaCl, 1 mM DTT, 0.2 mg/ml BSA, 0.01% CHAPS, 50 μM Calcium, and 5 μM TPEN
Final concentrations: 5 nM hPAD4 enzyme, 250 μM BAEE, and 0.5% DMSO
Total incubation time: 30 mins compound and enzyme preincubation at 37° C., 90 min enzyme/substrate reaction, 30 min reaction with phenyl glyoxal at 37° C.
Stop solution: 40 μl 5% TCA in ACN

0.13 μL of compound solution was added to 13 μL of 10 nM PAD4 in assay buffer. After 30 min 13 μl of 500 μM of BAEE was added in 25 mM hepes, pH 7.5, 5 mM NaCl, 1 mM DTT, 0.2 mg/ml BSA, 0.01% CHAPS, 50 M Calcium, 5 M TPEN was added and the reaction incubated for 90 min at 37° C. The enzymatic reaction was quenched by addition of 15 μl of 6.1N TCA, 100% Final Concentration is 20%, 35 μl of 8.5 mM phenyl glyoxal (final concentration 4 mM) is then added and the reaction is incubated for 30 min at 37° C.

After 30 minutes the plates are spun down to remove all precipitate. The enzyme reaction was quenched with an equal volume of methanol containing internal standard (modified citrulline). Samples were loaded onto the Rapid Fire RF300 system (Agilent) wherein they were first sipped for 1000 ms and then directly loaded to a C18 separations cartridge using a mixture of acetonitrile containing 0.01% formic acid for 3000 ms desalting. The flow rate of the mobile phase was 1.5 ml/min. Once the samples were eluted from the cartridge, a mobile phase of acetonitrile containing 0.01% formic acid was used to move the samples into the mass spectrometer for 4000 ms at a flow rate of 1.25 ml/min/Sciex AP15500 triple quadrupole mass spectrometer (Applied Biosystems) equipped with ESI was used to analyze the peptidyl citrulline and internal standard ions.

MRM transition of product and internal standard were monitored at m/z 424.5 to 350.4 and m/z 293 to 247 respectively. The dwell time for each transition was set at 200 ms, and the ESI voltage was used at 5500 with a source temperature of 400° C. Extracted ion peaks for each transition were integrated using the Rapid Fire Integrator software. Peak area of analyte was normalized with internal standard.).

### RFMS Assay 2 Conditions

Compound preparation:

Stock compounds were dissolved and stored in 100% DMSO. Compound solutions were prepared via serial dilution at 3-fold intervals in DMSO with top compound concentration at 20 M in each assay. 0.25 μl of compound solution was transferred from the compound plate to the assay plate by using an acoustic dispenser. Final assay conditions:
Total reaction volume: 25 μl
Assay buffer: 100 HEPES pH 7.4, 200 mM NaCl, 2 mM CaCl₂, 5 mM DTT
35 nM recombinant human PAD4
500 M TSTGGRQGSHH (SEQ ID NO: 1)
1.2% DMSO
Stop solution: 10% formic acid

Reaction mixtures were incubated at room temperature for 30 minutes. 10 μl each of the reaction mixtures was then mixed with 40 μl of 10% formic acid in a microtiter plate. The plate was frozen at −80° C. before shipping out on dry ice for RapidFire mass spectroscopy analysis.

Thawed samples were loaded onto the Rapid Fire 300 system (Agilent) wherein they were first sipped for 250 ms and then loaded onto a Agilent “C” (C18) cartridge using a mobile phase of water containing 0.09% formic acid/0.01% trifluoroacetic acid for 3000 ms desalting flowing at a rate of 1.5 ml/min. Once the samples were loaded and washed, a mobile phase of acetonitrile containing 0.09% formic acid/0.01% trifluoroacetic acid was used to elute the samples directly onto a Sciex API4000 triple quadrupole mass spectrometer for 3000 ms at a flow rate of 1.25 ml/min.

MRM transitions for substrate and product were monitored in positive ESI mode at m/z=562.3/969.7 and m/z=562.8/541.3 respectively. The dwell time for each transition was set at 100 ms, and the ESI voltage was used at 5500 with a source temperature of 650° C. Extracted ion peaks for each transition were integrated using the Rapid Fire Integrator software.

For a given compound example, the Table below shows the human PAD4 (hPAD4) IC₅₀ in the rapid-fire mass spectrum (RFMS) assay.

Table 13, below, shows the activity of selected compounds of this invention in the PAD4 assays described above. Compounds having an activity designated as “A” provided an IC₅₀≤10 nM; compounds having an activity designated as “B” provided an IC₅₀ 11-100 nM; compounds having an activity designated as “C” provided an IC₅₀ 101-500 nM; compounds having an activity designated as “D” provided an IC₅₀ 501-1000 nM; compounds having an activity designated as “E” provided an IC₅₀>1000 nM.

TABLE 13

| Example # | RFMS Assay 1 | RFMS Assay 2 |
|---|---|---|
| 1 | C | D |
| 2 | E | E |
| 3 | C | |
| 4 | C | |
| 5 | C | |
| 6 | C | |
| 7 | B | C |
| 8 | D | D |
| 9 | | C |
| 10 | | E |
| 11 | C | C |
| 12 | C | C |
| 13 | | D |
| 14 | B | D |
| 15 | B | C |
| 16 | C | C |
| 17 | | D |
| 18 | | D |
| 19 | D | E |
| 20 | E | D |
| 21 | E | |
| 22 | C | C |
| 23 | B | D |
| 24 | B | D |
| 25 | C | |
| 26 | C | C |
| 27 | D | E |
| 28 | D | |
| 29 | C | |
| 30 | B | A |
| 31 | B | |
| 32 | C | |
| 33 | B | D |
| 34 | A | B |
| 35 | | D |
| 36 | | E |
| 37 | | D |
| 38 | B | B |
| 39 | A | B |
| 40 | B | |
| 41 | A | B |
| 42 | B | C |
| 43 | A | B |
| 44 | A | B |
| 45a | A | |
| 45b | A | |
| 46 | A | B |
| 47 | A | B |
| 48 | B | C |
| 49a | B | B |
| 49b | C | C |
| 50 | A | |
| 51 | A | |
| 52 | B | |
| 53 | C | |
| 54 | B | B |
| 55 | C | |
| 56 | A | |
| 57 | B | |
| 58 | C | |
| 59 | B | B |
| 60 | | C |
| 61a | C | |
| 61b | C | |
| 62 | B | C |
| 63 | B | |
| 64 | | C |
| 65a | A | C |
| 65b | | C |
| 66 | A | |
| 67 | A | |
| 68 | C | |
| 69 | B | |
| 70 | A | B |
| 71 | B | C |
| 72 | B | C |
| 73a | A | |
| 73b | A | |
| 74a | A | B |
| 74b | A | B |
| 75a | A | B |
| 75b | A | B |
| 76a | B | |
| 76b | B | |
| 77a | B | |
| 77b | B | |
| 78a | B | |
| 78b | A | |
| 78c | B | |
| 78d | B | |
| 79a | B | |
| 79b | B | |
| 80 | B | |
| 81 | A | |
| 82 | C | C |
| 83 | A | |
| 84 | A | B |
| 85 | A | B |
| 86 | A | B |
| 87 | B | |
| 88 | C | |
| 89 | C | |
| 90 | A | |
| 91 | C | |
| 92 | B | |
| 93 | B | |
| 94 | C | |
| 95 | A | B |
| 96 | A | C |
| 97 | B | |
| 98 | C | |
| 99 | B | |
| 100 | D | |

### EMBODIMENTS

Embodiment 1. A compound of formula I:

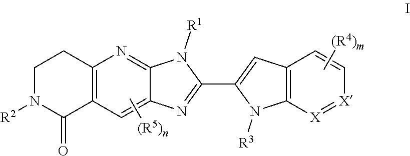

or a pharmaceutically acceptable salt, isomer, enantiomer, or tautomer thereof, wherein:
   X is selected from C—R⁶ and N;
   X′ is selected from C—R^{6′} and N, wherein X and X′ are not simultaneously N;
   R¹ is C₁₋₄ aliphatic;
   R² is C₁₋₆ aliphatic substituted by 0-4 instances of R⁷;
   R³ is C₁₋₆ aliphatic substituted by 0-3 instances of R⁸;
   R⁴ is halogen;
   R⁵ is halogen;
   each R⁶ and R^{6′} is independently selected from hydrogen, halogen, —OR, —N(R)₂, —OC(O)R, —N(R)C(O)R, —O-L-(R⁹)ₚ, —Cy, and optionally substituted C₁₋₆ aliphatic;
   each R⁷ is independently selected from halogen, —OR, —N(R)₂, and —Cy;
   each R⁸ is independently selected from halogen, —OR, —N(R)₂, and —Cy;
   each R⁹ is independently selected from halogen, —OR, —N(R)₂, and —Cy;
   L is a covalent bond or C1.4 aliphatic;
   each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
   each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
   each R is independently hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur;
   each m and n is independently 0 or 1; and
   p is independently 1-4.
Embodiment 2. The compound according to embodiment 1, wherein the compound is selected from a compound of formulae I-a, I-b, I-c, I-d, I-e, I-f, I-g, and I-h:

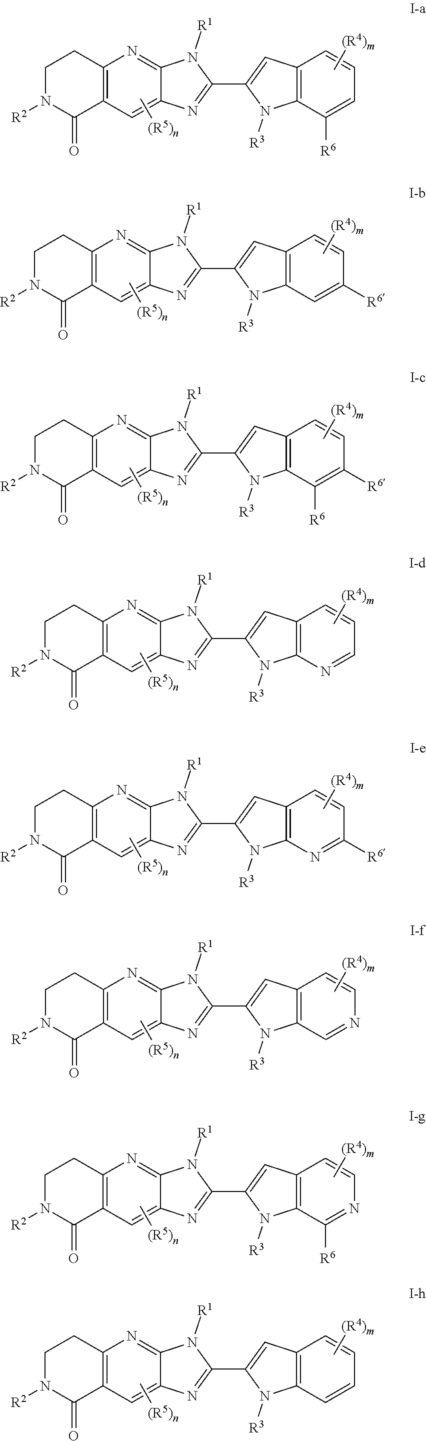

or a pharmaceutically acceptable salt thereof.
Embodiment 3. The compound according to any one of the preceding embodiments, wherein the compound is selected from a compound of formulae I-a-i, I-b-i, I-c-i, I-d-i, I-e-i, I-f-i, I-g-i, and I-h-i.

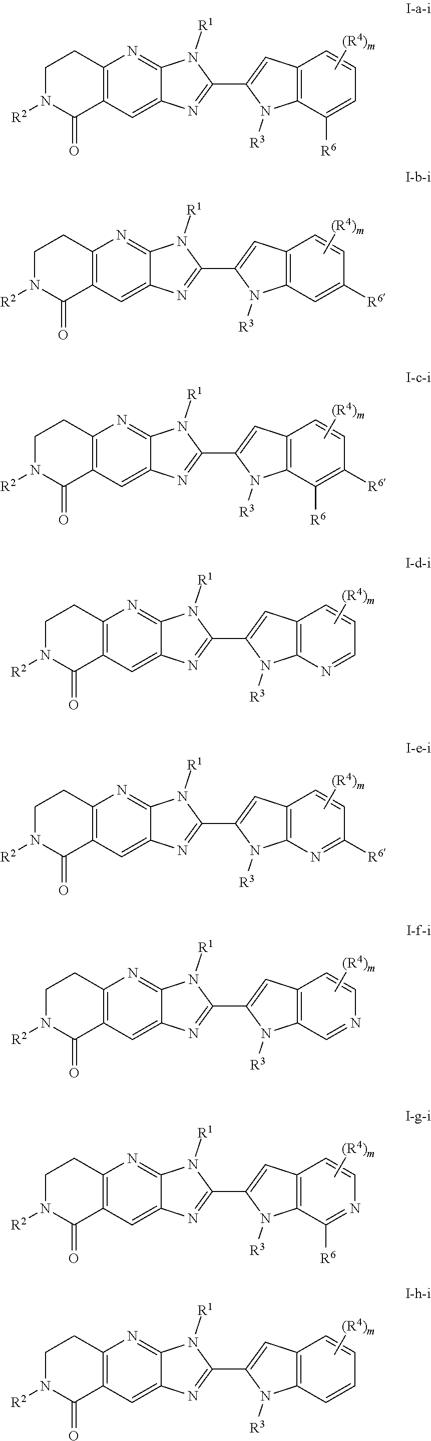

or a pharmaceutically acceptable salt thereof.
Embodiment 4. The compound according to any one of the preceding embodiments, wherein R¹ is —CH₃.
Embodiment 5. The compound according to any one of the preceding embodiments, wherein mis 1.
Embodiment 6. The compound according to any one of the preceding embodiments, wherein R⁴ is fluoro or chloro.
Embodiment 7. The compound according to any one of the preceding embodiments, wherein R⁴ is C₁₋₆ aliphatic.
Embodiment 8. The compound according to any one of the preceding embodiments, wherein R⁴ is —CH₃.
Embodiment 9. The compound according to any one of the preceding embodiments, wherein m is 0.
Embodiment 10. The compound according to any one of the preceding embodiments, wherein R² is C₁₋₆ aliphatic substituted by 1-4 instances of R⁷.
Embodiment 11. The compound according to any one of the preceding embodiments wherein R² is C₁₋₄ aliphatic substituted by 1-4 instances of R⁷.
Embodiment 12. The compound according to any one of the preceding embodiments, wherein R² is C₁₋₄ aliphatic substituted by 1-2 instances of R⁷.
Embodiment 13. The compound according to any one of the preceding embodiments, wherein at least one R⁷ is halogen.
Embodiment 14. The compound according to any one of the preceding embodiments, wherein the at least one R⁷ is fluoro.
Embodiment 15. The compound according to any one of the preceding embodiments, wherein at least one R⁷ is —N(R)₂.
Embodiment 16. The compound according to any one of the preceding embodiments, wherein the at least one R⁷ is selected from —NH₂ and —NHR.
Embodiment 17. The compound according to any one of the preceding embodiments, wherein R² is further substituted with at least one halogen.
Embodiment 18. The compound according to any one of the preceding embodiments, wherein at least one R⁷ is —Cy.
Embodiment 19. The compound according to any one of the preceding embodiments, wherein R² is selected from the group consisting of

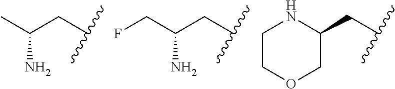

Embodiment 20. The compound according to any one of the preceding embodiments, wherein R³ is C₁₋₆ aliphatic substituted by 1-2 instances of R⁸.
Embodiment 21. The compound according to any one of the preceding embodiments, wherein R³ is C₁₋₄ aliphatic substituted by 1-2 instances of R⁸.
Embodiment 22. The compound according to any one of the preceding embodiments, wherein R³ is C₁₋₂ aliphatic substituted by 1 instance of R⁸.
Embodiment 23. The compound according to any one of the preceding embodiments, wherein R⁸ is —OR.
Embodiment 24. The compound according to any one of the preceding embodiments, wherein R⁸ is —Cy.
Embodiment 25. The compound according to any one of the preceding embodiments, wherein R⁸ is halogen.
Embodiment 26. The compound according to any one of the preceding embodiments, wherein R³ is selected from

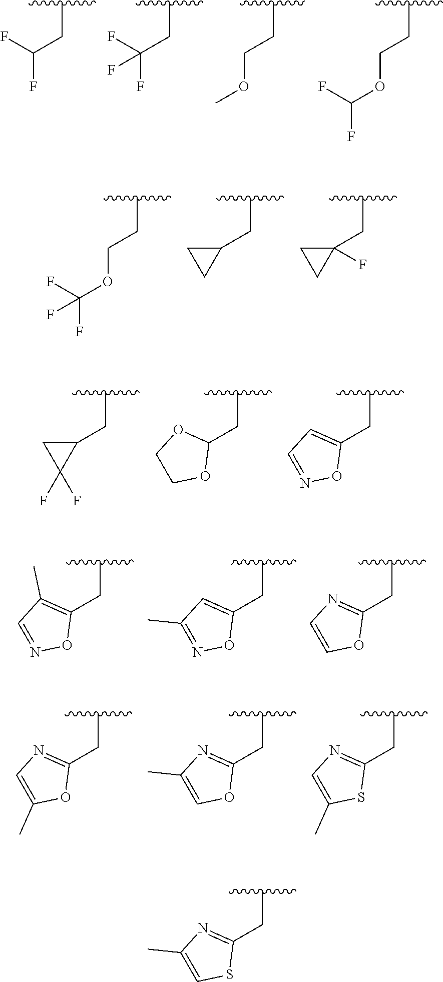

Embodiment 27. The compound according to any one of the preceding embodiments, wherein R⁶ is hydrogen.
Embodiment 28. The compound according to any one of the preceding embodiments, wherein R⁶ is —OR.
Embodiment 29. The compound according to any one of the preceding embodiments, wherein R⁶ is —N(R)C(O)R.
Embodiment 30. The compound according to any one of the preceding embodiments, wherein R⁶ is optionally substituted C₁₋₆ aliphatic.
Embodiment 31. The compound according to any one of the preceding embodiments, wherein R⁶ is —Cy.
Embodiment 32. The compound according to any one of the preceding embodiments, wherein —Cy is an optionally substituted 3- to 7-membered saturated or partially unsaturated heterocyclic ring having 1-2 heteroatoms independently selected from nitrogen, oxygen, and sulfur.
Embodiment 33. The compound according to any one of the preceding embodiments, wherein —Cy is an optionally substituted 6-membered saturated or partially unsaturated heterocyclic ring having 1-2 heteroatoms independently selected from nitrogen, oxygen, and sulfur.
Embodiment 34. The compound according to any one of the preceding embodiments, wherein R⁶ is —O-L-(R⁹)ₚ.
Embodiment 35. The compound according to any one of the preceding embodiments, wherein R⁶ is selected from hydrogen, —CH₃, —OCH₃,

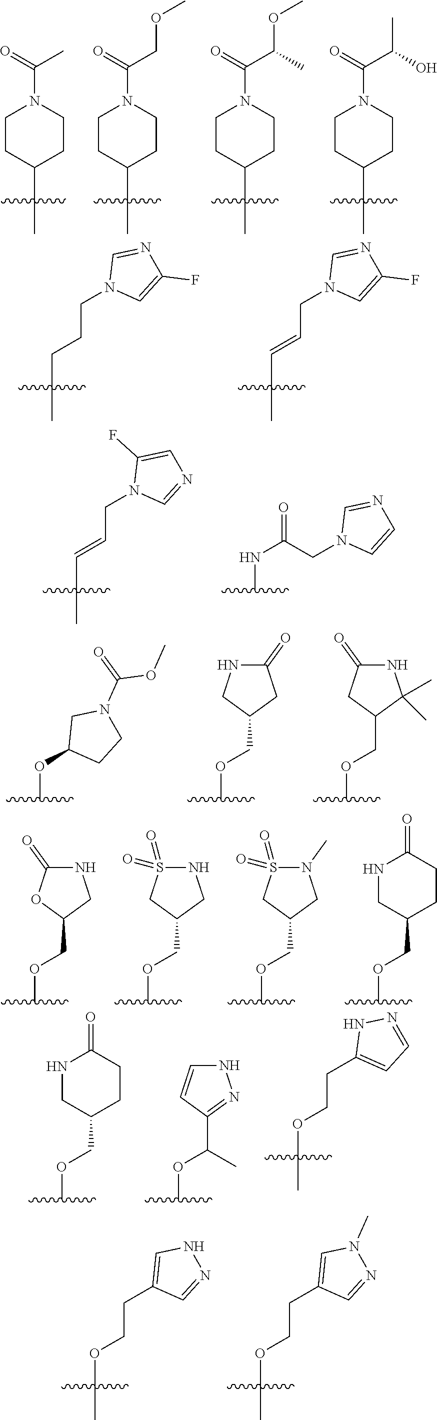

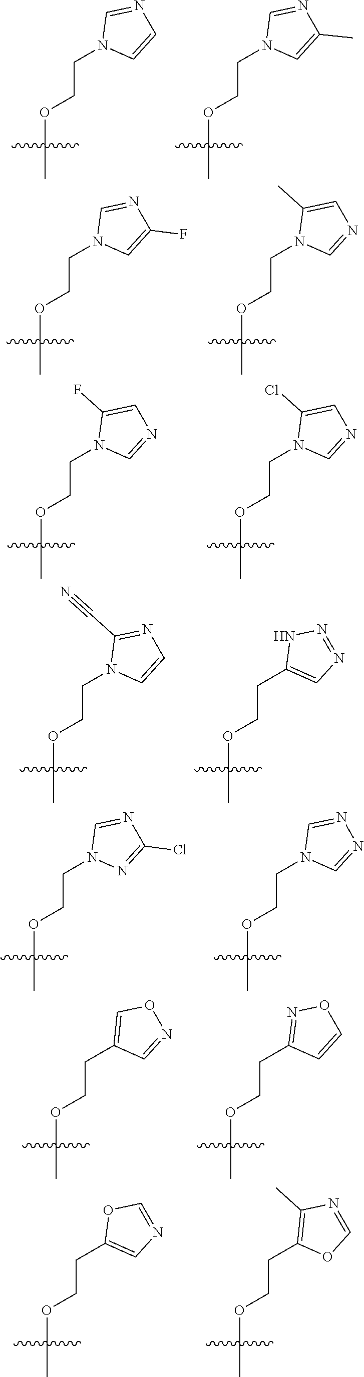

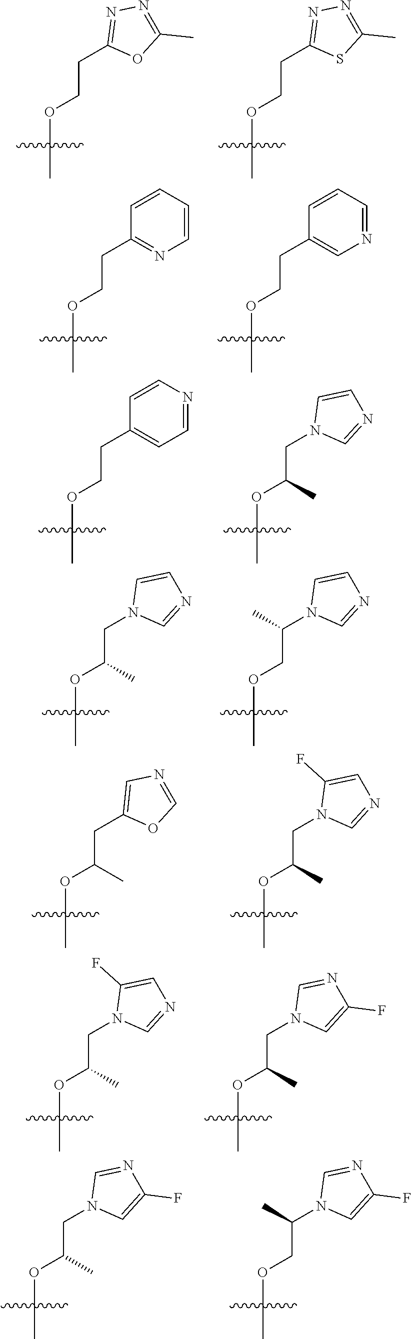

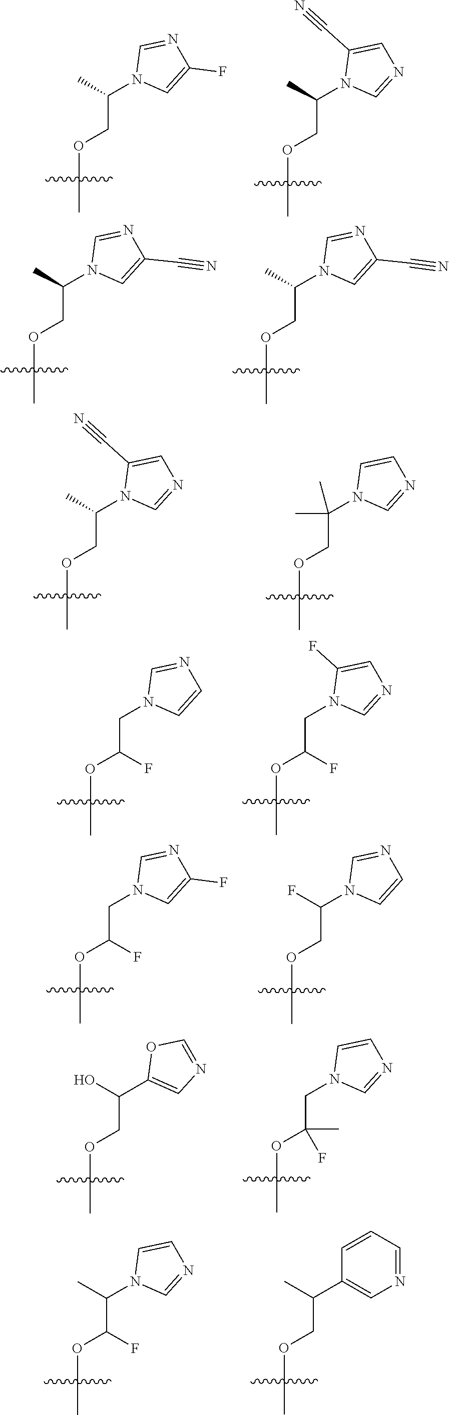

Embodiment 36. The compound according to any one of the preceding embodiments, wherein R^{6′} is hydrogen.
Embodiment 37. The compound according to any one of the preceding embodiments, wherein R^{6′} is —OR.
Embodiment 38. The compound according to any one of the preceding embodiments, wherein R^{6′} is halogen.
Embodiment 39. The compound according to any one of the preceding embodiments, wherein R⁶ is optionally substituted C₁₋₆ aliphatic.
Embodiment 40. The compound according to any one of the preceding embodiments, wherein R^{6′} is selected from hydrogen, fluoro, —OCH₃, and —C(CH₃)₂OH.
Embodiment 41. A pharmaceutically acceptable composition comprising the compound according to any one of the preceding embodiments, and a pharmaceutically acceptable carrier, adjuvant, or vehicle.
Embodiment 42. A method of inhibiting PAD4 in a subject or in a biological sample comprising the step of contacting the PAD4 with a compound according to any one of the preceding embodiments.
Embodiment 43. A method of treating a PAD4-mediated disease, disorder, or condition in a subject in need thereof comprising the step of administering to said subject the composition according to any one of the preceding embodiments.
Embodiment 44. The method according to any one of the preceding embodiments, wherein the PAD4-mediated disease, disorder, or condition is selected from the group consisting of acid-induced lung injury, acne (PAPA), acute lymphocytic leukemia, acute, respiratory distress syndrome, Addison's disease, adrenal hyperplasia, adrenocortical insufficiency, ageing, AIDS, alcoholic hepatitis, alcoholic hepatitis, alcoholic liver disease, allergen induced asthma, allergic bronchopulmonary, aspergillosis, allergic conjunctivitis, alopecia, Alzheimer's disease, amyloidosis, amyotropic lateral sclerosis, and weight loss, angina pectoris, angioedema, anhidrotic ecodermal dysplasia-ID, ankylosing spondylitis, anterior segment, inflammation, antiphospholipid syndrome, aphthous stomatitis, appendicitis, arthritis, asthma, atherosclerosis, atopic dermatitis, autoimmune diseases, autoimmune hepatitis, bee sting-induced inflammation, behcet's disease, Behcet's syndrome, Bells Palsey, berylliosis, Blau syndrome, bone pain, bronchiolitis, burns, bursitis, cancer, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic lymphocytic leukemia, chronic obstructive pulmonary disease, colitis, complex regional pain syndrome, connective tissue disease, corneal ulcer, crohn's disease, cryopyrin-associated periodic syndromes, cyrptococcosis, cystic fibrosis, deficiency of the interleukin-1-receptor antagonist (DTRA), dermatitis, dermatitis endotoxemia, dermatomyositis, diffuse intrinsic pontine glioma, endometriosis, endotoxemia, epicondylitis, erythroblastopenia, familial amyloidotic polyneuropathy, familial cold urticarial, familial mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Hodgkin's disease, Huntington's disease, hyaline membrane disease, hyperammonemia, hypercalcemia, hypercholesterolemia, hyperimmunoglobulinemia D with recurrent fever (HIDS), hypoplastic and other anemias, hypoplastic anemia, idiopathic thrombocytopenic purpura, incontinentia pigmenti, infectious mononucleosis, inflammatory bowel disease, inflammatory lung disease, inflammatory neuropathy, inflammatory pain, insect bite-induced inflammation, iritis, irritant-induced inflammation, ischemia/reperfusion, juvenile rheumatoid arthritis, keratitis, kidney disease, kidney injury caused by parasitic infections, kidney injury caused by parasitic infections, kidney transplant rejection prophylaxis, leptospiriosis, leukemia, Loeffler's syndrome, lung injury, lung injury, lupus, lupus, lupus nephritis, lymphoma, meningitis, mesothelioma, mixed connective tissue disease, Muckle-Wells syndrome (urticaria deafness amyloidosis), multiple sclerosis, muscle wasting, muscular dystrophy, myasthenia gravis, myocarditis, mycosis fungiodes, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osterarthritis, otitis media, paget's disease, pain, pancreatitis, Parkinson's disease, pemphigus, pericarditis, periodic fever, periodontitis, peritoneal endometriosis, pertussis, pharyngitis and adenitis (PFAPA syndrome), plant irritant-induced inflammation, pneumonia, pneumonitis, pneumosysts infection, poison ivy/urushiol oil-induced inflammation, polyarteritis nodosa, polychondritis, polycystic kidney disease, polymyositis, psoriasis, psoriasis, psoriasis, psoriasis, psychosocial stress diseases, pulmonary disease, pulmonary hypertension, pulmonayr fibrosis, pyoderma gangrenosum, pyogenic sterile arthritis, renal disease, retinal disease, rheumatic carditis, rheumatic disease, rheumatoid arthritis, sarcoidosis, seborrhea, sepsis, severe pain, sickle cell, sickle cell anemia, silica-induced disease, Sjogren's syndrome, skin diseases, sleep apnea, solid tumors, spinal cord injury, Stevens-Johnson syndrome, stroke, subarachnoid hemorrhage, sunburn, temporal arteritis, tenosynovitis, thrombocytopenia, thyroiditis, tissue transplant, TNF receptor associated periodic syndrome (TRAPS), toxoplasmosis, transplant, traumatic brain injury, tuberculosis, type 1 diabetes, type 2 diabetes, ulcerative colitis, urticarial, uveitis, and Wegener's granulomatosis.
Embodiment 45. The method according to any one of the preceding embodiments, wherein the PAD4-mediated disease, disorder, or condition is selected from rheumatoid arthritis, vasculitis, systemic lupus erythematosus, ulcerative colitis, cancer, cystic fibrosis, asthma, cutaneous lupus erythematosis, and psoriasis.
Embodiment 46. A compound of formula I′:

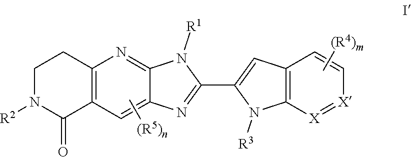

or a pharmaceutically acceptable salt, isomer, enantiomer, or tautomer thereof, wherein:
   X is selected from C—R⁶ and N;
   X′ is selected from C—R^{6′} and N, wherein X and X′ are not simultaneously N;
   R¹ is C₁₋₄ aliphatic;
   R² is C₁₋₆ aliphatic substituted by 0-4 instances of R⁷;
   R³ is C₁₋₆ aliphatic substituted by 0-3 instances of R¹;
   R⁴ is halogen;
   R⁵ is halogen;
   each R⁶ and R^{6′} is independently selected from hydrogen, halogen, —OR, —N(R)₂, —OC(O)R, —N(R)C(O)R, —N(R)C(O)-L-(R⁹)ₚ, —O-L-(R⁹)ₚ, —Cy, optionally substituted C₁₋₆ aliphatic, and C₁₋₆ aliphatic-OH;
   each R⁷ is independently selected from halogen, —OR, —N(R)₂, and —Cy;
   each R⁸ is independently selected from halogen, —OR, —N(R)₂, and —Cy;
   each R⁹ is independently selected from halogen, —OR, —N(R)₂, and —Cy;
   L is a covalent bond or C₁₋₄ aliphatic;
   each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
   each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
   each R is independently hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulphur, wherein each R is independently substituted with 0-3 instances of halogen, —OCH₃, or —OH;
   each m and n is independently 0 or 1; and
p is independently 1-4.

1. A compound of formula I: 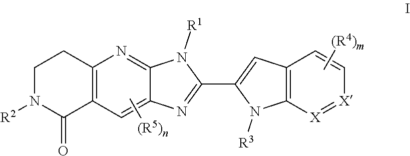
or a pharmaceutically acceptable salt, isomer, enantiomer, or tautomer thereof, wherein:
X is selected from C—R⁶ and N;
X′ is selected from C—R^{6′} and N, wherein X and X′ are not simultaneously N;
R¹ is C₁₋₄ aliphatic;
R² is C₁₋₆ aliphatic substituted by 0-4 instances of R⁷;
R³ is C₁₋₆ aliphatic substituted by 0-3 instances of R¹;
R⁴ is halogen;
R⁵ is halogen;
each R⁶ and R^{6′} is independently selected from hydrogen, halogen, —OR, —N(R)₂, —OC(O)R, —N(R)C(O)R, —O-L-(R⁹)ₚ, —Cy, and optionally substituted C₁₋₆ aliphatic;
each R⁷ is independently selected from halogen, —OR, —N(R)₂, and —Cy;
each R⁸ is independently selected from halogen, —OR, —N(R)₂, and —Cy;
each R⁹ is independently selected from halogen, —OR, —N(R)₂, and —Cy;
L is a covalent bond or C₁₋₄ aliphatic;
each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
each R is independently hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur;
each m and n is independently 0 or 1; and
p is independently 1-4.

2. The compound according to claim 1, wherein the compound is selected from a compound of formulae I-a, I-b, I-c, I-d, I-e, I-f, I-g, and I-h: 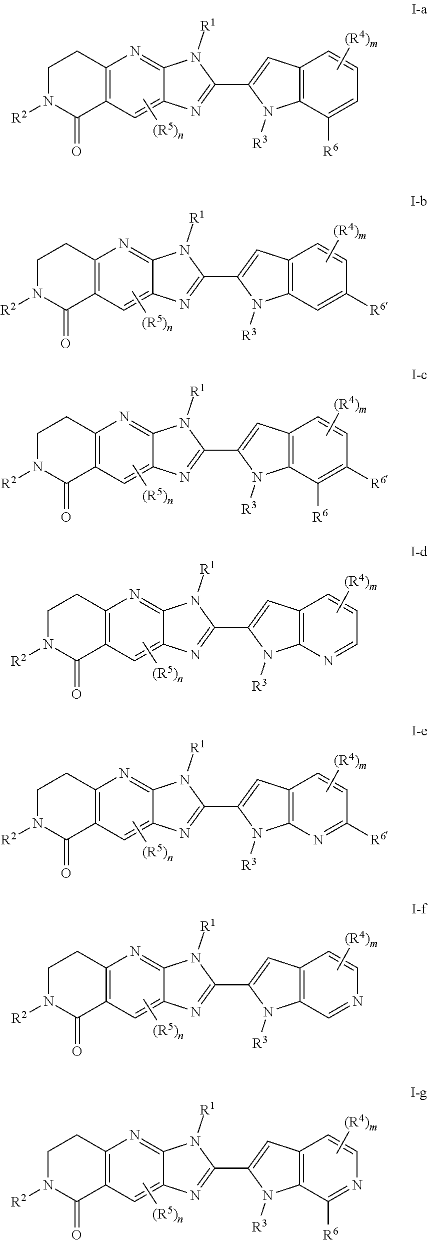 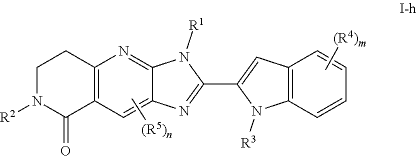
or a pharmaceutically acceptable salt thereof.

3. The compound according to claim 2, wherein the compound is selected from a compound of formulae I-a-i, I-b-i, I-c-i, I-d-i, I-e-i, I-f-i, I-g-i, and I-h-i: 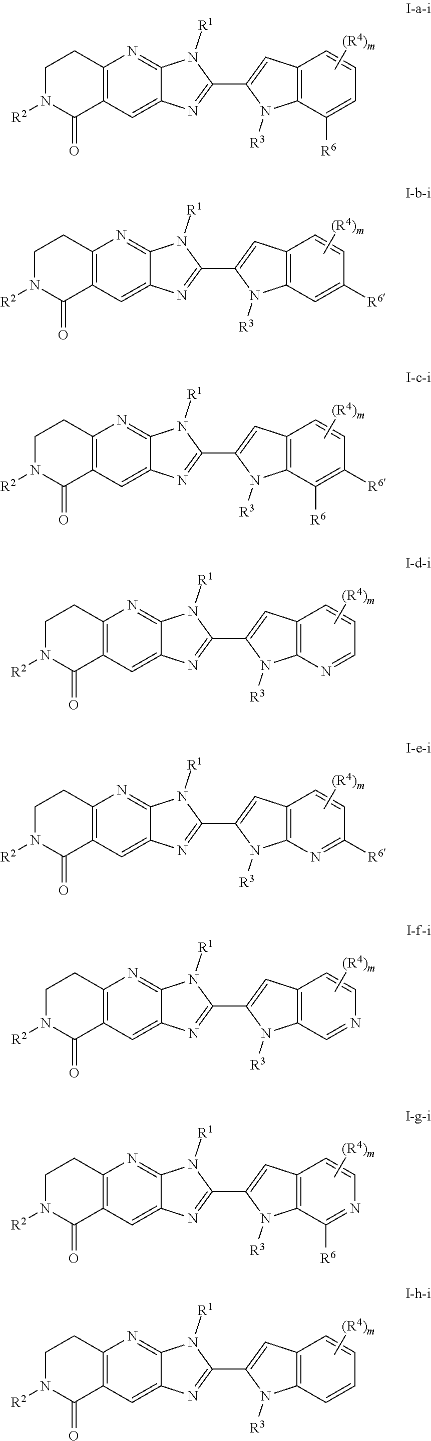
or a pharmaceutically acceptable salt thereof.

4. The compound according to claim 1, wherein R¹ is —CH₃.

5. The compound according to claim 1, wherein m is 1.

6. The compound according to claim 5, wherein R⁴ is fluoro or chloro.

7. The compound according to claim 1, wherein R⁴ is C₁₋₆ aliphatic.

8. The compound according to claim 1, wherein m is 0.

9. The compound according to claim 1, wherein R² is C₁₋₆ aliphatic substituted by 1-4 instances of R⁷.

10. The compound according to claim 9, wherein R² is C₁₋₄ aliphatic substituted by 1-2 instances of R⁷.

11. The compound according to claim 1, wherein at least one R⁷ is halogen.

12. The compound according to claim 11, wherein the at least one R⁷ is fluoro.

13. The compound according to claim 1, wherein R² is selected from the group consisting of

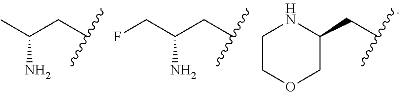

14. The compound according to claim 1, wherein R³ is selected from

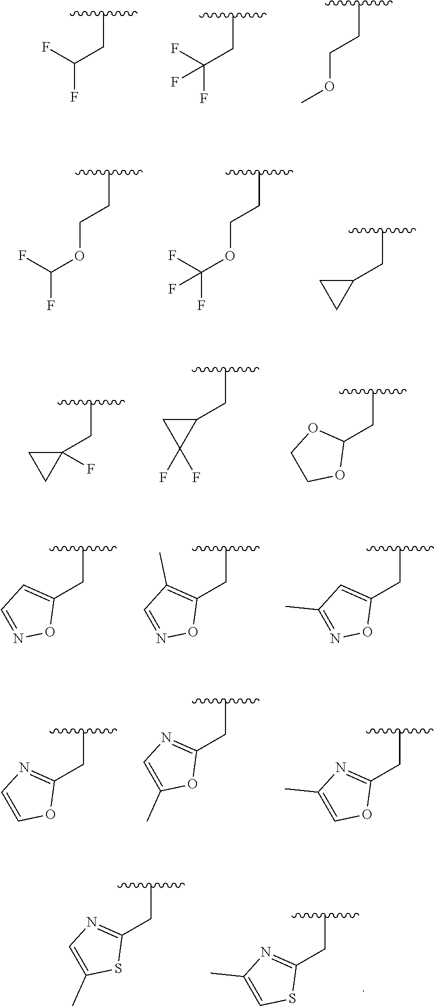

15. The compound according to claim 1, wherein R⁶ is selected from hydrogen, —CH₃, —OCH₃,

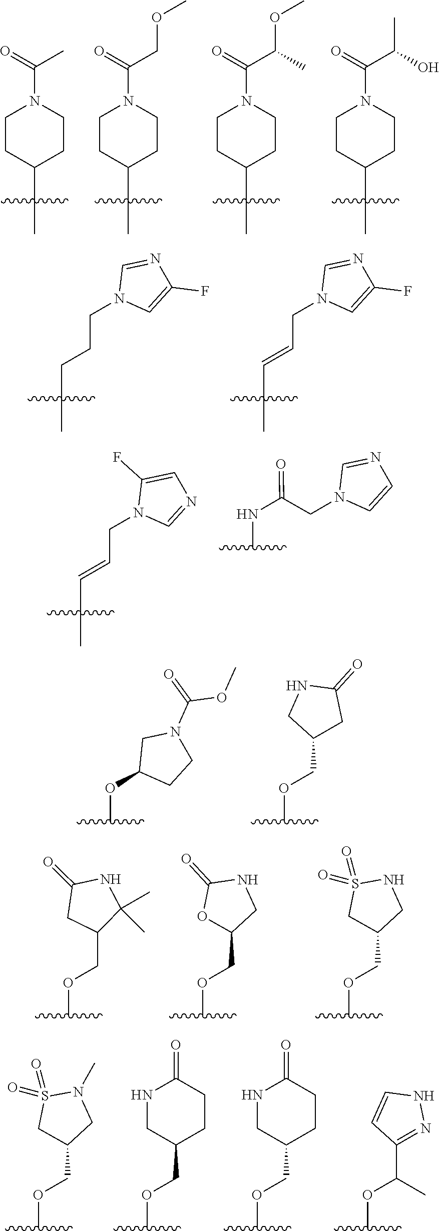

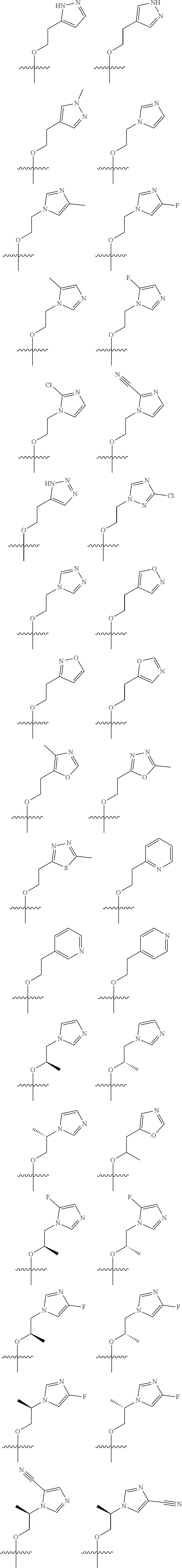

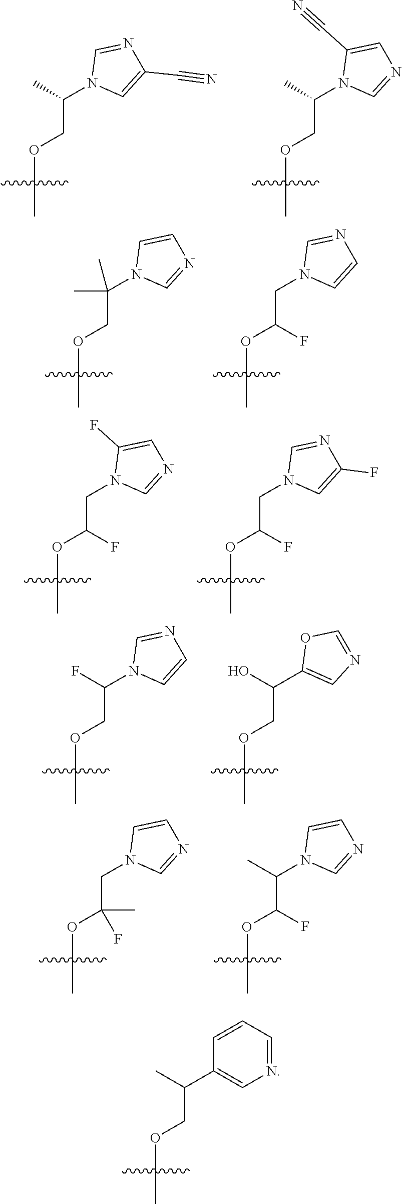

16. A pharmaceutically acceptable composition comprising the compound according to claim 1, and a pharmaceutically acceptable carrier, adjuvant, or vehicle.

17. A method of inhibiting PAD4 in a subject or in a biological sample comprising the step of contacting the PAD4 with a compound according to claim 1.

18. A method of treating a PAD4-mediated disease, disorder, or condition in a subject in need thereof comprising the step of administering to said subject the composition according to claim 16.

19. The method according to claim 18, wherein the PAD4-mediated disease, disorder, or condition is selected from the group consisting of acid-induced lung injury, acne (PAPA), acute lymphocytic leukemia, acute, respiratory distress syndrome, Addison's disease, adrenal hyperplasia, adrenocortical insufficiency, ageing, AIDS, alcoholic hepatitis, alcoholic hepatitis, alcoholic liver disease, allergen induced asthma, allergic bronchopulmonary, aspergillosis, allergic conjunctivitis, alopecia, Alzheimer's disease, amyloidosis, amyotropic lateral sclerosis, and weight loss, angina pectoris, angioedema, anhidrotic ecodermal dysplasia-ID, ankylosing spondylitis, anterior segment, inflammation, antiphospholipid syndrome, aphthous stomatitis, appendicitis, arthritis, asthma, atherosclerosis, atopic dermatitis, autoimmune diseases, autoimmune hepatitis, bee sting-induced inflammation, behcet's disease, Behcet's syndrome, Bells Palsey, berylliosis, Blau syndrome, bone pain, bronchiolitis, burns, bursitis, cancer, cardiac hypertrophy, carpal tunnel syndrome, catabolic disorders, cataracts, cerebral aneurysm, chemical irritant-induced inflammation, chorioretinitis, chronic heart failure, chronic lung disease of prematurity, chronic lymphocytic leukemia, chronic obstructive pulmonary disease, colitis, complex regional pain syndrome, connective tissue disease, corneal ulcer, crohn's disease, cryopyrin-associated periodic syndromes, cyrptococcosis, cystic fibrosis, deficiency of the interleukin-1-receptor antagonist (DTRA), dermatitis, dermatitis endotoxemia, dermatomyositis, diffuse intrinsic pontine glioma, endometriosis, endotoxemia, epicondylitis, erythroblastopenia, familial amyloidotic polyneuropathy, familial cold urticarial, familial mediterranean fever, fetal growth retardation, glaucoma, glomerular disease, glomerular nephritis, gout, gouty arthritis, graft-versus-host disease, gut diseases, head injury, headache, hearing loss, heart disease, hemolytic anemia, Henoch-Scholein purpura, hepatitis, hereditary periodic fever syndrome, herpes zoster and simplex, HIV-1, Hodgkin's disease, Huntington's disease, hyaline membrane disease, hyperammonemia, hypercalcemia, hypercholesterolemia, hyperimmunoglobulinemia D with recurrent fever (HIDS), hypoplastic and other anemias, hypoplastic anemia, idiopathic thrombocytopenic purpura, incontinentia pigmenti, infectious mononucleosis, inflammatory bowel disease, inflammatory lung disease, inflammatory neuropathy, inflammatory pain, insect bite-induced inflammation, iritis, irritant-induced inflammation, ischemia/reperfusion, juvenile rheumatoid arthritis, keratitis, kidney disease, kidney injury caused by parasitic infections, kidney injury caused by parasitic infections, kidney transplant rejection prophylaxis, leptospiriosis, leukemia, Loeffler's syndrome, lung injury, lung injury, lupus, lupus, lupus nephritis, lymphoma, meningitis, mesothelioma, mixed connective tissue disease, Muckle-Wells syndrome (urticaria deafness amyloidosis), multiple sclerosis, muscle wasting, muscular dystrophy, myasthenia gravis, myocarditis, mycosis fungiodes, mycosis fungoides, myelodysplastic syndrome, myositis, nasal sinusitis, necrotizing enterocolitis, neonatal onset multisystem inflammatory disease (NOMID), nephrotic syndrome, neuritis, neuropathological diseases, non-allergen induced asthma, obesity, ocular allergy, optic neuritis, organ transplant, osterarthritis, otitis media, paget's disease, pain, pancreatitis, Parkinson's disease, pemphigus, pericarditis, periodic fever, periodontitis, peritoneal endometriosis, pertussis, pharyngitis and adenitis (PFAPA syndrome), plant irritant-induced inflammation, pneumonia, pneumonitis, pneumosysts infection, poison ivy/urushiol oil-induced inflammation, polyarteritis nodosa, polychondritis, polycystic kidney disease, polymyositis, psoriasis, psoriasis, psoriasis, psoriasis, psychosocial stress diseases, pulmonary disease, pulmonary hypertension, pulmonary fibrosis, pyoderma gangrenosum, pyogenic sterile arthritis, renal disease, retinal disease, rheumatic carditis, rheumatic disease, rheumatoid arthritis, sarcoidosis, seborrhea, sepsis, severe pain, sickle cell, sickle cell anemia, silica-induced disease, Sjogren's syndrome, skin diseases, sleep apnea, solid tumors, spinal cord injury, Stevens-Johnson syndrome, stroke, subarachnoid hemorrhage, sunburn, temporal arteritis, tenosynovitis, thrombocytopenia, thyroiditis, tissue transplant, TNF receptor associated periodic syndrome (TRAPS), toxoplasmosis, transplant, traumatic brain injury, tuberculosis, type 1 diabetes, type 2 diabetes, ulcerative colitis, urticarial, uveitis, and Wegener's granulomatosis.

20. A compound of formula I′: 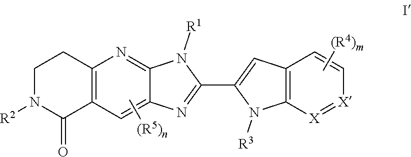
or a pharmaceutically acceptable salt, isomer, enantiomer, or tautomer thereof, wherein:
X is selected from C—R⁶ and N;
X′ is selected from C—R^{6′} and N, wherein X and X′ are not simultaneously N;
R¹ is C₁₋₄ aliphatic;
R² is C₁₋₆ aliphatic substituted by 0-4 instances of R⁷;
R³ is C₁₋₆ aliphatic substituted by 0-3 instances of R¹;
R⁴ is halogen;
R⁵ is halogen;
each R⁶ and R^{6′} is independently selected from hydrogen, halogen, —OR, —N(R)₂, —OC(O)R, —N(R)C(O)R, —N(R)C(O)-L-(R⁹)ₚ, —O-L-(R⁹)ₚ, —Cy, optionally substituted C₁₋₆ aliphatic, and C₁₋₆ aliphatic-OH;
each R⁷ is independently selected from halogen, —OR, —N(R)₂, and —Cy;
each R⁸ is independently selected from halogen, —OR, —N(R)₂, and —Cy;
each R⁹ is independently selected from halogen, —OR, —N(R)₂, and —Cy;
L is a covalent bond or C₁₋₄ aliphatic;
each Cy is independently selected from a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulfur, wherein Cy is substituted by 0-3 instances of R¹⁰;
each R¹⁰ is independently selected from halogen, —OR, —N(R)₂, —CN, —C(O)R, —C(O)OR, —C(O)N(R)₂, oxo, and an optionally substituted group selected from C₁₋₆ aliphatic and a 3- to 7-membered saturated or partially unsaturated heterocyclic ring having 1-2 heteroatoms independently selected from nitrogen, oxygen, and sulfur;
each R is independently hydrogen or an optionally substituted group selected from C₁₋₆ aliphatic, a 3- to 7-membered saturated or partially unsaturated carbocyclic ring, phenyl, a 3- to 7-membered saturated or partially unsaturated heterocyclic ring having 1-2 heteroatoms independently selected from nitrogen, oxygen, and sulfur, and a 5- to 6-membered heteroaryl ring having 1-4 heteroatoms independently selected from nitrogen, oxygen, and sulphur, wherein each R is independently substituted with 0-3 instances of halogen, —OCH₃, or —OH;
each m and n is independently 0 or 1; and
p is independently 1-4.
